(19) United States
(12) Patent Application Publication
(10) Pub. No.: US 2018/0292465 A1
(43) Pub. Date: Oct. 11, 2018

(54) SYSTEMS AND METHODS FOR DEGRADATION ANALYSIS

(71) Applicant: Board of Regents, The University of Texas System, Austin, TX (US)

(72) Inventors: Jude A. Osara, Austin, TX (US); Michael Bryant, Austin, TX (US)

(21) Appl. No.: 15/948,950

(22) Filed: Apr. 9, 2018

Related U.S. Application Data
(60) Provisional application No. 62/483,182, filed on Apr. 7, 2017.
(60) Provisional application No. 62/653,692, filed on Apr. 6, 2018.

(51) Int. Cl. G01R 31/36; G01N 33/28; G01M 5/00

(52) U.S. Cl. CPC G01R 31/3679; G01M 5/0033; G01N 33/2888

(57) ABSTRACT

Disclosed are methods and systems that facilitate the estimation of entropy in a dissipative process of a system, via a structured approach to degradation and failure modeling that solves the analysis as a geometric problem, to measure degradation and/or expected life or failure of a system. It was found that data collected to estimate entropies produced by dissipative processes in association with degradation or ageing of batteries, grease, and fatigue, exhibit linearity between related degradation measure and combination of specific accumulated entropies (e.g., joule dissipation entropy, heat storage entropy, heat transfer entropy, electrochemical entropy, shear work entropy, thermal entropy, oxidation entropy, and plastic strain entropy, thermal entropy). A universally consistent approach is further disclosed for characterizing lead-acid batteries of all configurations. An instantaneous model for analyzing battery degradation based on irreversible thermodynamics and the Degradation-Entropy Generation theorem is formulated and experimentally verified using commonly measured lead-acid battery operational parameters.

### CROSS-REFERENCE TO RELATED APPLICATIONS

This application claims benefit of U.S. Provisional Application No. 62/483,182, filed Apr. 7, 2017, and U.S. Provisional Application No. 62/653,692, filed Apr. 6, 2018, each of which is hereby incorporated herein by reference in its entirety.

### TECHNICAL FIELD

The present disclosure generally relates to the estimation of entropy in a dissipative process, to the measure of degradation, and/or expected life or failure of a system.

### BACKGROUND

Material degradation occurs as a result of irreversible dissipative processes and forces. Various forms of degradation mechanisms exist such as friction, chemical reactions, plasticity, dislocation movements and corrosion all irreversibly leading to failure of a particular system or component. The first and second laws of thermodynamics describe states of a system from the perspective of energy content and exchanges. The first law prescribes energy conservation while the second law introduces the concept of irreversibility in systems as thermodynamic energies decrease, also known as entropy.

Under the Degradation-Entropy Generation (DEG) Theorem formulated by Michael Bryant, Michael Khonsari, and Frederick Lin (e.g., as described in M. Bryant et al., “On the thermodynamics of degradation,” Proceedings of the Royal Society A: Mathematical, Physical and Engineering Sciences, vol. 464, no. 2096, pp. 2001-2014, 2008, which is incorporated by reference herein in its entirety), it has been shown severally that entropy generation accompanies all degradation mechanisms simply by the irreversible nature of the dissipative processes involved. Hence, predicting and quantifying the effect of these processes could be made based on accurate estimate of entropy produced.

Yet, prediction and quantification of degradation mechanisms in dissipative processes are still highly complex.

### SUMMARY

The exemplified methods and systems facilitate the estimation of entropy in a dissipative process of a system, via a structured approach to degradation and failure modeling that performs the analysis as a geometric problem, to measure degradation and/or expected life or failure of a system. It was found that data collected to estimate entropies produced by dissipative processes in association with degradation or ageing of batteries, grease, and fatigue, exhibit linear combination of related degradation measure with specific accumulated entropies (e.g., joule dissipation entropy, heat storage entropy, electrochemical entropy, shear work entropy, thermal entropy, oxidation entropy, and plastic strain entropy, thermal entropy). Indeed, the exemplified methods and systems facilitate analysis of degradation and failure resulting from such dissipative processes as a geometric problem in multi-dimensional entropy “failure” space.

A universally consistent approach is further disclosed for characterizing lead-acid batteries of all configurations, including capacity fade. It was discovered that the difference between reversible and irreversible Maximum work entropies is the entropy generated in the system. An instantaneous model for analyzing battery degradation based on irreversible thermodynamics and the Degradation-Entropy Generation theorem is formulated and experimentally verified using commonly measured lead-acid battery operational parameters based on this relationship. In the model, one or more reversible entropy parameters and the one or more irreversible entropy parameters are used to determine an entropy production parameter directly related to degradation and/or expected failure of the system.

Because the DEG model is purely physics-based (as compared a combination of half-physics-half-experimental models, for example), accurate formulation of the terms governing the dissipative processes is crucial. Proof of validity is based on a reproducible and repeatable interpretation. The instantaneous model can identify correct data by showing a more likely battery's natural response predicted by the model with the good data, which can be used to troubleshoot “bad” datasets and catch possible measurement equipment faults.

In aspect, a method to estimate entropy in a dissipative process of a system, wherein the estimation is used to measure degradation and/or expected failure of a system, the method comprising: obtaining, by a processor, in-situ control data set or experimental data set associated with a dissipative or thermal process of a system, wherein the control or experimental data set is acquired to assess a degradation measure and to assess an entropy production for the dissipative process; determining, by the processor, one or more degradation coefficients from the control or experimental data, wherein each of the one or more degradation coefficients is determined as a rate of change of one or more assessed degradation measure parameters (e.g., w) with respect to one or more assessed entropy production (e.g., Sᵢ′) parameters for the dissipative or thermal process (e.g., pᵢ), wherein the rate of change is determined as a slope of a first coordinate axis associated with the one or more assessed degradation measure parameters and of one or more second coordinate axes each associated with an assessed entropy production parameter associated with the dissipative or thermal process; and, determining, by the processor, one or more parameters associated with a measure of degradation and/or expected failure of the system, wherein the determination of the one or more parameters is based on an assessed estimated entropy parameter associated with estimated entropy produced by the dissipative process by linearly combining each of the one or more determined degradation coefficients with at least one corresponding assessed accumulated irreversible entropy parameter, and wherein the one or more parameters associated with the degradation and/or expected failure, or value(s) associated therewith, of the system is used in an evaluation of the system for use in engineering application or in the control, optimization, or maintenance of said system in said engineering application.

In some embodiments, the one or more assessed degradation measure parameters associated with the first coordinate axis and the one or more assessed entropy production parameters associated with the one or more second coordinate axes, collectively, correspond to a multi-dimensional surface, and wherein the slope assessed on said multi-dimensional surface corresponds to a degradation entropy generation (DEG) trajectory.

In some embodiments, the method further includes collecting, in a control loop of the system, the in-situ the control data associated with the dissipative process.

In some embodiments, the method further includes performing the experiment to collect experimental data for estimation of entropies in the dissipative process of the system.

In some embodiments, the dissipative process is selected from the group consisting of battery degradation, grease degradation, and structural degradation due to fatigue.

In some embodiments, the dissipative process is selected from the group consisting of degradation associated with friction, degradation associated with turbulence, degradation associated with spontaneous chemical reaction, degradation associated with inelastic deformation, degradation associated with fretting, degradation associated with free expansion of gas or liquid, degradation associated with flow of electric current through a resistance, and degradation associated with hysteresis, and wherein the estimation is used to measure degradation and/or expected failure of a system.

In some embodiments, the dissipative process is associated with battery degradation, wherein the obtained in-situ control data set or experimental data set is used to determine, by the processor (e.g., via the maximum work approach), a first set of degradation coefficients (e.g., BW and BT) based on linear dependence of i) capacity accumulation (e.g., accumulated discharge) on ii) irreversible entropies (e.g. ohmic entropies and thermal entropies, respectively); wherein the measure of degradation and/or expected failure of the system derived based on the first degradation coefficients set used to assess battery cycle life or remaining battery cycle life.

In some embodiments, the obtained in-situ control data set or experimental data set is associated with active thermal process of the system with respect to battery degradation, the method comprising: determining, by the processor (e.g., via the thermal approach), a second degradation set of coefficients (e.g., B_{HT} and B_{T}) based on linear dependence of i) capacity accumulation (e.g., accumulated discharge) on ii) thermal entropies (e.g. heat-transfer entropies and heat storage entropies, respectively); wherein the measure of degradation and/or expected failure of the system derived based on the second degradation coefficients set used to assess battery cycle life or remaining battery cycle life.

In some embodiments, the obtained in-situ control data set or experimental data set is associated with active dissipative or thermal process(es) of the system with respect to battery degradation, the method comprising: determining, by the processor (e.g., via the maximum work approach), a first set of degradation coefficients (e.g., B_{W} and B_{T}) based on linear dependence of i) capacity accumulation (e.g., accumulated discharge) on ii) irreversible entropies (e.g. ohmic entropies and thermal entropies, respectively); and determining, by the processor (e.g., via the thermal approach), a second degradation set of coefficients (e.g., B_{HT} and B_{T}) based on linear dependence of i) capacity accumulation (e.g., accumulated discharge) on ii) thermal entropies (e.g. heat-transfer entropies and on heat storage entropies, respectively); wherein the measure of degradation and/or expected failure of the system derived based on the first and second degradation coefficients sets are used to assess battery cycle life or remaining battery cycle life.

In some embodiments, the system comprises a lead-acid battery or a lithium-ion battery.

In some embodiments, the dissipative process is associated with grease degradation, wherein the obtained in-situ control data set or experimental data set is used to determine, by the processor, a first set of degradation coefficients (e.g., shear work degradation coefficient, B_{π}, and thermal degradation coefficient, B_{T}) based on linear dependence between i) assessed shear stress and ii) irreversible entropies (e.g. shear entropy and thermal entropy, respectively); wherein the measure of degradation and/or expected failure of the system derived based on the first degradation coefficients set is used to assess grease life or remaining grease life.

In some embodiments, the obtained in-situ control data set or experimental data set is associated with an active thermal process of the system with respect to grease degradation, the method comprising

determining, by the processor, a second set of degradation coefficients (e.g., B_{HT} and B_{T}) based on linear dependence of i) shear stress on ii) thermal entropies (e.g. heat-transfer entropies and heat storage entropies, respectively);

wherein the measure of degradation and/or expected failure of the system derived based on the second degradation coefficients set is used to assess grease life or remaining grease life.

In some embodiments, the obtained in-situ control data set or experimental data set is associated with active dissipative or thermal process(es) of the system with respect to grease degradation, the method comprising: determining, by the processor, a first set of degradation coefficients (e.g., shear work degradation coefficient, B_{τ}, and thermal degradation coefficient, B_{T}) based on linear dependence between i) assessed shear stress and ii) irreversible entropies (e.g. shear entropy and thermal entropy, respectively); determining, by the processor, a second set of degradation coefficients (e.g., B_{HT} and B_{T}) based on linear dependence of i) shear stress on ii) thermal entropies (e.g. heat-transfer entropies and heat storage entropies, respectively; wherein the measure of degradation and/or expected failure of the system derived based on the first and second degradation coefficients sets are used to assess grease life or remaining grease life.

In some embodiments, the obtained in-situ control data set or experimental data set is associated with the active dissipative process(es) of the system with respect to structural degradation due to fatigue, the method comprising: determining, by the processor, a first set of degradation coefficients (e.g., B_{W} and B_{T}) based on linear dependence between i) assessed mechanical stress (e.g. shear stress for torsional loading, normal stress for normal loading) and ii) irreversible entropies (e.g. plastic strain entropy and thermal entropy, respectively); wherein the measure of degradation and/or expected failure of the system derived based on the first degradation coefficients set is used to assess mechanical life or remaining mechanical life of a structure.

In some embodiments, the obtained in-situ control data set or experimental data set is associated with the active dissipative process(es) of the system with respect to structural degradation due to fatigue, the method comprising: determining, by the processor, a second set of degradation coefficients (e.g., B_{W} and BT_{D}) based on linear dependence between i) assessed CDM damage and ii) irreversible entropies (e.g. plastic strain entropy and thermal entropy, respectively); wherein the measure of degradation and/or expected failure of the system derived based on the second degradation coefficients set is used to assess mechanical life or remaining mechanical life of a structure.

In some embodiments, the obtained in-situ control data set or experimental data set is associated with the active dissipative process(es) of the system with respect to structural degradation due to fatigue, the method comprising: determining, by the processor, a third set of degradation coefficients (e.g., BW_{N} and BT_{N}) based on linear dependence between i) assessed normalized cycles (N/N_{f}) and ii) irreversible entropies (e.g. plastic strain entropy and thermal entropy, respectively); wherein the measure of degradation and/or expected failure of the system derived based on the third degradation coefficients set is used to assess mechanical life or remaining mechanical life of a structure.

In some embodiments, the obtained in-situ control data set or experimental data set is associated with an active thermal process of the system with respect to structural degradation due to fatigue, the method comprising: determining, by the processor, a fourth set of degradation coefficients (e.g., B_{HT} and B_{T}) based on linear dependence between i) assessed stress (e.g. shear stress for torsional loading, normal stress, or normal loading) and ii) thermal entropies (e.g. heat transfer entropy and heat storage entropy); wherein the measure of degradation and/or expected failure of the system derived based on the fourth degradation coefficients set is used to assess mechanical life or remaining mechanical life of a structure.

In some embodiments, the obtained in-situ control data set or experimental data set is associated with active dissipative or thermal process(es) of the system with respect to structural degradation due to fatigue, the method comprising: determining, by the processor, a first set of degradation coefficients (e.g., B_{W} and B_{T}) based on linear dependence between i) assessed mechanical stress (e.g. shear stress for torsional loading, normal stress, or normal loading) and ii) irreversible entropies (e.g. plastic strain entropy and thermal entropy, respectively); determining, by the processor, a second set of degradation coefficients (e.g., BW_{D} and BT_{D}) based on linear dependence between i) assessed CDM damage and ii) irreversible entropies (e.g. plastic strain entropy and thermal entropy, respectively); determining, by the processor, a third set of degradation coefficients (e.g., BW_{N} and BT_{N}) based on linear dependence between i) assessed normalized cycles (N/N_{f}) and ii) irreversible entropies (e.g. plastic strain entropy and thermal entropy, respectively); determining, by the processor, a fourth set of degradation coefficients (e.g., B_{HT} and B_{T}) based on linear dependence between i) assessed stress (e.g. shear stress for torsional loading, normal stress, or normal loading) and ii) thermal entropies (e.g. heat transfer entropy and heat storage entropy); wherein the measure of degradation and/or expected failure of the system derived based on the first, second, third, and fourth degradation coefficients sets are used to assess mechanical life or remaining mechanical life of a structure.

In some embodiments, the obtained in-situ control data set or experimental data set is associated with active dissipative or thermal process(es) of the system with respect to structural degradation due an assessed fatigue measure, and wherein the assessed fatigue measure is selected from the group consisting of: mechanical stress (e.g. normal or torsional), thermal stress, normalized number of cycles (N/N_{f}), Continuum Damage Mechanics-based damage parameter (D), and chemical degradation (e.g. corrosion).

In some embodiments, the estimation of entropy includes an estimation of entropy production/generation.

In some embodiments, the method further includes determining, by the processor, one or more irreversible entropy parameters for the dissipative process by combining an assessed active boundary work parameter associated with active boundary work with an internal dissipation parameter associated with internal dissipation of the system, wherein the internal dissipation parameter is estimated as a change in a potential of the system; and determining, by the processor, one or more reversible entropy parameters for the dissipative process based on assessed standard/ideal values of intensive and extensive phenomenological conjugate variables that define the dissipative process and an instantaneous boundary temperature associated with the active boundary work parameter, wherein the one or more reversible entropy parameters and the one or more irreversible entropy parameters are used to determine an entropy production parameter directly related to degradation and/or expected failure of the system.

In some embodiments, the method further includes determining, by the processor, the entropy production parameter, wherein the entropy production parameter is determined as a difference between the one or more reversible entropy parameters and the one or more irreversible entropy parameters.

In some embodiments, the method further includes determining, by the processor, a critical failure entropy parameter associated with a critical failure entropy, wherein the critical failure entropy parameter, or a value associated therewith, is used to detect instability in the system (e.g., for use in engineering application or in the control, optimization, or maintenance of said system in said engineering application).

In some embodiments, the critical failure entropy parameter is estimated as a value of the irreversible entropy parameter when the entropy production parameter transitions abruptly (e.g., from a positive value to a negative value).

In some embodiments, the method further includes determining, by the processor, a parameter associated with a measure of the system ideal state, wherein the determination is based on the estimated one or more reversible entropy parameters by linearly combining a determined reversible degradation coefficient with an assessed accumulated reversible entropy parameter, or values associated therewith, wherein the ideal state is used as an instantaneous reference in a real-time monitoring system and/or an evaluation of the system for use in engineering application and/or in the control, or optimization, or maintenance of said system in said engineering application.

In some embodiments, the dissipative process is associated with battery degradation, wherein the obtained in-situ control data set or obtained experimental data set is used to determine a first set of degradation coefficients (e.g., B_{Ω} and B_{VT}) based on linear dependence of i) capacity (e.g., accumulated charge/discharge) on ii) ohmic entropy and on electro-chemico-thermal (ECT) entropy, respectively; wherein an assessed battery ideal/reversible state is determined by i) measured open-circuit voltage values measured from the system and ii) estimated reversible current values determined as initial current values measured from the system having been adjusted by the measured open-circuit voltage values. In some embodiments, wherein the ECT entropy is evaluated as a charge content multiplied by voltage change, divided by temperature.

In some embodiments, the measure of degradation and/or expected failure of the system derived based on the first set of degradation coefficients is used to assess battery cycle life or remaining battery cycle life.

In some embodiments, the measure of degradation and/or expected failure of the system derived based on the first set of degradation coefficients is used to assess battery cycle life or remaining battery cycle life.

In some embodiments, the dissipative process is associated with rechargeable battery degradation, the method further comprises: determining, by the processor, a parameter associated with a measure of degradation (e.g. capacity fade) and/or expected failure of the system based on a difference between an estimated degraded state and the assessed battery ideal state, wherein determination is used to assess battery cycle life or remaining battery cycle life.

In another aspect, a system is disclosed comprising: a processor; and, a memory having instructions stored thereon, wherein execution of the instructions by the processor, cause the processor to: obtain in-situ control data set or experimental data set associated with a dissipative or thermal process of a system, wherein the control or experimental data set is acquired to assess a degradation measure and to assess an entropy production for the dissipative process; determine one or more degradation coefficients from the control or experimental data, wherein each of the one or more degradation coefficients is determined as a rate of change of one or more assessed degradation measure parameters (e.g., w) with respect to one or more assessed entropy production (e.g., Sᵢ′) parameters for the dissipative or thermal process (e.g., pᵢ), wherein the rate of change is determined as a slope of a first coordinate axis associated with the one or more assessed degradation measure parameters and of one or more second coordinate axes each associated with an assessed entropy production parameter associated with the dissipative or thermal process; and determine one or more parameters associated with a measure of degradation and/or expected failure of the system, wherein the determination of the one or more parameters is based on an assessed estimated entropy parameter associated with estimated entropy produced by the dissipative process by linearly combining each of the one or more determined degradation coefficients with at least one corresponding assessed accumulated irreversible entropy parameter, and wherein the one or more parameters associated with the degradation and/or expected failure, or value(s) associated therewith, of the system is used in an evaluation of the system for use in engineering application or in the control, optimization, or maintenance of said system in said engineering application.

In another aspect, a non-transitory computer readable medium is disclosed having instructions stored thereon, wherein execution of the instructions by a processor, cause the processor to: obtain in-situ control data set or experimental data set associated with a dissipative or thermal process of a system, wherein the control or experimental data set is acquired to assess a degradation measure and to assess an entropy production for the dissipative process; determine one or more degradation coefficients from the control or experimental data, wherein each of the one or more degradation coefficients is determined as a rate of change of one or more assessed degradation measure parameters (e.g., w) with respect to one or more assessed entropy production (e.g., Sᵢ′) parameters for the dissipative or thermal process (e.g., pᵢ), wherein the rate of change is determined as a slope of a first coordinate axis associated with the one or more assessed degradation measure parameters and of one or more second coordinate axes each associated with an assessed entropy production parameter associated with the dissipative or thermal process; and determine one or more parameters associated with a measure of degradation and/or expected failure of the system, wherein the determination of the one or more parameters is based on an assessed estimated entropy parameter associated with estimated entropy produced by the dissipative process by linearly combining each of the one or more determined degradation coefficients with at least one corresponding assessed accumulated irreversible entropy parameter, and wherein the one or more parameters associated with the degradation and/or expected failure, or value(s) associated therewith, of the system is used in an evaluation of the system for use in engineering application or in the control, optimization, or maintenance of said system in said engineering application.

### DESCRIPTION OF DRAWINGS

Embodiments of the present invention may be better understood from the following detailed description when read in conjunction with the accompanying drawings. Such embodiments, which are for illustrative purposes only, depict novel and non-obvious aspects of the invention. The drawings include the following figures:

FIG. 1 is a flow diagram of an exemplary method to estimate entropy (e.g., entropy production) in a dissipative process of a system, wherein the estimation is used to measure degradation and/or expected failure of a system in accordance with an illustrative embodiment.

FIG. 2 shows an example the rate of change determined as a slope of a first coordinate axis associated with the one or more assessed degradation measure parameters and of one or more second coordinate axes in which each axis is associated with an assessed entropy production parameter associated with the dissipative or thermal process.

FIG. 3 is a schematic illustration of a slider and counter surface enclosed in a tribo-control volume.

FIG. 4 is a plot showing the grease shear stress response to constant shear rate application.

FIG. 5 shows AFM images of lithium grease microstructure showing (a) breakdown from shearing and (b) the reverse buildup process after shearing.

FIG. 6 is a schematic illustration of grease sample undergoing work interaction, showing system boundary.

FIG. 7A is a schematic illustration of the mixer impellers used in the grease shearer.

FIG. 7B is a photograph showing the grease shearer in operation.

FIG. 7C is a photograph showing the grease shearer in operation. Lithium grease NLGI 4 is visible.

FIG. 8 is a plot showing the monitored parameters during a 3-hr grease shearing process at a constant shear rate of 123.4 s⁻¹. Temperatures are on the right axis, and instantaneous shear stress is on the left.

FIG. 9 is a plot showing the shear stress accumulation over time.

FIG. 10A is a plot showing the accumulated Helmholtz energy and its components during shearing.

FIG. 10B is a plot illustrating the rates of active processes (shear, thermal) taking place showing significant transients.

FIG. 11A is a plot illustrating the entropy accumulation from active processes as a function of time.

FIG. 11B is a plot illustrating the entropy accumulation from active processes as a function of accumulated shear stress.

FIG. 12 is a plot illustrating instantaneous shear stress versus entropy generation rates.

FIGS. 13A and 13B show two views of a 3D plot and linear surface fit of shear stress vs shear entropy and thermal entropy for grease during shearing for iteration 4.

FIG. 14A is a plot showing the accumulated heat generation and its components during shearing.

FIG. 14B is a plot illustrating the rates of active processes (heat transfer, heat storage) taking place showing significant transients.

FIG. 15A is a plot illustrating the entropy accumulation from active thermal processes with accumulated shearing.

FIG. 15B is a plot illustrating instantaneous shear stress versus entropy generation rates.

FIGS. 16A and 16B show two views of a 3D plot and linear surface fit of shear stress vs heat transfer entropy and thermal entropy for grease during shearing for iteration 4.

FIGS. 17A and 17B show multiple DEG trajectories from iterations 1 to 5 plotted on the same DEG surface.

FIG. 18A illustrates the cycling mechanisms in lithium-ion batteries.

FIG. 18B illustrates the cycling mechanisms in lead-acid batteries.

FIG. 19 is a diagram of the charge-discharge cycle circuit for the lithium-ion batteries.

FIG. 20 shows discharge and charge cycle circuit diagrams for the lead acid batteries.

FIG. 21A is a plot of measurement parameters showing the normal full discharge and the over-discharge regions for a lithium-ion battery.

FIG. 21B is a plot of measurement parameters showing the normal full discharge and the over-discharge regions for a lead-acid battery.

FIGS. 22A and 22B are plots showing the monitored parameters during cycling of a Li-ion battery.

FIG. 23 is a plot of the accumulated discharge and depth of discharge DoD during cycling of a Li-ion battery.

FIG. 24A is a plot of the accumulated Gibbs energy and its components during discharge of a Li-ion battery.

FIG. 24B is a plot of the accumulated Gibbs energy and its components during charge of a Li-ion battery.

FIG. 25A is a plot of the rates of active concurrent processes (ohmic, thermal) showing transients during discharge of a Li-ion battery.

FIG. 25B is a plot of the rates of active concurrent processes (ohmic, thermal) showing transients during charge of a Li-ion battery.

FIG. 26A is a plot showing the entropy accumulation from active processes with accumulated discharge of a Li-ion battery.

FIG. 26B is a plot showing the entropy accumulation from active processes with accumulated charge of a Li-ion battery.

FIG. 27A is a plot showing the entropy generation rates of active processes (ohmic and thermal) during discharge of a Li-ion battery.

FIG. 27B is a plot showing the entropy generation rates of active processes (ohmic and thermal) during charge of a Li-ion battery.

FIGS. 28A and 28B show two views of a 3D plot and linear surface fit of capacity (vertical axis) vs ohmic and thermal entropies (horizontal axes) during the discharge step of cycle 4 (li-ion battery), indicating a dependence on 2 active processes. As shown in FIG. 28A, the recorded data points during discharge trace a trajectory coincident with a linear plane. FIG. 28B shows an end view of the coincidence, visual of the goodness of fit R²=1. Discharge starts from upper left corner.

FIG. 29 is a plot of cyclic discharge accumulation versus cyclic entropy accumulation.

FIG. 30A is a plot showing the measured lead-acid battery parameters during discharge (Discharge rate: ˜11 A).

FIG. 30B is a plot showing the measured lead-acid battery parameters during charge (charge rate: 1.2 A).

FIG. 31 is a plot showing the accumulated discharge and depth of discharge DoD of a lead acid battery.

FIG. 32A is a plot of accumulated Gibbs energy and its components during discharge of a lead acid battery.

FIG. 32B is a plot of accumulated Gibbs energy and its components during charge of a lead acid battery.

FIG. 33A is a plot showing the rates of concurrently active processes (ohmic, thermal) during discharge of a lead acid battery.

FIG. 33B is a plot showing the rates of concurrently active processes (ohmic, thermal) during charge of a lead acid battery.

FIG. 34A is a plot showing the accumulated Gibbs entropy and its components during discharge of a lead acid battery. Note that the horizontal charge axis is negative.

FIG. 34B is a plot showing the accumulated Gibbs entropy and its components during charge of a lead acid battery.

FIG. 35A is a plot showing the entropy generation rates during discharge of a lead acid battery.

FIG. 35B is a plot showing the entropy generation rates during charge of a lead acid battery.

FIGS. 36A and 36B show two views of a 3D plot and linear surface fit of capacity versus ohmic entropy and thermal entropy for lead-acid battery during discharge (cycle 2) showing a perfectly linear combined dependence on 2 active processes. FIG. 36B shows a median slice through the surface fit for all the measured points. Discharge starts from upper left corner.

FIGS. 37A and 37B are plots showing monitored parameters during normal cycling of a lead acid battery.

FIG. 38A is a plot of accumulated heat generation and its components during discharge of a lead acid battery.

FIG. 38B is a plot of accumulated heat generation and its components during charge of a lead acid battery.

FIG. 39A is a plot of the rates of active concurrent processes (heat transfer, heat storage) during discharge of a lead acid battery.

FIG. 39B is a plot of the rates of active concurrent processes (heat transfer, heat storage) during charge of a lead acid battery.

FIG. 40A is a plot of entropy accumulation from active thermal processes with accumulated discharge of a lead acid battery.

FIG. 40B is a plot of entropy accumulation from active thermal processes with accumulated charge of a lead acid battery.

FIG. 41A is a plot of entropy generation rates during discharge of a lead acid battery.

FIG. 41B is a plot of entropy generation rates during charge of a lead acid battery.

FIGS. 42A and 42B show two views of 3D plot and linear surface fit of capacity versus heat transfer entropy and thermal entropy for a Li-ion acid battery during discharge, showing a linear combined dependence on 2 active processes. FIG. 42B shows a median slice through the surface fit for measured points. Discharge starts from apex corner.

FIG. 43A is a plot showing the monitored parameters during discharge of a lead-acid battery.

FIG. 43B is a plot showing the monitored parameters during charge of a lead-acid battery.

FIG. 44A is a plot of the accumulated heat generation and its components during discharge of a lead-acid battery.

FIG. 44B is a plot of the accumulated heat generation and its components during charge of a lead-acid battery.

FIG. 45A shows the rates of active processes (heat transfer, heat storage) during discharge of a lead-acid battery.

FIG. 45B shows the rates of active processes (heat transfer, heat storage) during charge of a lead-acid battery.

FIG. 46A is a plot of the entropy accumulation from active thermal processes with accumulated discharge of a lead-acid battery.

FIG. 46B is a plot of the entropy accumulation from active thermal processes with accumulated charge of a lead-acid battery.

FIG. 47A is a plot of entropy generation rates during discharge of a lead-acid battery.

FIG. 47B is a plot of entropy generation rates during charge of a lead-acid battery.

FIGS. 48A and 48B show two views of 3D plot and linear surface fit of capacity versus heat transfer and heat storage entropies for lead-acid batteries during discharge for cycle 2, showing a linear combined dependence on 2 active processes. FIG. 48B shows an end view of the median slice through the surface fit for measured points. Discharge trajectory starts from top left corner.

FIG. 49 is a plot showing the measured lead-acid battery parameters during cycling including severe overdischarging (Discharge rate: 11 A).

FIG. 50A shows the accumulated Gibbs energy and its components during normal discharge of a lead-acid battery.

FIG. 50B shows the accumulated Gibbs energy and its components during normal overdischarge of a lead-acid battery.

FIG. 51A is a plot of the accumulated Gibbs entropy and its components during normal discharge of a lead-acid battery.

FIG. 51B is a plot of the accumulated Gibbs entropy and its components during normal overdischarge of a lead-acid battery.

FIGS. 52A and 52B are 3D plots and linear surface fit of capacity vs ohmic entropy and thermal entropy for lead-acid batteries during normal discharge (FIG. 52A) and overdischarge (FIG. 52B), showing impact on goodness of fit by overdischarging. In overdischarge, the thermal entropy spans the positive and negative range of its axis due to ohmic heating followed by endothermic recovery in overdischarging.

FIG. 53 is a plot of monitored parameters during overdischarge of a lead-acid battery.

FIG. 54A is a plot of accumulated Gibbs energy and its components during normal discharge of a lead-acid battery.

FIG. 54B is a plot of accumulated Gibbs energy and its components during overdischarge of a lead-acid battery.

FIG. 55A is a plot of entropy accumulation from active processes during normal discharge of a lead-acid battery.

FIG. 55B is a plot of entropy accumulation from active processes during normal overdischarge of a lead-acid battery.

FIGS. 56A and 56B show 3D plots and linear surface fits of capacity vs ohmic entropy and thermal entropy for lithium-ion batteries showing the minimal impact on goodness of fit by overdischarging. FIG. 56A shows a plot for normal discharge and FIG. 56B shows a plot for overdischarge. Overdischarge slightly extends the DEG domain with larger entropy components but remains coincident with the same DEG surface. Discharge trajectory starts from upper left corner.

FIGS. 57A and 57B show multiple DEG lines from cycles 1 to 9 plotted on the same surface for lead-acid battery. FIG. 57B shows a median fit of different cycle DEG lines, coincident with the same DEG surface. Axes are not to scale.

FIGS. 58A and 58B show multiple DEG trajectories from cycles 1 to 29 plotted on the same surface for Li-ion battery. FIG. 58B shows all 29 cycles coincident with the same DEG surface; some trajectories overlap. Axes are not to scale.

FIG. 59 is a plot illustrating the typical non-linear response of steel to loading before failure.

FIG. 60 is a plot showing the monitored parameters during torsional fatigue at a constant frequency of 10 Hz, δ=33.02 mm. Temperatures are on the right axis, and shear stress per cycle is on the left.

FIG. 61 is a plot of accumulated shear stress versus number of load cycles N.

FIG. 62A is a plot of the accumulated Helmholtz energy and components versus cycles N during loading.

FIG. 62B is a plot of the cyclic amplitudes of active processes (loading, thermal) showing significant initial and end state transients in the thermal component.

FIG. 63A is a plot of the plastic strain entropy and thermal entropy accumulation versus accumulated shear stress.

FIG. 63B is a plot of the accumulated shear stress versus cyclic entropy generation.

FIG. 64A is a plot of the damage parameter D versus plastic strain entropy and thermal entropy accumulation showing logarithmic evolution with plastic strain entropy.

FIG. 64B is a plot of D versus cyclic entropy generation.

FIG. 65 is a plot of accumulated stress, plastic strain entropy, and thermal entropy accumulation versus N.

FIGS. 66A and 66B show two views of a 3D plot and linear surface fit of shear stress versus plastic strain entropy and thermal entropy during cyclic torsional loading for sample showing a R²=1 goodness of fit (linear dependence on 2 active processes). The loading trajectory starts from lowest corner of FIG. 66A.

FIGS. 67A and 67B show two views of a 3D plot and linear surface fit of damage D versus plastic strain entropy and thermal entropy during cyclic torsional loading for sample showing a R²=1 goodness of fit (linear dependence on 2 active processes). The loading trajectory starts from lowest corner of FIG. 67A.

FIGS. 68A and 68B show two views of a 3D plot and linear surface fit of N/N_{f} versus plastic strain entropy and thermal entropy during cyclic torsional loading for sample showing a R²=1 goodness of fit (linear dependence on 2 active processes). The loading trajectory starts from lowest corner of FIG. 68A.

FIG. 69 is a plot of the normalized entropy versus normalized cycles.

FIG. 70 is a plot of the damage parameter D versus N obtained from normalized entropy and normalized cycles.

FIG. 71A is a plot of the accumulated heat energy and its components versus N during loading.

FIG. 71B is a plot of cyclic active thermal processes taking place showing significant initial and end state transients in the heat storage component.

FIG. 72A is a plot of heat transfer entropy and thermal entropy accumulation versus accumulated shear stress.

FIG. 72B is a plot of accumulated shear stress versus cyclic heat generation entropy components.

FIG. 73A is a plot of damage parameter D versus heat generation and component entropies showing logarithmic evolution with heat transfer entropy.

FIG. 73B is a plot of D versus cyclic heat generation entropy components.

FIG. 74 is a plot of heat entropy and components versus N.

FIGS. 75A and 75B show two views of a 3D plot and linear surface fit of shear stress versus heat transfer entropy and thermal entropy during cyclic torsional loading for sample showing a R²=1 goodness of fit (linear dependence on 2 active processes). The loading trajectory starts from lowest corner of FIG. 75A.

FIG. 76 is a plot showing the relationship between D and S′_{D} as defined in equations (Equation 5.334)-(Equation 5.341).

FIG. 77A is a plot of the monitored parameters during cycle 2 showing a 4-hour discharge: first hour at ˜11 A and last 3 hours at 3 A.

FIG. 77B is a plot of the monitored parameters during a subsequent 14-hour charge at 1.2 A.

FIG. 78A is a plot of the Gibbs energy change components during discharge.

FIG. 78B is a plot of the Gibbs energy change components during charge.

FIG. 79A is a plot of the irreversible entropy components (Ohmic and ECT) versus measured charge during discharge.

FIG. 79B is a plot of the irreversible entropy components (Ohmic and ECT) versus measured charge during charge.

FIG. 80A is a plot of entropy generation component rates (Ohmic, ECT and Reversible Gibbs) over time during discharge.

FIG. 80B is a plot of entropy generation component rates (Ohmic, ECT and Reversible Gibbs) over time during charge.

FIGS. 81A-81D show 3D plots and linear surface fits of Charge (vertical axes) vs Ohmic and ECT entropies (horizontal axes) during (FIG. 81A) discharge (starts from upper left corner) and (FIG. 81B) charge (starts from lower right corner) steps of cycle 2, indicating a linear dependence on 2 active processes. FIG. 81C and FIG. 81D show end projections of FIG. 81A and FIG. 81B, respectively, a visual confirmation of the goodness of fit. Axes are not to scale.

FIG. 82A is a plot of the entropy generation components for cycle 2's charge step. The region between reversible and irreversible components are entropy generation S′.

FIG. 82B is a plot of the capacity fade components for cycle 2's charge step. The regions between reversible and irreversible components are capacity fade Δ [symbol] .

FIG. 83A is a plot of the entropy generation components for cycle 2's discharge step. The region between reversible and irreversible components are entropy generation S′.

FIG. 83B is a plot of the capacity fade components for cycle 2's discharge step. The regions between reversible and irreversible components are capacity fade Δ [symbol] .

FIG. 84A is a plot of the entropy generation components in the normal discharge ND region. The region between reversible and irreversible components are entropy generation S′.

FIG. 84B is a plot of the capacity fade components in the normal discharge ND region. The regions between reversible and irreversible components are capacity fade Δ [symbol] .

FIG. 85A is a plot of the entropy generation components in the transition T region. (T) marks the transition points. The region between reversible and irreversible components are entropy generation S′.

FIG. 85B is a plot of the capacity fade components in the transition T region. (T) marks the transition points. The regions between reversible and irreversible components are capacity fade Δ [symbol] .

FIG. 86A is a plot of the entropy generation components in the over-discharge region. The region between reversible and irreversible components are entropy generation S′.

FIG. 86B is a plot of the capacity fade components in the over-discharge region. The regions between reversible and irreversible components are capacity fade Δ [symbol] .

FIG. 87A is a plot of the monitored parameters during cycling showing a 40-minute discharge cycle of a battery.

FIG. 87B is a plot of the monitored parameters during cycling showing a 2-hour charge cycle of a battery.

FIG. 88A is a plot showing the discharge capacity, including DOD, during cycling.

FIG. 88B is a plot showing the charge capacity, including DOD, during cycling.

FIG. 89A is a plot of the accumulated Gibbs energy and its components during discharge.

FIG. 89B is a plot of the accumulated Gibbs energy and its components during charge.

FIGS. 90A-90B are plots of active processes (ohmic, thermal) taking place during discharge and charge, respectively, and their effect on net Gibbs energy.

FIGS. 91A-91B are plots showing the entropy accumulation from active processes with accumulated discharge and charge, respectively.

FIGS. 92A-92B are plots showing the impact of discharge rate on entropy generation rate during discharge and charge, respectively.

FIGS. 93A-93B are two views of a 3D plot and linear surface fit of capacity (vertical axis) versus ohmic and thermal entropies (horizontal axes) during the discharge step of cycle 4 (li-ion battery).

FIGS. 94A-94B are plots of multiple DEG trajectories from cycles 1 to 10 plotted on the same surface for a Li-ion battery. Axes are not to scale.

### DETAILED DESCRIPTION

Each and every feature described herein, and each and every combination of two or more of such features, is included within the scope of the present invention provided that the features included in such a combination are not mutually inconsistent.

It is understood that throughout this specification the identifiers “first”, “second”, “third”, “fourth”, “fifth”, “sixth”, and such, are used solely to aid in distinguishing the various components and steps of the disclosed subject matter. The identifiers “first”, “second”, “third”, “fourth”, “fifth”, “sixth”, and such, are not intended to imply any particular order, sequence, amount, preference, or importance to the components or steps modified by these terms.

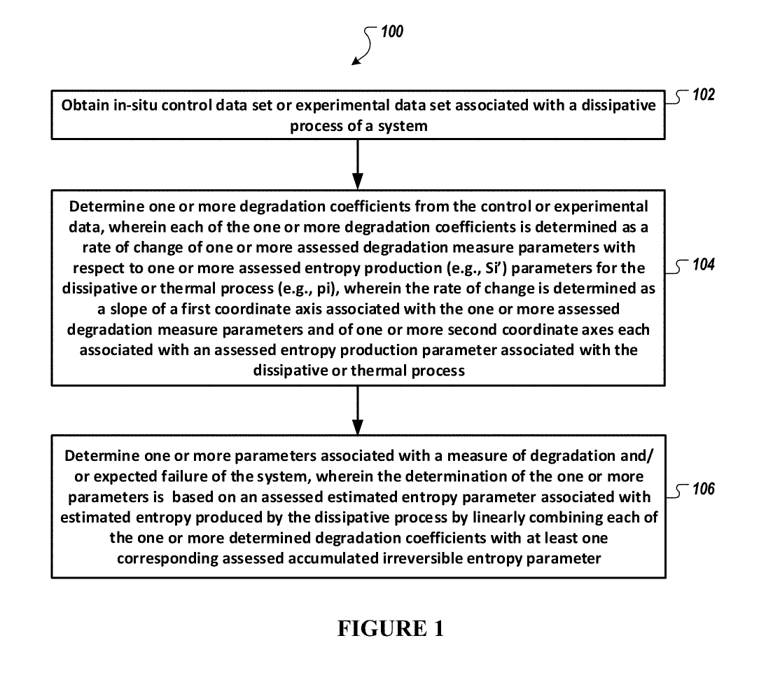

FIG. 1 is a flow diagram of an exemplary method 100 to estimate entropy (e.g., entropy production) in a dissipative process of a system, wherein the estimation is used to measure degradation and/or expected failure of a system in accordance with an illustrative embodiment.

The method 100 includes obtaining (e.g., by a processor) (step 102) in-situ control data set or experimental data set associated with a dissipative or thermal process of a system. The control or experimental data set is acquired to assess a degradation measure and to assess an entropy production for the dissipative process.

The method 100 further includes determining (e.g., by the processor) (step 104) one or more degradation coefficients from the control or experimental data. Each of the one or more degradation coefficients is determined as a rate of change of one or more assessed degradation measure parameters (e.g., w) with respect to one or more assessed entropy production (e.g., Sᵢ′) parameters for the dissipative or thermal process (e.g., pᵢ). In some embodiments, the rate of change is determined as a slope of a first coordinate axis associated with the one or more assessed degradation measure parameters and of one or more second coordinate axes each associated with an assessed entropy production parameter associated with the dissipative or thermal process. Table A is a summary of example sets of coefficients that can be generated form a system from a given approach described herein. Though shown as a set, it is contemplated that individual coefficient within a set maybe determined and subsequently used to assess the degradation measure of the system.

TABLE A
Summary of example sets of coefficients that can be generated form a system
from a given approach described herein

| System | Approach | Coefficient Sets |
|---|---|---|
| Battery | Maximum Work | B_{W} and B_{T}: based on linear dependence of i) |
| Degradation | | capacity accumulation (e.g., accumulated |
| | | discharge) on ii) ohmic entropies and on thermal |
| | | entropies, respectively |
| | Heat Balance | B_{HT} and B_{T}: based on linear dependence of i) |
| | | capacity accumulation (e.g., accumulated |
| | | discharge) on ii) heat-transfer entropies and on |
| | | thermal entropies, respectively |
| Grease Degradation | Maximum Work | shear work degradation coefficient B_{τ}, and thermal |
| | | degradation coefficient, B_{T}: based on linear |
| | | dependence between i) assessed shear stress and ii) |
| | | shear entropy and thermal entropy, respectively |
| | Heat Balance | B_{HT} and B_{T}: based on linear dependence of i) |
| | | capacity accumulation (e.g., accumulated |
| | | discharge) on ii) heat-transfer entropies and |
| | | thermal entropies, respectively |
| Structural Failure | Maximum Work | B_{W} and B_{T}: based on linear dependence between i) |
| due to Fatigue | | assessed mechanical stress (e.g. shear stress for |
| | | torsional loading) and ii) plastic strain entropy and |
| | | thermal entropy, respectively |
| | | BW_{D} and BT_{D}: based on linear dependence |
| | | between i) assessed CMD damage and ii) plastic |
| | | strain entropy and thermal entropy, respectively; |
| | | BW_{N} and BT_{N}: based on linear dependence |
| | | between i) assessed normalized cycles (N/N_{f}) and |
| | | ii) plastic strain entropy and thermal entropy |
| | Heat Balance | B_{HT} and B_{T}: based on linear dependence between |
| | | assessed stress (e.g. shear stress for torsional |
| | | loading, normal stress, or normal bending loading) |
| | | and heat transfer entropy and thermal entropy |

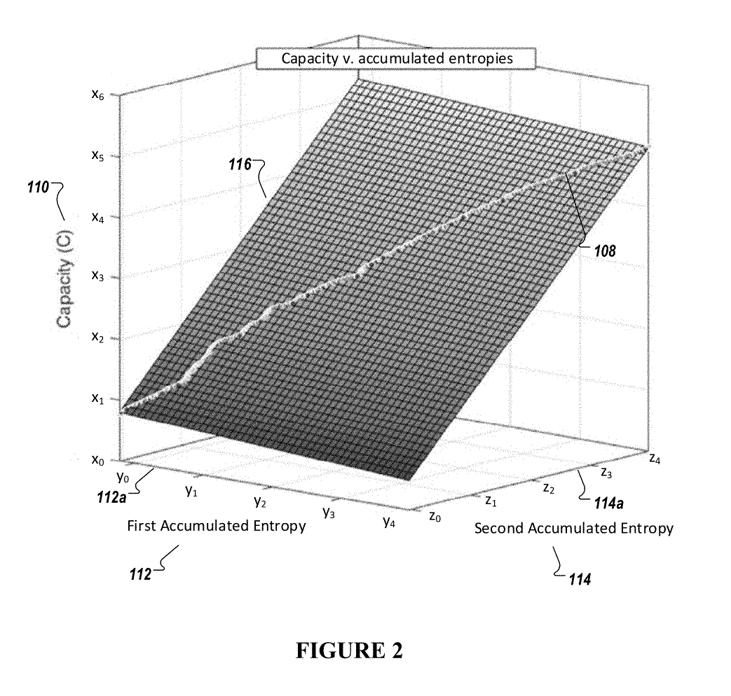

FIG. 2 shows an example the rate of change 108 determined as a slope of a first coordinate axis 110 associated with the one or more assessed degradation measure parameters and of one or more second coordinate axes (shown as 112a, 114a) in which each axis is associated with an assessed entropy production parameter (shown as 112, 114) associated with the dissipative or thermal process. Indeed, FIG. 2 shows an analysis of degradation and failure resulting from such dissipative processes as a geometric problem in multi-dimensional entropy “failure” space.

Referring still to FIG. 1, the method 100 further includes determining (e.g., by the processor) (step 106) one or more parameters associated with a measure of degradation and/or expected failure of the system, wherein the determination of the one or more parameters is based on an assessed estimated entropy parameter associated with estimated entropy produced by the dissipative process by linearly combining each of the one or more determined degradation coefficients with at least one corresponding assessed accumulated irreversible entropy parameter, and wherein the one or more parameters associated with the degradation and/or expected failure, or value(s) associated therewith, of the system is used in an evaluation of the system for use in engineering application or in the control, optimization or maintenance of said system in said engineering application.

In Situ-Control Data Set:

In situ-control data are real-time control sensor readings acquired during the course of control of a system operating environment. In situ-control can be part of the original sensor configuration of the operating environment (e.g., temperature sensors, voltage sensors, current sensors, rotation speed, etc.) In some embodiments, in situ-control sensors can be installed to augment a real-time monitoring and/or control application configured to evaluate or extent life of a system within the system operating environment.

Experimental Data Set:

Experimental data are collected from sensor readings often in design of experiments for the evaluation/selection of a system in a given engineering application and/or for the control, optimization, or maintenance of said system in said engineering application.

System:

As used herein, the term “system” refers to a component of interest that is subject to degradation. The system is used in a system operating environment. Examples of systems includes batteries, grease, and structural components in which the operating environment may be a vehicle or communication/electronic device equipped with such batteries. For a grease system, a vehicle or motor in which the grease is used would be its operating environment.

Entropy Production:

Entropy production, also referred to as entropy generation, measures the losses and irreversible transformations in real system-process interactions. Highly dissipative processes generate entropy at high rates and vice versa. Entropy production cannot be eliminated completely but can be reduced via design and optimization.

By way of example, methods related to the analysis of battery degradation are briefly summarized below; however, as demonstrated in the Examples, the methods described herein can be readily applied to model other degradative processes (e.g., grease degradation, fatigue, etc.).

Example DEG Application to Battery Degradation

Thermodynamic Formulations using Gibbs Free Energy are shown below

$$\begin{matrix}Gibbs Rate \dot{G} = - C \dot{T} - VI & ( Equation i .1 ) \\ Entropy production rate {\dot{S}}^{′} = \frac{C \dot{T}}{T} + \frac{VI}{T} & Equation ( i .2 )\end{matrix}$$

where the first RHS term in 1 is the thermal energy rate and the second RHS term is the ohmic work rate. In equation 2, the first RHS term is thermal entropy and the second ohmic entropy. To obtain total change in Gibbs energy and entropy generation during discharge (denoted by subscript d), both thermal and electrical energy changes are considered:

$$\begin{matrix}Δ G = - {\int }_{{t}_{0}}^{{t}_{d}} C \dot{T} dt - {\int }_{{t}_{0}}^{{t}_{d}} VI dt & Equation ( i .3 ) \\ {S}_{d}^{′} = {\int }_{{t}_{0}}^{{t}_{d}} \frac{C \dot{T}}{T} dt + {\int }_{{t}_{0}}^{{t}_{d}} \frac{VI}{T} dt & Equation ( i .4 )\end{matrix}$$

where t₀ is the start time and t_{d} the end time of the discharge process.

Degradation Entropy Generation (DEG) Analysis

Recall the DEG theorem

$$\frac{dw}{dt} = \sum B \frac{dS}{dt} \qquad Equation ( i .6 )$$

Equation 2 via the DEG theorem suggests a degradation rate is

$$\frac{dw}{dt} = {B}_{T} \frac{C \dot{T}}{T} + {B}_{W} \frac{VI}{T} \qquad Equation ( i .8 )$$

where B_{T} and B_{W} are Gibbs analysis degradation coefficients. In terms of entropy generation from a heat only analysis, the equation becomes

$$\frac{dw}{dt} = {B}_{T} \frac{C \dot{T}}{T} - {B}_{HT} [ \frac{( T - {T}_{∞} )}{{R}_{t} T} ] \qquad Equation ( i .1 )$$

where B_{T} and B_{HT} are heat generation analysis degradation coefficients. Equations 8 and 10 are the fundamental degradation relations. Degradation coefficients

$${B}_{i} = \frac{\partial w}{\partial {S}_{i}^{′}} {\vert}_{{p}_{i}} \qquad Equation ( i .11 )$$

can be evaluated from measurements, as slope of degradation measure w to entropy production Sᵢ′ for dissipative process pᵢ. Recall notation|ₚ_{ᵢ} refers top pᵢ being active. For one complete charge process,

$$\begin{matrix}w = {B}_{T} \int \frac{C \dot{T}}{T} dt + {B}_{I} \int \frac{VI}{T} dt & Equation ( i .12 ) \\ and & \\ w = {B}_{T} \int \frac{C \dot{T}}{T} dt - {B}_{HT} \int ⌈ \frac{( T - {T}_{∞} )}{RT} ⌉ dt & Equation ( i .13 )\end{matrix}$$

DEG Coefficients from Existing Models

Capacity as a Failure Parameter.

Letting accumulated discharge C be a degradation measure or performance parameter, equation (12), upon replacing C with w, becomes

$$C = {B}_{T} \int \frac{C \dot{T}}{T} dt + {B}_{W} \int \frac{VI}{T} dt \qquad Equation ( i .16 )$$

where the Gibbs capacity degradation coefficients

$${B}_{T} = \frac{\partial C}{\partial {S}_{T}^{′}} ; {B}_{W} = \frac{\partial C}{\partial {S}_{W}^{′}} \qquad ( Equation i .17 )$$

pertain to thermal entropy

$${S}_{T}^{′} = \int \frac{C \dot{T}}{T} dt$$

and ohmic entropy

$${S}_{W}^{′} = \int \frac{VI}{T} dt$$

respectively.

Similarly from equation (13),

$$C = {B}_{T} \int \frac{C \dot{T}}{T} dt - {B}_{HT} \int ⌈ \frac{( T - {T}_{∞} )}{RT} ⌉ dt \qquad ( Equation i .18 )$$

where the heat generation capacity degradation coefficients

$${B}_{T} = \frac{\partial C}{\partial {S}_{T}^{′}} ; {B}_{HT} = \frac{\partial C}{\partial {S}_{HT}^{′}} \qquad ( Equation i .19 )$$

pertain to entropies from thermal storage and heat transfer respectively.

Results and Data Analysis

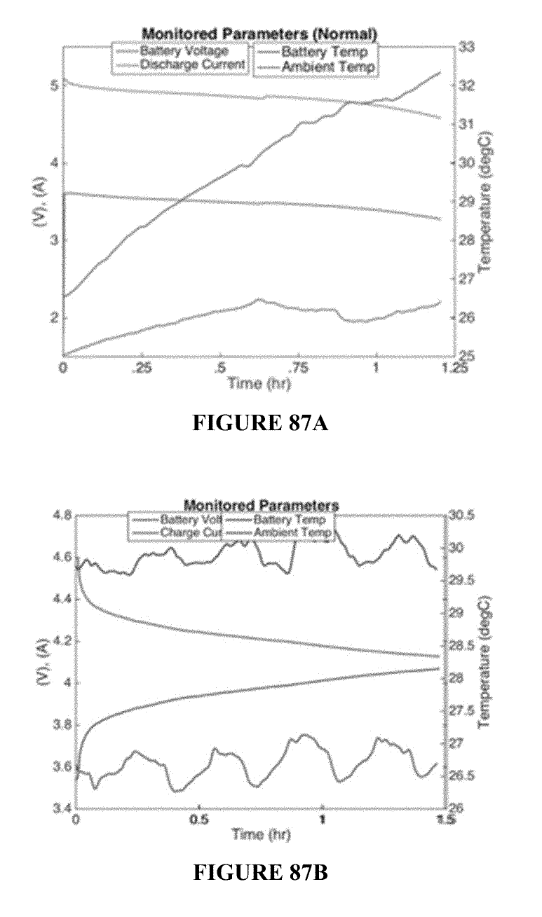

Charge/discharge current I, battery voltage V and temperature T were plotted versus time as a battery discharged and charged during cycle 4. FIGS. 87A-87B show a 40-minute discharge (FIG. 87A) with a monotonic rise in battery temperature, and a 2-hour charge cycle (FIG. 87B) with a slight change in temperature. Temperatures are on the right axis, while current and voltage are on the left. Temperature fluctuations arose from ambient conditions (students entering and exiting the laboratory).

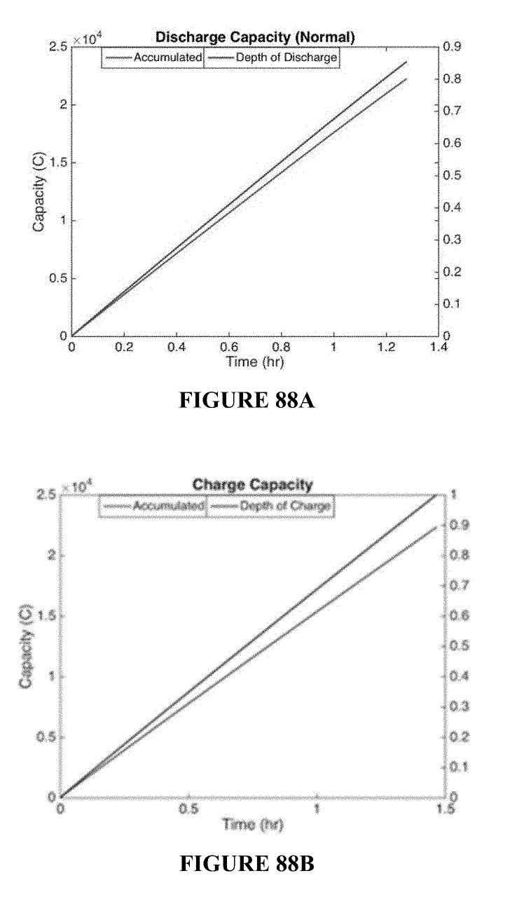

The accumulated charge (capacity) dropped during discharge (hence negative) and increased during charge (FIGS. 88A-88B).

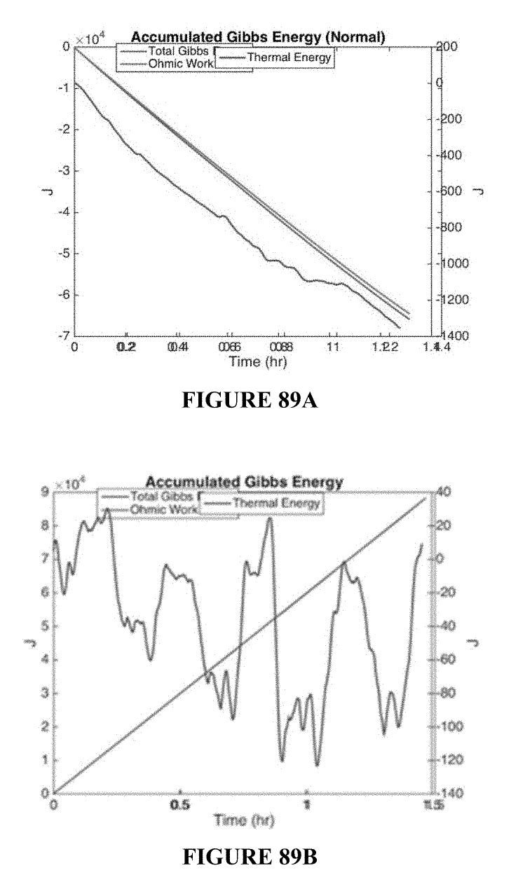

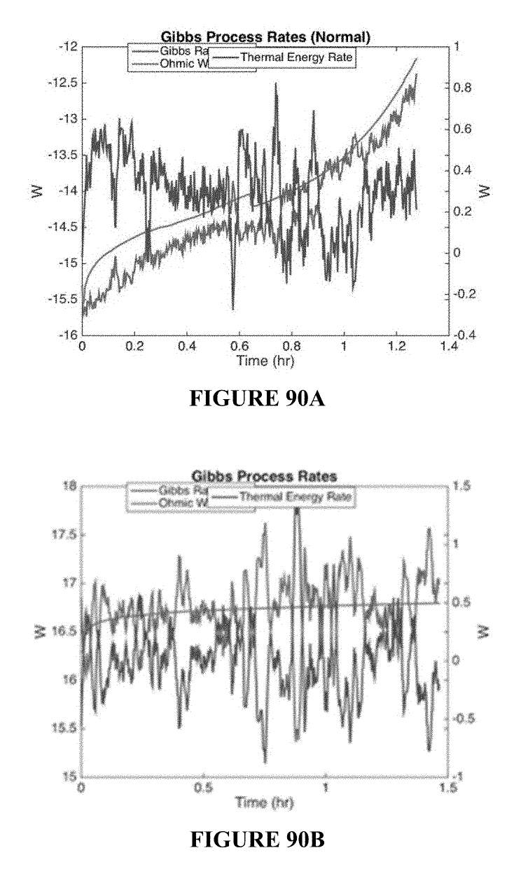

FIGS. 89A-89B plot accumulated Gibbs energy and components during discharge and charge. Ohmic work linearly decreased during discharge, and increased during charge. The thermal component changes the available Gibbs energy. With ohmic heating dominating heat removal mechanisms, including free convection to the environment, thermal energy in the battery increases, reducing available energy. The battery's Gibbs energy, dominated by ohmic work, behaved similarly. Thermal energy changes depend on heat capacity and change in battery temperature. Rates of processes influence energies.

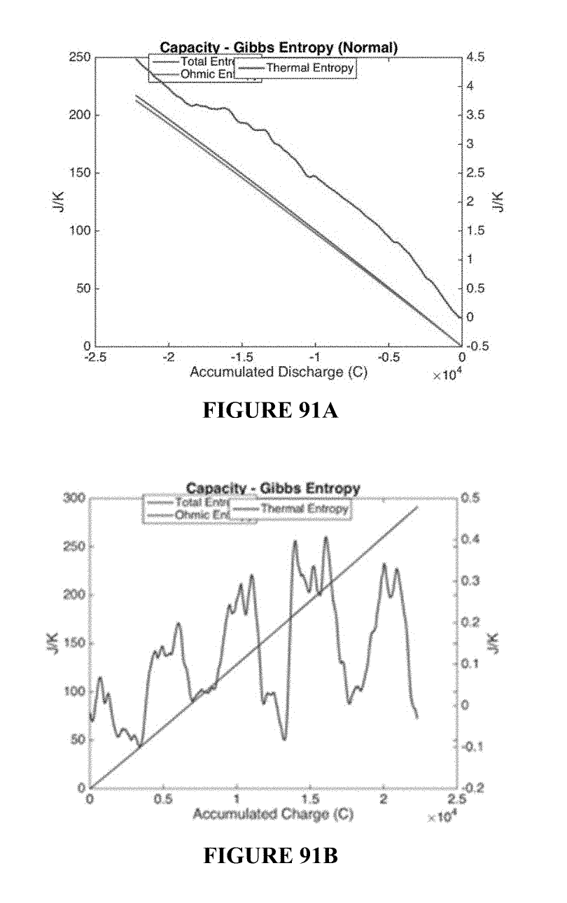

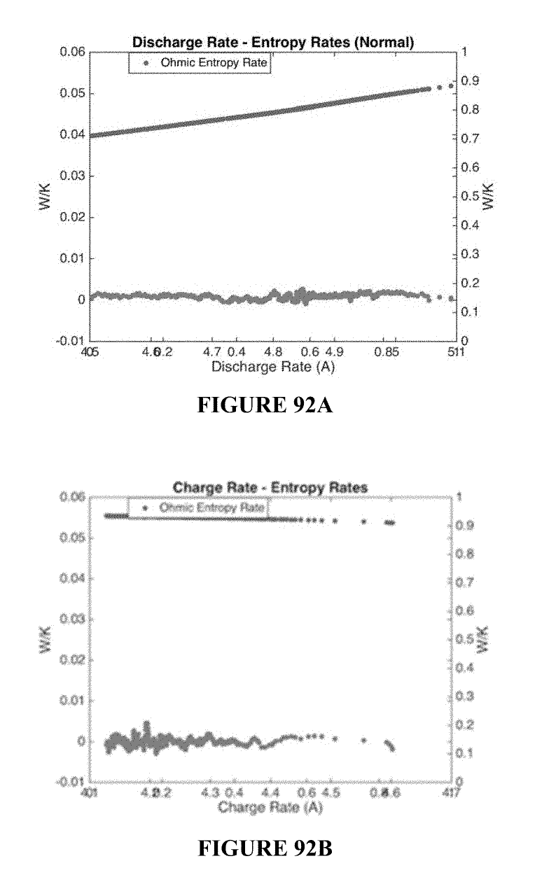

Ohmic, thermal and total entropies were plotted versus time. Accumulated charge/discharge (FIGS. 91A-91B) appears linear in Ohmic entropy (red plot) and nearly linear with thermal entropy (purple plot). With a discharge rate of 5 A and average charge rate of 4 A, entropy produced during discharge exceeded that during charge, for each cycle. Ohmic entropy generation rate decreased with decrease in current during cycling (FIGS. 92A-92B). With the relatively low temperature change rate observed, the thermal entropy change rate was also low. With both ohmic and thermal entropies linear, total Gibbs entropy was linear for both charging and discharging.

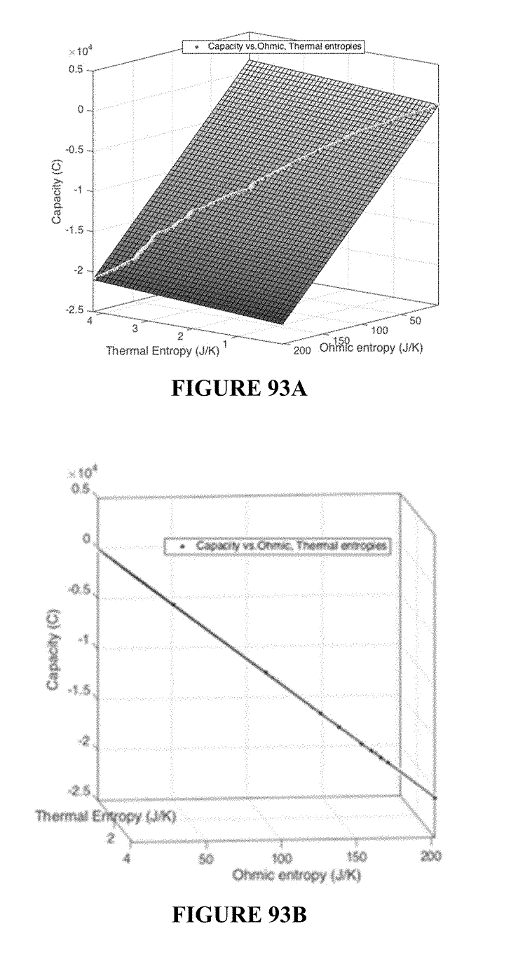

The actual partial contributions better visualize in the 3D surface plot, FIGS. 93A-93B. FIGS. 93A-93B indicate a dependence on two active processes. FIG. 93A shows the recorded data points during discharge trace a trajectory coincident with a linear plane. FIG. 93B gives an end view of the coincidence, visual of the goodness of fit R²>0.997. As with energy, the thermal contribution to entropy generation is two orders of magnitude less than the ohmic contribution, but contributes significantly to charge/discharge accumulation, and unlike thermal energy, should not be neglected. The significance of thermal entropy is underscored by the need to keep batteries cool during operation, for better and longer performance.

Degradation Coefficients Bᵢ

By associating data from the time instants, accumulated discharge (capacity) was plotted versus accumulated entropies. FIGS. 93A-93B plot this in a 3-dimensional space for data of one cycle. The battery's path through the space during discharge, from upper right corner to lower left corner—its Degradation Entropy Generation (DEG) trajectory—stays in a plane—its DEG surface. FIG. 93B, a view parallel to the plane, shows an almost perfect coincidence of the data points to the planar 2D surface, hence goodness of fit R²>0.997, rare for uncontrolled conditions and changing rates. This suggests a linear dependence of capacity accumulation on both ohmic and thermal entropies. Different dots on the trajectory represent different cycles. The thermal entropy is orders of magnitude less than the Ohmic entropy and shows scatter (from temperature sensitivity).

The 3-D space of the DEG surface characterizes the allowable regime in which the battery can operate. A battery's Degradation Entropy Generation (DEG) domain (here capacity versus ohmic and thermal entropy) can define consistent parameters for identifying desired characteristics from batteries of all configurations.

Degradation coefficients B_{W} and B_{T}, partial derivatives of capacity to ohmic and thermal entropies respectively, were estimated from the surface fit at each point of FIGS. 93A-93B. Here B_{W} is nearly constant over all cycles, with an average B_{W}=−108 equal to the slope of ohmic entropy versus discharge capacity. Average B_{T}=−327. The relative closeness in magnitude of both B_{T} and B_{W} for the lithium-ion batteries studied implies the same order of impact of both processes on accumulation, which can be deduced from the DEG trajectory. Proper formulation of the governing entropies of the active processes is required to accurately determine their contributions to overall accumulation and degradation.

DEG Trajectories, Surfaces, Domains

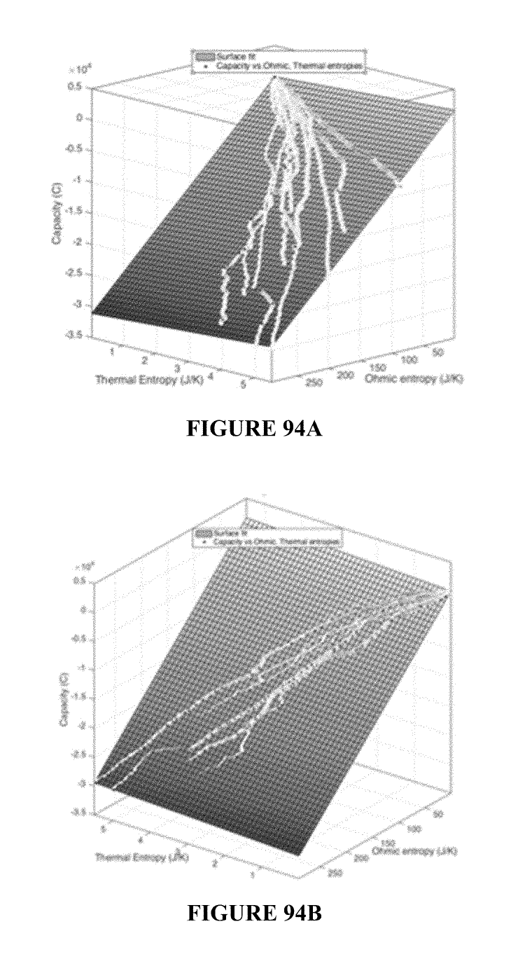

For a range of discharge rates, a set of DEG surfaces exist which define all possible DEG trajectories during operation. FIGS. 94A-94B, which plot DEG lines from cycles 1-10 of the li-ion battery (same discharge rate) supports a characteristic DEG surface containing all DEG lines the battery can “draw” at a given charge/discharge rate.

### Summary and Conclusion

Thermodynamic breakdown of the active processes in batteries during cycling were presented, including Gibbs-based and heat-based energy and entropy formulations during cycling. To these formulations was applied the DEG theorem to analyze battery degradation. Experimental results were applied to the DEG model.

A combination of thermodynamic analysis and the DEG theorem can be used by manufacturers to directly compare technologies, designs and materials used in battery manufacture. Also, without any prior information from the manufacturer about the battery, measurements and appropriate data analyses through the DEG theorem give a user an effective and consistent tool to compare various batteries to determine which is indeed most suitable for the intended application.

### EXAMPLES

While the methods and systems have been described in connection with certain embodiments and specific examples, it is not intended that the scope be limited to the particular embodiments set forth, as the embodiments herein are intended in all respects to be illustrative rather than restrictive.

By way of non-limiting illustration, examples of certain embodiments of the present disclosure are given below.

As noted above, material degradation occurs as a result of irreversible dissipative processes and forces. Various forms of degradation mechanisms exist such as friction, chemical reactions, plasticity, dislocation movements and corrosion all irreversibly leading to failure of a particular system or component. The first and second laws of thermodynamics describe states of a system from the perspective of energy content and exchanges. The first law prescribes energy conservation while the second law introduces the concept of irreversibility in systems as thermodynamic energies decrease, also known as entropy. It has been shown severally that entropy generation accompanies all degradation mechanisms simply by the irreversible nature of the dissipative processes involved. Hence, predicting and quantifying the effect of these processes based on accurate estimate of entropy produced led to the formulation of the Degradation-Entropy Generation (DEG) Theorem.

The DEG theorem also establishes that if a critical value of degradation measure exists, at which failure occurs, there must also exist critical values of accumulated irreversible entropies, and the relationship between them has also been formulated in an independent study in Russia. A close look at 2 classical theories: Holm's wear equation, w=kNx/H (subsequently modified to the more commonly used Archard's equation) and Coulomb friction law, F=μN, shows a direct proportionality between wear and energy dissipated by friction, w∝Fx. Application of the DEG theorem to a similar sliding friction between two surfaces and the resulting wear characterized by the accompanying entropy generated (or energy dissipated) is shown to define an equivalent wear coefficient k as the Holm-Archard equation.

The Examples below examine further developments and validation of the DEG theorem primarily in the area of its application to friction wear, grease degradation, battery ageing and fatigue analysis. A consistent thermodynamic approach for evaluating entropy generation accumulation is proposed. An investigation into the dissipative processes relevant to the degradation mechanisms is carried out for correlation to entropy generation. In addition to mathematical formulations, the examples include theorem verification using empirical fatigue data from previously published studies as well as seminal work—new battery and grease experiments to measure DEG parameters.

### Example 1. Overview of Thermodynamic Principles

Material degradation occurs as a result of irreversible dissipation, leading to failure of a system or component. Investigations to determine the critical stage at which failure occurs have been ongoing for several decades, and numerous theories and results have emanated over time. However, there remains a lack of a unified standard procedure for quantifying dissipative forces and rate of degradation to enable accurate prediction of failure. This study aims to formulate and apply a proposed theory, and develop experimental ways to verify and measure physical variables.

Dissipative processes drive a system towards equilibrium with the environment. After manufacture, every product in use tends towards failure over time. Highly dissipative processes accelerate system degradation, examples of which are: friction, turbulence, spontaneous chemical reaction, inelastic deformation, fretting, free expansion of a gas or liquid, flow of electric current through a resistance, and hysteresis among others. Reducing degradation by determining the prevalent dissipative processes, evaluating the resulting degradation and formulating ways to eliminate or reduce the effects is a major branch of manufacturing.

Relevant Concepts in Thermodynamics

Thermodynamics relates heat and work to the energy of a system and analyzes the state and effects of processes in the system. The first and second laws are discussed below.

First Law—Energy Conservation

The first law in differential form

dU=δQ−δW+ΣμdN (Equation

for a closed stationary thermodynamic system, neglecting gravity, balances dU the change in internal energy, δQ the heat exchange across the system boundary, δW the energy transfer across the system boundary by work, and ΣμdN the internal energy changes due to chemical reactions and diffusion. Inexact differentials indicate path dependence of heat and work transfers. The time rate form of equation (Equation 1.2) can be obtained by dividing through by dt, giving

\dot{U}=\dot{Q}−\dot{W}+Σμₖ\dot{N}ₖ (Equation

where \dot{Q} the rate of energy transferred in by heat flow at time t; \dot{W} is the rate of energy transferred out by work at time t; and \dot{N}ₖ is the rate of change of the number of moles of species k. For open systems,

\dot{N}ₖ=\dot{N}ₖ′+\dot{N}ₖ^{e} (Equation

where \dot{N}ₖ′ represents the rate of chemical composition change within the system, and \dot{N}ₖ^{e} is the rate of matter flow across system boundaries. For closed systems, \dot{N}ₖ^{e}4=0. Systems with significant internal diffusion effects and no chemical reactions have \dot{N}ₖ′=\dot{N}ₖ^{d} where \dot{N}ₖ^{d} represents diffusion rate. For chemical reactions,

ΣμdN=Adξ (Equation

where A is reaction affinity and dξ is reaction extent.

Work across a system boundary

W=∫₁²XdY (Equation

where X is a generalized force, usually an intensive property and dY is a generalized displacement, an extensive property. Work typical in engineering systems are listed in Table 1.1. A system can undergo multiple modes of work simultaneously in both directions during a process. In accordance with Clausius, net work done by the system is positive.

TABLE 1.1
Examples of work modes of energy transfer

| | Differential | |
|---|---|---|
| Work Mode | Form | Rate Form |
| Compression/Expansion of gas | δW = PdV | \dot{W} = P\dot{V} |
| Electrical | δW = Vdq | \dot{W} = VI |
| Shaft | δW = τdθ | \dot{W} = τω |
| Surface tension in liquid | δW = 2lτdx | \dot{W} = 2lτ\dot{x} |
| Tension in solid | δW = σAdx | \dot{W} = σA\dot{x} |

In integral form, the heat terms in equations (Equation 1.2) and (Equation 1.3) become

Q=∫₁²δQ (Equation

Q=∫ₜ_{₁}^{t}^{₂}\dot{Q}dt (Equation

Table 1.2 lists the three predominant modes of heat transfer. A system can exchange energy with the surroundings via multiple heat modes during a process. In accordance with Clausius, net energy transfer to the system via heat from the surroundings is positive.

TABLE 1.2
Examples of heat modes of energy transfer

| Heat Mode | Rate Form |
|---|---|
| Conduction | \dot{Q} = −kAdT/dx |
| Convection | \dot{Q} = −hA(T − T_{∞}) |
| Radiation | \dot{Q} = εσAT⁴ |

Second Law and Entropy Balance

For a closed system, the change in the entropy within the system is the sum of the entropy transferred across the system boundary and entropy generated within the system. For a domain that includes just the system,

ΔS_{sys}=Sᵢₙ−Sₒᵤₜ+S_{gen} (Equation

For an extended system including the system and immediate surroundings

ΔSₜₒₜₐₗ=ΔS_{sys}+ΔSₛᵤᵣᵣ (Equation

According to the second law, ΔSₜₒₜₐₗ≥0. For a reversible process,

ΔSₜₒₜₐₗ=0⇒ΔS_{sys}=−ΔSₛᵤᵣᵣ (Equation

Both equations (Equation 1.10) and (Equation 1.11) give the same entropy change for the system. A change in entropy between two states is the same for all possible ways the change can occur.

Δ_{sys}=ΔSᵣₑᵥ=ΔSᵢᵣᵣᵣ (Equation

where ΔSᵢᵣᵣᵣ is irreversible entropy change. Reversible entropy change

$$Δ {S}_{rev} = \int \frac{δ {Q}_{rev}}{T} \\ \text{?} \text{indicates text missing or illegible when filed} \qquad ( Equation \text{?}$$

Reversible heat transfer

δQᵣₑᵥ=C(T)dT (Equation

where C(T) is the heat capacity of the system, dependent on temperature T. For liquids and solids, the heat capacity is stable through a wide temperature range.

Irreversibilities—Entropy Generation

In an irreversible process, the system and/or all components of its surroundings remain altered at the end of the process. All real processes are irreversible. However, a system that has undergone an irreversible process can be restored to its initial state by making permanent irreversible changes to the surroundings. Internal irreversibilities occur within the system while external irreversibilities occur within the surroundings. The above classification is based on the location of the boundary; hence an extension of the boundary to enclose a portion of the surroundings will make all irreversibilities internal within the boundary considered. These examples generally focus on internal irreversibilities.

Irreversibilities are measured by the amount of entropy S′ generated. Also known as configurational or degradation entropy, S′ measures the permanent changes in the system when the process constraint is removed or reversed.

The second law is implied by the Clausius inequality

$$dS \geq {( \frac{δ Q}{T} )}_{b} \\ \text{?} \text{indicates text missing or illegible when filed} \qquad ( Equation \text{?}$$

for a closed system. In an open system, entropy transfer accompanies both heat flow and matter flow across system boundaries, [15]

$$dS \geq {( \frac{δ Q}{T} )}_{b} + \frac{{\sum }_{b} {μ}_{k} {dN}_{k}^{e}}{{T}_{b}} \\ \text{?} \text{indicates text missing or illegible when filed} \qquad ( Equation \text{?}$$

While the heat transfer mode and rate can be determined experimentally or modeled with reasonable accuracy using equations in Table 1.2, the entropy production is determined from energy and entropy balances, replacing the inequality with equality. In differential form, for a closed system

$${dS}_{sys} = {( \frac{δ Q}{T} )}_{b} + δ {S}^{′} \\ \text{?} \text{indicates text missing or illegible when filed} \qquad ( Equation \text{?}$$

where the first term on the right, the entropy transfer, may be positive or negative. The second law asserts that entropy generated δS′≥0. Here T_{b} is the temperature of the boundary where the heat transfer takes place. In rate form,

$$\frac{{dS}_{sys}}{dt} = {\sum }_{i} \frac{{\dot{Q}}_{i}}{{T}_{i}} + \dot{{S}^{′}} \\ \text{?} \text{indicates text missing or illegible when filed} \qquad ( Equation \text{?}$$

where \dot{Q}ᵢ/Tᵢ is the rate of entropy transfer through the portion of the boundary where instantaneous temperature is Tᵢ. For open systems,

$${dS}_{sys} = {( \frac{δ Q}{T} )}_{b} + \frac{{\sum }_{b} {μ}_{k} {dN}_{k}^{e}}{{T}_{b}} + δ {S}^{′} \\ \text{?} \text{indicates text missing or illegible when filed} \qquad ( Equation \text{?}$$

Combining equations (Equation 1.10) through (Equation 1.12) with (Equation 1.17), total entropy change for a closed system is

$$Δ {S}_{sys} = \int \frac{δ {Q}_{rev}}{T} = \int {( \frac{δ Q}{T} )}_{b} + {S}^{′} \\ \text{?} \text{indicates text missing or illegible when filed} \qquad ( Equation \text{?}$$

Substituting for δQᵣₑᵥ and rearranging,

$${S}^{′} = \int \frac{C ( T ) dT}{T} - \int {( \frac{δ Q}{T} )}_{b} \\ \text{?} \text{indicates text missing or illegible when filed} \qquad ( Equation \text{?}$$

Equation (Equation 1.21) is the entropy produced by the irreversible process given by the difference between reversible and irreversible entropy transfers.

Combining the First and Second Laws (the TδS′ Equation)

Eliminating δQ from equation (Equation 1.2) with (Equation 1.17) and rearranging gives

T_{b}δS′=T_{b}dS_{sys}−dU−δW+ΣμₖdNₖ′ (Equation

In rate format,

Tᵢ\dot{S}′=Tᵢ\dot{S}_{sys}−\dot{U}−\dot{W}+Σμₖ\dot{N}ₖ′ (Equation

Equations (Equation 1.22) and (Equation 1.23) are the fundamental thermodynamic relations for all closed systems undergoing real processes. Similar expressions for open systems can be obtained by including entropy transfer accompanying mass flow.

Thermodynamic Potentials—Closed System Analysis

The first and second laws can be reformulated for convenience using thermodynamic potentials. Relevant to these examples are enthalpy H, Helmholtz free energy A and Gibbs free energy G. Enthalpy

H=U+PV (Equation

measures the amount of thermal energy obtained from a closed thermodynamic system under constant pressure. In a chemical reaction, change in enthalpy is the heat absorbed by the reaction, in the form of change in internal energy and net work done by the system on the surroundings. Differentiating and substituting for dU from equation (Equation 1.2) gives the Enthalpy fundamental relation

dH=TdS+VdP+μdN′ (Equation

H=H(S,P,N) (Equation

For enthalpy, the equilibrium condition

dH|_{S,P,N}=0 (Equation

Equation (Equation 1.25) has no boundary work component, making it suitable for characterizing energy changes in systems undergoing chemical reactions and heat transfer at constant pressure. Combining equation (Equation 1.17) and (Equation 1.25),

$$δ {S}^{′} = {dS}_{sys} - \frac{{dH}_{rev}}{T} + \frac{VdP}{T} + \frac{\sum {μ}_{k} {dN}_{k}^{′}}{T} \\ \text{?} \text{indicates text missing or illegible when filed} \qquad ( Equation \text{?}$$

The Helmholtz free energy

A=U−TS (Equation

measures the maximum work obtainable from a thermodynamic process. Differentiating and substituting for dU gives the Helmholtz fundamental relation

dA=−SdT−PdV+μdN′ (Equation

A=A(T,V,N) (Equation

At equilibrium an incompressible system minimizes its Helmholtz potential

dA|_{T,V,N}=0 (Equation

Equation (Equation 1.30) includes the conjugate pair representing external boundary work. This enables re-formulation in terms of any other quasi-equilibrium work types such as listed in Table 1.1. The change in Helmholtz potential measures the maximum amount of useful work that can be extracted from any constant-volume, constant-composition system. Similar to enthalpy and internal energy, entropy production for Helmholtz energy changes within a system is given as

$$δ {S}^{′} = \frac{- d {A}_{rev}}{T} + \frac{SdT}{T} + \frac{XdY}{T} + \frac{μ {dN}^{′}}{T} \\ \text{?} \text{indicates text missing or illegible when filed} \qquad ( Equation \text{?}$$

Equation (Equation 1.33) gives entropy generation as the difference between reversible and irreversible entropies. At local Helmholtz equilibrium,

$$δ {S}^{′} = \frac{SdT}{T} + \frac{XdY}{T} + \frac{μ {dN}^{′}}{T} \\ \text{?} \text{indicates text missing or illegible when filed} \qquad ( Equation \text{?}$$

According to equations (Equation 1.30) and (Equation 1.34), dT increases with the other increasing work terms (usually due to heat produced by dissipation), which in turn adds to the process irreversibility—the faster and/or longer the processes take place, the more entropy generated. This is commonly observed in friction heating and battery operation.

Gibbs potential/free energy

G=U+PV−TS (Equation

can be used to measure process-initiating work obtained from an isothermal, isobaric thermodynamic system. For reactions such as phase transitions and chemical formation of substances, change in Gibbs energy can be used to calculate entropy change in the system. Differentiating and substituting for dU gives the Gibbs fundamental relation

dG=−SdT−VdP+μdN′ (Equation

G=G(T,P,N) (Equation

The equilibrium condition for constant-temperature, constant-pressure process minimizes the Gibbs potential

dG|_{T,P,N}=0 (Equation

Lack of a boundary work term or a quasi-static heat transfer term makes equation (Equation 1.38) a suitable measure of the maximum available chemical energy in a system. Electrochemical energy storage devices are characterized using Gibbs potential. Entropy production is

$$δ {S}^{′} = \frac{- {dG}_{rev}}{T} + \frac{SdT}{T} + \frac{VdP}{T} + \frac{μ d {N}^{′}}{T} \\ \text{?} \text{indicates text missing or illegible when filed} \qquad ( Equation \text{?}$$

Other non-chemical interactions within the system boundary like diffusion can be formulated and included in equations (Equation 1.36) and (Equation 1.39).

The above formulations hold at every instant of the appropriately described system, negating the need for steady state conditions as required by most models.

Heat-Only Analysis

In a process involving temperature changes, a heat generation analysis often provides better insight into the prevalent mechanisms. Applying the first law to a heat-only process,

dE=δQ+δE′ (Equation

where the change in thermal energy dE=CdT is the heat energy stored, δQ is the net heat transfer and δE′ is heat generated from work dissipation. Comparing equations (Equation 1.40) and (Equation 1.17),

$$δ {S}^{′} = \frac{CdT}{T} - \frac{δ Q}{T} \\ \text{?} \text{indicates text missing or illegible when filed} \qquad ( Equation \text{?}$$

which is same as equation (Equation 1.21) via another approach. Equation (Equation 1.41) expresses the entropy production from heat generation in a system having thermal storage and heat transfer entropies as components. While either term on the right of equation (Equation 1.41) can be negative, the left side must be non-negative via the second law. Heat transfer out of the system Q can be determined from

Q=ΔT/Rₜ (Equation

a ratio of the difference between system and ambient temperatures to the thermal resistance in between. Active work rate proceeds significantly faster than spontaneous heat transfer processes. For low heat-dissipation processes, heat transfer is not easily measurable, making the work transfer model more convenient.

Equation (Equation 1.41) evaluates entropy generation in systems using only temperature measurements and applies at every instant of the process.

Energy Dissipation Via Heat—the Heat Generation Term

Comparing equation (Equation 1.40) to (Equation 1.2) for non-reacting systems (i.e. ΣμdN=0) shows the existence of a common heat transfer term δQ. Hence if in equation (Equation 1.40), the thermal energy dE=CdT governs internal energy change from temperature change only, thermal energy conservation implies that the heat generation term δE′ is the thermal component of the boundary work interaction δW, commonly referred to as energy dissipation by heat (in frictional processes, viscous dissipation) [10], [11]. Hence the appropriateness of equation (Equation 1.40) and (Equation 1.41) in resolving the components of energy and entropy changes from heat-dominated processes is evident.

The Thermal Energy/Storage Term

The relations governing infinitesimal change in Helmholtz (equation (Equation 1.30)) and Gibbs (equation (Equation 1.36)) potentials introduce the thermal energy term SdT. This is the heat, not instantaneously transferred out during the work interaction, thereby raising system temperature. In a reacting tribo-control volume, SdT includes—in order of increasing magnitude—reaction heating, friction heating and significantly higher heating from a heat source. The temperature change dT is driven by the entropy content S which, in processes with relatively small temperature variations, Maxwell's relations give

$$S = {\int }_{0}^{T} \frac{dS}{dT} dt \approx T \frac{dS}{dT} = C \\ \text{?} \text{indicates text missing or illegible when filed} \qquad ( Equation \text{?}$$

where C is the heat capacity of the system, defined previously.

Work Vs Heat

A conceptual breakdown of both work-based and heat-based formulations have been presented above. While both formulations adequately characterize a system-process interaction at every instant, their suitability for entropy generation modeling or experimental measurements necessary to determine entropy generation from the active processes will depend on the availability or measurability of the intensive variables describing each process, respectively. Hence a knowledge of the system, active processes and available resources is necessary for optimal determination of the approach to be used. For example, an extreme-temperature thermal cycling process can be analyzed using the heat equation in conjunction with far-field temperature measurement equipment. Most processes in engineering fields such as mechanics have well defined boundary work terms, which have been historically measured with significant success by experimentalists, making the work approach more suitable in such fields.

Manufacturing Processes—Product Formation

The first law asserts that the internal energy content of a finished product is the energy required to form the product, consisting of the work done by the manufacturing processes (machining, etc.) on the raw material and the heat obtained from the environment.

ΔU=Q+W (Equation

Substituting Qᵣₑᵥ=TΔS

Wᵣₑᵥ=ΔU−TΔS=ΔA (Equation

The minimum external work done via manufacturing processes to form the product is the change in Helmholtz energy of the product from raw material. Considering the effects of the real processes taking place,

$$\begin{matrix} Δ A < W & ( Equation \text{?} \\ {S}^{′} = Δ {S}_{total} = \frac{W - Δ A}{T} \geq 0 \\ \text{?} \text{indicates text missing or illegible when filed} & ( Equation \text{?}\end{matrix}$$

Equation (Equation 1.47) is the entropy production in the manufacturing process, the difference between the actual work from the process and the minimum reversible work required to form the product. It is a measure of the wastage in the manufacturing process, a knowledge of which is critical to improving process efficiencies. Further sub-system analysis can be performed to determine the source(s) of the most significant irreversibilities.

Dissipating Processes—Product in Operation

After manufacture, a reverse process begins at product use. Manufacturers and consumers are primarily concerned about a product's usability and durability. As discussed, Helmholtz free energy measures the usability of the product while entropy production, a measure of its degradation, can be used to determine durability. Following a similar procedure as the manufacturing process analysis,

$${S}^{′} = Δ {S}_{total} = \frac{Δ A - W}{T} \geq 0 \qquad ( Equation$$

Discharging a battery from time t₁ to t₂,

$${S}^{′} = \frac{nFV - {\int }_{t 1}^{t 2} VIdt}{T} \geq 0 \qquad ( Equation$$

Using rotational shaft work Mω as work input, grease operation can be modeled using the above formulations. Maximum work that can be obtained from the grease

ΔA=ΔU−TΔS (Equation

and entropy production

$${S}^{′} = \frac{Δ A - {\int }_{t 1}^{t 2} M ω dt}{T} \geq 0 \qquad ( Equation$$

where M measures grease resistance to shearing, monitored as shaft torque, and ω is shaft speed.

### Example 2. The Degradation-Entropy Generation Theorem

Rayleigh through his dissipation function of mechanics was the first to characterize dissipative forces in terms of thermodynamic theories. In classical irreversible thermodynamics, Onsager developed his famous reciprocity theorem. A quantitative study of degradation of systems by dissipative processes formulated the Degradation-Entropy Generation (DEG) theorem which established a direct relationship between rates of entropy generation and degradation using irreversible thermodynamics. Formulations and interpretations of the DEG theorem are discussed below.

Degradation-Entropy Generation Theorem

Given an irreversible material transformation, consisting of i=1, 2, . . . , n dissipative processes pᵢ, which could describe an energy, work, or heat characteristic of the process. Assume effects of the mechanism can be described by a parameter or state variable that measures the effects of the transformation

w=w(pᵢ)=w(pᵢ,p₂, . . . , pₙ), i=1,2, . . . , n (Equation

that is monotonic in each pᵢ. Then the rate of change of the parameter or state

$$\frac{dw}{dt} = {\sum }_{i} {B}_{i} \frac{{dS}_{i}^{′}}{dt} \qquad ( Equation 2.53 )$$

is a linear combination of the irreversible entropies dSᵢ′/dt generated by the dissipative processes pᵢ, where the transform process coefficients

$${B}_{i} = \frac{\partial w}{\partial {S}_{i}^{′}} {\vert}_{{p}_{i}} \qquad ( Equation 2.54 )$$

are slopes of degradation w with respect to entropy generation Sᵢ′; the |pᵢ notation refers to the process pᵢ being active.

Proof:

Define degradation measure w which monotonically increases (or decreases) with progression of the degradation (and thus is a measure of that degradation); w must depend on all i=1, 2, . . . , n dissipative processes pᵢ that drive the degradation. In accordance with the second law of thermodynamics, each pᵢ must produce a non-negative irreversible entropy Sᵢ′=S′(pᵢ). The total entropy generated

S′=S′(pᵢ)=S′(p₁,p₂, . . . , pₙ), i=1,2, . . . , n (Equation 2.55)

sums the entropies produced by the pᵢ with “prime” indicating irreversible entropy generated. Applying the chain rule to equations (Equation 2.52) and (Equation 2.55), rates of entropy production and degradation are respectively

$$\begin{matrix} \frac{{dS}^{′}}{dt} = {\sum }_{i} \frac{\partial {S}_{i}^{′}}{\partial {p}_{i}} \frac{{dp}_{i}}{dt} = {\sum }_{i} \frac{{dS}_{i}^{′}}{dt} & ( Equation 2.56 ) \\ \frac{dw}{dt} = {\sum }_{i} \frac{\partial w}{\partial {p}_{i}} \frac{{dp}_{i}}{dt} = {\sum }_{i} \frac{\frac{\partial w}{\partial {p}_{i}}}{\frac{\partial {S}_{i}^{′}}{\partial {p}_{i}}} \frac{\partial {S}_{i}^{′}}{\partial {p}_{i}} \frac{{dp}_{i}}{dt} = {\sum }_{i} \frac{\partial w}{\partial {S}_{i}^{′}} {\vert}_{{p}_{i}} \frac{{dS}_{i}^{′}}{dt} = {\sum }_{i} {B}_{i} \frac{{dS}_{i}^{′}}{dt} & ( Equation 2.57 )\end{matrix}$$

In equation (Equation 2.57), the term of the second equality multiplied by 1=[∂Sᵢ′/∂pᵢ]⁻¹ [∂Sᵢ′/∂pᵢ] produced the third equality. Substitution of terms for dSᵢ′/dt from equation (Equation 2.56) into the third equality gave the fourth equality. The final equality defined the degradation coefficient Bᵢ=∂w/∂Sᵢ′=[∂w/∂pᵢ] [∂Sᵢ′/∂pᵢ]⁻¹.

Equation (Equation 2.57) relates states or parameters w associated with the material transformation to the entropies generated by the dissipative processes that cause the degradation. This can be applied to any material transformation monotonic in the actuating dissipative processes, including ageing, manufacturing, and healing processes. Embedded in the individual entropy production terms dS′ᵢ/dt are the dynamics of behavior of the individual dissipative processes pᵢ, often posed as the rate of energy dissipated divided by a temperature.

Statements of the DEG Theorem—the Degradation Force and Degradation Coefficient

Combining Prigogine's formulation of entropy generation Sᵢ′ from generalized thermodynamic forces Xᵢ and generalized flow rates Jᵢ with generalized degradation w, the Degradation-Entropy Generation Theorem, states that
1. the degradation rate is a linear combination \dot{w}=Σᵢ Bᵢ\dot{S}′ᵢ of the entropy generation components \dot{S}′ᵢ=XᵢJᵢ of the dissipative processes pᵢ,
2. the degradation components \dot{w}=Σᵢ YᵢJᵢ proceed at the same rates Jᵢ=\dot{ζ}ᵢ(t) as determined by the entropy production \dot{S}′=ΣᵢXᵢJᵢ of the dissipative processes pᵢ, where zᵢ are generalized displacements dependent on time t,
3. the generalized degradation forces Yᵢ are linear functions Yᵢ=BᵢXᵢ of the generalized thermodynamic forces Xᵢ, and
4. the degradation coefficient Bᵢ=Yᵢ/Xᵢ=∂w/∂Sᵢ′|ₚᵢ is the slope of w vs. S′, with process pᵢ active.

Integrating equation (Equation 2.57) yields the total degradation accumulated w=Σᵢ BᵢS′ᵢ, which is also a linear combination of the entropy accumulation components, S′ᵢ generated by the dissipative processes pᵢ.

Critical Entropy of Failure S_{f}: The DEG theorem also establishes that if a critical value of degradation measure exists, at which failure occurs, there must also exist critical values of accumulated irreversible entropies. Using exhaustive experimental data, the existence of a material-dependent fatigue fracture entropy (FFE) can also be demonstrated.

Degradation Analysis Procedure

Based on the above formulations, a systematic approach to degradation analysis using the DEG theorem was developed. The approach generalized forces in terms of entropy of dissipative processes. The forces and accompanying degradation are reformulated into terms for the specific dissipative processes relevant to the mechanism. The approach embeds the physics of the dissipative processes into the energies pᵢ=pᵢ(ζᵢ), derives entropy generation term \dot{S}′ᵢ as a function of pᵢ, and expresses the forces and the rate of degradation \dot{w}ᵢ as a linear combination of all entropy generation terms, see equation (Equation 2.57). Here ζᵢ are time-dependent phenomenological variables associated with process pᵢ. The degradation coefficients Bᵢ must be measured using equation (Equation 2.54). The proposed approach is:
1. Identify the degradation measure w, dissipative processes pᵢ and variables ζᵢ. Express pᵢ as energy dissipated, work lost, heat transferred, a thermodynamic energy (internal energy, enthalpy, Helmholtz or Gibbs free energy), or some other functional form of energy. Process energy pᵢ=pᵢ(ζᵢ) can be formulated via all macroscopic work-energy methods, a few of which are given in Table 1.1.
2. Obtain thermodynamic flows Jᵢ=\dot{ζ}ᵢ(t).
3. From the process functionality pᵢ=p(ζⁱ(t)) obtain ∂pᵢ/∂ζiᵢ(t) and if necessary, obtain thermodynamic forces Xᵢ.
4. Find entropy generation directly, or use \dot{S}′=Σᵢ XᵢJᵢ.
5. Evaluate coefficients Bᵢ by measuring increments or rates of degradation versus increments or rates of entropy generation, with process pᵢ active. Since pᵢ is an energy, it can be shown that ∂S′/∂pᵢ=1/Tᵢ, where Tᵢ is a temperature.
6. Obtain Yᵢ=BᵢXᵢ if necessary.
7. Using your estimated values of \dot{S}′ᵢ and Bᵢ, obtain degradation rate \dot{w}=Σᵢ Bᵢ\dot{S}′ᵢ.
8. Finally obtain the associated dissipative forces, e.g. friction and normal forces.

This approach can be used to solve problems consisting of one or many variegated dissipative processes as illustrated below.

### Thermodynamics of Dissipative Processes—Application of the DEG Theorem DEG Formulation for Friction and Normal Forces

The application of the DEG theorem is discussed below. This is presented formulations to estimate the magnitude of friction and normal forces in a dissipative process as follows.

Recall the thermodynamic fundamental relation in equation (Equation 1.22),

TδS′=TdS_{sys}−dU−δW+ΣμₖdNₖ′ (Equation

Work interaction during the mechanical process is defined by

δW=F_{η}dx+ηNdy (Equation

where F_{η} is the frictional force, η is the friction coefficient and N is the normal force, dx and dy are the orthogonal displacements associated with the tangential and normal forces respectively. Substituting (Equation 2.59) into (Equation 2.58) gives

$$T Σ ( \frac{\partial {S}^{′}}{\partial {p}_{i}} \frac{\partial {p}_{i}}{\partial {ζ}_{i}} ) \partial {ζ}_{i} = {TdS}_{sys} - dU - {F}_{η} dx - η Ndy + Σ {μ}_{k} {dN}_{k}^{′} \qquad ( Equation$$

The differentials dx, dy and dN′ₖ are linearly independent of each other, which then imposes a condition for the equation above that ζᵢ associated with the dissipative processes must be a function of x, y and N′ₖ, (i.e. ζᵢ=ζᵢ(x, y, N′ₖ)) or else the differentials and their coefficients will vanish. Assuming steady state process (dS=dE=0) and applying the chain rule and regrouping,

$$[ {F}_{η} + T {\sum }_{i} ( \frac{\partial {S}^{′}}{\partial {p}_{i}} \frac{\partial {p}_{i}}{\partial {ζ}_{i}} \frac{\partial {ζ}_{i}}{\partial x} ) ] dx + [ η N + T {\sum }_{i} ( \frac{\partial {S}^{′}}{\partial {p}_{i}} \frac{\partial {p}_{i}}{\partial {ζ}_{i}} \frac{\partial {ζ}_{i}}{\partial y} ) ] dy + [ - {μ}_{k} + T {\sum }_{i} ( \frac{\partial {S}^{′}}{\partial {p}_{i}} \frac{\partial {p}_{i}}{\partial {ζ}_{i}} \frac{\partial {ζ}_{i}}{\partial {N}_{k}^{′}} ) ] {dN}_{k}^{′} = 0 \qquad ( Equation$$

The independence of differentials dx, dy and dN′ₖ implies that each of their coefficients must equal zero, giving

$$[ {F}_{η} = - T {\sum }_{i} ( \frac{\partial {S}^{′}}{\partial {p}_{i}} \frac{\partial {p}_{i}}{\partial {ζ}_{i}} \frac{\partial {ζ}_{i}}{\partial x} ) ] , \\ [ N = - T / η {\sum }_{i} ( \frac{\partial {S}^{′}}{\partial {p}_{i}} \frac{\partial {p}_{i}}{\partial {ζ}_{i}} \frac{\partial {ζ}_{i}}{\partial y} ) ] , \\ [ {μ}_{k} = T {\sum }_{i} ( \frac{\partial {S}^{′}}{\partial {p}_{i}} \frac{\partial {p}_{i}}{\partial {ζ}_{i}} \frac{\partial {ζ}_{i}}{\partial {N}_{k}^{′}} ) = T \frac{\partial {S}^{′}}{\partial {N}_{k}^{′}} ] \qquad ( Equation$$

The first two sub equations in equation (Equation 2.62) above express friction and normal forces as functions of the active dissipative processes at the interface, while the third establishes the standard definition of chemical potential.

### Application to Experimental Sliding Friction and Wear—Rate Form

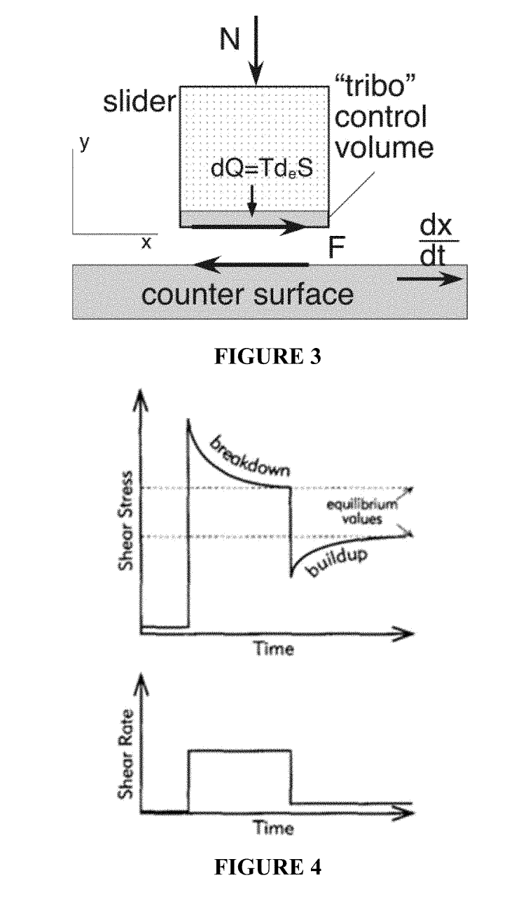

Dry sliding friction between two surfaces result in volumetric loss of material or wear. FIG. 3 shows a schematic diagram of two surfaces in sliding contact. The surface and near surface of the slider are enclosed in a “tribo” control volume. The counter surface applies friction force F onto the slider as indicated. Work done on the control volume is given by equation (Equation 2.59).

Considering the case of ductile metals, where the principal dissipative process p is due to plastic deformation, the following assumptions were made to simplify the analysis:
1. Assuming steady state (constant speed and force and \dot{S}=Ė=0).
2. Energy transport due to material loss μdNₖ^{e}=0 is negligible relative to the other terms in the fundamental relation.
3. No interfacial chemical reactions occur (dN′ₖ=0).
4. All friction work is dissipated within the control volume (dp=−dW).

Combining equations (Equation 2.56) and (Equation 2.59) and from step 4 of the DEG analysis procedure above,

$$\frac{{dS}^{′}}{dt} = \frac{\partial {S}^{′}}{\partial {p}_{i}} \frac{\partial {p}_{i}}{\partial t} = \frac{1}{T} \frac{{F}_{η} \partial x}{\partial t} \qquad ( Equation$$

where T is steady state contact temperature (from the relation ∂S′/∂p=dS′/dW=1/T). For dS′/dt≥0, since T≥0, +/−(F_{η})=+/−(dx/dt), as expected in agreement with direction of friction indicated in FIG. 3. From equation (Equation 2.63), the following relationships obtain: J=dx/dt, ζ=x, and X=∂S′/∂p (∂p/∂ζ)=F_{η}/T. DEG equation (Equation 2.57) gives for wear w

$$\dot{w} = BXJ = \frac{B}{T} \frac{{F}_{η} dx}{\partial t} = \frac{B}{T} \frac{η Ndx}{\partial t} ; Y = B η N / T \qquad ( Equation$$

DEG Coefficients from Existing Models

Defining wear w in equation (Equation 2.64) as the volumetric wear w_{V} as done by the prominent Archard's wear law, in rate form and under isothermal and constant load conditions, \dot{w}ᵥ=kN\dot{x}/H where k=wear constant and H=hardness of the softer contact surface (x and N are as defined previously). Compared to equation (Equation 2.64),

B=kT/ηH (Equation

which gives a value for B from known and measurable Archard wear law parameters. A reverse estimation of wear constant k from measured B (from wear tests) gave a 5% error.

This approach has been successfully verified with relatively minimal experimental error values. The procedure can be employed to different systems and degradation mechanisms as discussed below.

Further Contributions to the DEG Approach

Equations (Equation 2.58) and (Equation 2.60) require a knowledge of the internal energy and entropy changes in the control volume to evaluate entropy generation S′. These are often difficult to determine accurately in practice, necessitating the steady state assumption above.

Using the Helmholtz potential form of entropy generation given in equation (Equation 1.34), equation (Equation 2.60) is replaced by

TδS′=SdT+F_{η}dx+ηNdy+ΣμₖdkNₖ′ (Equation

conveniently absorbing dU and dS into SdT, the change in thermal energy of the system, which has a consistent meaning in every system (like the compositional change term). In this form, every term on the RHS of equation (Equation 2.66) has the same interpretation in all systems undergoing the same active boundary interaction and has the general form of product of a generalized force and a flow rate. Equation (Equation 2.66) measures the actual irreversible entropy generation pertaining to the dissipation of useful mechanical energy via friction at the tribological interface and is easily evaluated from measurable process interaction terms on the RHS.

Alternatively, equation (Equation 2.66) can be obtained directly from the DEG analysis procedure which suggests an accumulation of entropy generation from all active processes. In accordance with natural experience, frictional energy dissipation is predominantly via heat. Hence, measurable changes in system temperature indicate measurable changes in its thermal energy. Also, if the interface interaction proceeds long enough or its magnitude large enough, permanent measurable changes to its composition (via wear) will take place. Hence, representing all three concurrent processes,

TδS′=CdT+F_{η}dx+ηNdy+ΣμₖdNₖ′ (Equation

where the first RHS term represents thermal energy change, the middle terms, the mechanical work and the last term, the irreversible compositional change. Equations (Equation 2.66) and (Equation 2.67) are equivalent forms. The conversion between C and S is given above.

Hence, the steady state assumption used in previous applications of the DEG theorem can be neglected to take advantage of the instantaneous validity of the first and second laws of thermodynamics. However, when necessary, an order of magnitude analysis can be used to drop terms with minimal impact on total entropy generation estimated. While compositional changes can be easily neglected in non-reacting systems, care should be taken when making the isothermal assumption as shown below.

Entropy Generation Determination

With the demonstrated need for appropriate formulations for accurate degradation analysis, this section breaks down the significance of the various forms of the combined first and second laws given above in evaluating entropy generation in real systems.

Maximum Work

In addition to simplifying the analysis formulation, an understanding of the contribution of individual process terms pᵢ towards ‘useful’ entropy generation is necessary for proper and consistent application to system analysis. As shown in below, this is suggested by the linear dependency predicted by the DEG theorem of degradation on partial entropy contributions.

Internal Energy

The internal energy in equation (1.1) suggests a change in ‘total’ energy of the system independent of the system type. The universality of this term makes it convenient to use in theoretical thermodynamic analysis pertaining to all system state changes. However, in experimental work, a misunderstanding of the impact of measured internal energy changes on the intended application often results in a presumed inconsistency in energy/entropy approaches, and hence a pushback from experimentalists and industry engineers. In other words, if dU and dS in equation (Equation 2.58) are known, they indicate changes in the system state but give no information on what those changes represent from a system utility standpoint. Simply put, a battery with an 80% drop in internal energy is more useful (has more electrochemical potential or free energy) in supplying electric charge through direct interaction than a freshly cut diamond, so an internal energy analysis conducted for both components is subject to misinterpretation.

Free Energies

To combat the above dilemma, thermodynamic free energies, reviewed above, are recommended. These potentials represent different forms of energy changes in a component based on its utility (hence their definitions as maximum useful work obtainable).

In Example 1, the concept of maximum work was introduced briefly and applied to manufacturing and dissipating processes. The Helmholtz potential equation (Equation 1.30), by subtracting heat transfer from internal energy suggests that the useful work from a system (e.g., mechanical) is reduced by an increase in its thermal energy, boundary work out of it (the intended application and usually the prevalent process) and compositional changes to it, all simultaneously occurring, albeit at significantly different rates. Hence extracting the maximum boundary work from the system would require the first and last terms in equation (Equation 1.34) dropping off, indicating an isothermal and constant-composition process. According to the second law, this is only approximately achievable by progressing the boundary work quasi-statically or imposing a temperature and composition control on the system undergoing the process, the latter usually requiring energy input from an external source. Hence maximum work is an idealization described by reversible Helmholtz dAᵣₑᵥ and a difference between reversible Helmholtz and irreversible (real) Helmholtz gives a measure of the irreversibilities in the system as given by equation (Equation 1.33) (derived from first principles). Applying Prigogine's concept of local equilibrium sets dAᵣₑᵥ=0, giving the final form in equation (Equation 1.34) (applied above).

It is noted that the word “free” comes from the natural ability of the component to do work, without need for intermediate interaction, suggesting that a degradation of this particular “potential” represents actual degradation of the component for practical purposes. This is the portion of the total internal energy change relevant in application-based system degradation analysis.

Steady State Operation

Many systems when operated long enough approach equilibrium asymptotically. However, several processes progress with significant transients in both process rates and system responses (e.g., rechargeable battery cycling). In the same way, equilibrium assumption simplifies energy analysis, steady state operation simplifies experimental measurements and subsequent data analysis. When applied to thermodynamic formulations, \dot{S}=Ė=0 implies thermodynamic intensive variables such as T are unchanging.

Most thermodynamic formulations describing mechanical/chemical phenomena use the steady state assumption to simplify the equations. Most mechanical dissipation equations exclude temperature altogether. As discussed previously and shown below, this is acceptable based on relative order of magnitude. However, this is not universally true and hence a robust entropy formulation should include all instantaneously active terms. Also, as shown later, the DEG coefficients indicate the significance of the component entropy terms to actual degradation measure.

Heat-Only Analysis

Another contribution is the use of heat-only analysis to determine entropy generation, equation (Equation 1.41). This has advantage of using only temperature measurements to determine the components of entropy generation. In heat-dominated processes like non-reacting thermal cycling, the thermodynamic potentials do not always present convenient ways to evaluate the components of entropy generation. According to experience and as prescribed by the heat-only form of the first law, equation (Equation 1.40), a body in contact with the surroundings or other thermal reservoir will transfer heat out as its temperature rises above that of the surroundings. Hence more appropriate energy and entropy balances based on the prevalent and concurrent thermal processes, driven by the system dissipation processes, as given in equations (Equation 1.40) and (1.40) respectively, are likely to give more accurate description of entropy accumulation components, required by the DEG theorem.

Features of both work interaction and heat interaction approaches are analyzed and discussed below.

Other Common Dissipative Processes

Some common dissipative processes and the associated entropies they generate are summarized in Table. 2.1.

TABLE 2.1
Common processes and their boundary entropy generation terms.

| Mechanism | Entropy generation term |
|---|---|
| Adhesion | $Δ {S}^{′} = ( \frac{Δγ}{{T}_{m}} ) Δ {A}_{s}$ |
| Plastic Deformation and Viscous Dissipation | $Δ {S}^{′} = ( \frac{{U}_{c}}{{T}_{m}} ) ΔV ; {U}_{c} = ( \frac{{dW}_{p}}{d V} )$ |
| Fracture | $d {S}^{′} = ( \frac{G - 2 {γ}_{o}}{{T}_{cr}} ) da$ |
| Abrasion and Cutting | $Δ {S}^{′} = ( \frac{{U}_{i}}{T} )$ |
| Phase Changes | $Δ {S}^{′} = ( \frac{Δ H}{{T}_{phase}} )$ |
| Chemical Reactions | $d {S}^{′} = ( \frac{A}{T} ) d ξ$ |
| Mixing of Materials | $Δ {S}^{′} = - R {\sum }_{i}^{n} \frac{{N}_{i}}{N} ln \frac{{N}_{i}}{N} ; N = {\sum }_{i}^{n} {N}_{i}$ |
| Diffusion | $d {S}^{′} = ( \frac{{η}_{1} - {η}_{2}}{T} ) d ξ$ |
| Heat Transfer | $d {S}^{′} = ( \frac{1}{{T}_{c}} - \frac{1}{{T}_{h}} ) d Q$ |

Again, for each of these dissipative processes, it is noted that the entropy produced is a product of a driving potential (or weighting term) or force and a differential of the associated phenomenological variable or flow.

### Summary and Conclusion

In this example, the Degradation-Entropy Generation Theorem was reviewed from a thermodynamic standpoint. The mathematical formulation was derived and the current methods of application to dissipative processes and wear mechanisms in sliding friction were reviewed. Recommendations to modify the DEG approach to improve its robustness and universality in real-life applications were presented. The use of appropriate thermodynamic potential, as done in equation (Equation 2.67) and the heat generation formulations in Example 1 replace the steady state assumption, and employ the instantaneous applicability of the first and second laws of thermodynamics to all macro systems/processes, an applicability inherited by the DEG theorem to degradation analysis of all systems/processes. These form the deductive apparati upon which the validity of the DEG theorem is proven as demonstrated in the examples that follow.

In subsequent examples, detailed system analyses are presented combining thermodynamic formulations in Example 1 and DEG formulations and procedure, including the new conceptual contributions, discussed in Example 2. Experimental verifications of the formulations are also presented and discussed, which reverse-verify the second law of thermodynamics.

### Example 3. Grease Degradation

Grease mixes and disperses lubricating oils into a thickener to form a gelatinous product that lubricates surfaces in contact. High load applications such as rolling contact bearings and some gears are greased. Because the base oil is suspended in the high shear strength thickener, the base oil does not flow out of the clump, rendering grease as a semi-permanent lubrication method. However, grease lubricant properties degrade over time, which can result in catastrophic failure of equipment. Needed is improved insight into grease degradation mechanisms for better failure prediction.

Grease base oils are mineral oils with Naphthenic oils most common. Since thickeners determine overall properties, grease is classified based on its thickener. Desired properties also vary with operating conditions and environment. High-temperature applications require thickeners that withstand heat, food-processing machines need non-toxic thickeners and water applications require water-resistant thickeners. Most thickeners are soap and non-soap based. Most common soap-based thickeners contain soap made from fats, oils (e.g. animal fat) and alkali such as caustic soda NaOH. Non-soap clay-based greases contain either inorganic thickeners such as silica clays or organic thickeners such as amides. Additives that improve certain desired grease properties range from anti-oxidants, anti-wear and corrosion inhibitors, among others. Fillers such as graphite and metal oxides also improve grease performance. A typical general-purpose grease has about 85% base oil, 10% thickener and 5% additives/fillers.

Manufacturers perform in-house tests and studies and characterize greases based on application. Over the years, ASTM and NLGI have worked with researchers and manufacturers to establish consistent methods for classifying grease and predicting grease life.

Grease Rheology

Due to thickeners, grease behaves as a non-Newtonian fluid. Grease deforms under applied forces which change its rheological properties and impact performance. Understanding these properties is valuable to the grease industry, manufacturers and end users. Evolution and current state of understanding of grease behavior is reviewed.

Thixotropy

The microstructure of grease changes under mechanical shearing in operation. This change starts upon load application and tends towards a steady state at a time determined by the grease thickener type and content. After load removal, the grease sample tends slowly back to its original state. Thixotropy generally applies to isothermal viscoelastic changes in grease microstructure, observed in the particle distribution uniformity and bond density (e.g. intermolecular hydrogen bonds), as grease breaks down under shear and rebuilds during relaxation. Another explanation suggests recovery occurs due to effects of Brownian motion. FIG. 4 plots of applied shear rate and resulting change in shear stress versus time show breakdown in microstructure during shear, and subsequent buildup after shear. For thermodynamic analysis, the equilibrium and/or steady state points are approached asymptotically and not in finite time, hence pseudo-equilibrium points at appropriate time limits are usually specified for analyses and measurements. The inability of the sheared sample to return spontaneously to original state in finite time during relaxation increases permanent structural breakdown over time. This increases with shear rate until recovery is significantly diminished. Grease microstructure is determined primarily by thickener and to some extent, additives.

Viscoelasticity

Grease behavior, demonstrated via an oscillatory test, shows a complex response of elastic (real) and viscous (imaginary) parts expressed in terms of storage G′—in phase with the shear—and loss moduli G″−90 degrees out of phase with shear,

G*=G′+iG″ (Equation

where from Hooke's law,

$${G}^{′} = \frac{{τ}_{0}}{{γ}_{0}} cos δ and {G}^{″} = \frac{{τ}_{0}}{{γ}_{0}} sin δ \qquad ( Equation 3.69 )$$

In terms of viscosity,

η*=η′+iη″ (Equation

where the elastic and viscous components are

$${η}^{′} = \frac{{G}_{0} {G}^{′}}{{τ}_{0} ω} and {η}^{″} = \frac{{G}_{0} {G}^{″}}{{τ}_{0} ω} \qquad ( Equation 3.71 )$$

respectively. The phase shift angle is given by

$$tan δ = \frac{{G}^{″}}{{G}^{′}} \qquad ( Equation$$

Grease exhibits linear viscoelasticity at low strain amplitude, independent of strain, and becomes increasingly non-linear at higher amplitudes, above a critical strain γ_{c} where G′=G″.

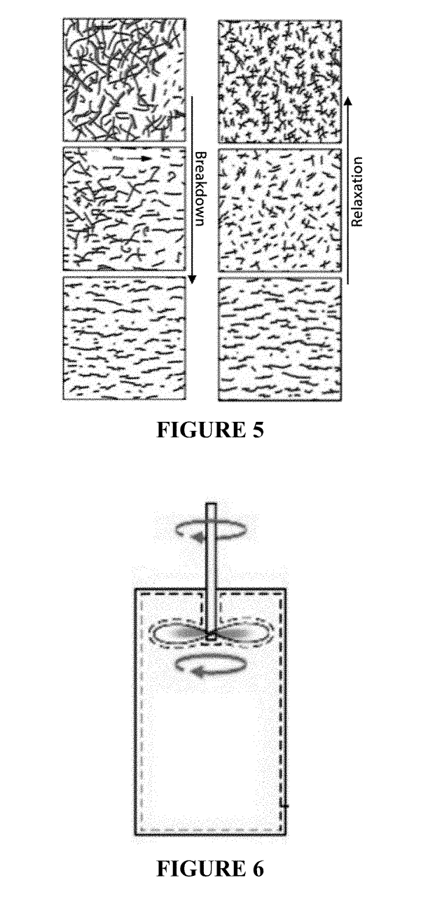

To study grease microstructure, Atomic Force Microscopy images (FIG. 5) show lithium grease microstructure (a) response from a fresh unsheared state through the first few minutes, to over 10 minutes of continuous shear; (b) the reverse buildup process after shearing [37].

Without a parameter that fully defines effects of breakdown in microstructure, experts have attempted to establish macroscopic properties directly related to thixotropy, including thixotropic index, viscosity, consistency, shear stress, modulus, and interparticle bonds, among others. Shear stress, viscosity and the ASTM-recommended consistency based on grease worker tests [34] are most common in the grease industry. Choice of parameter/model depends on convenience and consistency of measurement methods.

Degradation Measures

Multiple candidates for degradation measures for the DEG theorem will be overviewed.

Shear Stress

Resistance to shear is grease's most significant property, typically determined by strain response to stress or stress response to strain. Shear stress in grease is time-dependent, indicating grease thixotropy. At a given shear rate, shear stress increases up to the yield stress τ_{y} wherein grease completely breaks down and flows. Most applications require τ>τ_{y}. While an exact yield stress value cannot be determined experimentally, definitions are typical for macroscopic analysis. A consistent method for determining yield stress of grease under steady shear rate from a predefined transition from linear visco-elasticity has been recommended. Shear stress in grease, typically measured with a rheometer, has been related to other measures of grease degradation.

Apparent Viscosity

Time-dependent viscosity can measure grease performance. For liquids and semi-solids, viscosity has been related to shear stress and shear rate. Viscosity of thixotropic substances (e.g. grease) exhibits a time-dependent behavior similar to shear stress, that asymptotically approaches a steady-state value limited by base oil viscosity. Grease viscosity is typically determined in rheometric measurements.

Thixotropic Index

Thixotropic Index TI, a common experimental parameter for comparing thixotropy of different substances, compares viscosity responses at low (ηₛ) and high (η₁₀ₛ) shear rates. A factor of 10 is typical for shear rates.

$$TI = \frac{{η}_{s}}{{η}_{10 s}} \qquad ( Equation$$

Consistency

Grease consistency, which measures grease hardness, depends on the degree of aggregation of soap fibers. When grease loses consistency, load-carrying shear stress diminishes, rendering grease unsuitable. Loss of consistency results from thermal and mechanical operating conditions. However, some greases maintain consistency after degrading, e.g. Calcium-based greases. ASTM standard D217—Standard Test Methods for Cone Penetration of Lubricating Grease—details two standardized tests for consistency in terms of penetration depth (Pen in 1/10 mm) of a cone penetrometer and prescribes a method for working grease using the mechanical grease worker followed by another measurement of the worked grease consistency. National Lubricating Grease Institute (NLGI) classifies commercial greases based on consistency numbers correlated to worked penetration ranges. Consistency measurements are prone to error; with each manufacturer performing in-house measurements, penetration ranges are used. Each consistency number spans a penetration range of 30. Rheologists find penetration measurements inadequate, and thus use the more accurate rheometric measurements of viscosity and shear stress, in spite of equipment cost.

In the absence of a rheometer, consistency measurements can estimate yield stress and viscosity. Using published experimental data, Lugt gives

τ_{y}=3E10*Pen^{-3.17} (Equation

and

τ_{y}=4E16*Pen^{-5.58} (Equation

where Pen is cone penetration depth. In terms of viscosity at a shear rate of 10 1/s,

log₁₀ η₁₀=16.5882−5.58 log₁₀ Pen (Equation

Drop Point

Thermal stability of grease is determined by drop point, the temperature at which grease changes from an original gelatinous state to a liquid state, under prescribed conditions. Drop point is based on the type of thickener; hence thermal stability of grease is more a quality control parameter than a degradation variable. The ASTM-2265 standard test for measuring drop point heats a sample of grease in an oven while monitoring temperature, until the first drop of oil falls into a lower container through an opening in an upper cup.

Degradation Mechanisms

Grease degradation occurs mechanically, thermally and sometimes chemically. Mechanical and thermal degradation reduce grease consistency and break down thickener. Chemical degradation can oxidize base oil and thickener, separate/evaporate oil from thickener and/or breakdown the oil-thickener mixture. With multiple simultaneous degradation mechanisms, conditions determine which mechanisms dominate. Degradation proceeds irreversibly at a rate dependent on the dissipative process(es) active, typically oxidation and evaporation during storage, and mechanical shear work and heating during use. This study investigates these primary degradation modes, active simultaneously or individually. Even with special high-temperature greases, thermal instability from heat induces oxidation and evaporation of base oil.

Mechanical Shearing

Most significant to grease degradation is shearing between two solid boundaries. Reduction of friction and wear in tribology interfaces, e.g., bearings, is the primary function of lubricating greases. Under shear, grease structure breaks down as a function of time, shear stress and shear rate.

Despite various experimental and complex methods to study grease under shear, ASTM and NLGI recommends methods for platform-independent consistent measurement, and classification of different greases.

Thermal Breakdown

High temperatures damage grease microstructure. Viscous heating and heat from high-temperature operating environment, with shearing, can separate from thickener the base oil which then flows out of the lubricated interface, leading to failure. Greases are typically weakened at sufficiently high temperatures (with the exception of urea greases).

Most grease-lubricated applications operate at temperatures below 120° C., with special greases for high-temperature environments. Most grease formulations exclude the temperature variable (or specify a constant temperature), assume the grease operating temperature range far below drop point, and assume temperature has insignificant effect on microstructure. However, experiments on Lithium grease showed a 22% drop in viscosity and about 25° C. drop in dropping point, when held at 150° C. for 10 days, and over 50% drop in yield stress of seven different greases tested for a temperature rise of 75° C.

Oxidation

Grease oxidation is slow, but common during long-term storage or high-temperature applications. Grease has lower oxidation stability than mineral oils. Grease oxidation increases with temperature which generally shortens useful life. While formulations for lubricant oil oxidation are typically applied to grease analysis, thickeners can also oxidize. Oxidation breaks down structure of oil and grease and forms radicals in phases.

Oxidation tests are often accelerated. ASTM D-942 and ASTM D-5483-05 define standard testing procedures using the oxygen pressure vessel method and the pressure differential scanning calorimetry (PDSC) respectively. The latter method involves measuring oxidation induction time in an accelerated test with oxygen at 210° C. and 3.5 MPa.

At high temperatures and shear rates, thermal and chemical degradation can be as significant as mechanical degradation. A parameter to estimate the more significant mechanism is Peclet number

$$Pe = \frac{6 πη {a}^{3} \dot{γ}}{kT} \\ \text{?} \text{indicates text missing or illegible when filed} \qquad ( Equation \text{?}$$

where η is viscosity, a is particle radius, \dot{γ} is the shear rate, k=1.38 E-23 J/K is Boltzmann constant and T the grease temperature.

Physical Models and Limitations

Several models of grease behavior have been proposed, mostly experimental. A good thixotropic model includes the time dependence of grease to shearing. The models involve one degradation mechanism, illustrated in Tables 3.1 and 3.2.

TABLE 3.1
Physical grease models for mechanical degradation - theoretical and experimental.

| Degradation | Degradation | | |
|---|---|---|---|
| Mechanism | Measure | Published Model | Notes |
| Mechanical | Shear stress | Power-law: τ = k\dot{γ}ⁿ | Earliest widely adopted model, |
| | (Yield | | limited to a narrow range of |
| | stress) | | medium shear rates. k is |
| | | | consistency factor and n is flow |
| | | | index. |
| | | Herschel-Bulkley: | Currently the most widely used. |
| | | τ = τ_{y} + K\dot{γ}ⁿ | Good correlation with data at |
| | | | shear rates between 0.001 and |
| | | | 1000 s⁻¹. n ≈ 0.5 for greases. |
| | | Sisko: τ = K\dot{γ}ⁿ + η_{b}\dot{γ} | Typically applied to high shear |
| | | | rates (>1000 s⁻¹). |
| | | $Maxwell \text{:} \frac{d τ}{dt} = G ( \dot{γ} - \frac{τ}{η} )$ | Widely used to describe viscoelasticity using a spring in |
| | | | series with a viscous damper. |
| | | | Gives an accurate time-based |
| | | | shear stress response at constant |
| | | | shear but does not accurately |
| | | | describe constant shear stress |
| | | | behavior. |
| | | $Gecim and Winer \text{:} \dot{γ} = \frac{1}{{G}_{∞}} \frac{d τ}{dt} + \frac{{τ}_{L}}{μ} {tanh}^{- 1} ( \frac{τ}{{τ}_{L}} )$ | Adds a nonlinearity to the Newtonian component in the Maxwell model using the limiting shear stress concept. |
| | Shear strain | $Kevin \text{-} Voigt \text{:} \frac{d γ}{dt} = \frac{τ - γ G}{η}$ | Connects the above elements in parallel and accounts for the |
| | | | constant shear stress time- |
| | | | dependent strain response. Does |
| | | | not accurately predict relaxation. |
| | Viscosity | $Mewis \text{:} \frac{d η}{dt} = {k [ {η}_{e} ( {\dot{γ}}_{1} ) - η ]}^{n}$ | Gives rate of change of viscosity at constant shear rate. |
| | | $Cross \text{:} \frac{dN}{dt} = {k}_{2} P - ( {k}_{0} + {k}_{1} {{\dot{γ}}_{1}}^{m} ) N$ | Gives the rate of bond breakdown in grease in terms of number of |
| | | | linkages N*. |

TABLE 3.2
Physical grease models for mechanical degradation - theoretical and
experimental (* The number of links per chain N is further related to viscosity).

| Degradation | Degradation | | |
|---|---|---|---|
| Mechanism | Measure | Published Model | Notes |
| Thermal | Shear stress | $Arrhenius \text{:} τ = {γ}_{0} G [ exp ( \frac{{E}_{a}}{RT} ) ]$ | Uses the Arrhenius formulation to describe grease response to |
| | | | temperature changes. |
| | Yield stress | $Lugt \text{:} \frac{{τ}_{y}}{{τ}_{y 0}} = exp [ ( \frac{{T}_{0} - T}{b} ) ln 2 ]$ | Extends the Arrhenius formulation to yield stress. |
| | Viscosity | $Arrhenius \text{:} η = {η}_{0} [ exp ( \frac{{E}_{a}}{RT} ) ]$ | Arrhenius formulation - viscosity. |
| Chemical | Shear stress | $\frac{d τ}{dt} = - {τ}_{0} kexp ( - kt )$ | Based on Rhee's % degradation = e^{-kt} |
| | Viscosity | $\frac{d η}{dt} = - {η}_{0} kexp ( - kt )$ | Extends Rhee's % degradation = e^{-kt} |
| | | | to viscosity. |
| | Mass | $Lugt \text{:} \frac{d m}{dt} = - {m}_{0} kexp ( - kt )$ | Describes oxidation in grease via mass change. |

The Problems

Most models in Tables 3.1 and 3.2, inadequate to consistently model grease degradation over time, limit the range of shear rates and greases. Pre-shearing of greases makes difficult establishing a consistent initial condition for shearing tests. Other issues include wall slip for low shear rate tests, equipment inertia and dependence on soap composition. The mechanical shearing models are isothermal. Without temperature control, data must be normalized to an approximate constant temperature. Overall, the reviewing authors concluded these models do not consistently and adequately characterize observed trends, and are mostly empirical.

Existing Energy Models and Limitations

Energy-based formulations, which relate microstructure stability of grease to viscous energy density formulated from measured work input, are more consistent and less restricted. Kuhn's energy approach defined a rheological energy density

$${e}_{rh} = η \dot{γ} ( \frac{{\overline{d}}_{e}}{{h}_{0}} ) \\ \text{?} \text{indicates text missing or illegible when filed} \qquad ( Equation \text{?}$$

a quotient of rheological input work and grease volume, as a function of grease properties and operating conditions, which Kuhn related to a friction coefficient

$${η}_{f} = \frac{{e}_{rh}}{{p}_{r}} {i}_{rh} \\ \text{?} \text{indicates text missing or illegible when filed} \qquad ( Equation \text{?}$$

where η is the dynamic viscosity (Pa s), D is the shear rate (s⁻¹), dₑ is the middle diameter of micro contact (mm) and hₒ* is the central film thickness (mm). Kuhn defined structural degradation rate of grease

$$φ = \frac{{τ}^{2} {V}_{G}}{{De}_{rh}^{*} {h}_{0}^{*}} \\ \text{?} \text{indicates text missing or illegible when filed} \qquad ( Equation \text{?}$$

and established a maximum degradation point based on viscosity loss. The limiting viscosity nₗᵢₘ defined the minimum viscosity under specified operating conditions, with which an energy density threshold eₗᵢₘ was defined in terms of accumulated shear stress and viscosity strain β. These theories are linked through the apparent rheological frictional energy density eᵣₕ=Wᵣₕ/Vᵣₕ. Different energy levels for different greases remained a problem, making these formulations grease sample- and process-specific.

Kuhn evaluated eᵣₕ in equation (Equation 3.78) using mechanical dissipation with measured values of shear stress and shear rate. Frictional energy density

eᵣₕ=\dot{γ}∫ₜ₀^{tf}τ(t)dt (Equation

with a limiting value

$${e}_{lim} = {lim}_{t \to {t}_{lim}} {e}_{rh} ( t ) .$$

For an isothermal process with a steady state dissipation function, Kuhn defined specific energy e*=eᵣₕ/ϕ a measure of the unsteady dissipation of the grease's available friction energy with a limiting value of 1. In the experiment, the friction energy was a function of the shearing motion of the solid boundaries. Friction energy and the wear intensity parameter e* were then unified using the steady state value of the dissipation function.

Kuhn further investigated thixotropic behavior of grease. Using rheometric measurements of NGLI 2 grease, Kuhn identified elastic and plastic regions in shear. Defining a maximum degradation condition as degradation rate greater than or equal to −0.005, Kuhn formulated time dependence of shear stress and friction energy as

$$\begin{matrix} τ ( t ) = {{τ}_{lim} ( \frac{t}{{t}_{lim}} )}^{- n} & ( Equation \text{?} \\ e ( t ) = {τ}_{lim} ( \frac{1}{- n + 1} ) {( \frac{t}{{t}_{lim}} )}^{- n + 1} \\ \text{?} \text{indicates text missing or illegible when filed} & ( Equation \text{?}\end{matrix}$$

where n is structural degradation intensity. Kuhn had two issues: difficulty of isolating shear degradation from thermal and pressure effects, and effect of interface wear in boundary and mixed friction measurements. Kuhn's DEG-based approach compared previous frictional energy formulations with irreversible entropy generation. Using the open system entropy balance,

$$\frac{dS}{dt} = {\dot{S}}_{irr} ( t ) + {\dot{S}}_{Q} ( t ) + {\dot{m}}_{in} {\dot{s}}_{in} - {\dot{m}}_{out} {\dot{s}}_{out} \\ \text{?} \text{indicates text missing or illegible when filed} \qquad ( Equation \text{?}$$

Kuhn evaluated an apparent rheological energy density from the combined 1st and 2nd law equations at steady state, defining entropy generation in terms of frictional energy, and obtaining an expression for estimating frictional energy from entropy transfer by mass and heat

eᵣₕ=T_{f}(ρₒᵤₜsₒᵤₜ)−T_{f}(\dot{m}ᵢₙ\dot{s}ᵢₙ−S_{Q})/Vₒᵤₜ (Equation

Kuhn measured two different greases under similar conditions and plotted normalized degradation versus normalized entropy flow but excluded oxidation. Correlations between friction and changing rheological properties of grease during loading have been reported. Measurements of friction factor for different greases under different load conditions show a linear dependence of friction factor on consistency, viscosity, storage energy, limiting energy and cohesion energy. Accumulated energy density as a function of grease composition and shear rate and an asymptotic tendency in the energy density and shear stress. Frictional and rheological tests on specially manufactured grease samples with different soap concentrations experimentally verified a fitted form of the Leider-Bird model, which gives time-dependent shear stress

$$τ ( t ) = k {\dot{γ}}^{n} [ 1 + ( b \dot{γ} t - 1 ) {\sum }_{1}^{i} {w}_{i} {{e}_{i}}^{( - t / λ n} ] \\ \text{?} \text{indicates text missing or illegible when filed} \qquad ( Equation \text{?}$$

and showed the “yielding” energy density, eₗ depends on shear rate, soap concentration and/or base oil viscosity, while τ_{y} depends only on soap concentration and base oil viscosity.

Others have used frictional power dissipation, evaluated from oscillation speed and normal torque, to assess wear rate

\dot{w}ₐᵥ=ψ_{w}P_{d} (Equation

with wear energy dissipation coefficient ψ_{w}. To relate wear to temperature rise, they established a linear relationship between power dissipation and temperature rise

P_{d}=ψ_{T}ΔT (Equation

with coefficient ψ_{T}. Via finite element thermal analysis, they predicted ΔT for wear rate.

Using internal energy, entropy generation can be formulated in terms of shear stress, shear rate and temperature,

$${S}_{g} = \dot{γ} ( t ) {\int }_{t 0}^{tf} \frac{τ ( t ) dt}{T} \\ \text{?} \text{indicates text missing or illegible when filed} \qquad ( Equation \text{?}$$

for mechanical degradation of grease. By using the DEG theorem, a linear relationship between net penetration Pen, the degradation measure used, and viscous energy accumulated/entropy produced can be obtained

Pen=0.014ε+0.069 (Equation

Pen=4.162S_{g}+0.071 (Equation

Experiments to determine S_{g} verified the above formulations. These results underscore the need for a steady operating temperature below oxidation temperature, to satisfy the entropy formulation and isolate degradation due to mechanical shearing only. From these findings, an engineering model has been proposed to predict grease degradation under mechanical shear. The constant temperature assumption in the previous work was addressed and a less restrictive formulation of entropy production was

$${S}_{g} = {\int }_{t 0}^{tf} \frac{τ ( t ) \dot{γ} ( t )}{T ( t )} dt \\ \text{?} \text{indicates text missing or illegible when filed} \qquad ( Equation \text{?}$$

Khonsari et al defined and measured shear stress-based degradation, linear in generated entropy, with which they developed a time-dependent shear stress model for mechanical shearing

$$τ ( t ) = {e}^{- \int \frac{α \dot{γ} ( t )}{T ( t )} dt} ( \int β ( t ) , {e}^{\int \frac{α \dot{γ} ( t )}{T ( t )} dt} dt + C ) \qquad ( Equation 3.93 )$$

At constant temperature and shear rate, equation (Equation 3.93) reduces to

$$τ ( t ) = {τ}_{∞} + ( {τ}_{0} - {τ}_{∞} ) exp ( - \frac{∝ \dot{γ}}{T} t ) \\ \text{?} \text{indicates text missing or illegible when filed} \qquad ( Equation \text{?}$$

Equation (Equation 3.94) was experimentally verified with different combinations of grease type, shear rate and temperature. Although the work illustrated the above formulations and the entropy versus consistency relationship to evaluate practical lifetime performance for grease, the result is not applicable when other degradation modes are significant.

Others have proposed a time-dependent model for grease degradation which gave a steady state shear stress τ at time t from start of shearing as

$$τ ( t ) = {[ {k}_{1} t ( m - 1 ) {{τ}_{o}}^{1 - m} + {( {τ}_{0} - {τ}_{r} )}^{1 - m} ]}^{\frac{1}{1 - m}} + {τ}_{r} \\ \text{?} \text{indicates text missing or illegible when filed} \qquad ( Equation \text{?}$$

The Problem

Grease degradation models have evolved from physical and manufacturer-specific empirical models to thermodynamics-based models. Most models struggle combining effects of different dissipative mechanisms (mechanical, chemical and thermal). Using thermodynamics and the DEG formulations, a more universal and consistent model can be formulated to account for simultaneous degradation modes, and an alternate formulation using entropy interactions by heat.

Analysis

Grease is usually confined between solid boundaries, as in bearing housings. In experiments and tests, a sample is placed in a grease cup with energy transfer via mechanical work, heat transfer, chemical reactions or concurrent combined modes. In high temperatures, external heat also transfers to the grease from surroundings.

Thermodynamic Analysis

Established will be thermodynamic analyses that include mechanical, chemical and thermal interactions: a first considers work and heat, and a second considers only heat. For both, after the work interactions cease, the system spontaneously settles to a new equilibrium state.

System: Grease Undergoing Elasto-Hydrodynamic Shearing, Heating and Oxidation.

Infinitesimal Model—Maximum Work Model

Helmholtz Analysis: Assumptions:
1. The system is the grease sample only, enclosed in the bearing housing (the boundary), FIG. 6.
2. System is closed.
3. Heat transfers with surroundings.
4. The system is at equilibrium before and after operation.
5. A lumped capacity models the grease (no spatial variation in properties).
   The infinitesimal change in Helmholtz free energy of the grease sample during breakdown (i.e. doing work) is given by equation (Equation 1.30)

dA_{b}=−SdT−XdY+μdN′ (Equation

where for thermal energy S≈C, see equation (Equation 1.43). Mechanical shearing work involves angular displacement θ and shear torque M

XdY=Mdθ (Equation

where

dθ=ωdt (Equation

In terms of steady displacement and varying torque, a more convenient form for constant-rate shearing of grease,

XdY=θdM (Equation

From this,

dN′=dN′_{react}dN′ₑᵥₐₚ (Equation

Also,

$${dN}^{ ′} = \frac{d m}{{M}_{m}} \\ \text{?} \text{indicates text missing or illegible when filed} \qquad ( Equation \text{?}$$

where Mₘ is the grease molecular mass.

Combining gives the maximum useful work obtainable from the grease sample, the change in Helmholtz free energy

$${dA}_{b} = - CdT + θ d M + \frac{μ}{{M}_{m}} dm \\ \text{?} \text{indicates text missing or illegible when filed} \qquad ( Equation \text{?}$$

To satisfy dA≤0 as the grease energy decreases, dT≥0, dM≤0 and dm≤0 and equation (Equation 3.102) follows the Clausius convention. Subscript b denotes breakdown. The system has three independent properties when all three modes occur simultaneously and independently, which suggests

A=A(T,M,m) (Equation

Equation (Equation 3.102) also applies to an open-system in which grease that flows out of the lubrication interface is continuously replaced by fresh grease. Oxidation does not begin until significant heating, hence for closed-system applications below the drop point,

dA_{b}=−CdT−θdM (Equation

Mechanical shearing is the most significant degradation process. If the grease returns to initial temperature after every process step, dT=0, and the change in available grease energy is

dA_{b}=θdM (Equation

At breakdown equilibrium, dA|_{T,M,m}=0 and every process energy term vanishes, from which equilibrium values of grease properties T, M, m can be evaluated.

After shearing work and heat sources are removed, the grease cools to surrounding temperature and the microstructure rebuilds. The resulting Helmholtz energy regained from this relaxation is governed by

dAᵣ=dAᵣₑᵥ−CdT (Equation

where the cooling process −CdT can be evaluated similar to the heating process. Energy regained from microstructure rebuilding dAᵣₑᵥ, the reversible component of the energy change during breakdown, can be estimated from stress values at recovery start and end states, or from existing models like Maxwell's shear stress relaxation equation. During recovery dAᵣ≥0, verified by dT≤0 and dAᵣₑᵥ≥0. After relaxation is recovery equilibrium where dA|_{T}=0, determined experimentally as dT→0. The Helmholtz formulation requires only work interactions, and links entropy production and temperature change.

Entropy generation from equation (Equation 1.34) is

$$δ {S}_{b}^{′} = \frac{SdT}{T} + \frac{θ dM}{T} + \frac{μ {dN}^{′}}{T} \qquad ( Equation$$

Substituting heating, shearing and oxidation terms,

$$δ {S}_{b}^{′} = \frac{CdT}{T} + \frac{θ dM}{T} + \frac{μ dm}{{M}_{m} T} \qquad ( Equation$$

which suggests

S′=S′(T,M,m) (Equation

Equation (Equation 3.108) accumulates entropy generation of three simultaneous independent processes, and can be used for open-systems. For the more common shearing and heating only combinations for a closed system,

$$δ {S}_{b}^{′} = \frac{CdT}{T} + \frac{θ dM}{T} \qquad ( Equation$$

When work and heat sources are removed, for the relaxation process,

$$δ {S}_{b}^{′} = \frac{CdT}{T} + \frac{{dA}_{rev}}{T} ( relaxation ) \qquad ( Equation 3.111 )$$

At recovery equilibrium (from beginning of one iteration to the next), entropy generation

$$\begin{matrix}δ {S}^{′} = δ {S}_{b}^{′} + δ {S}_{r}^{′} & ( Equation \\ δ {S}^{′} = {[ \frac{CdT}{T} + \frac{θ dM}{T} + \frac{μ dm}{{M}_{m} T} ]}_{b} + {[ \frac{CdT}{T} + \frac{{dA}_{rev}}{T} ]}_{r} & ( Equation\end{matrix}$$

If the grease cools to initial temperature after shearing, the thermal components from breakdown and recovery cancel out. And without oxidation, equation (Equation 3.113) becomes

$$δ {S}^{′} = {[ \frac{θ dM}{T} ]}_{b} + {[ \frac{{dA}_{rev}}{T} ]}_{r} \qquad ( Equation$$

Equation (Equation 3.111) suggests that during relaxation, thermal entropy, the first term on the right hand side (RHS) reduces entropy production as dT≤0. If the breakdown process proceeds much faster than the spontaneous recovery process, as in most regular-use applications,

δS_{b}′>>δSᵣ′ (Equation

giving entropy production in grease undergoing shear below drop point,

$$δ {S}^{′} = \frac{CdT}{T} + \frac{θ dM}{T} \qquad ( Equation$$

The formulations above can be solved numerically or integrated from known functions.

Active Shearing Versus Relaxation

To compare significance of active grease shearing and the subsequent recovery process in thermodynamic formulations, from equation (Equation 3.114),

$$δ {S}_{b}^{′} = {[ \frac{θ dM}{T} ]}_{b} ; δ {S}_{r}^{′} = {[ \frac{{dA}_{rev}}{T} ]}_{r} \qquad ( Equation$$

The shear work is \dot{γ}_{V}τ, where \dot{γ}_{V}=V\dot{γ} is the product of grease volume and shear rate, and i is the shear stress. Equation (Equation 3.117) becomes

$${\dot{S}}_{b}^{′} = {[ \frac{{\dot{γ}}_{V} τ}{T} ]}_{b} ; {\dot{S}}_{r}^{′} = {[ \frac{{\dot{γ}}_{V} {τ}_{rev}}{T} ]}_{r} \qquad ( Equation$$

where τᵣₑᵥ is the reversible shear stress in the grease, recovered during relaxation. Grease in machine lubrication is sheared at shear rates between 10³ and 10⁷ s⁻¹. Recalling Sisko's model from Table 3.1, steady state shear stress during breakdown can be estimated as

τ=K\dot{γ}ⁿ+η\dot{γ} (Equation

Stress relaxation after constant shear is given by Maxwell's exponential response

$${τ}_{rev} ( t ) = η {\dot{γ}}_{0} exp ( - \frac{t}{{t}_{c}} ) \qquad ( Equation$$

where relaxation time t_{c}=η/G is a material-dependent characteristic and \dot{γ}₀=\dot{γ} is the constant shear rate. Grease sheared continuously for 12 hours will not recover fully and may take several weeks (or months) to relax. If shearing is followed by overnight rest (a relaxation observation time t=0.5 day), using t_{c}=40 days, equation (Equation 3.120) gives τ=0.988η\dot{γ}₀. Direct comparison between equations (Equation 3.119) and (Equation 3.120) shows about 1% recovery.

The above is a liberal estimate as experimental results indicate much less recovery in shear stress, in which after 1 hour of shearing lithium grease at 8.1 s⁻¹, a relaxation time of 24 hours gave very minimal shear stress recovery in three different greases tested.

Grease recovery is even much lower at high shear rates. Shearing at over 1000 s⁻¹ for several hours, as in many applications, takes grease close to its asymptotic steady state (engineering yield) shear stress value. Here recovery is negligible. If grease is iteratively sheared, recovery ability further degrades and relaxation entropy diminishes with iterations, making equation (Equation 3.115) increasingly true.

Considering the above, the relaxation term is negligible.

Infinitesimal Model—Heat-Only Analysis

Assumptions:
1. The system is the grease sample enclosed in the bearing housing (the boundary).
2. System is closed (grease sealed in bearing prevents mass flow).
3. Heat transfers between grease and immediate surroundings via free convection.
4. The system is at equilibrium before and after operation.

From equation (Equation 1.40), the viscous dissipation (heat generation from shearing)

δE′=CdT−δQ (Equation

From equation (Equation 1.41), entropy generation in the grease sample from viscous dissipation

$${δS}^{′} = \frac{CdT}{T} - \frac{δ Q}{T} \qquad ( Equation$$

where the RHS terms are grease thermal energy storage and heat transfer entropies respectively. The heat storage term is equivalent to the thermal energy term in the Helmholtz formulation in equation (Equation 3.102). Heat transfer out of the grease is negative, according to Clausius. Rate of heat transfer out of the grease via equation (Equation 1.42)

\dot{Q}=ΔT/Rₜ (Equation

is the ratio of the difference between grease and ambient temperatures DT to the thermal resistance Rₜ in between. For a 1-dimensional lumped-capacity heat transfer model, thermal resistance including conduction through the grease cup wall of thickness Δx and free convection with the surroundings is given by

$${R}_{t} = ( \frac{1}{{h}_{air} {A}_{s}} ) + ( \frac{Δ x}{{kA}_{s}} ) \qquad ( Equation$$

where Aₛ is the cup surface area, hₐᵢᵣ the average heat transfer coefficient of air (or surrounding medium) and k the thermal conductivity of cup material. The heat formulation equation (Equation 1.41) applies at every instant of the grease life cycle, including relaxation after shearing, during which the first RHS term is negative as dT≤0, reducing entropy change during relaxation, independently verifying equation (Equation 3.115).

The heat capacity of grease (or the heat transfer coefficient of air, if heat capacity is known) can be estimated from the heat transfer balance for relaxation process, giving

$$C = \frac{δ Q}{dT} ( relaxation ) \qquad ( Equation 3.125 )$$

Experimental Model—Work and Heat

Here rate forms are presented. Parameters can be directly measured to determine energy changes and entropy production.

Control Parameters:
1. The grease sample is a closed system.
2. Heat transfers with the surroundings via natural convection.

Rewriting equations (Equation 3.102) and (Equation 3.108) in rate form,

$$\begin{matrix}\dot{A} = - C \dot{T} - M ω + \frac{μ}{{M}_{m}} \dot{m} & ( Equation \\ {\dot{S}}^{′} = \frac{C \dot{T}}{T} + \frac{M ω}{T} + \frac{μ \dot{m}}{{M}_{m} T} & ( Equation\end{matrix}$$

The rate of irreversible entropy production in the grease undergoing mechanical, thermal and chemical interactions is the sum of the individual rates of work inputs and process energies divided by the temperature at the heat exchange boundary. Entropy production during the initial transient response from process start is given by the rate form of (Equation 3.110)

$${\dot{S}}^{′} = \frac{C \dot{T}}{T} + \frac{M ω}{T} \qquad ( Equation$$

where the oxidation term was dropped, due to low initial temperatures. If thermal equilibrium is reached below the drop point as required by most applications, the first RHS term eventually vanishes to give the steady state entropy generation

$${\dot{S}}^{′} = \frac{M ω}{T} \qquad ( Equation$$

To obtain total entropy generation during breakdown (subscript b), contributions from heat and shear (equation (Equation 3.128)) give

$${S}_{b}^{′} = {\int }_{{t}_{0}}^{{t}_{b}} \frac{C \dot{T}}{T} dt + {\int }_{{t}_{0}}^{{t}_{b}} \frac{M ω}{T} dt \qquad ( Equation$$

With negligible recovery after long-duration shearing at high shear rate, the relaxation process has been dropped. Using heat generation entropy from equation (Equation 1.41),

$$\dot{{S}^{′}} = \frac{C \dot{T}}{T} - \frac{\dot{Q}}{T} \qquad ( Equation$$

Total entropy generation,

$${S}_{b}^{′} = {\int }_{{t}_{0}}^{{t}_{b}} \frac{C \dot{T}}{T} dt - {\int }_{{t}_{0}}^{{t}_{b}} \frac{\dot{Q}}{T} dt \qquad ( Equation$$

Cycle Analysis

Grease is repeatedly sheared and relaxed, hence an equilibrium analysis using initial and final states of each iteration can be performed via equations (Equation 3.130) and (Equation 3.132). Extending equation (Equation 3.130) to include oxidation, accumulated entropy production after N iterations (number of times grease sample is sheared),

$${S}_{total}^{′} = {\sum }_{N} ( {\int }_{Δ {t}_{N}} \frac{C \dot{T}}{T} dt + {\int }_{Δ {t}_{N}} \frac{M ω}{T} dt + {\int }_{Δ {t}_{N}} \frac{μ \dot{m}}{{M}_{m} T} dt ) \qquad ( Equation$$

where Δt_{N} is the time duration of the Nth iteration.

Similarly, via heat,

$${S}_{total}^{′} = {\sum }_{N} ( {\int }_{Δ {t}_{N}} \frac{C \dot{T}}{T} dt - {\int }_{Δ {t}_{N}} \frac{\dot{Q}}{T} dt ) \qquad ( Equation$$

Degradation-Entropy Generation (DEG) Analysis

DEG formulations are applied to grease degradation. Both thermodynamic and heat balance approaches give similar forms of irreversible entropy production, a quotient of process energy to temperature for each active process.

The maximum frictional energy in grease, similar to those obtained for constant shear rate, is

\dot{A}=\dot{γ}_{V}τ (Equation

where τ can arise from a time-dependent shear model, see Tables 3.1 and 3.2. Combining with equation (Equation 3.126),

\dot{A}=\dot{γ}_{V}τ=−C\dot{T}−Mω+μ\dot{m} (Equation

from which a time-dependent shear stress can be obtained as

$$τ ( t ) = \frac{- C \dot{T} - M ω + μ \dot{m}}{{\dot{γ}}_{V}} \qquad ( Equation$$

Identifying entropy production for active processes via equation (Equation 3.127), and applying this to the DEG theorem equation (Equation 2.53) gives

$$\frac{dw}{dt} = {B}_{T} \frac{C \dot{T}}{T} + {B}_{W} \frac{M ω}{T} + \frac{{B}_{m}}{{M}_{m}} \frac{μ \dot{m}}{T} \qquad ( Equation$$

For entropy generation heat analysis, equation (Equation 3.131) and the DEG theorem give

$$\frac{dw}{dt} = {B}_{T} \frac{C \dot{T}}{T} - {B}_{Q} [ \frac{( T - {T}_{∞} )}{RT} ] \qquad ( Equation$$

where B can be evaluated via appropriate measurements of tribological and/or rheological parameters, via equation (Equation 2.54)

$${B}_{i} = \frac{\partial w}{\partial {S}_{i}^{′}} \qquad ( Equation$$

the ratio of the slope of the rate of w to the specific process entropy production rate.

Cyclic Analysis

Many grease formulations and measurements (yield stress, consistency, thixotropic index, etc.) only apply at the end of a breakdown process and/or the beginning of the next breakdown process, hence successive equilibrium measurements can be used for cyclic analysis. In iterative applications, since entropy accumulates, degradation during the Nth iteration relates to entropy production through an integral

$${w}_{N} = {B}_{T} {\int }_{{t}_{0}}^{{t}_{f}} \frac{C \dot{T}}{T} dt + {B}_{W} {\int }_{{t}_{0}}^{{t}_{f}} \frac{M ω}{T} dt + {B}_{m} {\int }_{{t}_{0}}^{{t}_{f}} \frac{μ \dot{m}}{{M}_{m} T} dt \qquad ( Equation$$

The total accumulated degradation sums over N iterations,

$${w}_{total} = {\sum }_{N} \{ {B}_{T} {\int }_{{t}_{0}}^{{t}_{f}} \frac{C \dot{T}}{T} dt + {B}_{{W}_{N}} {\int }_{{t}_{0}}^{{t}_{f}} \frac{M ω}{T} dt + {B}_{{\dot{m}}_{N}} {\int }_{{t}_{0}}^{{t}_{f}} \frac{μ \dot{m}}{{M}_{m} T} dt \} \qquad ( Equation$$

In heat generation terms from equation (Equation 3.140),

$${w}_{N} = {B}_{T} {\int }_{{t}_{0}}^{{t}_{b}} \frac{C \dot{T}}{T} dt - {B}_{Q} {\int }_{{t}_{0}}^{tf} ( \frac{T - {T}_{∞}}{{R}_{t} T} ) dt \qquad ( Equation$$

Using Shear Stress as Degradation Measure

With shear stress t as degradation parameter, equation (Equation 3.141) becomes

$${τ}_{N} = {B}_{T} {\int }_{{t}_{0}}^{{t}_{f}} \frac{C \dot{T}}{T} dt + {B}_{W} {\int }_{{t}_{0}}^{{t}_{f}} \frac{M ω}{T} dt + {B}_{m} {\int }_{{t}_{0}}^{{t}_{f}} \frac{μ \dot{m}}{{M}_{m} T} dt \qquad ( Equation$$

where the Helmholtz-shear stress coefficients

$${B}_{T} = \frac{\partial τ}{\partial {S}_{T}^{′}} ; {B}_{W} = \frac{\partial τ}{\partial {S}_{W}^{′}} ; {B}_{m} = \frac{\partial τ}{\partial {S}_{m}^{′}} \qquad ( Equation$$

pertain to thermal entropy

$${S}_{T}^{′} = \int \frac{C \dot{T}}{T} dt ,$$

shear entropy

$${S}_{W}^{′} = \int \frac{M ω}{T} dt$$

and oxidation entropy

$${S}_{m}^{′} = \int \frac{μ \dot{m}}{{M}_{m} T} dt$$

respectively. Summarily via equation (Equation 3.143),

$${τ}_{N} = {B}_{T} {\int }_{{t}_{0}}^{{t}_{b}} \frac{C \dot{T}}{T} dt - {B}_{Q} {\int }_{{t}_{0}}^{{t}_{f}} ( \frac{T - {T}_{∞}}{{R}_{t} T} ) dt \qquad ( Equation$$

with heat generation-shear stress coefficients

$${B}_{T} = \frac{\partial τ}{\partial {S}_{T}^{′}} ; {B}_{Q} = \frac{\partial τ}{\partial {S}_{Q}^{′}} \qquad ( Equation$$

pertain to entropies from heat storage and heat transfer respectively.

DEG Coefficients from Existing Models

Mechanical Degradation Coefficient B_{W}

Rewriting equation (Equation 3.138) for shearing only in terms of the frictional energy formulation,

$$\frac{dw}{dt} = {B}_{w} \frac{M ω}{T} = {B}_{W} \frac{{\dot{γ}}_{V} τ}{T} \qquad ( Equation$$

From Maxwell's model,

$$\frac{d τ}{dt} = G ( \dot{γ} - \frac{τ}{η} ) \qquad ( Equation$$

Equating the RHS of equations (Equation 3.148) and (Equation 3.149) and solving for B_{W} give

$${B}_{W} = \frac{TG}{{\dot{γ}}_{V} τ} ( \dot{γ} - \frac{τ}{η} ) \qquad ( Equation$$

shown in the first row, last column of Table 3.3. In like manner, Tables 3.3 and 3.4 contain other B coefficients derived from the non-DEG models in Tables 3.1 and 3.2.

Thermal Degradation Coefficient B_{T}

Rewriting equation (Equation 3.141) for degradation from heating only,

$${w}_{N} = {B}_{{T}_{N}} {\int }_{Δ {t}_{N}} \frac{C \dot{T}}{T} \qquad ( Equation$$

which for constant heat capacity approximates to

$${w}_{N} = {B}_{{T}_{N}} Cln \frac{T}{{T}_{0}} \qquad ( Equation$$

Chemical Degradation Coefficient Bₘ

Rewriting equation (Equation 3.138) for degradation from shearing only,

$$\frac{{dw}_{m}}{dt} = \frac{{B}_{m}}{{M}_{m}} \frac{μ \dot{m}}{T} \qquad ( Equation$$

via Rhee's model with percent degradation equal to e^{-kt},

$$\frac{{dw}_{m}}{dt} = - {w}_{0} k exp ( - kt ) \qquad ( Equation$$

TABLE 3.3
Table [symbol] degradation coefficients derived from prior models.

| Degradation | Degradation | | |
|---|---|---|---|
| Mechanism | Measure | Published Model | Degradation Coefficient |
| Mechanical | Shear stress | $Maxwell \text{:} \frac{d τ}{dt} = G ( \dot{γ} - \frac{τ}{η} )$ | ${B}_{M} = \frac{TG}{{\dot{γ}}_{V} τ} ( \dot{γ} - \frac{τ}{η} )$ |
| | Shear strain | $Kevin \text{-} Voigt \text{:} \frac{d γ}{dt} = \frac{τ - γ G}{η}$ | ${B}_{M} = \frac{T}{{\dot{γ}}_{V} τη} ( τ - γ G )$ |
| | Viscosity | $Mewis \text{:} \frac{d η}{dt} = {k [ {η}_{e} ( {\dot{γ}}_{1} ) - η ]}^{n}$ | ${B}_{M} = \frac{{kT [ {η}_{e} - η ]}^{n}}{{\dot{γ}}_{V} τ}$ |
| | | $Cross \text{:} \frac{dN}{dt} = {k}_{2} P - ( {k}_{0} + {k}_{1} {{\dot{γ}}_{1}}^{m} ) N$ | ${B}_{M} = \frac{T [ {k}_{2} P - ( {k}_{0} + {k}_{1} {γ}^{m} ) ]}{{\dot{γ}}_{V} τ}$ |
| | Yield stress | H-B: τ = τ_{y} +K\dot{γ}ⁿ | ${B}_{{M}_{N}} = \frac{{T τ}_{y}}{{\dot{γ}}_{V} {\int }_{{t}_{N}} {τ}_{t}}$ |
| | Consistency | Pen = (32.5 * 10^{31.7}τ_{y}^{-.315)} Pen = 4.162S_{g} + 0.071 | ${B}_{{M}_{N}} = \frac{T ( 32.5 * {10}^{31.7} {{τ}_{y}}^{- .315} )}{{\dot{γ}}_{V} {\int }_{{t}_{N}} {τ}_{t}} {B}_{{M}_{N}} = 4.162$ |
| | Thixotropic Index | $TI = \frac{{η}_{s}}{{η}_{10 s}} = \frac{10 {( τ - γ G )}_{s}}{{( τ - γ G )}_{10 s}}$ | ${B}_{{τ}_{N}} = \frac{10 T {τ}_{s}}{{\dot{γ}}_{V} {τ}_{10 s} {\int }_{{t}_{N}} {τ}_{t}}$ |

TABLE 3.4
Table of degradation coefficients derived from prior models (continued from
Table 3.3). Grease molecular mass Mₘ influences secondary bonding between chains,
hence lubricating properties. Viscosity can be related to Mₘ as $η = \frac{{R}^{2} {N}_{φ} A}{36 M } {\int }_{t} .$

| Degradation | Degradation | | |
|---|---|---|---|
| Mechanism | Measure | Published Model | Degradation Coefficient |
| Thermal | Yield stress | $\frac{{τ}_{y}}{{τ}_{y 0}} = exp [ ( \frac{{T}_{0} - T}{b} ) ln 2 ]$ | ${B}_{{T}_{N}} = \frac{{τ}_{y 0} exp [ \frac{{T}_{0} - T}{b} ln 2 ]}{Cln [ T / {T}_{0} ]}$ |
| | Viscosity | $η = {η}_{0} [ exp ( \frac{{E}_{a}}{RT} ) ]$ | ${B}_{{T}_{N}} = \frac{{η}_{0} [ exp ( \frac{{E}_{a}}{RT} ) ]}{Cln [ T / {T}_{0} ]}$ |
| Chemical | Shear stress | $\frac{d τ}{dt} = - {τ}_{0} kexp ( - kt )$ | ${B}_{m} = \frac{- {τ}_{0} kM ° Texp ( - kt )}{μ \dot{m}}$ |
| | Viscosity | $\frac{d η}{dt} = - {η}_{0} kexp ( - kt )$ | ${B}_{m} = \frac{- {η}_{0} kM ° Texp ( - kt )}{μ \dot{m}}$ |
| | Mass | $\frac{d m}{dt} = - {m}_{0} kexp ( - kt )$ | ${B}_{m} = \frac{- {m}_{0} kM ° Texp ( - kt )}{μ \dot{m}} * {B}_{m} = \frac{{M}_{m} T}{μ}$ |

Combining formulations renders a degradation model with coefficients—calibrated via the pre-existing models in Tables 3.3 and 3.4—that weigh influence of energy changes of individual dissipative processes. Substituting the degradation coefficients from the viscosity rows of Table 3.3 and 3.4 into equation (Equation 3.138)—note the Mewis model selected for mechanical—yields rate of degradation gauged with viscosity

$$\frac{d η}{dt} = {[ \frac{{η}_{0} [ exp ( \frac{{E}_{a}}{RT} ) ]}{ln ( T / {T}_{0} )} ]}_{0} \frac{\dot{T}}{T} + {[ \frac{{kT ( {η}_{e} - η )}^{n}}{τ} ]}_{0} \frac{τ}{T} + {[ \frac{- {η}_{0} {kM}^{o} T exp ( - kt )}{\dot{m}} ]}_{0} \frac{\dot{m}}{T} \qquad ( Equation$$

for constant C, \dot{γ}_{V} and μ, which divided out. Similarly, degradation rate via shear stress

$$\frac{d τ}{dt} = {[ \frac{{τ}_{y 0} exp [ \frac{{T}_{0} - T}{b} ln 2 ]}{ln [ T / {T}_{0} ]} ]}_{0} \frac{\dot{T}}{T} + {[ \frac{TG}{τ} ( \dot{γ} - \frac{τ}{η} ) ]}_{0} \frac{τ}{T} + {[ \frac{- {τ}_{0} {kM}^{o} T exp ( - kt )}{\dot{m}} ]}_{0} \frac{\dot{m}}{T} \qquad ( Equation$$

The terms in square brackets can be evaluated from prior properties or models of grease, or measured on samples. In service, the coefficients weigh influence of individual dissipative processes. In equations (Equation 3.155) and (Equation 3.156), only changing values of temperature, shear stress and mass need be monitored to determine degradation rate.

Comparison to Existing Energy Models

Kuhn's frictional energy density at constant shear rate in equation (Equation 3.81)

eᵣₕ=\dot{γ}∫ₜ₀^{tf}τ(t)dt

is equivalent to the integral of the rate form of equation (Equation 3.135) divided by volume,

$${A}_{V} = \frac{A}{V} = \dot{γ} {\int }_{{t}_{N}} τ dt \qquad ( Equation$$

which gives the Helmholtz energy density for a system undergoing isothermal constant-rate shearing work only. Kuhn's entropy-based formulation in equation (Equation 3.85)

eᵣₕ=T_{f}(ρₒᵤₜsₒᵤₜ)−T_{f}(\dot{m}ᵢₙ\dot{s}ᵢₙ−S_{Q})/Vₒᵤₜ

is analogous to equation (Equation 3.108) for an open system per volume, rearranged as

$${dA}_{V} = γ d ρ = \frac{T}{V} δ {S}_{b}^{′} + \frac{μ}{{VM}_{m}} d m + \frac{C}{V} dT \qquad ( Equation$$

Equation (Equation 3.85) uses specific entropy transfer by mass and heat, equivalent at steady state to the irreversible entropy formulation above. Kuhn's experimental data showed a drop in energy density with increase in specific entropy out of the system, and proportionality between structural degradation and entropy transfer.

Khonsari et al's entropy production in equation (Equation 3.92), equivalent to equation (Equation 3.116) for a unit volume without significant thermal energy effects, as accumulated entropy generation becomes

$${S}_{V}^{′} = {\int }_{{t}_{0}}^{{t}_{b}} \frac{\dot{γ} τ}{T} dt \qquad ( Equation$$

Comparing their shear stress-based degradation parameter

$$∝ = \frac{d τ *}{{dS}_{g}} \qquad ( Equation$$

to the mechanical shearing degradation model in equation (Equation 3.148)

$$\frac{dw}{dt} = {B}_{W} \frac{{\dot{γ}}_{V} τ}{T} \qquad ( Equation$$

gives B_{W}=∝. Using experimental measurements, Khonsari et al showed ∝ constant throughout the shearing process. This parameter was used in deriving equations (Equation 3.93) and (Equation 3.94) for mechanical shearing.

As mentioned, oxidation was not included in any existing energy model. In equations (Equation 3.155) and (Equation 3.156), the last term models the oxidation process.

Grease Experiments

ASTM standards allow slight modifications to the apparatus/setup, provided the modified work shows the expected trend in actual service and an appropriate definition of observed change in performance measure, e.g. consistency. Here, mechanical and thermal degradation experiments were performed to verify analyses and evaluate degradation coefficients. Oxidation experiments were not performed due to the expensive equipment. However, experimental results, if available, can be applied to grease degradation using the same approach proposed for mechanical and thermal interactions.

Choice of measurement parameters depends directly on degradation measure, prevalent degradation mechanism, availability, accuracy and convenience of measurement methods. Two types of measurements are performed:
Continuous measurement of
   ongoing work interactions for accurate determination of the process terms in the entropy production equations.
   degradation measure to determine operational degradation coefficient.
Equilibrium measurements of the degradation measure to determine total iteration degradation.
   In accordance with most industry and laboratory publications, shear stress was chosen as a degradation measure. Inconsistency in available measurement techniques due to parametric sensitivity are well documented. For degradation analysis, repeatable measurements can calibrate the degradation coefficients. This work used the engineering yield stress defined above, determined by two approaches:
For lack of a rheometer and other equipment, available empirical models were used in conjunction with measured work parameters.
Predefined asymptotic values of shear stress under constant shear rate, and post-relaxation values were used to determine yield stress.

A rotational grease shearing test was performed. Via measured speeds and torques, energy rate due to applied shear work was estimated.

The Mechanical Shearer

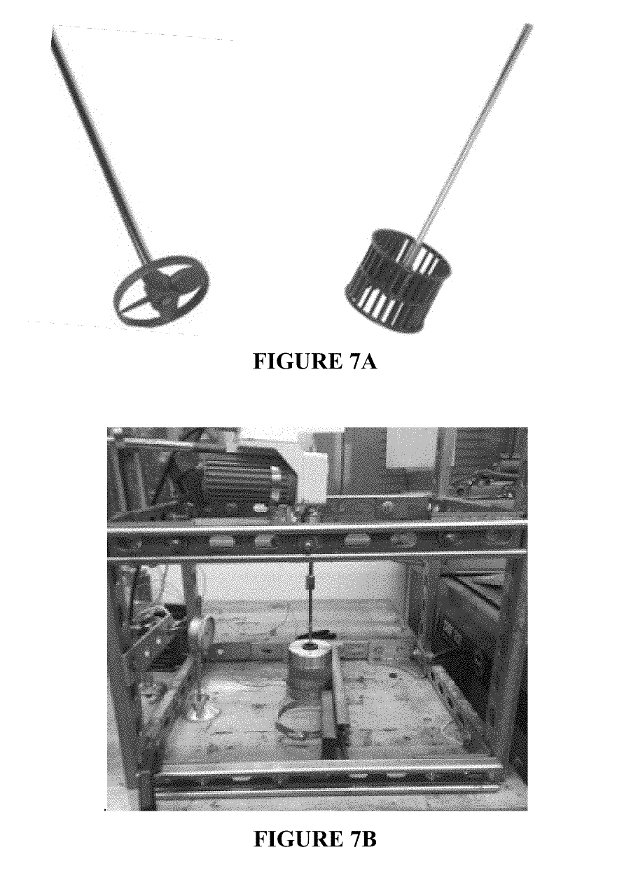

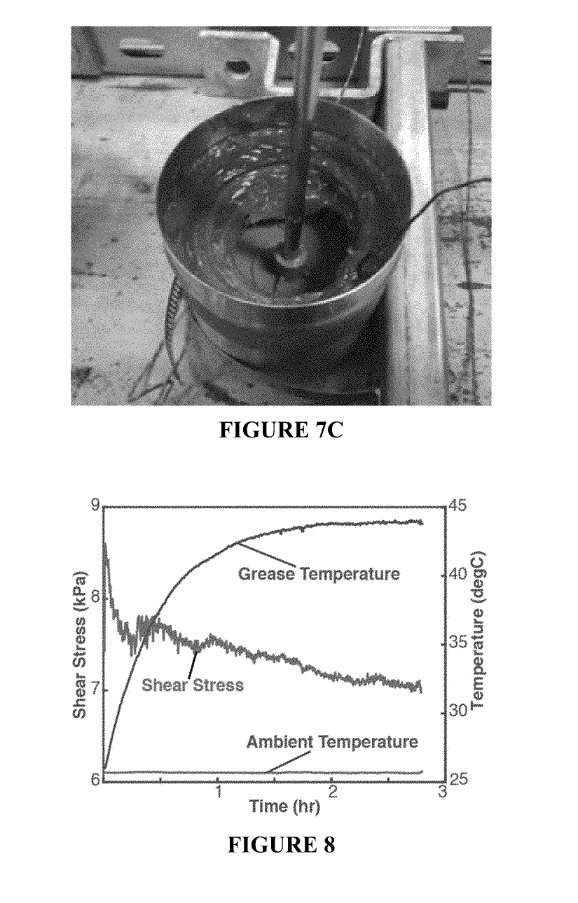

Methods for shearing grease in EHD applications and measuring loss of consistency are described in ASTM D1831 and D3527-07—Standard Test Methods for Cone Penetration of Lubricating Grease. A motorized stirrer system sheared a sample of grease in a cup continuously, and the resulting temperature rise determined heat energy absorbed by the grease. The frictional energy of the grease was determined from the flow curve (shear stress versus shear rate) or directly from the stirrer's power output (torque and rotational speed). Two paint mixers, both with impeller diameter 63.5 mm (2.5 in) and a 9.5 mm (0.375 in) shaft which extended through a hole in the cup cover to the motor (see FIGS. 7A-7C), sheared the grease.

A Fisher Scientific overhead stirrer driven by a brushless DC motor capable of keeping the set frequency to within 1% as the grease viscosity changes, powered the system and established a constant shear rate. The change in viscosity/shear stress was obtained from motor torque. A current probe with a voltage output estimated the current.

Below are empirical formulations expressing shear stress and shear rate in terms of measured torque and speed. Instantaneous power input into the grease by the shearer gives the frictional energy during shearing. Extending rate form of equation (Equation 3.99),

\dot{W}=2πω(M−M₀) (Equation

In terms of measured current and voltage,

\dot{W}=V(I−I₀)cos φ (Equation

where φ is the motor constant. Subscript 0, for values measured with the impeller rotating in air, indicates torque dissipated by the driving actuator. Hence equations (Equation 3.162) and (Equation 3.163) pertain to power dissipated in the grease. Shear rate for a stirrer is

$$\dot{γ} = \frac{4 π ω}{1 - {k}^{2}} \qquad ( Equation$$

where k=d/D, d is impeller diameter and D is grease cup diameter. Instantaneous shear stress can be obtained from

$$τ = \frac{M}{2 π {r}^{2} l} \qquad ( Equation$$

to give the time-dependent viscosity

$$η = \frac{τ}{\dot{γ}} \qquad ( Equation$$

The above equations are used due to the simplicity of experiment used. Other empirical formulations based on more accurate rheometric measurements are available, including the widely used Metzner-Otto formulation for grease shearing and Nguyen's shear rate equation.

National Instruments CompactRIO device and Labview software allowed continuous recording of process parameters for both the grease shearer.

Grease Selection

Grease composition varies widely. Common commercial greases are calcium and lithium-soap greases. Lithium greases are more suited to extreme temperature and pressure. Calcium greases find wide use in water-resistant, low to medium-duty applications. Water resistance and good thermal and mechanical stability enable wide use of lithium greases in heavy-duty applications. Two types of lithium greases were used:
Valvoline multi-purpose NLGI 2 grease and
Aeroshell 14 aircraft NLGI 4 grease.

Procedure

The shearer assembly was set on a test rig, see FIG. 7B. The test area was ventilated, while avoiding air current in the direction of the experiment setup.
1. Installed thermocouples.
   wire thermocouple in the cup through the hole in the grease cup cover.
   another thermocouple attached to the exterior of the cup.
   a third thermocouple placed about 80 mm away from the cup with sensing electrode in the air.
2. Connected thermocouples to the CompactRIO thermocouple module.
3. Connected current probe to the CompactRIO differential analog module.
4. CompactRIO recorded time, temperatures and probe's voltage output.
5. With mixer attached to stirrer, operated stirrer at constant speeds to determine the no-load condition.
6. Following ASTM D217 recommendation, 0.5 kg of grease in cup.
7. Started data logger and recorded initial state of system for 3 mins.
8. Shearing:
   Inserted impeller in grease and operated stirrer at 1 Hz for 10 minutes, to establish the grease's pre-shear history (initial condition), and in subsequent steps estimate the equilibrium properties of grease.
   Sheared the grease at 3 Hz continuously for about 60 mins or more.
   Stopped and restarted the stirrer at 1 Hz for 10 mins to estimate post-breakdown equilibrium properties.
9. With data logger still running, allowed grease samples to cool to surrounding temperature.
10. Stopped data logger.
11. Repeated steps 8-11 continuously until the grease sample degraded, indicated by a drop in yield stress value below required values for a particular operation.

Results and Data Analysis

Using equations for energy loss in grease and entropy production via work and thermal energy changes, the columns in Tables 3.5 and 3.6 were evaluated. Details of data processing during an iteration of grease shearing will be presented next, followed by a summary of results. Observed trends in the data will be discussed. Except for temperature changes, integrals were evaluated using the trapezoidal rule on data over time increment Dt. Time-based data of shear stress in grease as a function of torque M (via equation (Equation 3.165)), grease temperature T and ambient temperature Tₐ were recorded as grease was sheared. Sampling at 0.1 Hz rendered the time interval between data points Δt=10 s for all data.

The results presented here are for Aeroshell 14 aircraft lithium grease, NLGI 4.

Constants

Appropriate constants required in the above formulations include:

Estimated heat transfer coefficient of still ambient air, hₐᵢᵣ=4 W/m² K.

Thermal conductivity of grease cup k=15.1 W/m-K.

Cup surface area A_{S}=0.026 m².

Grease sample mass m=0.5 kg.

Cup wall thickness Δx=0.001 m.

Specific heat capacity of grease used

$${C}_{grease} = 381 \frac{J}{kg} K$$

Tables 3.5 and 3.6 were populated using the above equations and template, from which a sample dataset from iteration 4 is broken down. Each iteration N was a separate data collection test on the same grease sample. Energy loss and heat transfer out of the grease are represented on negative axes. The format here was used throughout the results section.

### Helmholtz Thermodynamic Analysis (Maximum Work)

Grease shearing, carried out at irregular intervals with different iteration durations, shows robustness of the DEG theorem in naturally occurring conditions. For brevity, iteration 4 is used to break down observed trends in grease shearing.

TABLE 3.5
Processed parameters for lithium grease NLGI 4 showing consistent DEG coefficients
for shearing iterations of different durations (shear rate = 123.4 s⁻¹).
Each iteration N was a different data collection experiment on the same grease sample.

| | | | | | | | | | B_{W} | B_{T} | | |
|---|---|---|---|---|---|---|---|---|---|---|---|---|
| | Duration | τ | A_{N}\|W | | ΔA_{N} | S′_{N}\|W | S′_{N}\|T | S′_{N} | MPa- | MPa- | Residual | GoF |
| N | (min) | Pa-s | J | ΔA_{N}\|T | kJ | J/K | J/K | J/K | s/J/K | s/J/K | MPa-s | R² |
| 1 | 61 | 29.7 | −183.7 | −6.1 | −189.7 | 595.2 | 19.8 | 615.0 | 51.4 | −39.8 | −71.8 | 1 |
| 2 | 55 | 25.8 | −159.7 | −5.9 | −165.5 | 518.1 | 19.2 | 537.3 | 51.1 | −32.6 | −46.8 | 1 |
| 3 | 40 | 18.3 | −112.9 | −5.1 | −118.0 | 367.9 | 16.6 | 384.5 | 50.5 | −20.7 | −28.3 | 1 |
| 4 | 168 | 74.3 | −459.8 | −6.8 | −466.6 | 1465.2 | 22.1 | 1487.3 | 51.3 | −34.5 | −26.0 | 1 |
| 5 | 79 | 35.8 | −221.6 | −6.2 | −227.8 | 718.1 | 10.2 | 728.3 | 50.9 | −34.5 | −25.7 | 1 |

FIG. 8 shows three hours of grease shearing at constant shear rate 123.4 s⁻¹. Past static inertia, the shear stress shows an initial transient region in which microstructure quickly breaks down. After the first half hour, the instantaneous shear stress diminishes at a reduced rate under constant-rate shearing, as a result of grease thixotropy. An initial steep rise in grease temperature during the first hour eventually levels off exponentially towards a steady state, attributed to the initially high apparent viscosity dissipating more heat. Noise in the stress data is primarily from shearer movement.

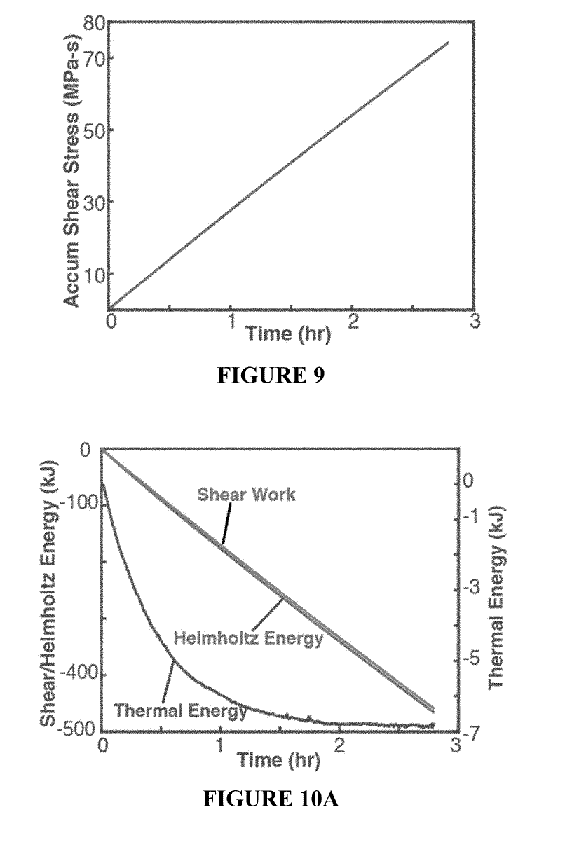

For a process occurring from t₀ to t_{f}, accumulated shear stress (FIG. 9)

$${τ}_{N} = {\int }_{{t}_{0}}^{{t}_{f}} τ ( t ) dt \approx {\sum }_{1}^{n} [ \frac{{τ}_{n} + {τ}_{n - 1}}{2} ] Δ t \qquad ( Equation$$

where n=1, 2, 3, . . . is a vector index corresponding to time t₁, t₂, t₃, etc and Δt=tₙ−tₙ₋₁. The time duration, column 2, for different iterations N was uncontrolled and irregular, to show the robustness of the DEG approach at every instant. The accumulated shear stress, column 3, in the grease increased during shearing, hence positive (FIG. 9). The unit MPa-s which measures stress accumulated over time is not to be confused with viscosity.

Total shear work during operation from equation (Equation 3.162), column 4

$${A}_{N} \vert W = {\int }_{{t}_{0}}^{{t}_{f}} 2 π ω ( M - {M}_{0} ) dt = {\sum }_{1}^{n} \{ [ \frac{2 π ω ( {M}_{n} + {M}_{n - 1} - {M}_{0} )}{2} ] Δ t \} \qquad ( Equation$$

where index n=1, 2, 3, . . . corresponds to t₁, t₂, t₃, etc. and Δt=tₙ−tₙ₋₁.

Thermal energy, column 5

ΔA_{N}|T=∫ₜ_{₀}^{t}^{_{f}}C\dot{T}dt=C(T_{f}−T₀) (Equation

Accumulated Helmholtz energy loss during operation in column 6

ΔA_{N}=−A_{N}|W−ΔA_{N}|T (Equation

To monitor and plot changes in thermal energy at times t₁, t₂, t₃, . . . , integrals were decomposed. The first term, from t₀ to t₁,

ΔA₁|T=∫ₜ_{₀}^{t}^{₁}C\dot{T}dt=C(T₁−T₀) (Equation

and the nth term

ΔAₙ|T=∫ₜ_{₀}^{t}^{ₙ}C\dot{T}dt=ΔA₁|T+ . . . +ΔAₙ₋₁|T+C(Tₙ−Tₙ₋₁) (Equation

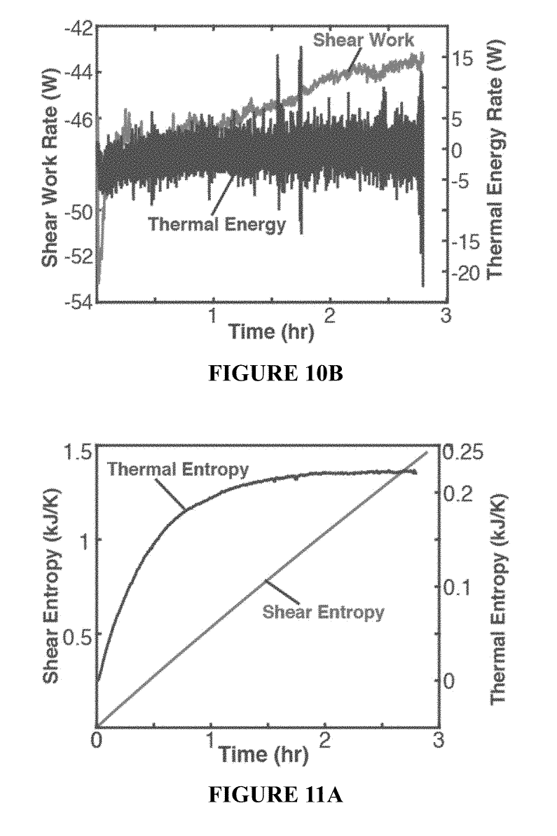

The shear component of the total Helmholtz energy, which represents the real useful work, linearly decreases during shearing (FIGS. 10A-10B). The thermal component is the change in the grease's available Helmholtz energy due to thermal energy changes during shearing. With viscous heating dominating other thermal mechanisms, including free convection to the environment (especially at high shear rates), the thermal component increases in magnitude, and thus has a negative effect on available energy (FIGS. 10A-10B). The thermal energy change is consistent throughout Table 3.5 as grease tends to a steady temperature over time at a constant shear rate. The grease's Helmholtz (total) energy decreases during shearing (FIGS. 10A-10B). The shear work is most significant during grease shearing, with a value that directly depends on shear rate. The contribution from the thermal component, 2 orders of magnitude less than the shear contribution (Table 3.5), could be neglected. Thermal energy changes depend directly on grease composition (vis-à-vis the heat capacity) and the overall change in grease temperature during shearing.

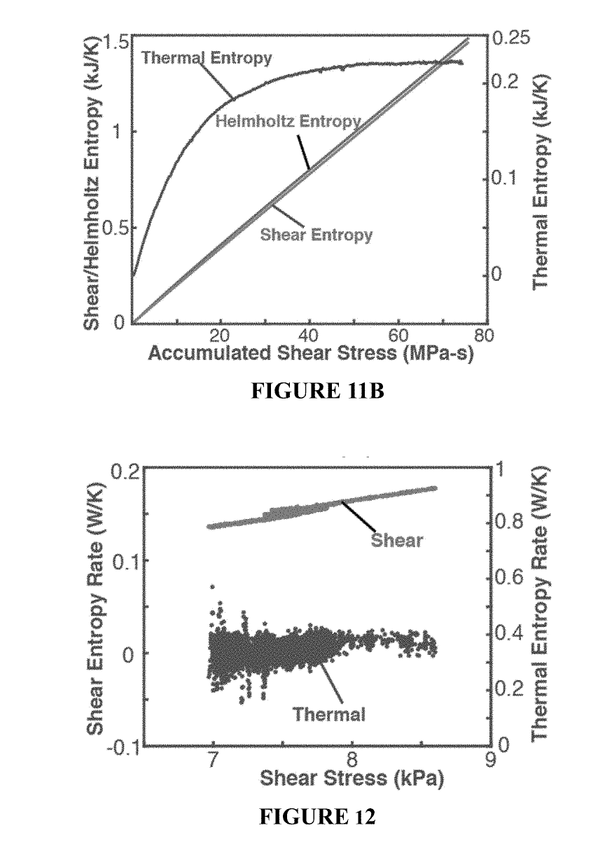

FIGS. 11A and 11B plot shear S′_{N}|W (column 7), thermal (column 8) ΔS′_{N}|T and total S′_{N} (column 9) entropies versus time and stress, calculated via S′_{N}=S′_{N}|W+ΔS′_{N}|T where

$$\begin{matrix}{S}_{N}^{′} \vert W = {\int }_{{t}_{0}}^{{t}_{f}} \frac{2 πω ( M - {M}_{0} )}{T} dt = {\sum }_{1}^{n} \{ [ \frac{2 π ω ( {M}_{n} + {M}_{n - 1} - {M}_{0} )}{{T}_{ave}} ] \frac{Δ t}{2} \} & ( Equation \\ Δ {S}_{N}^{′} \vert T = {\int }_{{t}_{0}}^{{t}_{f}} \frac{C \dot{T}}{T} dt = {\sum }_{1}^{n} [ \frac{C ( {T}_{n} - {T}_{n - 1} )}{{T}_{ave}} ] & ( Equation\end{matrix}$$

where instantaneous non-isothermal temperature for accurate determination of entropy

$${T}_{ave} = \frac{{T}_{n} + {T}_{n - 1}}{2}$$

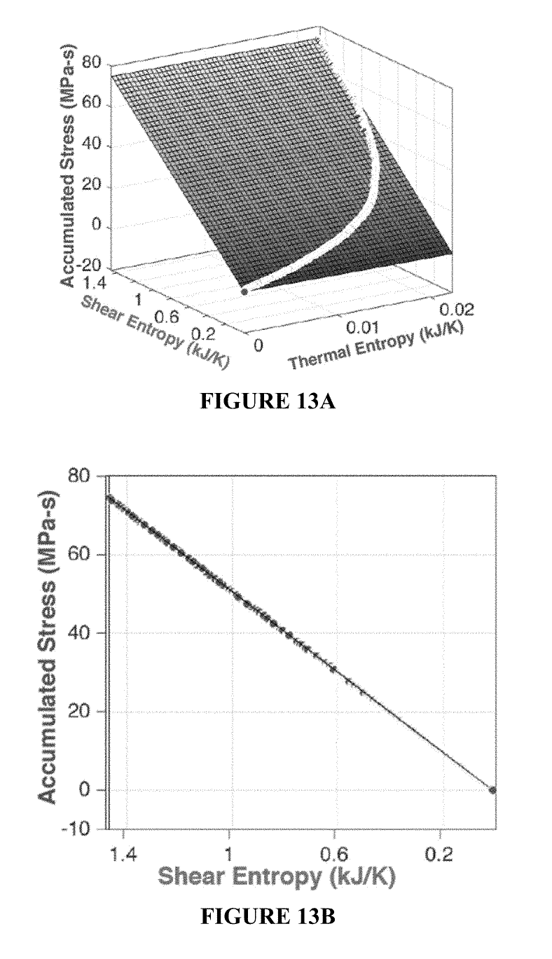

Table 3.5 and FIGS. 11A and 11B show the shear work-generated entropy. A linear relationship is observed between the shear entropy and accumulated shear/shearing, consistent with the DEG theorem. A corresponding decrease in shear entropy generation rate is observed with decrease in rate of change of shear stress in the grease shearing (FIGS. 11A-11B and FIG. 12). Thermal entropy change progresses similar to grease temperature change, like thermal energy, see FIGS. 11A-11B. With the relatively low temperature change rate, the thermal entropy change rate is also low (FIGS. 11A-11B and FIG. 12). Helmholtz (total) entropy generation during shearing from both active processes are shown in FIGS. 11A-11B. Instantaneous shear stress depends on entropy generation rate (FIG. 12). The thermal contribution to total entropy is about 1 to 2 orders of magnitude less than the shear contribution. The partial contributions better visualize in the 3D surface plot in FIGS. 13A-13B, which plot the data points (S′_{N}|W, ΔS′_{N}|T, τ). As shown by the coefficients below, the thermal entropy contributes significantly to the accumulated shear stress and unlike thermal energy, should not be neglected. The need to keep grease below its drop point for continuous shearing underscores the significance of thermal entropy. FIGS. 13A-13B show a R²=1 goodness of fit (linear dependence on 2 active processes). As shown in FIG. 13A, shearing trajectory starts from lowest corner and proceeds to apex. As shown in FIG. 13B, all points are coincident with the surface.

Degradation Coefficients Bᵢ

Shear work degradation coefficient, column 10, using shear stress x from equation (Equation 3.145)

$${B}_{W} = \frac{\partial τ}{\partial {S}_{W}^{′}} .$$

Thermal degradation coefficient, column 11, from equation (Equation 3.145)

$${B}_{T} = \frac{\partial τ}{\partial {S}_{T}^{′}} .$$

By associating data from the time instants, accumulation vectors (a series of sum of adjacent values) obtained from equations (Equation 3.173) and (Equation 3.174) were fitted to accumulated shear stress from equation (Equation 3.167) to obtain the DEG relation formulated in equation (Equation 3.144). Residual stress from each fit, column 12, is the difference between the measured shear stress and that computed via the DEG theorem,

$${τ}_{res} = τ - {\sum }_{i} {B}_{i} {S}_{i} \qquad ( Equation$$

FIGS. 13A-13B plot the iteration 4 accumulated shear stress versus entropy data in a three-dimensional space, to separate out individual entropies. FIG. 13B shows measured points on the surface, coincident with a linear 2D surface fit, hence R²=1 goodness of fit, column 13, rare for most experiments under uncontrolled conditions, especially dissipation measurements. A combined linear dependence of shear stress on both entropy components is observed.

Degradation coefficients B_{W} and B_{T}, partial derivatives of shear stress to shear and thermal entropies respectively (via the DEG theorem), were estimated as coefficients from the surface fit. FIGS. 13A-13B show the grease draws a path—its Degradation Entropy Generation (DEG) trajectory—during shearing, marked by the measured points characteristically coincident with a linear plane—its DEG surface. The DEG surface suggests a linear dependence of shear stress accumulation on both shear and thermal entropies. The 3-D space, the grease DEG domain enclosing the DEG surface characterizes the complete regime in which the particular grease can be sheared. The DEG domain, spanned by Shear Stress, Shear Entropy and Thermal Entropy can define consistent parameters for identifying desired characteristics of grease. FIGS. 13A-13B are a direct visual verification of the DEG theorem.

The DEG theorem suggests a constant B_{W} during shearing, verified in Table 3.5 with slight variation over different iterations, due to measurement inconsistencies. A lower shear coefficient B_{W} implies lower impact of shear entropy on stress accumulation. Table 3.5 shows values of B_{T} the same order as B_{W}. The lower thermal entropy values keep overall thermal degradation low from iteration to iteration. Iteration 4 data in Table 3.5 show that for a temperature rise of 20 degK, thermal entropy change is 22 J/K, and B_{T} is about the same as B_{W} with shear entropy of 1465 J/K. Grease manufacturers specify temperature ranges outside of which catastrophic degradation can occur (e.g., oil separating from thickener at drop point), as suggested by high B_{T} and recommend low shearing rates to minimize viscous heating. Many grease studies involve low-rate shearing to minimize thermal entropy during shearing, and use the isothermal assumption.

The values of B_{T} show more variation than shear counterparts, due to temperature measurement sensitivity. Low-cost thermocouples are prone to significant measurement uncertainties. Also, a fixed thermocouple with no physical interference from the rotating shearer will improve consistency of B_{T} values. A process with significant temperature changes will be less susceptible to equipment sensitivity and give more consistent B_{T} values. Measurements using rheometers and advanced temperature measurement equipment would give more representative data from which a constant pair of coefficients should be evaluated. As the overall thermal entropy here is relatively negligible, results were not considerably affected.

Heat-Only Thermodynamic Analysis

Thermal analysis-based degradation coefficients will be evaluated using grease shearing data from the mechanical shearer experiment. Heat transfer is conduction through cup wall and free convection spontaneously driven by the difference between grease and ambient temperatures.

Important Notes about the Tables and Figures

Tables and figures follow the same convention as the Helmholtz analysis. Signs indicate direction of the energy or entropy process. Plots show actual process directions. Appropriate formulations for each column of Table 3.6 are:
Column 3: Shear stress as defined in Helmholtz analysis above.
Column 4: Accumulated heat transfer out of the grease from equation (Equation 3.123),

$${Q}_{N} = - {\int }_{{t}_{0}}^{tf} ( \frac{T - {T}_{∞}}{{R}_{t}} ) dt$$

where Rₜ is given in equation (Equation 3.124).
Column 5: The heat storage term ΔE_{N} is the same as the thermal energy term in the Helmholtz analysis.
Column 6: Heat generation from equation (Equation 3.121), E′_{N}=ΔE_{N}−Q_{N}
Column 7: Entropy transfer by heat from equation (Equation 3.132)

$${S}_{N}^{′} \vert Q = - {\int }_{{t}_{0}}^{tf} ( \frac{T - {T}_{∞}}{{R}_{t} T} ) dt$$

where T is grease instantaneous temperature.
Column 8: Heat storage entropy from equation (Equation 3.132), same as the thermal entropy in Helmholtz analysis.
Column 9: Accumulated heat generation entropy, equation (Equation 3.132), S′_{N}=ΔS′_{N}|T−S′_{N}|Q.
Column 10: Shear stress-heat transfer coefficient from equation (Equation 3.147)

$${B}_{Q} = \frac{\partial τ}{\partial {S}_{Q}^{′}}$$

Column 11: Shear stress-heat storage coefficient from equation (Equation 3.147)

$${B}_{T} = \frac{\partial τ}{\partial {S}_{T}^{′}}$$

Values in the tables that follow were calculated using the above heat analysis equations with time-based data; the trapezoidal rule estimated integrals of accumulated heat transfer and heat transfer entropy.

Columns 12 and 13 show the residual from each fit (equation (Equation 3.175)) τᵣₑₛ=τ−Σᵢ BᵢSᵢ and the goodness of fit as in Helmholtz analysis.

Mechanical Shearing

TABLE 3.6
Processed parameters for lithium grease NLGI 4 showing consistent heat transfer/storage
DEG coefficients for shearing iterations of different durations (shear rate = 123.4 s⁻¹).

| | | | | | | | | | B_{Q} | B_{T} | | |
|---|---|---|---|---|---|---|---|---|---|---|---|---|
| | Duration | τ | Q_{N} | ΔE_{N} | E′_{N} | S′_{N}\|Q | ΔS′_{N}\|T | S′_{N} | MPa- | MPa- | τᵣₑₛ | GoF |
| N | (min) | MPa-s | kJ | kJ | kJ | J/K | J/K | J/K | s/J/K | s/J/K | MPa-s | R² |
| 1 | 61 | 29.7 | −3.1 | 6.1 | 9.2 | −10.1 | 19.8 | 29.9 | −1607 | 614 | 1018 | 0.9998 |
| 2 | 55 | 25.8 | −2.6 | 5.9 | 8.5 | −8.4 | 19.2 | 27.6 | −1713 | 560 | 738 | 0.9999 |
| 3 | 40 | 18.3 | −1.7 | 5.1 | 6.8 | −5.5 | 16.6 | 22.1 | −1616 | 570 | 226 | 0.9988 |
| 4 | 168 | 74.3 | −12.1 | 6.8 | 18.9 | −38.3 | 22.1 | 60.5 | −1585 | 625 | 15 | 0.9999 |
| 5 | 79 | 35.8 | −4.3 | 6.2 | 10.6 | −13.9 | 10.2 | 24.1 | −1657 | 615 | 336 | 0.9999 |

In this analysis, only temperature changes. Data from iteration 4 is given.

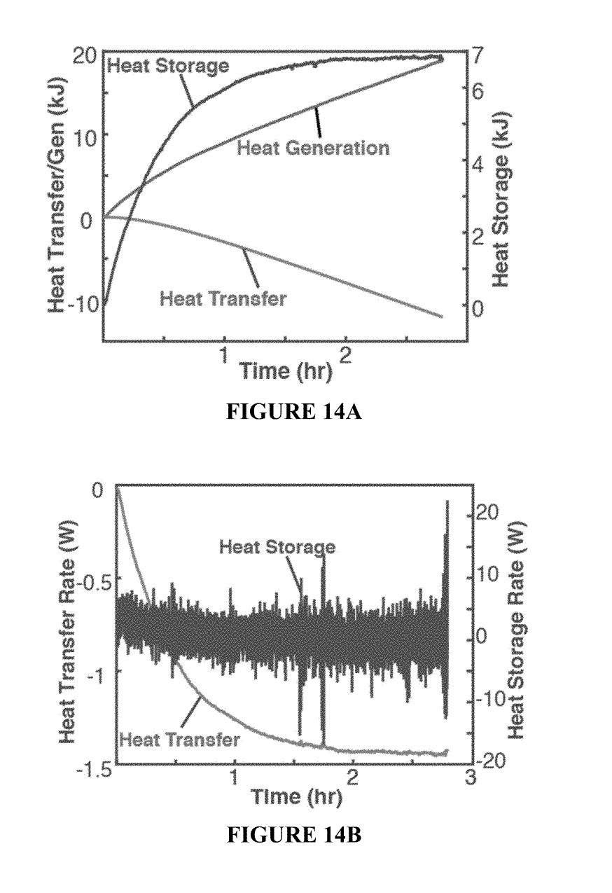

Heat transfer was predominantly out of the grease. A slightly linear trend was observed during shearing. The variation in cyclic accumulation values in Table 3.6 is consistent with the variation in ambient conditions. The heat storage component is the same as the thermal component in the Helmholtz formulations. Table 3.6 and FIGS. 14A-14B show that most of the heat generated was transferred out for long-duration constant shearing as the grease asymptotically approached steady temperature; shorter durations of shear show heat stored more significant. The measurement sensitivities discussed earlier, which apply to the heat generation values, are more significant in this approach. Note heat transfer and heat generation proceed in opposite directions (FIGS. 14A-14B).

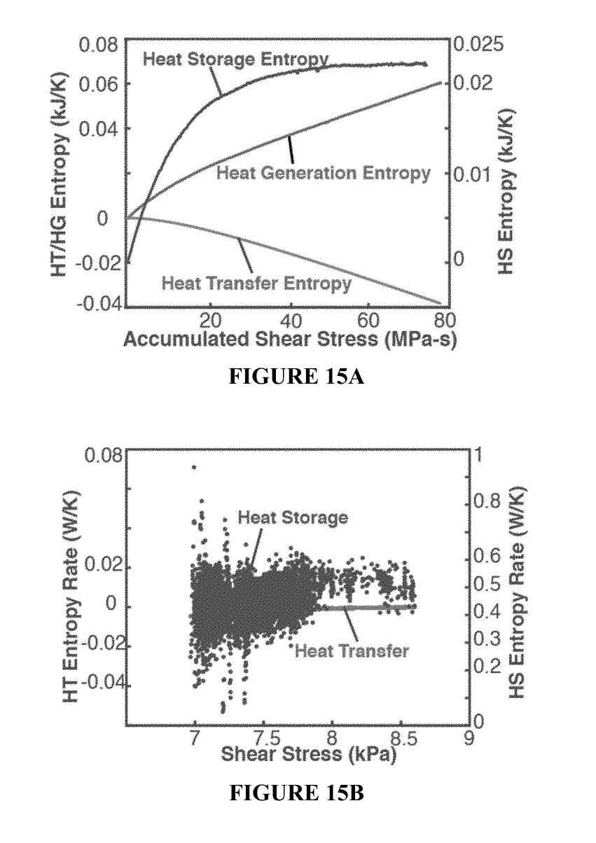

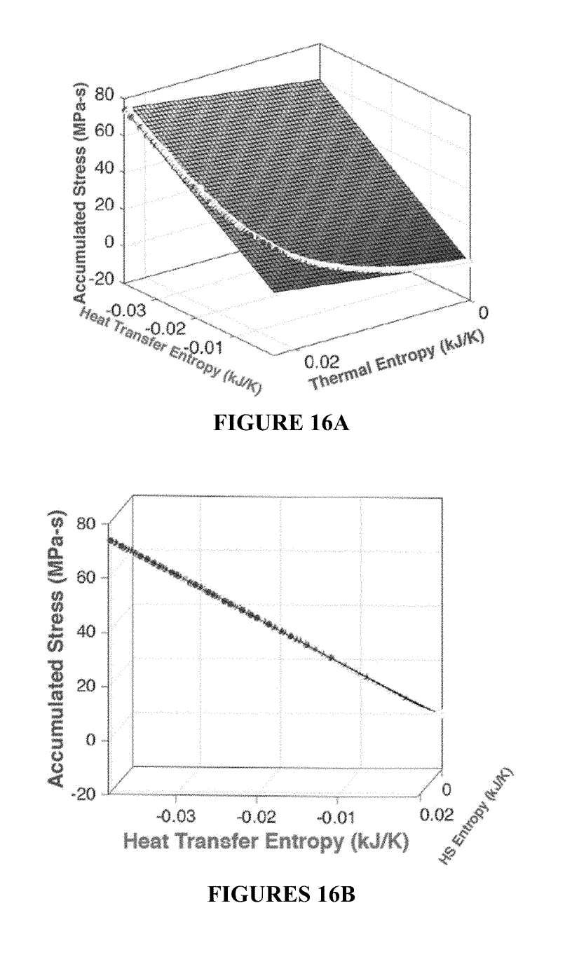

According to the entropy balance equation (Equation 1.41), heat transfer out of the grease reduces entropy, while heat transfer in raises temperature, thereby increasing entropy. Table 3.6 and FIGS. 15A-15B show significance of entropy transfer by heat. The heat storage contribution to total entropy generation is predominant during the initial temperature rise. The partial contributions of both entropy components to overall degradation measure are comparable in the heat-only analysis (FIGS. 15A-15B). Heat generation entropy proceeds opposite heat transfer entropy, as prescribed by equation (Equation 3.132). With both active heat processes significant, the linear partial variation of stress accumulation is shown in FIGS. 16A-16B. FIGS. 16A-16B shows a R²=0.9999 goodness of fit (linear dependence on 2 active processes). As shown in FIG. 16A, shearing trajectory starts from lowest corner and proceeds to apex. As shown in FIG. 16B, all points are coincident with the surface.

Degradation Coefficients and the Degradation Surface

The surface models each had R²≥0.999 with coefficient predictions at 95% confidence interval. FIG. 16B shows all data points coincident with the DEG surface.

Table 3.6 shows consistent trend in heat transfer degradation coefficient B_{Q} with slight variations attributed to variant ambient conditions and measurement sensitivity. B_{T} values show consistent order with slightly varying magnitude from iteration to iteration. B_{T}≈−0.4B_{Q}.

Discussion

Characteristic Nature of the DEG Elements (Lines, Surfaces and Domains)

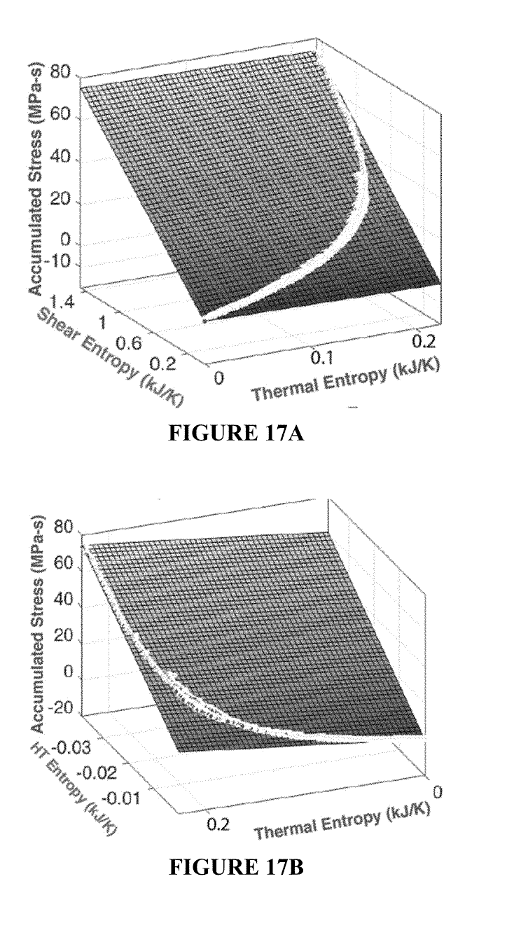

FIGS. 17A and 17B, which plot DEG trajectories from iterations 1-5 of the NLGI 4 grease, suggests a characteristic DEG surface containing all DEG trajectories the grease can “draw” at a given shear rate. The trajectories overlap with slight offset observed in iteration 3 (also evident in its DEG coefficients, see Table 3.5).

FIGS. 17A and 17B show the data points from all the iterations coincident with a common DEG surface, for both Helmholtz and heat-based analyses. Axes are not to scale.

In FIGS. 17A and 17B, the short DEG trajectories indicate shorter shearing duration, hence smaller accumulation. FIG. 17A shows the Helmholtz-based analysis. Trajectories start from lowest corner and proceed to apex. FIG. 17B shows the heat-based analysis. Trajectories from lower right corner and proceed to upper left. The long trajectory belongs to iteration 4 with the longest shearing duration, see Tables 3.5 and 3.6. As shown, the inclination of the DEG surface is dominated by B_{W} (Helmholtz) and B_{Q} (heat), constant for all lines plotted. Note that the axes are not to scale. The DEG domain would be narrower and longer if drawn to scale. Different trajectory lengths indicate different durations. The trajectories overlap.

Prediction Analysis

Four months after data in Tables 3.5 and 3.6 were collected, the same grease sample was again sheared under same conditions and the same DEG coefficients were obtained, see Tables 3.7 and 3.8.

Helmholtz Analysis:

TABLE 3.7
Processed parameters for lithium grease NLGI 4 showing consistent DEG coefficients for shearing
iterations of different durations (shear rate = 123.4 s⁻¹) after a 4-month recovery period.

| | | | | | | | | | B_{W} | B_{T} | | |
|---|---|---|---|---|---|---|---|---|---|---|---|---|
| | Duration | τ | A_{N}\|W | ΔA_{N}\|T | ΔA_{N} | S′_{N}\|W | S′_{N}\|T | S′_{N} | MPa- | MPa- | Residual | GoF |
| N | mins | MPa-s | kJ | kJ | kJ | J/K | J/K | J/K | s/J/K | s/J/K | MPa-s | R² |
| 6 | 168 | 70.6 | −436.9 | −5.6 | −442.5 | 1398.0 | 73.1 | 1471.1 | 51.0 | −33.8 | 3.2 | 1 |
| 7 | 142 | 60.4 | −373.1 | −6.1 | −379.2 | 1196.8 | 39.7 | 1236.5 | 51.0 | −35.2 | −22.3 | 1 |
| 8 | 67 | 28.4 | −175.2 | −4.4 | −179.6 | 570.8 | 28.6 | 599.4 | 50.4 | −28.7 | −15.4 | 1 |
| 9 | 162 | 69.6 | −430.0 | −7.0 | −437.0 | 1369.1 | 41.3 | 1410.4 | 51.5 | −39.0 | −42.5 | 1 |

Heat-Only Analysis:

TABLE 3.8
Processed parameters for lithium grease NLGI 4 showing consistent heat transfer DEG coefficients for
shearing iterations of different durations (shear rate = 123.4 s−1) after a 4-month recovery period.

| | | | | | | | | | B_{Q} | B_{T} | | |
|---|---|---|---|---|---|---|---|---|---|---|---|---|
| | Duration | τ | Q_{N} | ΔE_{N} | E′_{N} | S′_{N}\|Q | ΔS′_{N}\|T | S′_{N} | MPa- | MPa- | τᵣₑₛ | GoF |
| N | mins | MPa-s | kJ | kJ | kJ | J/K | J/K | J/K | s/J/K | s/J/K | MPa-s | R² |
| 6 | 168 | 70.6 | −11.8 | 5.6 | 17.4 | −37.7 | 73.1 | 110.8 | −1569 | 668 | −686 | 0.9999 |
| 7 | 142 | 60.4 | −9.3 | 6.1 | 15.4 | −29.6 | 39.7 | 69.3 | −1613 | 623 | 303 | 1 |
| 8 | 67 | 28.4 | −2.3 | 4.4 | 6.7 | −7.4 | 28.6 | 35.9 | −2433 | 695 | 358 | 0.9999 |
| 9 | 162 | 69.6 | −11.1 | 7.0 | 18.1 | −35.3 | 41.3 | 76.6 | −1540 | 652 | 340 | 0.9999 |

Using Helmholtz-stress coefficient pair and residual from iteration 4 in Table 3.5, Table 3.9 shows measured (column 2) and predicted (column 3) accumulated shear stress for all the iterations including the post 4-month recovery runs. Error, column 4, shows a high prediction accuracy of 98% or more, given the significant experimental error anticipated from the measurement approach.

TABLE 3.9
Measured and predicted accumulated stress
in lithium grease NLGI 4 showing ≤2% error
for shearing iterations (past and future) of different durations.
N = 4, B_{W} = 51.3, B_{T} = −34.5

| | τ | τ_{pred} | |
|---|---|---|---|
| N | MPa-s | MPa-s | % error |
| 1 | 29.7 | 29.8 | −0.4 |
| 2 | 25.8 | 25.9 | −0.2 |
| 3 | 18.3 | 18.3 | −0.1 |
| 4 | 74.3 | 74.3 | 0.0 |
| 5 | 35.8 | 36.4 | −1.7 |

4 months recovery

| | | | |
|---|---|---|---|
| 6 | 70.6 | 69.1 | 2.1 |
| 7 | 60.4 | 60.0 | 0.6 |
| 8 | 28.4 | 28.3 | 0.4 |
| 9 | 69.6 | 68.8 | 1.2 |

Heat-Only Analysis and DEG

Applying the DEG theorem to a heat-only analysis gives further insight. While B_{T} measures the influence of thermal entropy rise due to the shear rate (lower shear rate implies lower viscous heating, hence lower thermal entropy), B_{Q} measures the influence of the surroundings. A constant value of both coefficients from iteration to iteration can be obtained by keeping the surrounding temperature constant throughout, and using accurate high-resolution temperature measurement equipment.

Important Features of the DEG Coefficients Observed
To ensure accurate coefficients, representative thermodynamic formulations of the active processes should be properly determined.
DEG coefficients determined from any iteration, e.g. iteration 1, can accurately predict accumulated shear/shear in subsequent iterations. This suggests the constant degradation coefficients can be determined at any point in grease life using simple measurements, without prior history from the manufacturer/supplier. However, sensitivity to evaluation data suggests coefficient values more consistent under controlled conditions with high-accuracy measurements. Remedies for estimating coefficients under uncontrolled conditions (as in this study) include averages over several iterations with statistical analysis of errors.
DEG trajectories are characteristic of iterations and overlap under consistent operating conditions, DEG surfaces are characteristic of shear rates and the DEG domain characterizes the grease (all iterations and all shear rates). A grease having a domain with large shear stress dimension and small thermal and shear entropy dimensions is able to accumulate more shear stress, operate in service longer and/or carry more load more efficiently.

### Summary and Conclusion

Combined first and second laws of thermodynamics with the Helmholtz potential were used to analyze grease under shear, including transients. Analyses of grease degradation via the DEG theorem was tested by experimental results. DEG coefficients and elements (trajectories, surface and domain) appear to fully and consistently characterize grease for a given shear rate. Sensitivity of the DEG surface orientations and coefficients to shear rate was observed, and sensitivity of the heat-only analysis coefficients to surrounding temperature (each iteration maintained a high level of accuracy with its own coefficient pair).

Applicatory breakdown and prediction analyses show that an appropriate combination of thermodynamic analysis and the DEG theorem could allow manufacturers to directly compare thickener and base oil compositions during grease manufacture. Measurements and appropriate data analyses via the DEG theorem give users a tool to compare various grease types, to determine suitability for an intended application (high temperatures, high shear rates, etc.).

### Example 4. Battery Degradation

Battery issues include low specific energy, self-discharge and ageing. Models to predict performance over time have limitations. Some use electrical parameters and theories, others combine electrical and chemical phenomena. The battery industry lacks a consistent and effective approach to predict performance and ageing. For lead acid batteries and lithium ion batteries, failure mechanisms are discussed, thermodynamic and DEG analyses are formulated, and measurements of operational parameters are presented, for ageing and performance predictions.

Lead-Acid Battery

Lead-acid batteries, important in automobiles, have a common basic chemistry. At the negative electrode,

PbO₂+3H⁺+HSO₄⁻+2e⁻⇄PbSO₄+2H₂O (Equation

with a potential of +1.69V. At the positive electrode,

Pb+HSO₄⁻⇄PbSO₄+F⁺+2e⁻ (Equation

with a potential of −0.358V. This gives an overall reversible reaction

PbO₂+Pb+2H₂SO₄⇄2PbSO₄+2H₂O (Equation

with an overall cell voltage of +2.048V. The forward reaction discharges the battery and is exothermic. The backward reaction charges the battery and is ideally endothermic at low rates. The quantity of heat produced varies with reaction rates, with charging more exothermic with charge rate. In addition to changes in molar species in the battery during cycling, significant changes in temperature are observed. During discharge, the forward reaction has hydrogen ions and lead sulfate produced at the negative electrode, with water and lead sulfate produced simultaneously at the positive electrode. In equations (Equation 4.176)-(Equation 4.178), the reverse reaction during the charge cycle produces sulfuric acid and lead at the negative electrode, and hydrogen ions, lead oxide and sulfuric acid at the positive electrode.

Cyclic changes in chemical and thermal states during electrical charge-discharge cycles give rise to measurable parameters to determine the state of charge and overall health of the battery. Specific gravity (to indicate proportions of water/acid in the aqueous electrolyte), and temperature (a surrogate for heat added/rejected) of the electrolyte are macro measurements presented in this work.

Conventional lead-acid batteries (flooded with H₂SO₄ having Pb and PbO₂ electrodes) include starter batteries (short-duration, high-current power) for engines, and deep-cycle batteries (slow, steady, long-running power) for marine vessels and golf carts.

Changes in battery charge-holding capacity can be determined from measurements of electrical current and voltage, which change during subsequent charge and discharge processes. Manufacturers specify nominal electrical charge-holding capacity using Cold Cranking Amps (CCA) for starter batteries, and Reserve Capacity (RC) for both starter and deep-cycle batteries. These values are usually large for most 6V and 12V batteries. More general is the 20-hr capacity rating, the maximum current the battery can output consistently for 20 hours. The capacity and energy content of a battery can be determined from measured current I, terminal voltage V, resistance R and time t during cycling.

Lithium-Ion Battery

High energy density, minimal maintenance, low self-discharge and long cycle life make lithium-ion batteries ideal rechargeable batteries. Chemistry at the cathode

Li₁₋ₓMO₂+xLi⁺+xe⁻⇄LiMO₂ (Equation

and at the anode

LiₓC ⇄C+xLi⁺+xe⁻ (Equation

with a nominal cell voltage of ˜3.6-4.2V depending on the transition metal Mused. Here 0≤x<1. Transition metals M include Cobalt, Manganese, Nickel, etc. In a typical Li-ion cell, the electrodes are active materials Li₁₋ₓMO₂ and LiₓC, bonded to current collectors by the electrolyte, usually liquid, gel or lithium metal polymer, which facilitates transport of Lithium ions (Li⁺) between electrodes. During charging, Li⁺ are deintercalated (extracted) at the cathode and the active material is oxidized, whereas the anode active material is reduced and Li⁺ extracted from the cathode are intercalated (inserted) into the anode. The phenomena reverse for discharge.

Charging applies a constant current which energizes the battery to just below maximum voltage, followed by a constant-voltage process during which charge current decreases to 3% of the battery's rated current. Cycling a battery dissipates a variable amount of heat depending on the charge/discharge rates. This work investigates battery response to unsteady cycling rates. End of discharge voltage for typical commercial Li-ion batteries is 2.7V/cell to avoid damage from deep discharge. An intermediate step, settling, allows the transport phenomena and reaction kinetics to stabilize and establish a steady state initial reference for the next charge/discharge step. The complete regime is charge-settle-discharge-settle-charge. Manufacturers specify nominal and typical electrical charge-holding capacity of Li-ion batteries at a specific discharge rate in Ampere-hours.

Relevant Battery Parameters

Capacity [symbol] (t), the maximum number of Ampere-hours (Ah) a battery can output at a specified rate starting from time t, is the charge

[symbol]

(t)=∫ₜ^{t+Δt}I(t)dt (Equation

where Δt is the time increment or duration. The definition implies the left-hand side (LHS) of equation (Equation 4.181) is known. Manufacturers' nominal capacity [symbol] (ampere-hours) implies need to know a battery's degradation over time to satisfy power requirements. For experiments the LHS is determined by the RHS (area under discharge current versus time curve), making capacity in practice the total discharge [symbol] (Δt) over time duration Δt. Equation (Equation 4.181) is redefined as

[symbol]

[symbol]

(Δt)=∫ₜ^{t+Δt}I(t)dt (Equation

In-use capacity [symbol] (Δt,n) is the total number of Ampere-hours output from a battery at the nth cycle. State of health SOH of a battery, a primary degradation measure, is the ratio

$$SOH = \frac{𝒞 ( Δ {t}_{c} , n )}{𝒞 ( Δ {t}_{c} , 0 )} \qquad ( Equation$$

of capacity [symbol] (Δt_{c},n) at the nth cycle to initial capacity [symbol] (Δt_{c},0). Here Δt_{c}, the constant duration used for all cycles, can be determined from cycle 0 (the initial reference cycle). Equation (Equation 4.183) requires the battery be fully charged before every discharge. For a new fully charged battery, SOH is 100%. Manufacturers consider a Li-ion battery degraded when SOH is 60-65%. Lead-acid batteries are often considered degraded at 80% SOH. Cycle life, the number of charge-discharge cycles completed before a battery is considered degraded, can be denoted by a plot of C or SOH versus number of cycles. For rechargeable batteries, inconsistent cycling, vis-a-vis incomplete charge and varied discharge, requires a depth of discharge definition

$$DOD = \frac{𝒞 ( Δ t , n )}{𝒞 ( Δ {t}_{f} , n )} \qquad ( Equation$$

the ratio of accumulated discharge [symbol] (Δt,n) to total discharge capacity [symbol] (Δt_{f},n), where Δt_{f} is the time for a full discharge. Both equations (Equation 4.182) and (Equation 4.184) require consistent charge-discharge rates for all cycles. State of charge

SOC=1−DOD, (Equation 4.185)

the percentage of charge accumulated in the battery is 100% after a full recharge and rest. Other measures for battery suitability include specific energy with units W-h/kg given by

$${E}_{m} = \frac{𝒞 ( t ) V}{m} \qquad ( Equation$$

and specific power in W/kg given by

$${\dot{E}}_{m} = \frac{𝒞 ( t ) V}{mt} = \frac{IV}{m} \qquad ( Equation$$

Battery internal impedance

$${Z}_{i} = \frac{{V}_{oc} - V}{I} = \frac{{V}_{oc}}{I} - {R}_{load} \qquad ( Equation$$

(via a Thevenin equivalent circuit of the battery as a voltage source V_{oc} in series with Zᵢ) can be determined via measurements of open circuit voltage V_{oc}, voltage V across the external resistive load R_{load}, and current I through R_{load}. Unclear is how Zᵢ increases with cycling: different studies give different conclusions.

Degradation Measures

Lead-Acid Battery

After many charges and discharges, a lead-acid battery cannot hold charge over time due to gradual, permanent changes in materials. Failure mechanisms include sulfation on the anode, water loss due to gassing and evaporation, expansion of the cathode, acid stratification and grid corrosion. With modern materials, cell design and proper maintenance, lead-acid batteries can be cycled over 1000 times and still hold adequate charge. However, users often subject batteries to non-ideal conditions, which encourage failure mechanisms. High rates of discharge and recharge, wide ranges of depth of discharge DoD, overcharging, storing batteries for long periods in a discharged state, and high temperatures among others accelerate battery degradation. Design and materials also determine useful life.

The Electrolyte

Specific Gravity Measurements

Typical acid to water ratio in a fresh battery electrolyte is 3.5:6.5. Concentration of constituents vary depending on the state of charge SO [symbol] . At full charge, specific gravity of fresh electrolyte is about 1.25; at full discharge when the electrolyte is mostly water, specific gravity is between 1.15 to 1.175. Values decrease with battery degradation, impurities and water loss. Via a temperature-compensated battery hydrometer, electrolyte specific gravity can be determined.

Lithium-Ion Battery

Even with proper maintenance, capacity of a li-ion cell fades with cycling. After several charge-discharge cycles, the internal impedance increases, producing more dissipative losses which generate heat. Other degradation mechanisms include lithium corrosion and plating on the anode, and excessive growth or disconnection of the solid-electrolyte interface (SEI) layer on or from the anode respectively, resulting in loss of contact. On the cathode, a passivation layer forms, grows during cycling and reduces capacity over time. Structural changes in the electrodes and irreversible decomposition of the electrolyte over time also limit intercalation and diffusion of Li⁺.

Li-ion batteries are very sensitive to charge rates and Depth of Discharge DOD. Improper charging overheats batteries causing catastrophic failure. Over-charging facilitates migration of Li⁺ from the layered structure, building up metallic lithium on the anode and releasing excess oxygen at the cathode. As this continues, pressure in the battery increases and more heat is released, which can eventually cause an explosion. Over-discharging causes similar irreversible damage.

Efforts to predict li-ion battery ageing have included experimental cycle ageing of batteries, but none present a complete dataset including changes in temperature during cycling. Battery capacity and cycle life depend on design and operating conditions.

Review of Available Models

Feinberg used an extended system boundary that included battery and charger and summed entropy change over charge and discharge cycles

$${dS}_{tot} = ( \frac{dU}{T} - \frac{Vdq}{T} ) + ( \frac{{dU}_{en}}{{T}_{en}} - \frac{{Edq}_{en}}{{T}_{en}} ) \qquad ( Equation 4.189 )$$

Equation (Equation 4.189) used internal energy change, considered total entropy change of the extended system, and neglected diffusion—most difficult to measure experimentally. As noted above, this is easily subject to misinterpretation.

Process irreversibilities can be presented through overpotentials:
charge transfer over-potential using Tafel's equations, from Butler-Volmer's equation:

$${η}_{t} ≫ \frac{RT}{zF} ; {η}_{t} > 0 , {η}_{t} = \frac{RT}{z α F} ln \frac{i}{{i}_{0}} \\ \vert {η}_{t} \vert ≫ \frac{RT}{zF} ; {η}_{t} < 0 , {η}_{t} = \frac{Rt}{z ( 1 - α ) F} ln \frac{{i}_{0}}{\vert i \vert} \qquad ( Equation 4.190 )$$

which combine to give the ohmic work (charge transfer) term. The Tafel equations are valid for high charge/discharge rates j.
the diffusion over-potential at the electrode/electrolyte interface when concentration gradient dc/dx is constant

$${η}_{d} = \frac{RT}{nF} ln {\prod }_{j} {( 1 - \frac{i}{{i}_{1 , j}} )}^{{v}_{j}} \qquad ( Equation 4.191 )$$

neglecting diffusion within the electrolyte. The thermal model starts with Gibbs free energy

dG=−SᵣₑᵥdT+VdP+EdQ (Equation 4.192)

defined for reversible processes only, acceptable given the sources of irreversibilities were previously represented with over-potentials.

$${S}_{rev} = - {( \frac{\partial G}{\partial T} )}_{p} = Q ( \frac{\partial E}{\partial T} ) \qquad ( Equation 4.193 )$$

is the reversible entropy from thermal energy change. The irreversible entropy production from the ohmic loss can be represented as

T\dot{S}ᵢᵣᵣ=ηI (Equation 4.194)

and entropy transfer out as

T\dot{S}ₑₓₜ=HA(Tₐ−T) (Equation 4.195)

This seems to represent all the prevalent mechanisms.

Other have presented

$${C}_{p} \frac{dT}{dt} = - {hA}_{s} ( T - {T}_{∞} ) + {q}_{i} + {q}_{j} + {q}_{c} + {q}_{r} , \qquad ( Equation 4.196 )$$

as heat changes in the battery, a function of convection, chemical reaction heat, Joule heat, contact resistance heat and reversible heat and then combined chemical and Joule heat into one expression, while the contact heating was separated. An ion conservation and transport model has also been presented in conjunction with the electrochemical analysis presented below.

A coupling of both the chemical and electrical models of the battery has also been presented. The diffusion work was neglected in the Gibbs relation

$$AJ = \sum {μ}_{i} {\dot{N}}_{i} = - \frac{dG}{dt} \qquad ( Equation$$

where A is affinity and J is reaction extent. The entropy from energy dissipated as heat from either ohmic or chemical reaction work was represented as

T\dot{S}′=AJ=VI (Equation

This approximate model describes an adiabatic operation.

A battery can also be modeled considering charge conservation and transport and using a similar approach to diffusion, including the effective diffusion coefficient, as in the Butler-Volmer equation for charge transfer. These models often fail under unsteady operation, over-discharging and other non-linear system response; often cannot accurately predict useful life; and cannot adequately account for battery ageing and/or parasitic losses.

Bond graphs of lead-acid battery dynamics during cycling include primary and secondary electrochemical reactions at both electrodes, and thermal energy dissipation. Others give a similar model for one electrode of the lithium-ion battery. The relevant accumulation, ohmic and diffusion phenomena power balances were represented at 1-junctions and a transformer element TF represented the conversion of electro-chemical forms of power. Relationships and energy formulations can be directly obtained from the appropriate junctions. From relevant temperatures and the power dissipated at resistance elements in the bond graph model, an overall rate of irreversible entropy generation in li-ion batteries can be obtained as

$$\frac{{dS}^{′}}{dt} = \frac{- Δ {G}_{diff} {J}_{diff}}{{T}_{diff}} + \frac{{V}_{act} I}{{T}_{act}} + \frac{{V}_{elec} I}{{T}_{elec}} \qquad ( Equation$$

neglecting thermal entropy, adequate for very low cycling rates.

Analysis

Batteries degrade chemically through electrode corrosion and evolution of gases; electrically as observed through capacity fade; and thermally via hot environments and joule heating, which often accelerates chemical degradation.

Thermodynamic Analysis

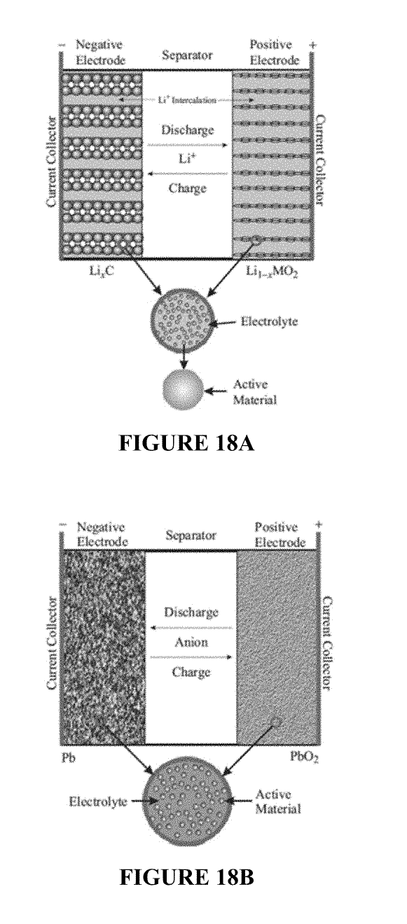

Characterizing the cyclic operation of a battery requires appropriate formulations for the charge and discharge processes. Since electrochemistry couples the chemical reaction with the ohmic work interaction, boundary work can be represented by a more convenient form. A complete formulation includes an ion diffusion component, often negligible in energy analysis of most charge/discharge applications but more significant in applications with slow charging and long settling times. Heat generated by the charge and discharge work transfers out of the system into the surroundings. After the work interaction is removed, the system spontaneously settles to a new equilibrium state. FIGS. 18A and 18B show charge and discharge mechanisms in li-ion and lead-acid batteries, respectively.

Infinitesimal System Model—Gibbs Analysis

Electrical Work and Thermal Energy Change

Assumptions:
1. The boundary encloses the battery only.
2. System is closed (battery mass stays in the battery).
3. Heat transfers between battery and surroundings.
4. The system is at equilibrium before and after charging or discharging.

From equation (Equation 1.36) at constant pressure, the change in the Gibbs free energy

dG=−SdT+μdN′ (Equation

For convenience, the chemical reaction is replaced by the directly coupled electrical boundary work given by the ohmic process

δW=Vdq (Equation

where V is the terminal voltage and dq=Idt is the charge transferred. An oft neglected but simultaneous diffusion process can be accounted for using

(μ_{high}−μ_{low})dN_{d} (Equation

where μ_{high},μ_{low} are diffusion potentials or chemical potentials in the high and low potential regions respectively, and dN_{d} is the change in ion concentration. For the discharge process, equation (Equation 4.200) becomes

dG=−CdT−Vdq+(μ_{high}−μ_{low})dN_{d}, (Equation 4.203)

where SdT was replaced by CdT via equation (Equation 1.43). Here, dT≥0, dq≥0 and dN_{d}≤0 according to IUPAC convention. Equation (Equation 4.203) suggests

G=G(T,q,N_{d}) (Equation 4.204)

Equation (Equation 4.203) gives the quasi-static change in Gibbs potential, the maximum electrochemical energy obtainable from a battery. Entropy production from equation (Equation 1.39) is restated as

$${δδ}^{′} = - \frac{{dG}_{rev}}{T} + \frac{CdT}{T} + \frac{Vdq}{T} + \frac{( {μ}_{high} - {μ}_{low} ) {dN}_{d}}{T} \qquad ( Equation$$

a difference between reversible and irreversible entropies. In the foregoing equation,

dGᵣₑᵥ=μdN′|ᵣₑᵥ=Vdq|ᵣₑᵥ (4.206)

Established formulations for dGᵣₑᵥ for ideal electrochemical storage include

G=−nFV_{OC} (Equation

where n is number of species, e.g. electrons involved in charge transfer (2 for lead-acid batteries and x for lithium-ion batteries) and F=96,485 C mol⁻¹ is Faraday's constant. For a constant voltage (Vᵢₙᵢₜᵢₐₗ=V_{final}) process,

dGᵣₑᵥ=0 (Equation

Relaxation/Settling

During active charging/discharging, any heat generated and not instantaneously transferred out builds up. Upon work removal, that heat transfers out as the Gibbs potential proceeds to a new equilibrium state. During settling the cell voltage relaxes and the battery transfers entropy to the atmosphere spontaneously. The change in Gibbs during settling is

dGᵣ=dGᵣₑᵥ−CdT+(μ_{high}−μ_{low})dN_{d}(relaxation) (Equation 4.209)

where dGᵣₑᵥ denotes voltage relaxation, CdT thermal relaxation and (μ_{high}−μ_{low})dN_{d} diffusion during settling, all of which proceed spontaneously and significantly slower than the active ohmic processes. Entropy production during settling is

$$δ {S}_{r}^{′} = \frac{{dG}_{rev}}{T} + \frac{CdT}{T} + \frac{( {μ}_{high} - {μ}_{low} ) {dN}_{d}}{T} \\ ( relaxation ) \qquad ( Equation 4.210 )$$

In a typical charge-discharge cycle, settling proceeds in opposite directions and essentially cancel out in an energy analysis. With the voltage relaxation component of entropy subtracting out during a complete charge-discharge cycle, entropy production during settling proceeds at the same rate as diffusion of the charge species which, for entropy analysis with active processes, is negligible.

The Gibbs equilibrium condition for a spontaneous process dG≤0 holds as dT≤0, a verification of the second law δS′≥0. Relaxation equilibrium is approached asymptotically, taking several hours to weeks, and

δS′>>δSᵣ′ (Equation

Dropping the reversible accumulation and diffusion terms accordingly, entropy production during the ohmic charge/discharge cycling of a battery becomes

$${δδ}^{′} = \frac{CdT}{T} + \frac{Vdq}{T} \qquad ( Equation$$

Infinitesimal Model—Heat Only

If temperature parameters or measurements are more readily available for the battery, or to study the effects of the surroundings on entropy generation, a heat-based model can be used.

Assumptions:
1. The boundary encloses the battery.
2. System is closed.
3. Heat transfers between battery and immediate surroundings via free convection.
4. The system is at equilibrium before and after operation.

From equation (Equation 1.40), heat generation

δE′=CdT−δQ (Equation

and entropy generation from heat, equation (1.40),

$${δδ}^{′} = \frac{CdT}{T} - \frac{δ Q}{T} \qquad ( Equation$$

where RHS terms are the battery's thermal energy storage and heat transfer entropies respectively. The heat storage term is equivalent to the thermal energy term in the Gibbs formulation equation (Equation 4.203). Rate of heat transfer out of the battery Q via equation (1.41)

\dot{Q}=ΔT/Rₜ (Equation

is a ratio of the difference DT between battery and ambient temperatures to the thermal resistance Rₜ in between. For a one-dimensional lumped-capacity heat transfer model using electrolyte temperature, thermal resistance including conduction through the battery housing of thickness Δx and free convection with the surroundings is given by

$${R}_{t} = ( \frac{1}{{h}_{air} {A}_{s}} ) + ( \frac{Δ x}{{kA}_{s}} ) \qquad ( Equation$$

where Aₛ is the housing surface area and hₐᵢᵣ the average heat transfer coefficient of air and k the thermal conductivity of housing material. Active ohmic work rate proceeds significantly faster than the spontaneous heat transfer processes. For low discharge rate applications, heat transfer is not easily measurable, making the model based on work more convenient.

Infinitesimal Model—Electrochemical Work and Diffusion

The Gibbs fundamental relation equation (1.35) for a battery

dG=−CdT+(Σμ_{P}dN_{P}−Σμ_{R}dN_{R})+(μ_{high}−μ_{low})dN_{d} (Equation

has chemical reaction and diffusion work terms. Subscripts P and R refer to the products and reactants in a chemical reaction. From Faraday's first law, the consumption or production rate of a species is

$$dN = \frac{Idt}{nF} \qquad ( Equation$$

With known chemical potentials (μ), equation (Equation 4.218) allows evaluation of the chemical reaction work. To use specific gravity measurements for lead-acid batteries, a mass basis is employed. Using

$${dN}_{i} = \frac{{dm}_{i}}{{v}_{i} {M}_{i}} , \qquad ( Equation 4.219 )$$

equation (Equation 4.217) becomes

$$dG = - CdT + \sum {μ}_{i} \frac{{dm}_{i}}{{v}_{i} {M}_{i}} + ( {μ}_{high} - {μ}_{low} ) {dN}_{d} \qquad ( Equation$$

where mᵢ is mass, vᵢ is the stoichiometric coefficient and Mᵢ is molecular mass of active material i. If i is H₂SO₄ (the active component in lead-acid batteries) and using initial electrolyte composition, m_{H2SO4}=0.35 m_{electrolyte}, dmᵢ is rewritten as

Δm_{H2SO4}=0.35ρ_{H2O}(ΔSG*∀)_{electrolyte}, (Equation 4.221)

where Δm_{H2SO4} is the change in acid mass, ρ is density, SG is specific gravity and ∀ is volume. Using the chemical reaction's affinity,

A=Σμ_{P}v_{P}−Σμ_{R}v_{R} (Equation

where n_{P} and n_{R} arising from equation (Equation 4.219) are coefficients for product and reactant terms in the stoichiometric equations, such as (Equation 4.176)-(Equation 4.180). The reaction extent

$$d ξ = \frac{{dN}_{i}}{{v}_{i}} \qquad ( Equation$$

can reformulate equation (Equation 4.217) as

dG=−CdT+Adξ+(μ_{high}−μ_{low})dN_{d} (Equation

Also, entropy production

$$δ {S}^{′} = - \frac{{dG}_{rev}}{T} + \frac{CdT}{T} + \frac{Ad ξ}{T} + \frac{( {μ}_{high} - {μ}_{low} ) {dN}_{d}}{T} \qquad ( Equation$$

With reactions taking place at different potentials at both electrodes, more appropriate is the average electrochemical affinity

Ă=Σμ_{P}v_{P}−Σμ_{R}v_{R}−V_{OC} (Equation

In terms of forward and reverse reaction rates \dot{R}_{f} and \dot{R}ᵣ,

$$A = RTln \frac{{\dot{R}}_{f}}{{\dot{R}}_{r}} \qquad ( Equation$$

and

dξ=(\dot{R}_{f}−\dot{R}ᵣ)∀dt (Equation

Diffusion Work

Fick's laws of diffusion and ion conservation govern the diffusion work. The diffusion component in equation (Equation 4.217) given in establishes ion transport driven by diffusion affinity (μ_{high}−μ_{low}), the difference in species chemical potentials in regions inside the battery. Using activity aₖ and reformulating in terms of molalities mₖ₊^{high},mₖ₊^{low} (mol/kg) of both regions

$$( {μ}_{high} - {μ}_{low} ) = RT ln ( \frac{{m}_{k +}^{low}}{{m}_{k +}^{high}} ) \qquad ( Equation$$

The electrolyte ion flux density \dot{N}_{d} (x, t) (mol/cm²-s) in terms of concentration M_{C}(x, t) (mol/cm³) and the diffusion coefficient D (cm²/s) can be written as

$${\dot{N}}_{d} = - D \frac{\partial {M}_{C}}{\partial x} \qquad ( Equation$$

From the Stokes-Einstein equation,

$$D = \frac{{k}_{B} T}{{η}_{v}} \qquad ( Equation$$

where ηᵥ is electrolyte dynamic viscosity and k_{B} is Boltzmann's constant. Concentration M_{C} refers to the electrolyte active component, e.g. H₂SO₄ in lead-acid batteries. Values of D are available. The flux density gradient according to Fick's second law,

$$\frac{\partial {N}_{d}}{\partial x} = J - ɛ \frac{\partial {M}_{C}}{\partial t} \qquad ( Equation$$

includes diffusion rate J (mol/cm³-s) from the electrodes of porosity E. When J is used, an effective coefficient derived from Bruggeman's relation (taking into account the effect of electrode porosity) may be recommended:

D_{eff}=Dε^{1.5} (Equation

Using equations (Equation 4.218) and (Equation 4.226) for electrochemical work and (Equation 4.229) and (Equation 4.232) for diffusion, equation (Equation 4.225) evaluated from known or measurable thermal and electrochemical quantities becomes

$${S}^{′} = \frac{nF Δ {V}_{OC}}{T} + \frac{C Δ T}{T} + \frac{( \sum {μ}_{P} {v}_{P} - \sum {μ}_{R} {v}_{R} - {V}_{OC} ) I Δ t}{nFT} + R Δ x ( J Δ t - ɛΔ {M}_{C} ) ln ( \frac{{m}_{k +}^{low}}{{m}_{k +}^{high}} ) \qquad ( Equation$$

where differentials were replaced by differences, e.g., dT replaced by ΔT. Current I in the electrochemical work term in equation (Equation 4.234) and the definition of the electrochemical affinity verify the chemical reaction coupling to the electrical work, and suggest the diffusion process to be much slower than the electrochemical reaction. The reversible Gibbs accumulation (first term) depends on the difference between initial and final battery voltage for charge or discharge and usually cancels out for a full cycle. Thermal entropy change (second term) depends on temperature change and active component (electrode/electrolyte) material. Dependence of temperature rise on ohmic heating and the latter's dependence on current gives the thermal term a coupled dependence on current. The electrochemical entropy (third term) is the primary interaction and most significant term, depending on chemical potential of active material and current. Solid-phase Li⁺ has a chemical potential μ of about −293.8 kJ/mol and H₂SO₄ has μ=−690 kJ/mol. Diffusion (last term) is spontaneous and depends on the diffusion rate and the rate of change of species concentration. Diffusion coefficients of H⁺ and solid-phase Li⁺ are about 5×10⁻⁵ cm²/s and 1.3×10⁻⁸ cm²/s respectively. Equations (Equation 4.219) and (Equation 4.221) show that a current of 1 A would consume H₂SO₄ at each electrode of a lead-acid battery at a rate (hence the rate of change of concentration) of about 5.2×10⁻⁶ mol/s. These numbers indicate that diffusion proceeds at least 5 orders of magnitude slower than the other active processes, and can be neglected. Also, lead-acid batteries take over 24 hours to settle after charge, especially when saturation charge is applied. After discharge, batteries are not left to settle before recharging, so settling/relaxation effects are negligible. Dropping the accumulation and diffusion terms, equation (Equation 4.234) becomes

$${S}^{′} = \frac{C Δ T}{T} + \frac{( \sum {μ}_{P} {v}_{P} - \sum {μ}_{R} {v}_{R} - {V}_{OC} ) I Δ t}{nFT} \qquad ( Equation$$

In terms of reaction rates,

$${S}^{′} = \frac{C Δ T}{T} + \frac{R ( {\dot{R}}_{f} - {\dot{R}}_{r} ) ∀ Δ t}{nF} ln \frac{{\dot{R}}_{f}}{{\dot{R}}_{r}} \qquad ( Equation$$

and mass,

$${S}^{′} = \frac{C Δ T}{T} + μ \frac{Δ m}{vM} \qquad ( Equation$$

Equations (Equation 4.235)-(Equation 4.237) describe entropy generation from chemical reaction analysis.

Experimental Model—Gibbs and Heat

Rates for battery cycling are described herein. Parameters are measured to determine energy changes and entropy production. As discussed, diffusion can be neglected. The unsteady charge profile suggests that an instantaneous approach may be useful for accurate entropy component determination.

Control Parameters:
1. Only the battery is considered in the closed system.
2. Heat transfers with the surroundings.

Using rate forms of equations (Equation 4.203) and (Equation 4.205) and neglecting the diffusion terms, entropy production in the battery during discharging is given by

$$\begin{matrix}\dot{G} = - C \dot{T} - VI & ( Equation \\ \dot{{S}^{′}} = - \frac{{\dot{G}}_{rev}}{T} + \frac{C \dot{T}}{T} + \frac{VI}{T} & ( Equation\end{matrix}$$

To obtain total change in Gibbs energy and entropy generation during discharge (denoted by subscript d), both thermal and electrical energy changes are considered:

$$\begin{matrix}Δ G = - {\int }_{{t}_{0}}^{{t}_{d}} C \dot{T} dt - {\int }_{{t}_{0}}^{{t}_{d}} VIdt & ( Equation \\ {S}_{c}^{′} = - {\int }_{{t}_{0}}^{{t}_{d}} \frac{\dot{G}}{T} dt + {\int }_{{t}_{d}}^{{t}_{0}} \frac{C \dot{T}}{T} dt + {\int }_{{t}_{0}}^{{t}_{d}} \frac{VI}{T} dt & ( Equation\end{matrix}$$

where t₀ is the start time and t_{d} the end time of the discharge process. In cycle analysis, settling is negligible as discussed previously. If discharge depth is made equal to charge depth, the first term on the right side of equation (Equation 4.241) drops out, giving

$${S}_{cycle}^{′} = {\int }_{{t}_{c}}^{{t}_{d}} \frac{C \dot{T}}{T} dt + {\int }_{{t}_{c}}^{{t}_{d}} \frac{VI}{T} dt + {\int }_{{t}_{0}}^{{t}_{c}} \frac{C \dot{T}}{T} dt + {\int }_{{t}_{0}}^{{t}_{c}} \frac{VI}{T} dt \qquad ( Equation$$

for a cycle, where t_{c} is the end time of the charge process.

Recalling the heat only model in equation (Equation 1.41), entropy generation rate

$$\dot{{S}^{′}} = \frac{C \dot{T}}{T} - \frac{\dot{Q}}{T} \qquad ( Equation$$

and for an entire cycle,

$${S}_{cycle}^{′} = {\int }_{{t}_{c}}^{{t}_{d}} \frac{C \dot{T}}{T} dt - {\int }_{{t}_{c}}^{{t}_{d}} \frac{\dot{Q}}{T} dt + {\int }_{{t}_{0}}^{{t}_{c}} \frac{C \dot{T}}{T} dt - {\int }_{{t}_{0}}^{{t}_{c}} \frac{\dot{Q}}{T} dt \qquad ( Equation$$

where \dot{Q} can be evaluated from equation (Equation 1.42).

Measurements of concentrations required to experimentally validate equations (Equation 4.235)-(Equation 4.236) for Li-ion batteries require expensive equipment. In lead-acid batteries, hydrometers measure H₂SO₄ concentration in the electrolyte, which can crudely estimate or verify the chemical model. Specific gravity measurements, while prone to significant error, can help estimate battery degradation when made at equilibrium states (end of charge/discharge). Substituting specific gravity measurements into equation (Equation 4.221) and combining with equation (Equation 4.237), entropy generation can be estimated. The external electrical work interaction in section 4.6.1.1 will give the most accurate formulation in unsteady applications, including overcharging/over-discharging. When accurate measurements of changing chemical reaction parameters are available, chemical models provide more insight.

Degradation Entropy Generation (DEG) Analysis

For the discharge process, from equation (Equation 4.239),

S′=S′{G,T,I} (Equation 4.245)

Recall the measurable degradation parameter w and the DEG theorem

$$\frac{dw}{dt} = \sum B \frac{{dS}^{′}}{dt} \qquad ( Equation$$

Equations (Equation 4.245) and (Equation 4.246) together suggest

w=w{G,T,I} (Equation

Equation (Equation 4.239) via the DEG theorem suggests a degradation rate

$$\frac{dw}{dt} = - {B}_{G} \frac{Δ \dot{G}}{T} + {B}_{T} \frac{C \dot{T}}{T} + {B}_{W} \frac{VI}{T} \qquad ( Equation$$

where B_{G}, B_{T} and B_{W} are Gibbs analysis degradation coefficients. In terms of entropy generation from heat generation analysis, equation (Equation 4.243),

S′=S′{T,T_{∞}} and w=w{T,T_{∞}} (Equation 4.249)

giving

$$\frac{dw}{dt} = {B}_{T} \frac{C \dot{T}}{T} - {B}_{Q} [ \frac{( T - {T}_{∞} )}{{R}_{t} T} ] \qquad ( Equation$$

where B_{T} and B_{Q} are heat generation analysis degradation coefficients. Equations (Equation 4.248) and (Equation 4.250) are the fundamental degradation relations. Degradation coefficients

$${B}_{i} = \frac{\partial w}{\partial {S}_{i}^{′}} {\vert}_{{p}_{i}} \qquad ( Equation$$

can be evaluated from measurements, as slope of degradation measure w to entropy production Sᵢ′ for dissipative process pᵢ. Recall notation|ₚ_{ᵢ} refers to pᵢ being active. For one complete discharge process,

$$\begin{matrix}w = - {B}_{G} \int \frac{Δ \dot{G}}{T} dt + {B}_{T} \int \frac{C \dot{T}}{T} dt + {B}_{I} \int \frac{VI}{T} dt \\ and & ( Equation \\ w = {B}_{T} \int \frac{C \dot{T}}{T} dt - {B}_{HT} \int [ \frac{( T - {T}_{∞} )}{RT} ] dt & ( Equation\end{matrix}$$

Cycle Analysis

Equation (Equation 4.242) gives the entropy produced over a charge-discharge cycle, from which the DEG theorem suggests the degradation per cycle to be

$${w}_{cycle} = {B}_{{T}_{c}} {\int }_{{t}_{0}}^{{t}_{c}} \frac{C \dot{T}}{T} dt + {B}_{{W}_{c}} {\int }_{{t}_{0}}^{{t}_{c}} \frac{VI}{T} dt + {B}_{{T}_{d}} {\int }_{{t}_{c}}^{{t}_{d}} \frac{C \dot{T}}{T} dt + {B}_{{W}_{d}} {\int }_{{t}_{c}}^{{t}_{d}} \frac{VI}{T} dt \qquad ( Equation$$

giving an accumulated degradation over N cycles

$${w}_{total} = {\sum }_{N} [ {B}_{{T}_{c}} {\int }_{{t}_{0}}^{{t}_{c}} \frac{C \dot{T}}{T} dt + {B}_{{W}_{c}} {\int }_{{t}_{0}}^{{t}_{c}} \frac{VI}{T} dt + {B}_{{T}_{d}} {\int }_{{t}_{c}}^{{t}_{d}} \frac{C \dot{T}}{T} dt + {B}_{{W}_{d}} {\int }_{{t}_{c}}^{{t}_{d}} \frac{VI}{T} dt ] \qquad ( Equation$$

DEG Coefficients from Existing Models

Using Capacity and SOH as Failure Parameters

Letting accumulated discharge [symbol] defined in equation (Equation 4.181) be a degradation measure or performance parameter, equation (Equation 4.252) with [symbol] replacing w becomes

$$𝒞 = - {B}_{G} \int \frac{Δ \dot{G}}{T} dt + {B}_{T} \int \frac{C \dot{T}}{T} dt + {B}_{W} \int \frac{VI}{T} dt \qquad ( Equation$$

where the Gibbs capacity coefficients

$${B}_{G} = \frac{\partial 𝒞}{\partial {S}_{G}^{′}} ; {B}_{T} = \frac{\partial 𝒞}{\partial {S}_{T}^{′}} ; {B}_{W} = \frac{\partial 𝒞}{\partial {S}_{W}^{′}} \qquad ( Equation$$

pertain to accumulation/activation entropy

$${S}_{G}^{′} = \int \frac{Δ \dot{G}}{T} dt ,$$

thermal entropy

$${S}_{T}^{′} = \int \frac{C \dot{T}}{T} dt$$

and ohmic entropy

$${S}_{W}^{′} = \int \frac{VI}{T} dt$$

respectively. Similarly from equation (Equation 4.253),

$$𝒞 = {B}_{T} \int \frac{C \dot{T}}{T} dt - {B}_{Q} \int [ \frac{( T - {T}_{∞} )}{RT} ] dt \qquad ( Equation$$

where the heat generation capacity coefficients

$${B}_{T} = \frac{\partial 𝒞}{\partial {S}_{T}^{′}} ; {B}_{Q} = \frac{\partial 𝒞}{\partial {S}_{Q}^{′}} \qquad ( Equation$$

pertain to entropies from thermal storage and heat transfer respectively. Defining state of health SOH as a degradation measure over the battery's operational life,

$$SOH = \frac{𝒞 ( Δ {t}_{c} , n )}{𝒞 ( Δ {t}_{c} , 0 )} = \frac{{[ - {B}_{G} \int \frac{Δ \dot{G}}{T} dt + {B}_{T} \int \frac{C \dot{T}}{T} dt + {B}_{W} \int \frac{VI}{T} dt ]}_{n}}{{[ - {B}_{G} \int \frac{Δ \dot{G}}{T} dt + {B}_{T} \int \frac{C \dot{T}}{T} dt + {B}_{W} \int \frac{VI}{T} dt ]}_{0}} \qquad ( Equation$$

Dropping the reversible Gibbs component (first terms in numerator and denominator of equation (Equation 4.260)) which cancel over a charge-discharge cycle, and with negligible thermal effects (second terms in numerator and denominator of (Equation 4.260)),

$$SOH = \frac{𝒞 ( Δ {t}_{c} , n )}{𝒞 ( Δ {t}_{c} , 0 )} = \frac{{[ {B}_{W} \int \frac{VI}{T} dt ]}_{n}}{{[ {B}_{W} \int \frac{VI}{T} dt ]}_{0}} \qquad ( Equation$$

With constant B_{W},

$$SOH = \frac{{[ \int \frac{VI}{T} dt ]}_{n}}{{[ \int \frac{VI}{T} dt ]}_{0}} \qquad ( Equation$$

which using irreversible entropy generation notation becomes

$$SOH = \frac{{S}_{n}^{′}}{{S}_{0}^{′}} \qquad ( Equation$$

a measure of accumulated entropy generation after n cycles, with reference to an initial entropy accumulation in the first cycle.

SOH declines with cycling. Inconsistency in SOH arises from different depth of discharge and uncontrolled charging from cycle to cycle. Thus, the internal resistance, state of charge, operating conditions and discharge rate have made accurate SOH estimation an issue in the battery industry. Equation (Equation 4.263) prescribes another approach to determine the state of health of the battery.

Using Internal Resistance Z as Failure Parameter

Recalling equation (Equation 4.188) for internal impedance

$${Z}_{i} = \frac{{V}_{oc}}{I} - {R}_{load} \qquad ( Equation$$

Comparing to equation (Equation 4.254) for a complete cycle,

$$Z = {B}_{{T}_{c}} {\int }_{{t}_{0}}^{{t}_{c}} \frac{C \dot{T}}{T} dt + {B}_{{W}_{c}} {\int }_{{t}_{0}}^{{t}_{c}} \frac{VI}{T} dt + {B}_{{T}_{d}} {\int }_{{t}_{c}}^{{t}_{d}} \frac{C \dot{T}}{T} dt + {B}_{{W}_{d}} {\int }_{{t}_{c}}^{{t}_{d}} \frac{VI}{T} dt \qquad ( Equation$$

where internal resistance coefficients for each of charge and discharge

$${B}_{{T}_{c}} = \frac{\partial Z}{\partial {S}_{{T}_{c}}^{′}} ; {B}_{{W}_{c}} = \frac{\partial Z}{\partial {S}_{{W}_{c}}^{′}} ; {B}_{{T}_{d}} = \frac{\partial Z}{\partial {S}_{{T}_{d}}^{′}} ; {B}_{{W}_{d}} = \frac{\partial Z}{\partial {S}_{{W}_{d}}^{′}} \qquad ( Equation$$

Experimental Setup and Procedure

Experimental parameters to evaluate the energy and entropy formulations were selected based on relevance, convenience and accuracy of available measurement methods. From the above analyses, V_{OC}, V, I, T_{B} and T_{∞} were monitored during cycling.

Lithium-Ion Battery

To obtain sufficient data for statistical significance and establish repeatability, 4 batteries were cycled at two separate and independent discharge rates. 100% cycling (full charge and full discharge) schedule was used.

Apparatus

Each setup had:
1. A single-cell 3.7V Lithium-ion Polymer battery (LiNiMnCo) with graphite anode and aluminum current-collectors.
   Model PL-9059156 manufactured by Batteryspace with 10 Ah nominal capacity rating.
2. A Hitech X48 Multi-charger, powered by a DC power supply for the charge cycle.
3. A set of Dale RH-50 Ω, 50 W resistors for a standardized uniform resistive load.
4. Lead wires with known gages.
5. A current, voltage and resistance meter.
6. Two OMEGA K-type thermocouples, one to measure ambient temperature and the other to measure battery temperature.
7. A National Instrument Data acquisition system (CompactRIO with an analog input module to monitor battery and resistor voltages, an analog output module to automate the cycling process and a thermocouple module to monitor ambient and battery temperatures).
8. Weight scale to measure battery mass before and after testing.

Procedure

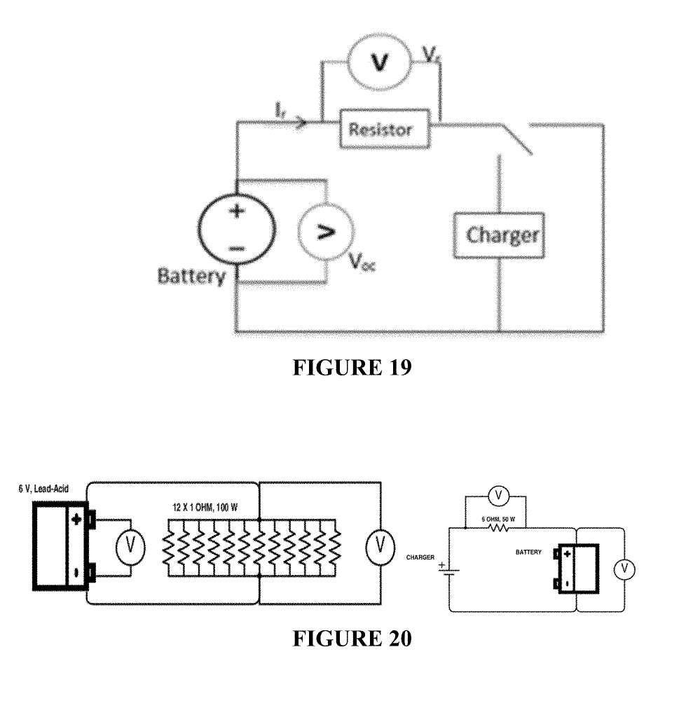

Tests were conducted in a well-ventilated area. All battery tests followed the procedure of
1. Record manufacturer capacity ratings.
2. Set up the charge-discharge cycling circuit on the board provided. The test bench included resistor loads for cell discharging, current measurements and a battery charger (FIG. 19).
3. Measure the operational resistance value of the load resistor network.
4. Attach the wire thermocouple to the battery, connect the terminals of the battery and thermocouple to the CompactRIO system, and verify the initial readings with a meter.
5. Via the CompactRIO, simultaneously record time, open circuit voltage, resistive load voltage, battery temperature, ambient temperature and resistor temperature.
6. Run the NI Labview/CompactRIO data logger for a few minutes to capture the initial steady state of the measurement parameters.
7. Charge the battery to full capacity and record data at a rate of 0.1 Hz.
8. Let battery sit for 20 mins (for voltage relaxation) while continuing data acquisition.

Initial Capacity Testing:
9. Measure the initial weight of the battery using the weight scale.
10. Set up the resistive load as follows:
   For 2 batteries use three 1-ohm resistors in parallel arrangement (theoretical resistance Rₜₕ=⅓Ω; actual resistance is slightly higher due to wiring).
   For the remaining 2 batteries, use two 1-ohm resistors in parallel (Rₜₕ=0.5Ω).
11. Connect the resistor network to the differential analog module to log the voltage drop across the load (the battery's output voltage).
12. Using relays and an analog output module, set up an automatic charge-discharge cycle with overcharge/overdischarge protection (via the smart charger and programmatically). Minimum discharge voltage is 2.7V for full discharge and 2V for overdischarge. For further safety, the setup was closely monitored.
13. Run the data logger a few minutes to capture the initial steady state.
14. Connect the resistor network load to the battery to begin the discharge cycle.
15. Record transient measurements as battery discharges.

Cycling:
16. After the first cycle, repeat steps 7 and 8 to recharge the battery.
17. With automation, continue to cycle the battery until it degrades, observed in one of two ways:
   the battery's capacity falls to two-thirds the initial capacity (determined visually by the duration of discharge) or
   the battery begins to inflate in geometric volume (close monitoring of Li-ion batteries is required during cycling)

Lead-Acid Battery

To promote statistical significance, repeatability and reproducibility, established were two separate and independent deep cycle battery test setups, and two separate and independent starter battery test setups. In addition to electrical and thermal parameters, equation (Equation 4.237) requires measurements of H₂SO₄ concentration at end states.

Apparatus

Each setup (FIG. 20) included:
1. A 3-cell 6V lead-acid battery (starter or deep cycle).
   Deka 901 mf starter battery: 65 Ah (20 hr capacity rating).
   US 2200 XC2 deep cycle battery: 215 Ah (20 hr capacity rating).
2. A Schumacher battery charger SC-600A for the charge portion of the cycle.
3. A set of HS100 1R J 1 ohm, 100 W resistors for a standardized uniform resistive load.
4. A current, voltage and resistance meter.
5. An E-Z RED battery hydrometer to measure electrolyte specific gravity in both end cells.
6. An OMEGA pH meter PHH-5012 to measure pH of both end cells.
7. An OMEGA Digi-Sense compact PFA-coated (corrosion-resistant) K-type thermocouple to measure electrolyte temperature.
8. An OMEGA K-type ambient thermocouple to measure ambient temperature.
9. A National Instruments CompactRIO 9014 with an analog input module to monitor battery voltages and a thermocouple module to monitor ambient and battery temperatures.
10. Weight scale to measure electrolyte loss by evaporation.

Procedure

Appropriate safety protocols were observed. The test area was ventilated, while avoiding air current in the direction of the experiment setup. For each test,
1. Record manufacturer capacity ratings.
2. Measure the actual resistance of the load resistors and the initial battery voltages.
3. Install the corrosion-resistant thermocouple in the battery's middle cell via a small hole drilled through the cell cap, with a tight fit to prevent electrolyte evaporation.
4. Place the ambient thermocouple in the air outside but near the battery, with the sensing terminal making no contact with any surface.
5. Connect both thermocouples to the CompactRIO and configure for K-type measurements.
6. Connect the battery terminals to the CompactRIO differential analog input to log open circuit voltage.
7. Program the CompactRIO to simultaneously record time, open circuit voltage, resistive load voltage, electrolyte temperature, battery temperature, ambient temperature and resistor temperature.
8. Record the initial state of the battery in terms of open circuit voltage, ambient temperature, specific gravity. Measurement procedures ensured
   Battery hydrometer vertical, with bubbles removed from samples by tapping the hydrometer sharply against the chamber side.
9. Run the NI Labview/cRio data logger for a few minutes to capture the initial steady state of the electrical parameters. Log data at a rate of 0.1 Hz as the battery charges.
10. Charge the battery to full capacity. The SC-600A charger defaults to a 2 A charge current for 6V batteries.
11. After the battery has charged, disconnect the charger and let the battery sit for an hour to stabilize, while data logging continues.
12. After settling, take another set of manual readings, then stop the data-logger.

Initial Capacity Testing:
13. Measure the initial weight of the battery using the weight scale.
14. Set up the resistive load as follows:
   For starter batteries, use four 1-Ω resistors in parallel (theoretical resistance Rₜₕ=0.25Ω; actual resistance is higher due to wiring and is accounted for in the data processing).
   For deep-cycle batteries, use twelve 1-ohm resistors in parallel arrangement (Rₜₕ=0.083Ω).
15. Connect the resistor network to the differential analog module to log the voltage drop across the load (the battery's output voltage).
16. Run the data logger a few minutes to capture the initial steady state.
17. Connect the resistor network load to the battery to begin the discharge cycle.
18. Record transient data as battery discharges.
19. Disconnect the battery from resistors after the open circuit voltage falls below 5V (full discharge) or below 2V (overdischarge), to begin the settling process. Keep data logger running until battery is settled (i.e. voltage is slightly fluctuating around 6V). An aged battery after a full discharge settles to a voltage less than initial voltage. Since this settling voltage gradually lessens with cycling, no specific duration is specified for discharge settling. For this work, when the voltage rise is less than 0.01V per 15-minute duration, the battery is considered settled.

Cycling:
20. Repeat steps 9 to 12 to recharge and settle the battery.
21. Repeat steps 16-19 to discharge and settle the battery.
22. Continue steps 20 and 21 until battery degrades. In this work the battery is considered degraded if its terminal voltage begins to drop immediately after connecting the load, i.e. the battery is incapable of supplying steady power at 6V.

Results and Data Analysis

For each battery type (lithium-ion or lead-acid), data was measured and degradation parameters calculated. Data were separated into methods of Gibbs and Heat. A battery cycle consisted of discharge followed by charge, and was not at steady state. Monitored parameters changed with time at unsteady rates. In tables will be data for discharge (left side) and charge (right side). Signs indicate a decrease in a parameter during the process and the direction of the process rate, as in negative capacity and ohmic work for discharge, and positive for charge. Except for temperature, integrals were evaluated using the trapezoidal rule on data over time increment Dt. With data sampled at 0.1 Hz, Δt=10 s. Data processing was automated via Matlab. Figures will have multiple curves. Plots show direction of accumulation and rates (negative accumulation are on the negative axes and vice versa). Plots pertaining to discharge are the “a” part of the figure on the left, and plots pertaining to charge are the “b” part of the figure on the right.

Constants

Values of constants in the formulations include:

Lead-Acid:

Estimated heat transfer coefficient of air, hₐᵢᵣ=20 W/m²K

Thermal conductivity of plastic k=0.22 W/m-K.

Battery mass m=14.5 kg (starter battery), 28 kg (deep cycle battery).

Electrolyte mass mere=3061 g (starter), 2200 g (deep cycle).

Specific heat capacity of acid-water electrolyte

Cp_{elect}=0.65Cp_{H2O}+0.35Cp_{H2SO4} (Equation

was estimated as a sum of individual contributions from water (Cp_{H2O}=4.181/g K) and acid (Cp_{H2SO4}=0.87 J/g K).

Lithium-Ion:

Battery mass m=0.23 kg

Specific heat capacity of polymer electrolyte Cp_{elect}=0.95 J/gK

Thermal resistance for free convection with the surroundings is given by

$${R}_{t} = ( \frac{1}{{h}_{air} {A}_{s}} ) \qquad ( Equation$$

Estimated heat transfer coefficient of air, hₐᵢᵣ=2 W/m²K

Gibbs Thermodynamic Analysis (Maximum Work)

Using equations for estimating battery capacity, Gibbs energy and entropy, data from lead-acid and lithium-ion battery cycling experiments are presented in Table 4.1 for Li-ion batteries and in Table 4.2 for lead acid batteries. In these tables, column 1 variable N numbers the discharge-charge cycles. Other column variables are:
Column 2: Capacity from equation (Equation 4.181)

| | |
|---|---|
| Discharge: C(t) = − ∫ₜₒ^{t}_{d} I (t)dt | Charge: C(t) = ∫ₜₒ^{t}_{c} I(t)dt |

Column 3: Ohmic work from equation (Equation 4.240)

| | |
|---|---|
| Discharge: G_{N}\|W = − ∫ₜₒ^{t}_{d} VI dt | Charge: G_{N}\|W = ∫ₜₒ^{t}_{c} VI dt |

Subscripts denote cycle number N, end of discharge d and end of charge c.
Column 4: Thermal energy from equation (Equation 4.240)

| | |
|---|---|
| Discharge: ΔG_{N}\|T = − ∫ₜₒ^{t}_{d} CT dt | Charge: ΔG_{N}\|T = − ∫ₜₒ^{t}_{c} CT dt |

Column 5: Accumulated Gibbs energy from equation (Equation 4.240)

ΔG_{N}=G_{N}|W+ΔG_{N}|T
Column 6: From equation (Equation 4.241), entropy generation from ohmic work S′_{N}|W

| | |
|---|---|
| $Discharge \text{:} {{S}^{′}}_{N} \text{\textbar} W = {\int }_{{t}_{0}}^{{t}_{d}} \frac{VI}{T} dt$ | $C harge \text{:} {{S}^{′}}_{N} \text{\textbar} W = {\int }_{{t}_{0}}^{{t}_{c}} \frac{VI}{T} dt$ |

Column 7: Thermal entropy S′_{N}|T from equation (Equation 4.241)

| | |
|---|---|
| $Discharge \text{:} {{ΔS}^{′}}_{N} \text{\textbar} T = {\int }_{{t}_{0}}^{{t}_{d}} \frac{C \dot{T}}{T} dt$ | $C harge \text{:} {{ΔS}^{′}}_{N} \text{\textbar} T = {\int }_{{t}_{0}}^{{t}_{c}} \frac{C \dot{T}}{T} dt$ |

Column 8: Gibbs (total) entropy generation from equation (Equation 4.241)

S′_{N}=S′_{N}|W+ΔS′_{N}|T

Contributions from

$$\frac{Δ \dot{G}}{T} = \frac{- n F Δ {\dot{V}}_{OC}}{T}$$

for constant-voltage charge and discharge processes (Δ\dot{V}_{OC}|ch=−Δ\dot{V}_{OC}|disch) or ΔV_{OC}=0 which sum to zero over each cycle, were dropped.
Column 9: capacity-ohmic degradation coefficient from equation (Equation 4.257)

$${B}_{W} = \frac{\partial C}{\partial {S}_{W}^{′}} .$$

Column 10: capacity-thermal degradation coefficient from equation (Equation 4.257)

$${B}_{T} = \frac{\partial C}{\partial {S}_{T}^{′}} .$$

Column 11: residual capacity from the surface fit of entropy components to capacity

[symbol]

ᵣₑₛ= [symbol] −ΣᵢBᵢS′ᵢ (Equation 4.269)

the difference between measured capacity and the capacity predicted by the DEG theorem.

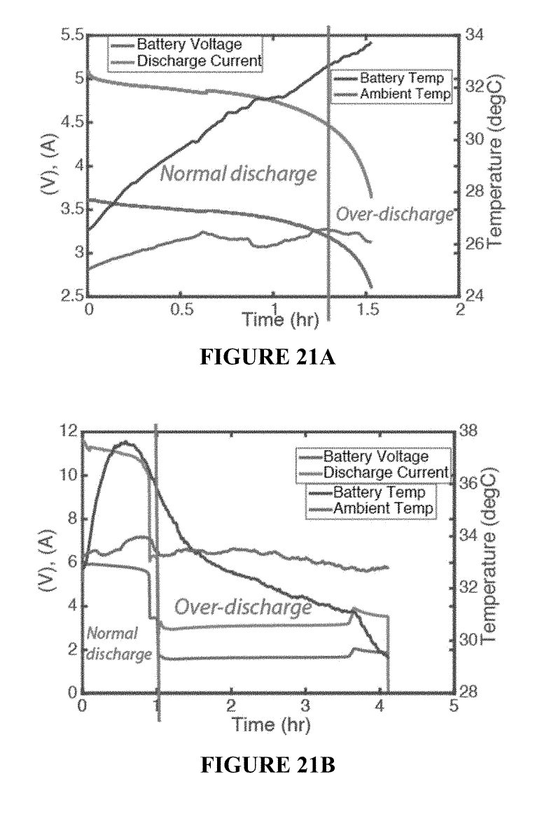

Tables 4.1 and 4.2 were populated using the above equations. Current, voltage and temperatures versus time during cycling are shown in FIGS. 21A and 21B. The vertical lines in FIGS. 21A and 21B demarcate the normal discharge and over-discharge regions for both li-ion and lead-acid batteries.

Li-Ion Battery

A sample dataset, cycle 4 of Li-ion battery #2 data in Table 4.1 (with bold font) was used. Focus is on the normal discharge region, see FIG. 21A, with the over-discharge region treated later. Data and plots for discharge are to the left, and data and plots for charge to the right. The lithium-ion batteries were discharged to the manufacturer-specified lower limit of 2.7V for 100% depth of discharge DOD. Similar trends were observed for all batteries tested.

TABLE 4.1
Processed parameters for li-ion battery (Discharge rate: 5 A, Charge rate: 4 A). Cycle 4 (in bold)
is used in the breakdown in this section. Subsequent charges before next discharge are combined.

| | Normal Discharge | | | | | | | | | | Charge |
|---|---|---|---|---|---|---|---|---|---|---|---|
| | [symbol] | G_{N}\|W | ΔG_{N}\|T | ΔG_{N} | S′_{N}\|W | ΔS′_{N}\|T | S′_{N} | B_{W} | B_{T} | [symbol] ᵣₑₛ | [symbol] |
| | A-hr | kJ | kJ | kJ | J/K | J/K | J/K | AhrK/J | AhrK/J | A-hr | A-hr |
| 1 | −4.84 | −51.8 | −1.2 | −53.0 | 170.2 | 4.0 | 174.1 | −0.030 | −0.018 | 0.073 | 11.30 |
| 2 | −6.70 | −70.1 | −1.9 | −72.0 | 232.1 | 6.2 | 238.4 | −0.031 | −0.012 | 0.107 | 11.23 |
| 3 | −8.26 | −86.8 | −1.1 | −87.9 | 281.8 | 3.7 | 285.5 | −0.030 | 0.019 | 0.049 | 8.38 |
| 4 | −6.15 | −64.2 | −1.4 | −65.5 | 211.6 | 4.5 | 216.1 | −0.031 | 0.006 | 0.102 | 9.68 |
| 5 | −5.83 | −61.6 | −1.3 | −62.9 | 203.9 | 4.2 | 208.1 | −0.030 | −0.003 | 0.092 | 18.21 |
| 6 | −8.52 | −90.3 | −1.7 | −92.0 | 299.1 | 5.6 | 304.7 | −0.030 | −0.024 | 0.112 | 7.89 |
| 7 | −5.31 | −55.6 | −1.2 | −56.8 | 184.3 | 4.1 | 188.4 | −0.030 | 0.041 | 0.038 | 3.86 |
| 8 | −8.58 | −91.5 | −1.6 | −93.1 | 300.4 | 5.1 | 305.5 | −0.029 | 0.068 | −0.006 | 10.65 |
| 9 | −4.31 | −45.7 | −1.5 | −47.2 | 152.5 | 5.2 | 157.7 | −0.030 | 0.027 | 0.045 | 11.92 |
| 10 | −7.04 | −74.5 | −1.1 | −75.7 | 245.3 | 3.7 | 249.1 | −0.030 | 0.035 | 0.091 | 13.75 |
| 11 | −9.59 | −104.9 | −1.2 | −106.1 | 340.9 | 3.9 | 344.8 | −0.028 | 0.070 | 0.038 | 6.13 |
| 12 | −5.09 | −53.4 | −1.6 | −55.0 | 177.7 | 5.2 | 182.9 | −0.031 | 0.041 | 0.070 | 9.90 |
| 13 | −7.05 | −75.3 | −1.4 | −76.7 | 249.4 | 4.8 | 254.2 | −0.031 | −0.003 | 0.145 | 9.59 |
| 14 | −6.81 | −72.5 | −1.6 | −74.1 | 239.9 | 5.3 | 245.2 | −0.030 | 0.007 | 0.078 | 11.80 |
| 15 | −8.46 | −91.8 | −1.2 | −92.9 | 302.0 | 3.8 | 305.9 | −0.029 | 0.028 | 0.102 | 3.08 |
| 16 | −5.36 | −56.9 | −0.9 | −57.9 | 188.0 | 3.1 | 191.1 | −0.030 | 0.012 | 0.111 | 9.56 |
| 17 | −7.11 | −76.5 | −1.2 | −77.7 | 252.3 | 4.0 | 256.3 | −0.029 | 0.033 | 0.078 | 11.00 |
| 18 | −7.37 | −79.7 | −1.4 | −81.0 | 262.1 | 4.5 | 266.6 | −0.030 | −0.013 | 0.155 | 8.14 |
| 19 | −5.21 | −55.0 | −1.2 | −56.2 | 181.5 | 3.8 | 185.3 | −0.029 | 0.033 | 0.034 | 9.26 |
| 20 | −6.08 | −62.4 | −0.9 | −63.3 | 205.8 | 3.0 | 208.8 | −0.030 | 0.004 | 0.041 | 9.86 |
| 21 | −6.36 | −66.4 | −1.8 | −68.2 | 220.9 | 6.1 | 227.0 | −0.030 | 0.030 | 0.064 | 11.28 |
| 22 | −3.31 | −35.2 | −0.7 | −35.9 | 116.3 | 2.4 | 118.7 | −0.029 | 0.010 | 0.034 | 13.64 |
| 23 | −9.96 | −105.0 | −1.1 | −106.1 | 342.9 | 3.7 | 346.6 | −0.031 | 0.004 | 0.164 | 9.69 |
| 24 | −5.36 | −54.8 | −1.0 | −55.9 | 181.1 | 3.4 | 184.5 | −0.031 | 0.008 | 0.087 | 4.18 |
| 25 | −3.57 | −36.1 | −1.0 | −37.1 | 118.8 | 3.2 | 122.0 | −0.032 | 0.003 | 0.062 | 19.43 |
| 26 | −2.56 | −26.9 | −1.7 | −28.6 | 89.0 | 5.6 | 94.6 | −0.032 | −0.005 | 0.049 | 4.16 |
| 27 | −3.92 | −40.1 | −1.2 | −41.3 | 132.3 | 3.9 | 136.2 | −0.033 | −0.001 | 0.117 | 14.43 |
| 28 | −2.14 | −22.3 | −1.7 | −24.0 | 73.9 | 5.6 | 79.5 | −0.033 | −0.002 | 0.049 | 16.81 |
| 29 | −2.06 | −21.4 | −1.7 | −23.1 | 70.9 | 5.6 | 76.6 | −0.033 | −0.001 | 0.045 | 11.11 |

| | Charge | | | | | | | | |
|---|---|---|---|---|---|---|---|---|---|
| | G_{N}\|W | ΔG_{N}\|T | ΔG_{N} | S′_{N}\|W | ΔS′_{N}\|T | S′_{N} | B_{W} | B_{T} | [symbol] ᵣₑₛ |
| | kJ | kJ | kJ | J/K | J/K | J/K | AhrK/J | AhrK/J | A-hr |
| 1 | 161.6 | −0.2 | 161.4 | 531.5 | 0.6 | 532.1 | 0.021 | 0.067 | 0.304 |
| 2 | 158.6 | −0.1 | 158.4 | 522.0 | 0.5 | 522.5 | 0.021 | 0.050 | 0.290 |
| 3 | 119.0 | 0.6 | 119.5 | 389.6 | −1.9 | 387.7 | 0.021 | 0.061 | −0.019 |
| 4 | 138.0 | 0.0 | 138.0 | 455.2 | 0.0 | 455.2 | 0.021 | 0.065 | 0.056 |
| 5 | 263.1 | 0.0 | 263.1 | 870.6 | 0.0 | 870.6 | 0.021 | 0.078 | 0.189 |
| 6 | 114.3 | −0.2 | 114.1 | 377.4 | 0.8 | 377.5 | 0.021 | 0.138 | 0.017 |
| 7 | 53.8 | 0.3 | 54.2 | 176.2 | −1.1 | 175.0 | 0.022 | 0.016 | −0.048 |
| 8 | 152.0 | −0.1 | 151.9 | 502.0 | 0.2 | 502.2 | 0.021 | 0.062 | 0.049 |
| 9 | 171.0 | −0.4 | 170.6 | 565.8 | 1.4 | 567.2 | 0.021 | 0.016 | 0.115 |
| 10 | 197.8 | 0.0 | 197.8 | 648.9 | 0.0 | 649.0 | 0.021 | 0.110 | 0.188 |
| 11 | 86.2 | 0.1 | 86.3 | 280.9 | −0.3 | 280.6 | 0.022 | 0.042 | 0.007 |
| 12 | 141.0 | 0.0 | 140.9 | 464.9 | 0.1 | 465.1 | 0.021 | 0.070 | 0.137 |
| 13 | 136.3 | 0.1 | 136.4 | 451.1 | −0.3 | 450.9 | 0.021 | 0.074 | −0.036 |
| 14 | 168.5 | 0.0 | 168.5 | 553.6 | −0.1 | 553.4 | 0.021 | 0.056 | 0.095 |
| 15 | 43.7 | 0.1 | 43.8 | 143.7 | −0.3 | 143.4 | 0.021 | 0.061 | 0.008 |
| 16 | 136.6 | 0.0 | 136.6 | 452.5 | 0.0 | 452.5 | 0.021 | 0.061 | 0.114 |
| 17 | 157.5 | −0.2 | 157.4 | 517.5 | 0.5 | 518.1 | 0.021 | 0.062 | 0.106 |
| 18 | 115.6 | 0.1 | 115.7 | 381.0 | −0.3 | 380.7 | 0.021 | 0.058 | −0.070 |
| 19 | 132.0 | −0.1 | 131.9 | 436.3 | 0.3 | 436.6 | 0.021 | 0.070 | 0.005 |
| 20 | 140.5 | −0.4 | 140.1 | 464.8 | 1.2 | 466.0 | 0.021 | 0.054 | −0.047 |
| 21 | 161.0 | 0.0 | 161.0 | 529.5 | 0.1 | 529.6 | 0.021 | 0.072 | 0.042 |
| 22 | 199.7 | −0.2 | 199.5 | 664.6 | 0.6 | 665.2 | 0.020 | 0.017 | 0.096 |
| 23 | 138.1 | −0.4 | 137.7 | 457.4 | 1.3 | 458.7 | 0.021 | 0.068 | 0.000 |
| 24 | 60.2 | −0.1 | 60.1 | 198.2 | 0.1 | 198.3 | 0.021 | 0.055 | 0.021 |
| 25 | 245.5 | −0.5 | 245.0 | 814.4 | 1.7 | 816.1 | 0.024 | 0.014 | −0.002 |
| 26 | 59.2 | −0.1 | 59.1 | 195.8 | 0.2 | 195.6 | 0.021 | 0.045 | −0.034 |
| 27 | 208.9 | −0.4 | 208.5 | 690.2 | 1.2 | 691.4 | 0.021 | 0.057 | 0.113 |
| 28 | 245.6 | −1.5 | 244.0 | 817.5 | 5.1 | 822.6 | 0.020 | 0.000 | 0.025 |
| 29 | 158.3 | −0.2 | 158.1 | 522.4 | 0.7 | 523.0 | 0.021 | 0.132 | 0.036 |

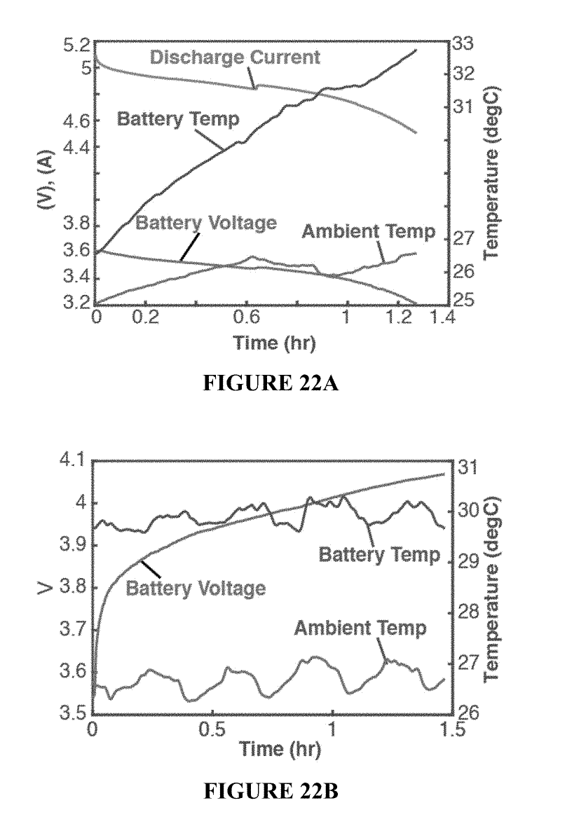

In FIGS. 22A-22B, charge/discharge current I, battery voltage V and temperature T were plotted versus time as a battery discharged and charged during cycle 4. FIG. 22A shows an hour discharge with a monotonic rise in battery temperature, and FIG. 22B shows a 1.5-hour charge cycle with a slight change in temperature. Ambient temperature fluctuations arose from surrounding conditions (students entering and exiting the laboratory). Temperatures are plotted on the right axis, while current and voltage are plotted on the left axis. Discharge rate=5 A, charge rate=4 A.

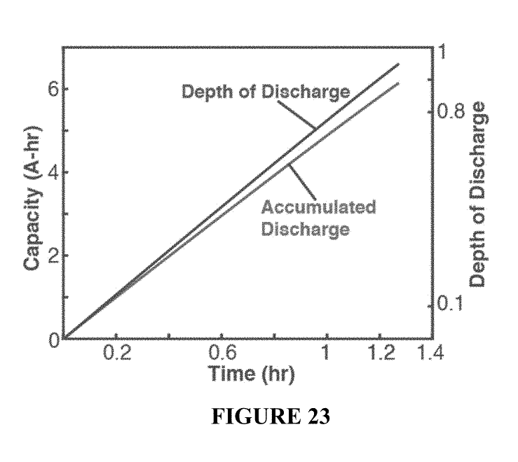

FIG. 23 plots discharge and charge capacity, where discharge capacity

$$C ( t ) = - {\int }_{{t}_{0}}^{{t}_{d}} I ( t ) dt \approx - {\sum }_{1}^{n} [ \frac{{I}_{n} + {I}_{n - 1}}{2} ] Δ t \qquad ( Equation$$

where n=1, 2, 3, . . . is a vector index corresponding to t₁, t₂, t₃, etc and Δt=tₙ−tₙ₋₁. The accumulated charge (capacity) dropped during discharge and increased during charge (FIG. 23).

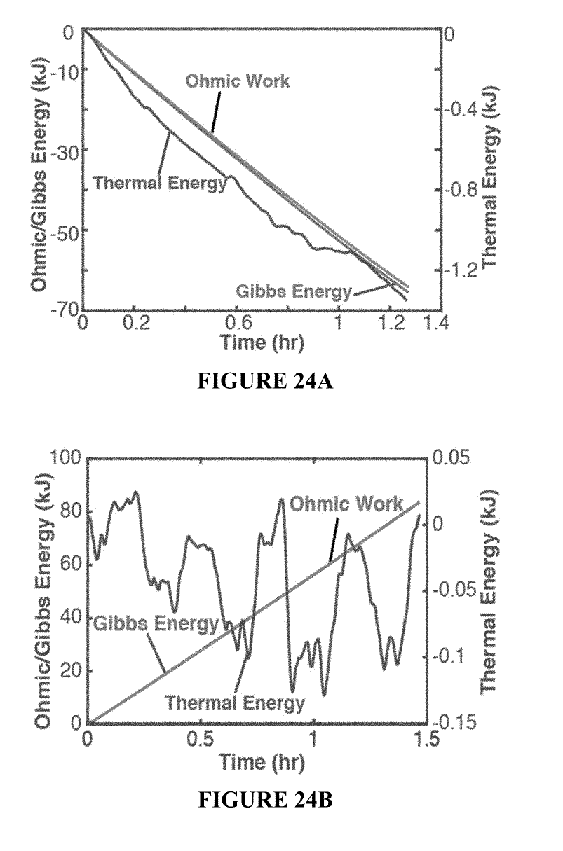

FIGS. 24A and 24B plot accumulated Gibbs energy and its components during discharge and charge, with Gibbs Ohmic work, column 3

$${G}_{N} \vert W = {\int }_{{t}_{0}}^{{t}_{d}} IVdt = {\sum }_{1}^{n} [ \frac{{I}_{n} {V}_{n} + {I}_{n - 1} {V}_{n - 1}}{2} ] Δ t , \qquad ( Equation 4.271 )$$

Gibbs thermal energy, column 4

ΔG_{N}|T=∫ₜ_{₀}^{t}^{_{d}}C\dot{T}dt=C(T_{d}−T₀) (Equation 4.272)

and total Gibbs energy ΔG_{N}=G_{N}|W+ΔG_{N}|T in column 5. To monitor and plot changes in thermal energy at times t₁, t₂, t₃, . . . , integrals were decomposed. For times t₁ and tₙ,

ΔG₁|T=∫ₜ_{₀}^{t}^{₁}C\dot{T}dt=C(T₁−T₀). (Equation 4.273)

ΔGₙ|T=∫ₜ_{₀}^{t}^{ₙ}C\dot{T}dt=ΔG₁|T+ . . . +ΔGₙ₋₁|T+C(Tₙ−Tₙ₋₁) (Equation

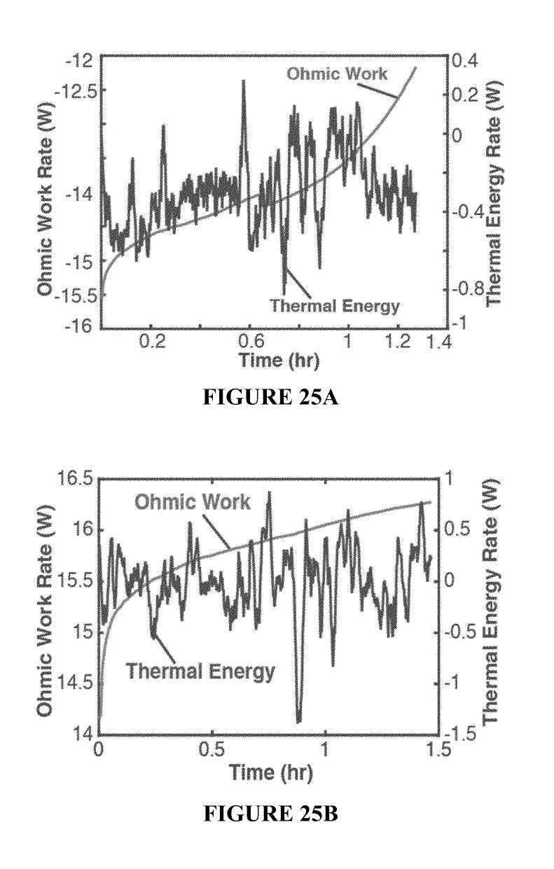

Ohmic work ΔG_{N}|W (FIGS. 24A and 24B) linearly decreased during discharge, and increased during charge. Thermal component ΔG_{N}|T changes the available Gibbs energy. With ohmic heating dominating heat removal mechanisms, including free convection to the environment, thermal energy in the battery increases, reducing available energy. The consistent trend in ΔG_{N}|T on the discharge side of Table 4.1 suggests a consistent thermal response for all cycles (relatively consistent discharge rate, DOD varying cycle to cycle). The battery's Gibbs energy, dominated by ohmic work, behaved similarly. The thermal energy, 1 to 2 orders of magnitude less than ohmic (Table 4.1), could be neglected. Thermal energy changes depend on heat capacity and change in battery temperature. Rates of processes (FIGS. 25A and 25B) influence energies.

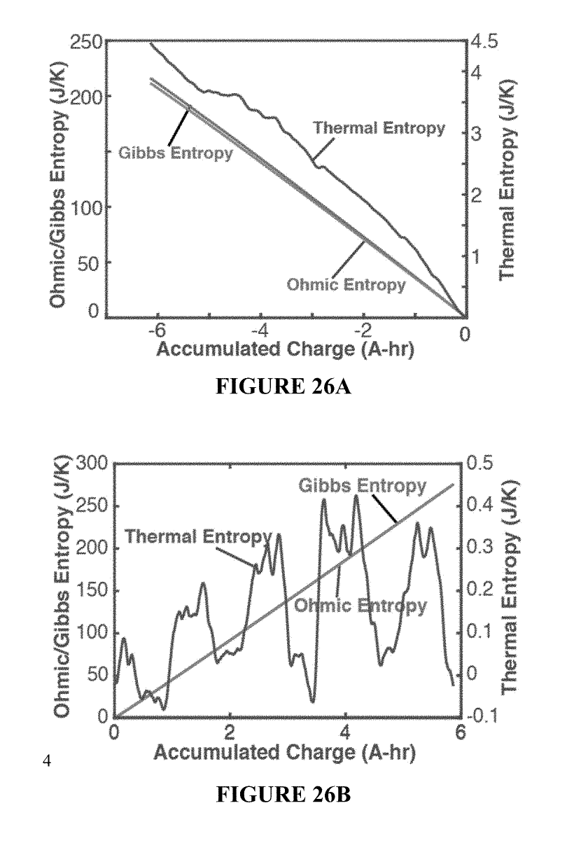

FIGS. 26A and 26B plot Ohmic S′_{N}|W (column 6), thermal (column 7) ΔS′_{N}|T and total S′_{N} (column 8) entropies versus time, calculated via ΔS′_{N}=ΔS′_{N}|W+ΔS′_{N}|T where

$$\begin{matrix}{S}_{N}^{′} \vert W = {\int }_{{t}_{0}}^{{t}_{d}} \frac{IV}{T} dt = {\sum }_{1}^{n} [ \frac{{I}_{n} {V}_{n} + {I}_{n - 1} {V}_{n - 1}}{{T}_{ave}} ] \frac{Δ t}{2} & ( Equation \\ Δ {S}_{N}^{′} \vert T = {\int }_{{t}_{0}}^{{t}_{d}} \frac{C \dot{T}}{T} dt = {\sum }_{1}^{n} [ \frac{C ( {T}_{n} - {T}_{n - 1} )}{{T}_{ave}} ] & ( Equation\end{matrix}$$

for a process from t₀ to t_{d}. Entropies are generated at the instantaneous non-isothermal temperature, estimated via an average

$${T}_{ave} = \frac{{T}_{n} + {T}_{n - 1}}{2}$$

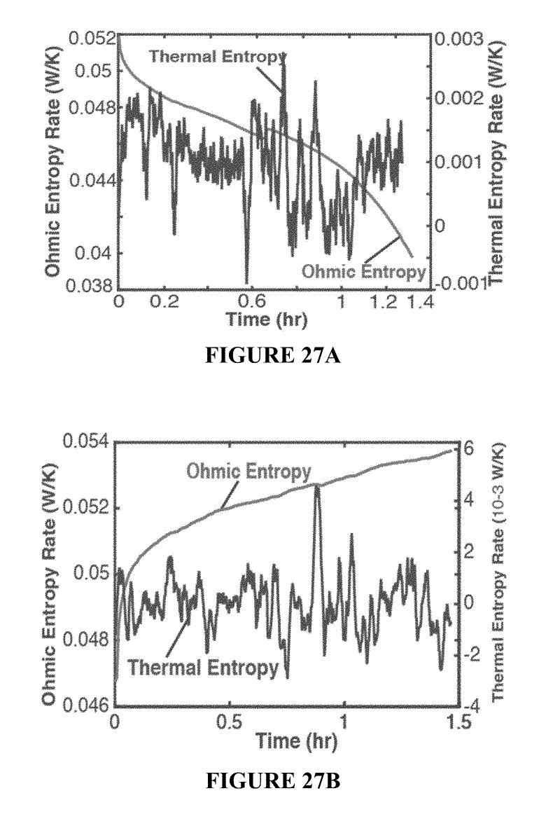

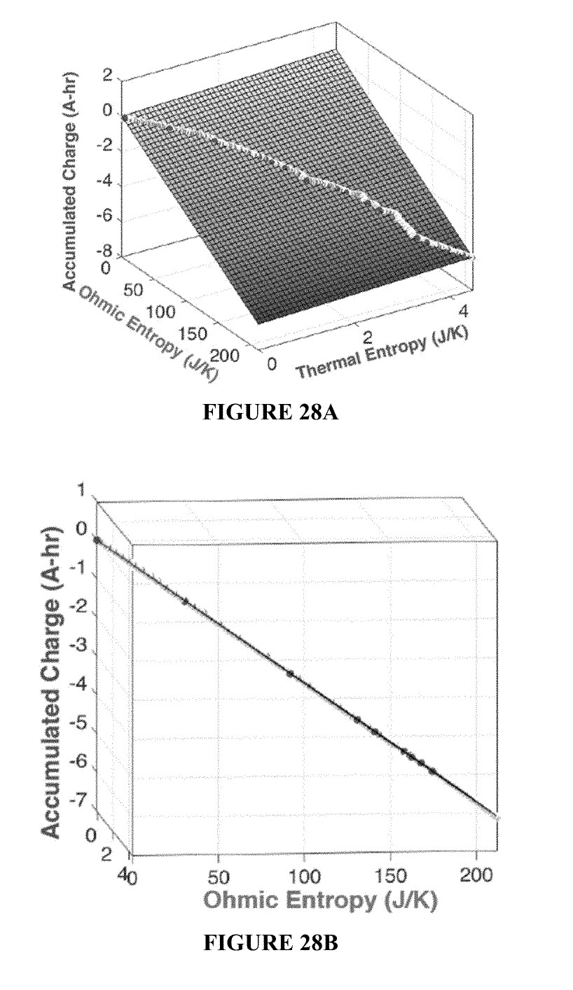

Accumulated charge appears linear in Ohmic entropy and nearly linear with thermal entropy. Ohmic entropy generation rate decreased with decrease in current during cycling (FIGS. 27A-27B). With the relatively low temperature change rate observed, the thermal entropy change rate was also low (FIGS. 27A-27B). With both accumulated ohmic and thermal entropies linear, total Gibbs entropy was linear for both charging and discharging (FIGS. 26A-26B). The actual partial contributions better visualize in the 3D surface plot, FIGS. 28A and 28B. As with energy, the thermal contribution to entropy generation is 2 orders of magnitude less than the ohmic contribution, but contributes significantly to charge/discharge accumulation, and unlike thermal energy, should not be neglected. The significance of thermal entropy is underscored by the need to keep batteries cool during operation, for better and longer performance.

Degradation Coefficients Bᵢ

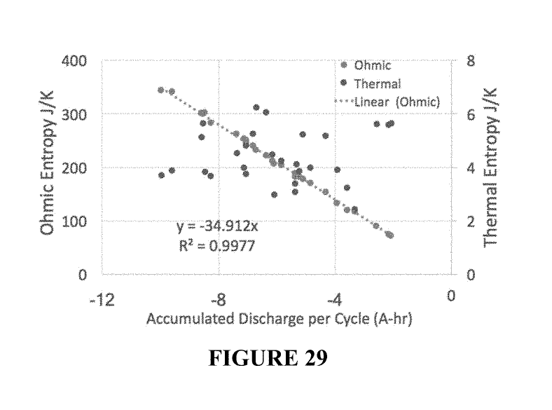

By associating data from the time instants, accumulated discharge (capacity) was plotted versus accumulated entropies. FIGS. 28A-28B plot this in a 3-dimensional space for data of cycle 4. The battery's path through the space during discharge, from upper left corner to lower right corner—its Degradation Entropy Generation (DEG) trajectory—stays in a plane—its DEG surface. FIG. 28B shows a perfect coincidence of the data points to the planar 2D surface, hence goodness of fit R²=1, rare for uncontrolled conditions and changing rates. This suggests a linear dependence of capacity accumulation on both ohmic and thermal entropies, confirmed in FIG. 29, which plots the final capacity accumulation after each cycle of Table 4.1 versus the final accumulated ohmic and thermal entropies. Different dots represent different cycles. In FIG. 29, the thermal entropy is orders of magnitude less than the Ohmic entropy and shows scatter (from temperature sensitivity).

The 3-D space of the DEG surface characterizes the allowable regime in which the battery can operate. A battery's DEG domain (here Capacity versus Ohmic & Thermal Entropy) can define consistent parameters for identifying desired characteristics from batteries of all configurations, similar to the Ragone plot.

Degradation coefficients B_{W} and B_{T}, partial derivatives of capacity to ohmic and thermal entropies respectively, see equation (Equation 4.257), were estimated from the surface fit at each point of FIGS. 28A-28B, see columns 9 and 10 of Table 4.1. Here B_{W} is nearly constant over all cycles, with an average B_{W}=−0.03. The values of B_{T} show more variation, probably due to temperature measurement sensitivity and surrounding conditions. Although not required by this approach, a controlled environment can give more consistent B_{T}. The results are not considerably affected as overall thermal entropy here is negligible. It is also noted that heat capacity used is an estimate based on electrolyte only. The relative closeness in magnitude of both B_{T} and B_{W} for the lithium-ion batteries studied implies the same order of impact of both processes on accumulation, which can be deduced from the DEG trajectory. Hence, proper formulation of the governing entropies of the active processes is required to accurately determine their contributions to overall accumulation and degradation.

FIG. 29 predicts a constant B_{W}≈−0.029 applies to all cycles in Table 4.1.

B_{W} values from charge (right side of Table 4.1) are positive to indicate an energy-adding transformation.

Lead-Acid Battery

The batteries used were previously cycled and slightly degraded before measurements. Battery lives such as the heavy duty Deka 6V batteries used are a few hundreds of cycles. To accelerate measurable degradation, batteries were significantly discharged below manufacturer-intended levels and the discharge rate (current) was increased from 11 A to 35 A after the first 9 cycles. For lead-acid batteries, discharge up to the initial cliff drop is useful; after this the battery cannot deliver power at the nominal voltage (6V), and is considered fully discharged. This section is similar to the lithium-ion battery, with cycle 2 of Table 4.2 for the Deka Starter battery presented, and lead-acid battery trends emphasized. Similar trends were observed for the other lead acid batteries.

TABLE 4.2
Processed parameters for lead-acid starter battery (Discharge rate: ~11 A, bold
line between cycles 9 and 10 marks the start of 35 A discharge rate. Charge rate:
1.2 A). Cycle 2 (in bold) is used in the detail breakdown in this section.

| | Normal Discharge | | | | | | | | | | Charge |
|---|---|---|---|---|---|---|---|---|---|---|---|
| | [symbol] | G_{N}\|W | ΔG_{N}\|T | ΔG_{N} | S′_{N}\|W | ΔS′_{N}\|T | S′_{N} | B_{W} | B_{T} | [symbol] ᵣₑₛ | [symbol] |
| N | A-hr | kJ | kJ | kJ | J/K | J/K | J/K | AhrK/J | AhrK/J | A-hr | A-hr |
| 1 | −54.56 | −961.1 | 2.3 | −958.9 | 3096.9 | −7.5 | 3089.4 | −0.018 | 0.204 | 0.005 | 6.12 |
| 2 | −9.87 | −176.1 | −34.8 | −210.9 | 569.2 | 112.9 | 682.1 | −0.018 | 0.019 | 0.001 | 16.55 |
| 3 | −10.22 | −176.6 | −11.9 | −188.5 | 576.0 | 38.8 | 614.8 | −0.018 | 0.014 | 0.001 | 15.19 |
| 4 | −12.43 | −224.3 | −40.2 | −264.5 | 711.9 | 129.9 | 841.8 | −0.017 | 0.045 | 0.000 | 15.30 |
| 5 | −8.31 | −145.0 | −43.7 | −188.6 | 465.7 | 140.9 | 606.6 | −0.018 | 0.052 | 0.001 | 25.36 |
| 6 | −14.75 | −266.7 | −9.6 | −276.3 | 853.1 | 31.0 | 884.1 | −0.017 | 0.026 | 0.000 | 19.16 |
| 7 | −12.80 | −230.9 | −30.9 | −261.9 | 730.7 | 99.7 | 830.5 | −0.018 | 0.028 | 0.000 | 16.88 |
| 8 | −11.16 | −189.8 | 3.7 | −186.1 | 615.0 | −12.0 | 603.0 | −0.018 | 0.006 | 0.031 | 18.62 |
| 9 | −9.00 | −175.1 | 13.9 | −161.2 | 571.5 | −45.2 | 526.3 | −0.017 | 0.018 | −0.019 | 25.23 |
| 10 | −13.10 | −258.7 | 13.1 | −245.6 | 844.3 | −42.7 | 801.7 | −0.016 | 0.025 | −0.010 | 29.65 |
| 11 | −10.21 | −197.1 | 15.0 | −182.1 | 644.4 | −48.9 | 595.5 | −0.015 | 0.048 | 0.004 | 19.49 |
| 12 | −11.16 | −218.2 | 16.6 | −201.7 | 711.6 | −54.0 | 657.6 | −0.015 | 0.059 | 0.008 | 26.69 |
| 13 | −9.93 | −190.2 | 17.7 | −172.5 | 619.7 | −57.6 | 562.1 | −0.015 | −0.010 | 0.015 | 25.13 |
| 14 | −7.68 | −145.4 | 14.8 | −130.6 | 476.8 | −48.7 | 428.1 | −0.015 | −0.005 | 0.012 | 19.59 |
| 15 | −4.86 | −90.3 | 16.1 | −74.2 | 297.3 | −53.0 | 244.3 | −0.015 | −0.008 | 0.007 | 20.69 |
| 16 | −3.95 | −72.4 | 14.1 | −58.3 | 237.4 | −46.4 | 191.0 | −0.016 | −0.001 | 0.005 | 7.98 |
| 17 | −2.51 | −45.6 | 8.8 | −36.7 | 149.4 | −29.0 | 120.4 | −0.016 | −0.003 | 0.006 | 11.26 |

| | Charge | | | | | | | | |
|---|---|---|---|---|---|---|---|---|---|
| | G_{N}\|W | ΔG_{N}\|T | ΔG_{N} | S′_{N}\|W | ΔS′_{N}\|T | S′_{N} | B_{W} | B_{T} | [symbol] ᵣₑₛ |
| N | kJ | kJ | kJ | J/K | J/K | J/K | AhrK/J | AhrK/J | A-hr |
| 1 | −11.6 | −13.4 | −25.1 | 38.2 | 44.1 | 82.3 | 0.172 | 0.025 | −0.013 |
| 2 | −32.3 | −44.0 | −76.2 | 106.3 | 145.2 | 251.5 | 0.163 | −0.049 | −0.006 |
| 3 | −29.6 | −27.0 | −56.6 | 97.2 | 88.3 | 185.5 | 0.151 | 0.083 | 0.004 |
| 4 | −29.9 | −36.8 | −66.7 | 98.7 | 120.8 | 219.5 | 0.150 | 0.048 | 0.003 |
| 5 | −39.4 | −60.7 | −100.1 | 129.5 | 199.3 | 328.8 | 0.230 | −1.757 | −0.020 |
| 6 | −36.9 | −59.4 | −96.2 | 121.2 | 194.7 | 315.8 | 0.145 | −0.082 | 0.008 |
| 7 | −32.8 | −51.1 | −83.9 | 107.7 | 167.2 | 274.9 | 0.150 | 0.019 | 0.002 |
| 8 | −124.7 | −31.9 | −156.6 | 408.9 | 104.0 | 512.9 | 0.075 | −6.375 | 0.043 |
| 9 | −153.0 | −21.5 | −174.5 | 498.4 | 70.1 | 568.5 | 0.047 | 5.466 | 0.109 |
| 10 | −19.4 | −112.0 | −131.4 | 63.8 | 373.0 | 436.8 | 0.474 | 0.167 | −0.003 |
| 11 | −13.0 | −118.5 | −131.5 | 43.1 | 395.7 | 438.8 | 0.460 | −0.009 | −0.001 |
| 12 | −17.9 | −123.5 | −141.4 | 59.0 | 409.5 | 468.5 | 0.461 | 0.018 | −0.001 |
| 13 | −16.0 | −59.0 | −74.9 | 52.4 | 194.5 | 246.9 | 0.502 | −0.115 | −0.008 |
| 14 | −11.7 | −8.8 | −20.5 | 38.5 | 28.7 | 67.2 | 0.485 | −0.284 | 0.009 |
| 15 | −13.9 | −51.7 | −65.6 | 46.0 | 171.9 | 217.9 | 0.454 | −0.003 | −0.001 |
| 16 | −15.6 | −31.2 | −46.8 | 51.7 | 120.1 | 171.8 | 0.480 | −0.134 | 0.000 |
| 17 | −21.9 | −5.6 | −27.5 | 72.2 | 23.9 | 96.1 | 0.474 | 0.015 | −0.003 |

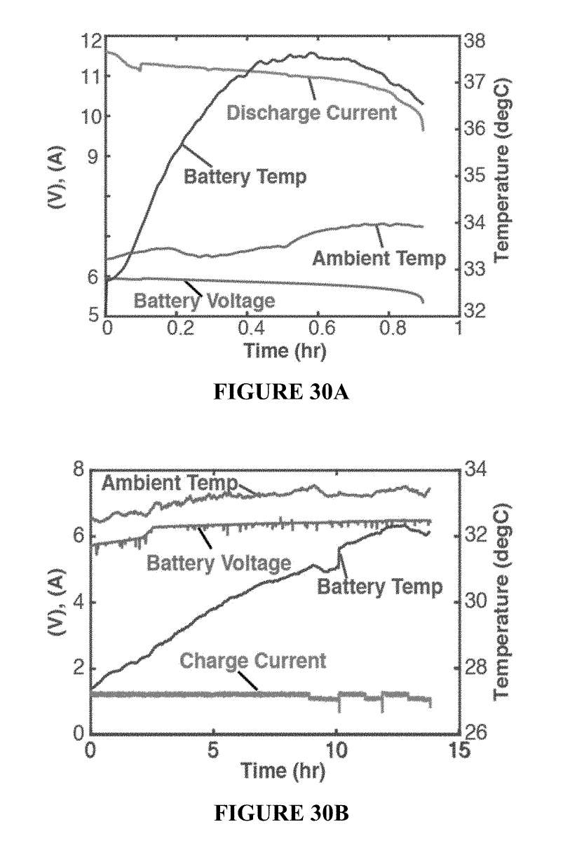

The lead-acid batteries showed slightly different thermal behavior from the lithium-ion batteries during cycling, due to differences in underlying mechanisms and material compositions. Overall cycle-to-cycle trends and current-voltage characteristics are similar. FIGS. 30A-30B show an hour discharge followed by a 14-hour charge for cycle 2. Battery temperature initially rose during the first half hour of discharge followed by a drop, with the discharge rate still high and ambient temperature stable. This recovery feature, more significant in starter batteries, helps a battery quickly equilibrate after the initial response to load.

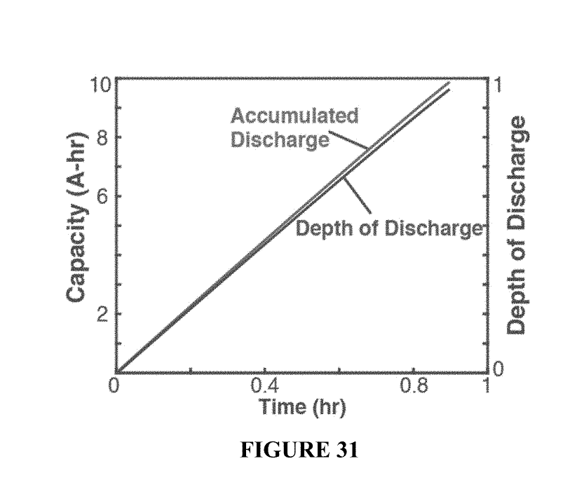

Capacity dropped during discharge and increased during charge (FIG. 31).

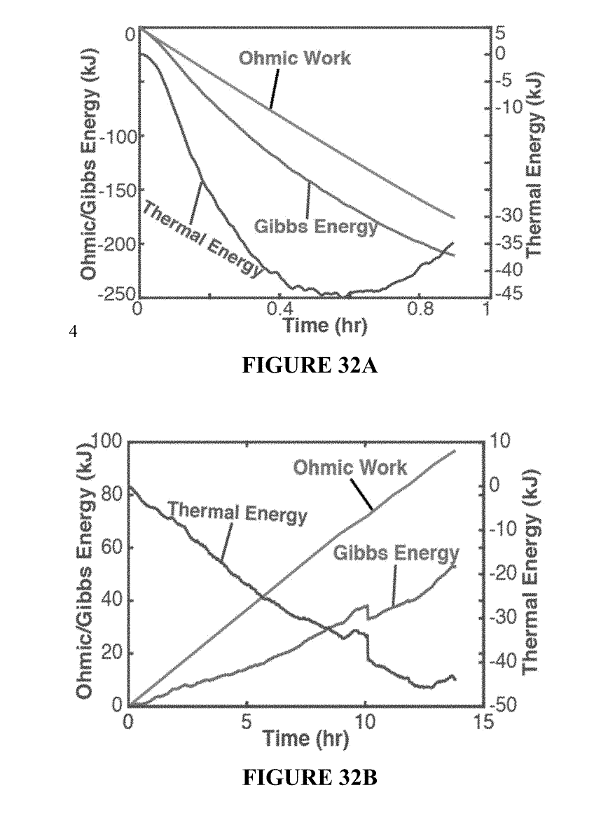

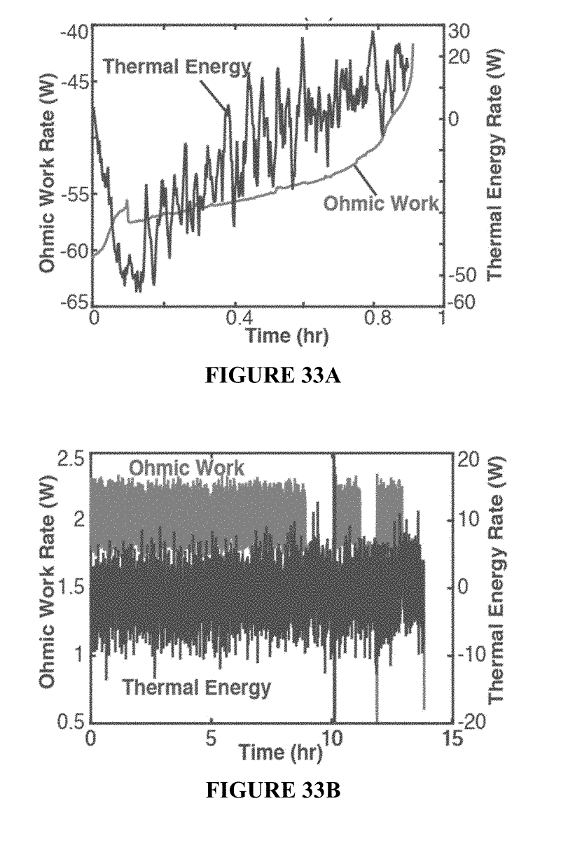

Ohmic work linearly decreased during discharge and increased during charge (FIGS. 32A-32B). The thermal energy change during cycling mimicked the nonlinear temperature variation in FIGS. 30A-30B. This differs from the linear trend observed for the lithium-ion batteries, and is a possible reason current models do not apply across battery types. FIGS. 32A-32B and Table 4.2 show that ohmic work dominates lead-acid battery cycling, and determines overall trends in total Gibbs energy change. Also, unlike the lithium-ion batteries, the thermal energy change is significant to total Gibbs energy and gives it a curvature (FIGS. 32A-32B). Gibbs process rates are plotted in FIGS. 33A-33B.

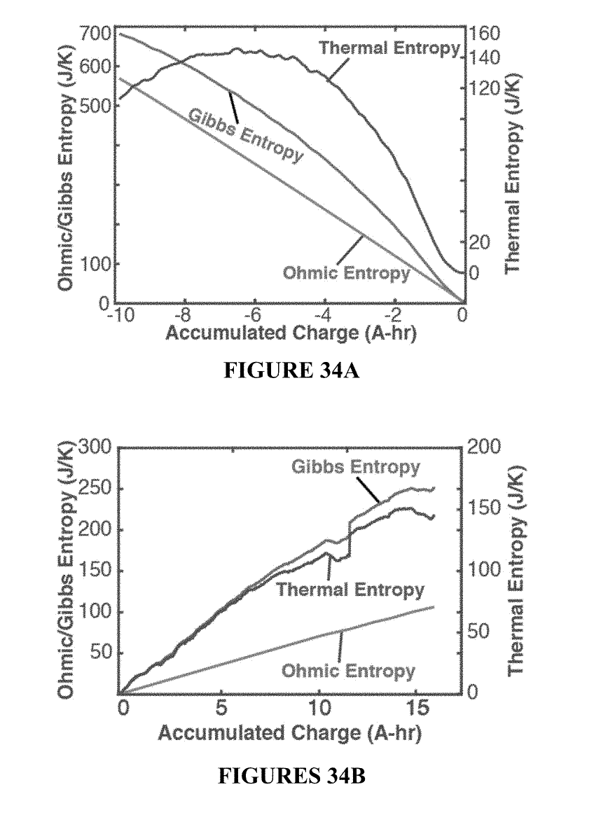

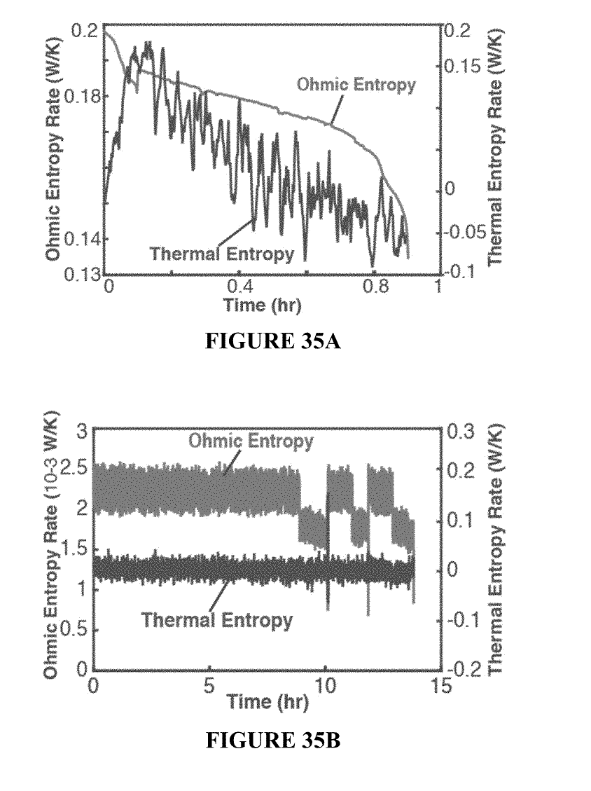

Accumulated capacity was linear in ohmic entropy (FIGS. 34A-34B). Influence of unsteady charge/discharge rate on entropy generation is shown in FIGS. 35A-35B. Entropy production from discharging at rate 11 A per hour was significantly higher than entropy production from charging at 1.2 A per 14 hours (FIGS. 34A-34B). FIG. 35A shows a higher ohmic entropy generation rate with the higher discharge rate. Total thermal entropies during charge and discharge have similar magnitude. The thermal entropy change depended only on temperature change, with an average constant heat capacity. Electrolyte heat capacity was used but a more accurate heat capacity should include electrode materials. As with lithium-ion batteries, the thermal entropy change proceeded considerably slower than ohmic counterpart. In Table 4.2, a trend is not readily evident, particularly due to the small values of overall thermal entropy change, accentuating measurement sensitivities. As with energy, ohmic entropy dominates total Gibbs entropy (FIGS. 34A-34B), with significant thermal entropy contribution.

Degradation Coefficients and the Degradation Surface

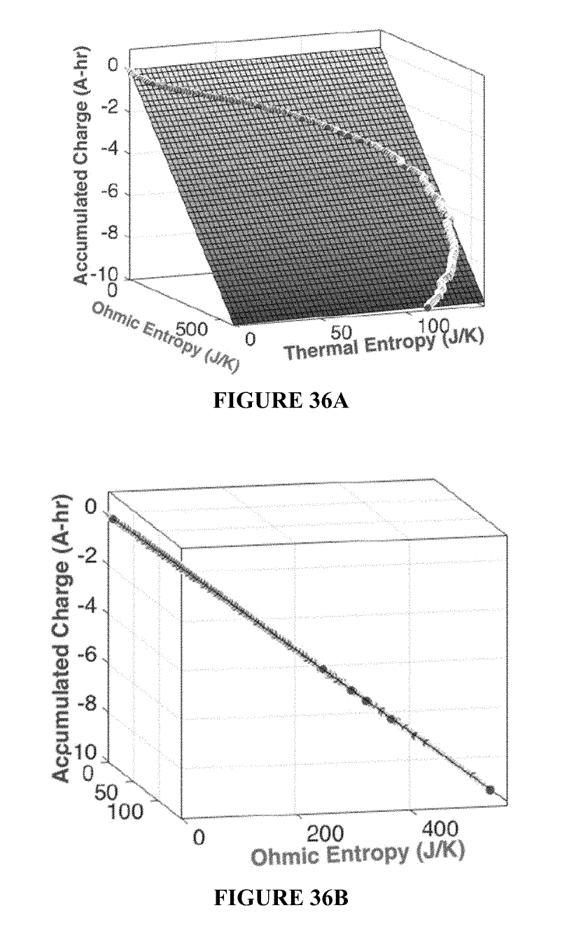

The DEG surface (FIGS. 36A-36B) from the lead-acid battery data had goodness of fit R²=1, similar to lithium-ion battery, with coefficient predictions at 95% confidence interval. The DEG trajectory, while perfectly coincident with the DEG surface, has a curved profile from the thermal component. The recovery indicated by the change in direction of the thermal entropy limits the extent along the thermal dimension, and the volume spanned in the DEG domain.

Table 4.2 shows consistent B_{W} values. After the first 9 cycles, an average drop in B_{W} of about 16% is seen with an increase in discharge rate of 300%. Values of B_{T} in Table 4.2 show more scatter than B_{W} counterparts, seemingly due to inconsistent ohmic heating (across cycles) and temperature measurement sensitivity. Initial values of B_{T} are positive and become negative as the battery undergoes more endothermic recovery with significant increase in discharge rate, an artefact of manufacturer design.

As with li-ion battery, positive B_{W} values (right side of Table 4.2) from charge indicate an energy-adding transformation.

Heat-Only Thermodynamic Analysis

Heat transfer is free convection spontaneously driven by differences in battery and ambient temperatures. This section's formulations and methods, same as chapter 3, were omitted. As before, the trapezoidal rule evaluated the heat transfer and entropy integrals. Plots of discharge are to the left, and charge plots to the right. Only normal discharge cycling results are discussed here for both lithium-ion and lead-acid batteries.

Lithium-Ion Battery

TABLE 4.3
Processed heat-only analysis parameters for lithium-
ion battery. Cycle 4 is used in data breakdown.

| | Normal Discharge | | | | | | | | | | Charge |
|---|---|---|---|---|---|---|---|---|---|---|---|
| | [symbol] | Q_{N} | ΔE_{N} | E′_{N} | S′_{N}\|Q | ΔS′_{N}\|T | S′_{N} | B_{Q} | B_{T} | [symbol] ᵣₑₛ | [symbol] |
| N | A-hr | J | J | J | J/K | J/K | J/K | AhrK/J | AhrK/J | A-hr | A-hr |
| 1 | −4.84 | −0.5 | 1.2 | 1.7 | −1.61 | 3.97 | 5.58 | 2.156 | 0.076 | −0.402 | 22.61 |
| 2 | −6.70 | −0.6 | 1.9 | 2.5 | −1.89 | 6.23 | 8.12 | 1.604 | 0.074 | −0.626 | 11.23 |
| 3 | −8.26 | −1.1 | 1.1 | 2.3 | −3.69 | 3.66 | 7.34 | 1.914 | −0.129 | −0.382 | 8.38 |
| 4 | −6.15 | −0.5 | 1.4 | 1.8 | −1.62 | 4.46 | 6.08 | 1.788 | 0.011 | −0.772 | 9.68 |
| 5 | −5.83 | −0.5 | 1.3 | 1.8 | −1.65 | 4.24 | 5.89 | 1.699 | 0.166 | −0.794 | 18.21 |
| 6 | −8.52 | −0.7 | 1.7 | 2.4 | −2.36 | 5.63 | 7.99 | 2.131 | 0.131 | −0.681 | 78.86 |
| 7 | −5.31 | −0.4 | 1.2 | 1.6 | −1.28 | 4.09 | 5.38 | 1.940 | −0.283 | −0.652 | 3.86 |
| 8 | −8.58 | −0.8 | 1.6 | 2.4 | −2.62 | 5.10 | 7.72 | 1.314 | −0.078 | −1.023 | 10.65 |
| 9 | −4.31 | −0.3 | 1.5 | 1.8 | −0.84 | 5.16 | 6.00 | 2.487 | −0.256 | −0.393 | 11.92 |
| 10 | −7.04 | −0.6 | 1.1 | 1.7 | −1.86 | 3.75 | 5.61 | 1.683 | −0.048 | −1.096 | 13.75 |
| 11 | −9.59 | −0.9 | 1.2 | 2.1 | −2.96 | 3.87 | 6.82 | 2.388 | −0.139 | −0.780 | 6.13 |
| 12 | −5.09 | −0.3 | 1.6 | 1.9 | −1.06 | 5.21 | 6.27 | 1.905 | −0.225 | −0.550 | 9.90 |
| 13 | −7.05 | −0.6 | 1.4 | 2.0 | −1.83 | 4.80 | 6.63 | 1.642 | 0.138 | −0.889 | 9.59 |
| 14 | −6.81 | −0.5 | 1.6 | 2.1 | −1.79 | 5.26 | 7.05 | 2.076 | −0.019 | −0.612 | 11.80 |
| 15 | −8.46 | −0.7 | 1.2 | 1.9 | −2.41 | 3.82 | 6.24 | 2.071 | −0.012 | −1.006 | 30.76 |
| 16 | −5.36 | −0.4 | 0.9 | 1.4 | −1.39 | 3.08 | 4.47 | 1.738 | 0.023 | −1.033 | 9.56 |
| 17 | −7.11 | −0.6 | 1.2 | 1.8 | −1.93 | 3.98 | 5.91 | 1.775 | 0.086 | −1.037 | 11.00 |
| 18 | −7.37 | −0.5 | 1.4 | 1.9 | −1.77 | 4.51 | 6.27 | 1.786 | 0.178 | −1.023 | 8.14 |
| 19 | −5.21 | −0.5 | 1.2 | 1.6 | −1.50 | 3.85 | 5.35 | 2.115 | −0.250 | −0.530 | 9.26 |
| 20 | −6.08 | −0.6 | 0.9 | 1.5 | −1.94 | 2.97 | 4.91 | 1.912 | 0.073 | −0.883 | 9.86 |
| 21 | −6.36 | −0.5 | 1.8 | 2.3 | −1.60 | 6.06 | 7.66 | 1.770 | −0.224 | −0.555 | 11.28 |
| 22 | −3.31 | −1.9 | 0.7 | 2.6 | −6.21 | 2.42 | 8.63 | 0.486 | −0.018 | −0.118 | 13.64 |
| 23 | −9.96 | −6.5 | 1.1 | 7.6 | −21.06 | 3.70 | 24.76 | 0.428 | 0.010 | −0.288 | 9.69 |
| 24 | −5.36 | −3.2 | 1.0 | 4.2 | −10.58 | 3.39 | 13.96 | 0.451 | −0.018 | −0.185 | 41.78 |
| 25 | −3.57 | −2.2 | 1.0 | 3.2 | −7.37 | 3.23 | 10.59 | 0.436 | −0.004 | −0.122 | 19.43 |
| 26 | −2.56 | −1.5 | 1.7 | 3.2 | −4.86 | 5.60 | 10.46 | 0.409 | 0.013 | −0.109 | 41.56 |
| 27 | −3.92 | −2.4 | 1.2 | 3.6 | −7.82 | 3.90 | 11.72 | 0.401 | 0.001 | −0.205 | 14.43 |
| 28 | −2.14 | −1.2 | 1.7 | 2.9 | −4.03 | 5.58 | 9.61 | 0.411 | 0.004 | −0.090 | 16.81 |
| 29 | −2.06 | −1.2 | 1.7 | 2.9 | −3.88 | 5.62 | 9.50 | 0.391 | 0.000 | −0.096 | 11.11 |

| | Charge | | | | | | | | |
|---|---|---|---|---|---|---|---|---|---|
| | Q_{N} | ΔE_{N} | E′_{N} | S′_{N}\|Q | ΔS′_{N}\|T | S′_{N} | B_{Q} | B_{T} | [symbol] ᵣₑₛ |
| N | J | J | J | J/K | J/K | J/K | AhrK/J | AhrK/J | A-hr |
| 1 | −1.02 | 0.35 | 1.37 | −3.36 | 1.15 | 4.51 | −6.370 | 0.294 | 0.932 |
| 2 | −0.64 | 0.15 | 0.78 | −2.09 | 0.49 | 2.58 | −5.339 | 0.022 | 0.097 |
| 3 | −0.56 | −0.58 | −0.02 | −1.82 | −1.89 | −0.07 | −4.432 | −0.386 | −0.199 |
| 4 | −0.45 | −0.01 | 0.44 | −1.48 | −0.03 | 1.45 | −6.521 | −0.052 | 0.080 |
| 5 | −0.75 | 0.00 | 0.75 | −2.49 | 0.01 | 2.49 | −7.138 | 0.033 | −0.514 |
| 6 | −2.45 | 0.24 | 2.70 | −8.11 | 0.81 | 8.91 | −9.601 | 0.448 | −2.791 |
| 7 | −0.27 | −0.35 | −0.08 | −0.87 | −1.13 | −0.27 | −4.892 | −0.078 | 0.327 |
| 8 | −0.53 | 0.07 | 0.60 | −1.76 | 0.23 | 1.99 | −6.037 | −0.117 | −0.103 |
| 9 | −4.99 | 0.42 | 5.41 | −16.50 | 1.40 | 17.89 | −0.727 | −0.006 | −0.075 |
| 10 | −0.68 | 0.01 | 0.69 | −2.23 | 0.04 | 2.27 | −6.215 | −0.560 | −1.010 |
| 11 | −0.38 | −0.10 | 0.28 | −1.23 | −0.33 | 0.91 | −4.968 | −0.088 | −0.035 |
| 12 | −0.48 | 0.04 | 0.52 | −1.60 | 0.12 | 1.72 | −6.212 | −0.102 | −0.094 |
| 13 | −0.46 | −0.08 | 0.38 | −1.51 | −0.26 | 1.25 | −6.409 | −0.232 | 0.268 |
| 14 | −0.65 | −0.05 | 0.60 | −2.12 | −0.15 | 1.97 | −5.479 | 0.016 | 0.081 |
| 15 | −1.34 | −0.10 | 1.24 | −4.42 | −0.33 | 4.09 | −6.699 | −1.014 | −1.592 |
| 16 | −3.27 | −0.01 | 3.26 | −10.84 | −0.03 | 10.81 | −0.812 | 1.014 | 1.763 |
| 17 | −0.49 | 0.16 | 0.64 | −1.60 | 0.52 | 2.12 | −6.846 | −0.097 | −0.094 |
| 18 | −0.45 | −0.09 | 0.36 | −1.48 | −0.30 | 1.17 | −5.606 | −0.227 | 0.433 |
| 19 | −0.44 | 0.10 | 0.55 | −1.47 | 0.34 | 1.81 | −6.114 | −0.192 | 0.288 |
| 20 | −0.47 | 0.38 | 0.85 | −1.57 | 1.25 | 2.81 | −5.972 | −0.181 | 0.410 |
| 21 | −3.70 | 0.02 | 3.71 | −12.17 | 0.05 | 12.22 | −0.821 | 1.735 | 0.311 |
| 22 | −5.25 | 0.17 | 5.42 | −17.47 | 0.56 | 18.03 | −0.782 | −0.004 | −0.068 |
| 23 | −0.38 | 0.39 | 0.77 | −1.25 | 1.30 | 2.54 | −8.848 | −2.139 | 0.629 |
| 24 | −18.08 | 0.50 | 18.58 | −59.59 | 1.65 | 61.24 | −0.701 | 0.007 | −0.024 |
| 25 | −8.97 | 0.50 | 9.47 | −29.75 | 1.67 | 31.42 | −0.654 | −0.048 | 0.006 |
| 26 | −17.60 | 0.54 | 18.14 | −58.19 | 1.79 | 59.98 | −0.713 | 0.018 | 0.056 |
| 27 | −6.05 | 0.37 | 6.43 | −20.00 | 1.23 | 21.24 | −0.726 | −0.030 | −0.072 |
| 28 | −6.44 | 1.53 | 7.97 | −21.44 | 5.12 | 26.56 | −0.758 | 0.041 | 0.104 |
| 29 | −0.67 | 0.21 | 0.88 | −2.22 | 0.69 | 2.91 | −4.913 | 0.113 | 0.211 |

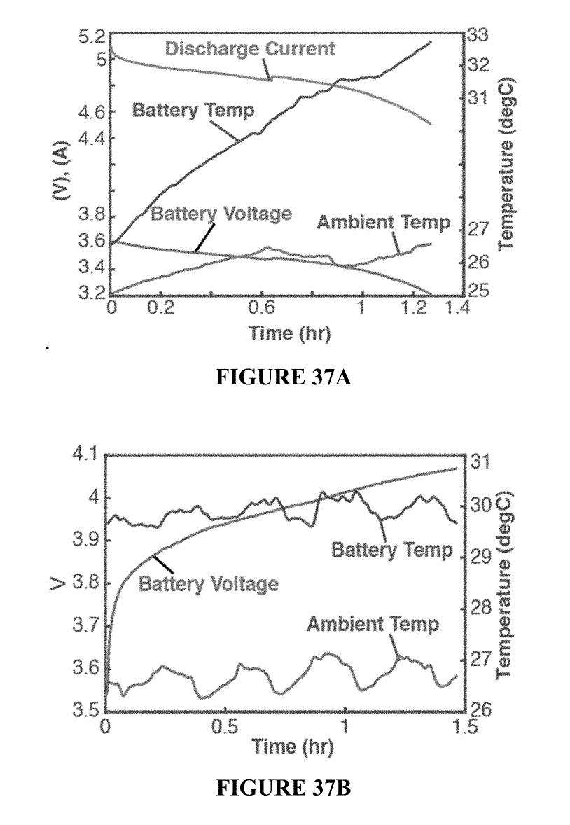

Cycle 4 data will be presented. FIGS. 37A-37B plot current, voltage and temperatures versus time. Only battery temperature changed significantly during discharge.

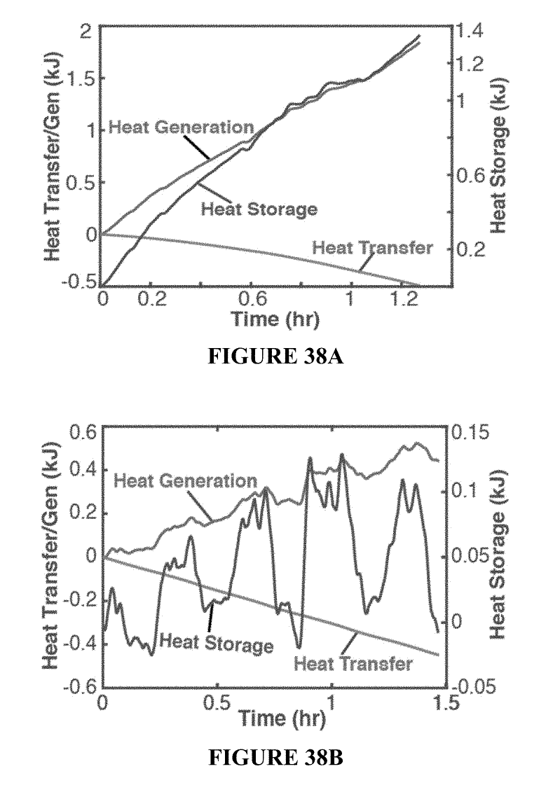

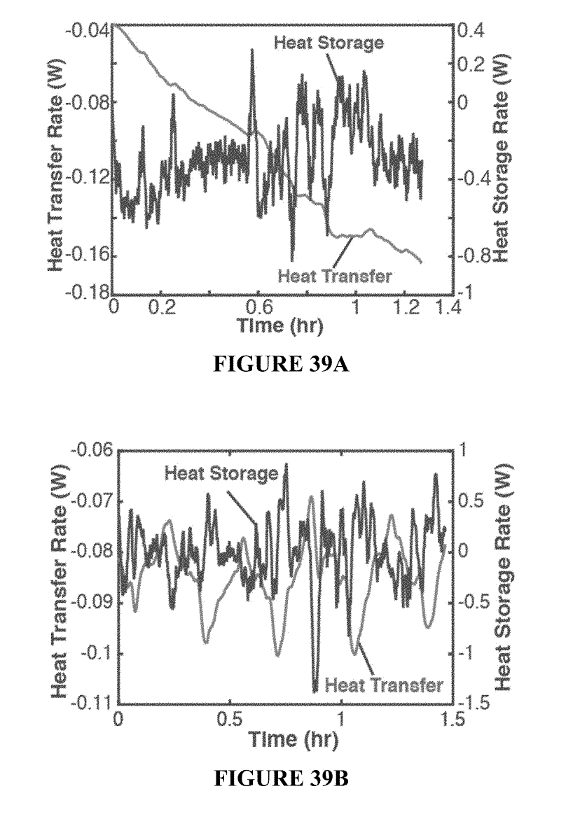

Heat transferred mostly out of the lithium-ion battery, as high cycling rates induced significant ohmic heating. A near linear trend (FIG. 37A) was observed during cycling. Variation in cyclic accumulation values in Table 4.3 are consistent with variation in the cycling schedule and ambient conditions. The heat storage component is the same as the thermal component in the Gibbs formulations. Most of the heat generated was stored in the battery as observed in Table 4.3 and FIGS. 38A-38B. This implies that heat transfer and heat generation proceed in opposite directions. The heat process rates, while fluctuating significantly are steady about a mean (FIGS. 39A-39B).

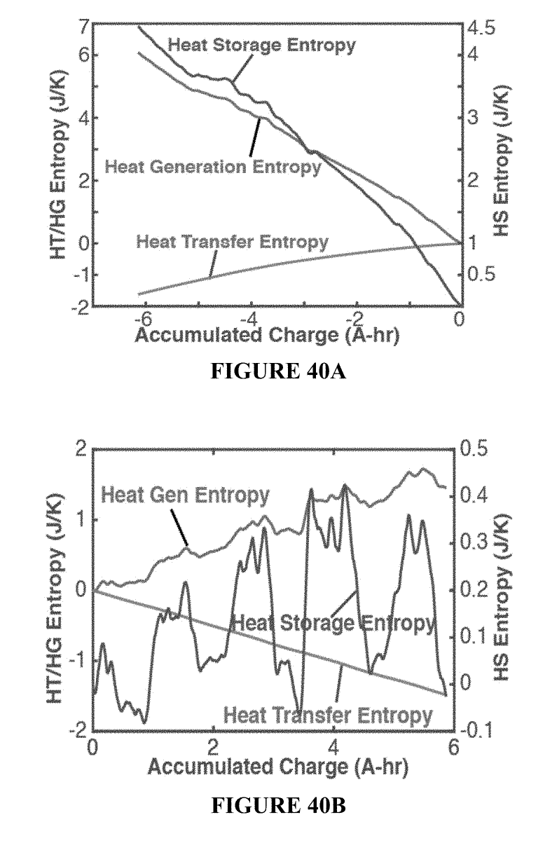

Table 4.3 and FIGS. 40A-40B show the entropy transfer by heat has a slightly linear relationship with accumulated charge. In FIGS. 40A-40B, total accumulated heat transfer is about the same for both charging and discharging, notwithstanding the significant difference in durations, indicating a dependence on cycling rates. The contribution to total entropy generation during cycling by the heat storage process, see FIGS. 40A-40B, right axis, is lower during charge than discharge, as expected (lower overall temperature change in the former).

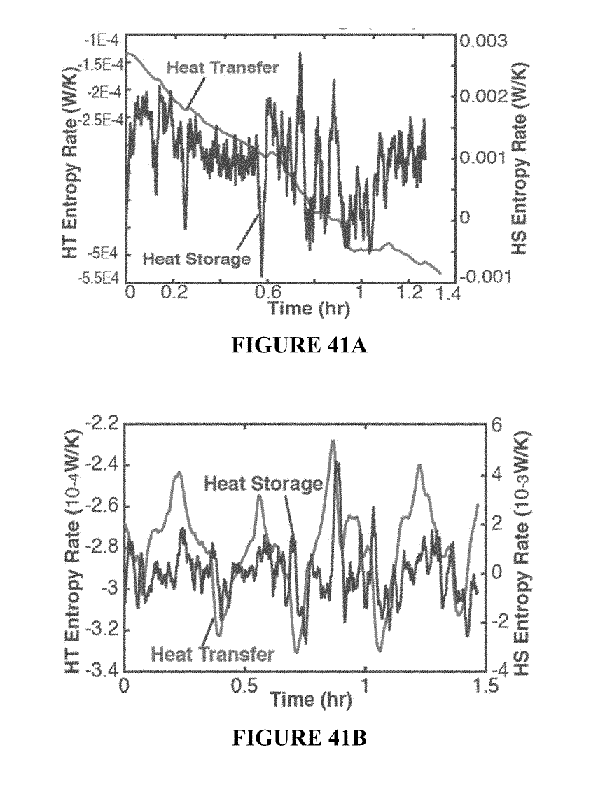

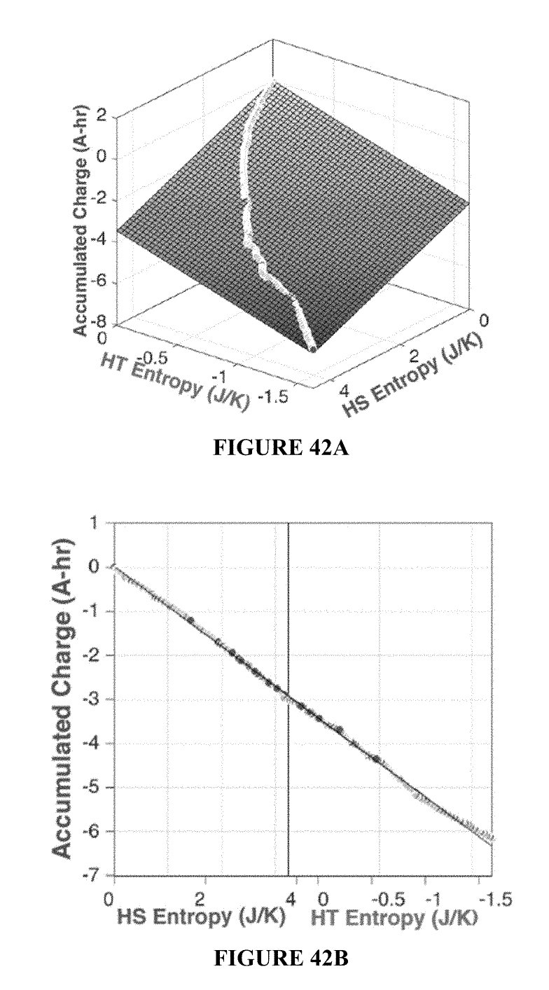

With the heat generation approach, a linear partial variation of charge accumulation with respect to one process is more adequately visualized in a DEG domain 3D axes, as shown in FIGS. 42A-42B below. Like grease, this also underscores properly formulating the entropy equations in the accurate determination of DEG coefficients. Entropy generation rates (FIGS. 41A-41B) show an increasing entropy transfer rate out of the battery (negative) and a relatively steady scatter for thermal entropy.

Degradation Coefficients and the Degradation Surface

The surface models each had R²>0.98 with coefficient predictions at 95% confidence interval. The DEG trajectory has a linear profile not apparent in 2D, see FIG. 42B, the view on the right which shows a coincidence of data points with the DEG surface.

Table 4.3 shows consistent trend in the heat transfer degradation coefficient B_{Q} with slight variations attributed to changes in ambient conditions from cycle to cycle, irregular cycling schedule and measurement error. B_{T} values are consistently less than B_{Q} and share similar overall trend.

Lead-Acid Battery

This discussion is similar to the lithium-ion battery but emphasizes lead-acid specific trends. For lead-acid batteries, a wider margin of error is anticipated in the heat-only results. The lead acid batteries had 3 separate cells, but only the center cell electrolyte temperature was measured. This had no impact on the Gibbs analysis due to significant difference in ohmic and thermal process rates.

TABLE 4.4
Processed heat-only analysis parameters for lead-acid starter
battery. Cycle 2 (in bold) is used in analysis breakdown.

| | Normal Discharge | | | | | | | | | | Charge |
|---|---|---|---|---|---|---|---|---|---|---|---|
| | [symbol] | Q_{N} | ΔE_{N} | E′_{N} | S′_{N}\|Q | ΔS′_{N}\|T | S′_{N} | B_{Q} | B_{T} | [symbol] ᵣₑₛ | [symbol] |
| N | A-hr | kJ | kJ | kJ | J/K | J/K | J/K | AhrK/J | AhrK/J | A-hr | A-hr |
| 1 | −54.56 | −120.3 | −2.3 | 118.0 | −385.6 | −7.5 | 378.1 | 0.100 | −8.105 | 0.024 | 6.12 |
| 2 | −9.87 | −27.3 | 34.8 | 62.0 | −87.9 | 112.9 | 200.8 | 0.084 | −0.698 | −0.012 | 16.55 |
| 3 | −10.22 | 0.4 | 11.9 | 11.5 | 1.3 | 38.8 | 37.6 | −3.721 | −1.030 | 0.030 | 15.19 |
| 4 | −12.43 | −87.0 | 40.2 | 127.2 | −274.7 | 129.9 | 404.6 | 0.038 | −1.099 | 0.000 | 15.30 |
| 5 | −8.31 | −33.0 | 43.7 | 76.7 | −105.8 | 140.9 | 246.7 | 0.056 | −0.625 | −0.010 | 25.36 |
| 6 | −14.75 | −57.0 | 9.6 | 66.6 | −181.7 | 31.0 | 212.7 | 0.068 | −1.103 | 0.003 | 19.16 |
| 7 | −12.80 | −102.6 | 30.9 | 133.5 | −322.5 | 99.7 | 422.2 | 0.033 | −1.163 | 0.000 | 16.88 |
| 8 | −11.16 | −2.8 | −3.7 | −0.9 | −9.2 | −12.0 | −2.8 | 0.234 | −0.014 | 0.818 | 18.62 |
| 9 | −9.00 | −3.1 | −13.9 | −10.7 | −10.2 | −45.2 | −35.0 | −0.197 | 0.026 | 0.249 | 25.23 |
| 10 | −13.10 | 2.5 | −13.1 | −15.6 | 8.2 | −42.7 | −50.9 | −0.309 | −0.527 | 0.196 | 29.65 |
| 11 | −10.21 | 6.8 | −15.0 | −21.8 | 22.2 | −48.9 | −71.1 | −0.048 | −0.249 | 0.178 | 19.49 |
| 12 | −11.16 | 7.3 | −16.6 | −23.8 | 23.7 | −54.0 | −77.7 | −0.098 | −0.163 | 0.167 | 26.69 |
| 13 | −9.93 | −4.0 | −17.7 | −13.7 | −13.0 | −57.6 | −44.6 | 0.158 | −0.121 | 0.136 | 25.13 |
| 14 | −7.68 | 0.6 | −14.8 | −15.4 | 1.8 | −48.7 | −50.5 | 0.642 | −0.119 | 0.174 | 19.59 |
| 15 | −4.86 | 3.2 | −16.1 | −19.3 | 10.5 | −53.0 | −63.5 | −1.005 | −0.043 | −0.102 | 20.69 |
| 16 | −3.95 | 0.7 | −14.1 | −14.8 | 2.2 | −46.4 | −48.6 | 3.688 | −0.357 | 0.243 | 7.98 |
| 17 | −2.51 | 0.6 | −8.8 | −9.4 | 1.9 | −29.0 | −30.9 | −4.825 | 0.004 | −0.217 | 11.26 |

| | Charge | | | | | | | | |
|---|---|---|---|---|---|---|---|---|---|
| | Q_{N} | ΔE_{N} | E′_{N} | S′_{N}\|Q | ΔS′_{N}\|T | S′_{N} | B_{Q} | B_{T} | [symbol] ᵣₑₛ |
| N | kJ | kJ | kJ | J/K | J/K | J/K | AhrK/J | AhrK/J | A-hr |
| 1 | 144.6 | 13.4 | −131.2 | 474.7 | 44.1 | −430.6 | 0.015 | 0.017 | −0.021 |
| 2 | 430.6 | 44.0 | −386.6 | 1422.2 | 145.2 | −1277.1 | 0.001 | −1.012 | 0.092 |
| 3 | 215.5 | 27.0 | −188.5 | 708.2 | 88.3 | −619.9 | 0.015 | 0.843 | 0.037 |
| 4 | 531.8 | 36.8 | −495.0 | 1758.1 | 120.8 | −1637.2 | 0.006 | 0.411 | 0.026 |
| 5 | 486.1 | 60.7 | −425.4 | 1600.4 | 199.3 | −1401.1 | 0.014 | −4.668 | 0.021 |
| 6 | 349.4 | 59.4 | −290.0 | 1152.2 | 194.7 | −957.6 | 0.008 | −0.717 | 0.049 |
| 7 | 331.7 | 51.1 | −280.6 | 1094.7 | 167.2 | −927.6 | 0.008 | 0.730 | 0.033 |
| 8 | 400.2 | 31.9 | −368.3 | 1314.1 | 104.0 | −1210.1 | 0.010 | 2.660 | 0.019 |
| 9 | 473.0 | 21.5 | −451.6 | 1550.1 | 70.1 | −1480.0 | 0.011 | −0.676 | 0.024 |
| 10 | 1185.4 | 112.0 | −1073.5 | 3934.1 | 373.0 | −3561.1 | 0.017 | −4.948 | −0.087 |
| 11 | 1079.3 | 118.5 | −960.8 | 3601.1 | 395.7 | −3205.4 | 0.025 | −3.391 | −0.169 |
| 12 | 1032.2 | 123.5 | −908.7 | 3424.6 | 409.5 | −3015.0 | −0.011 | −6.199 | 0.165 |
| 13 | 983.0 | 59.0 | −924.0 | 3238.4 | 194.5 | −3043.9 | 0.010 | −4.710 | −0.021 |
| 14 | 389.0 | 8.8 | −380.2 | 1278.1 | 28.7 | −1249.5 | 0.013 | −0.645 | 0.037 |
| 15 | 658.3 | 51.7 | −606.5 | 2183.9 | 171.9 | −2012.0 | 0.030 | −1.017 | −0.244 |
| 16 | 218.5 | 31.2 | −249.7 | 711.3 | 120.1 | −591.2 | 0.018 | −2.124 | −0.076 |
| 17 | 240.2 | 5.6 | −245.8 | 919.3 | 23.9 | −895.4 | 0.010 | −4.812 | −0.028 |

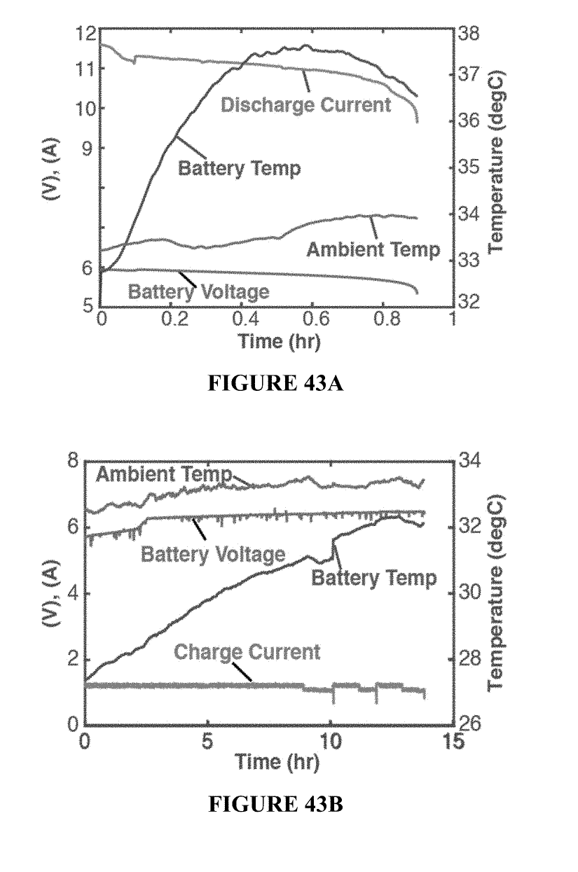

Only temperature was monitored. The charge plot (FIG. 43B) shows ambient temperature higher than battery temperature, suggesting an endothermic phenomenon during the prior over-discharge process.

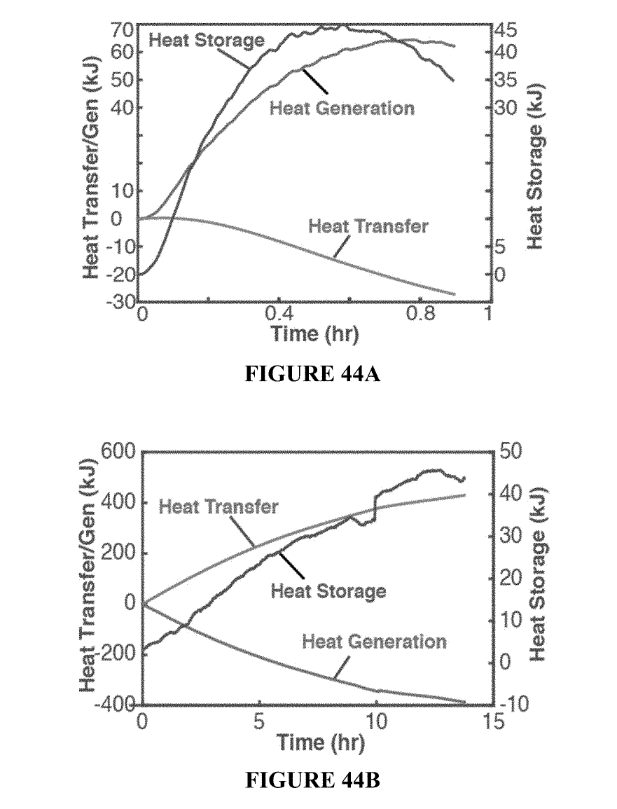

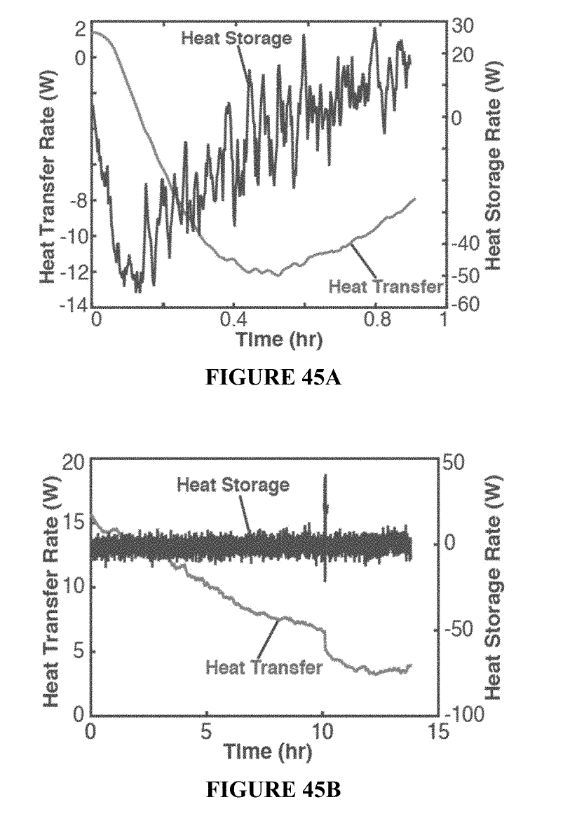

Heat transferred predominantly out of the lead-acid batteries during discharge (FIG. 44A) in the normal region, due to initial ohmic heating elevating temperatures. During charge (FIG. 44B) heat transferred into the battery due to sub-ambient battery temperatures (FIG. 43B). Unlike lithium-ion batteries the cooling effect in the starter lead-acid batteries (discussed in the Gibbs analysis) introduced more non-linearity into the heat storage accumulation component (FIGS. 44A-44B). From Table 4.4, the heat transferred exceeded the heat stored at the low cycling rate. Vice versa when the rate was increased. This agrees with the Gibbs analysis, wherein the same trend appears in the thermal energy term. With comparable magnitudes of both partial entropy contributions, the fluctuating rate of change of battery temperature becomes more significant to heat analysis, as shown in FIGS. 45A-45B (the thermal contribution is considerably less than the ohmic in the Gibbs approach).

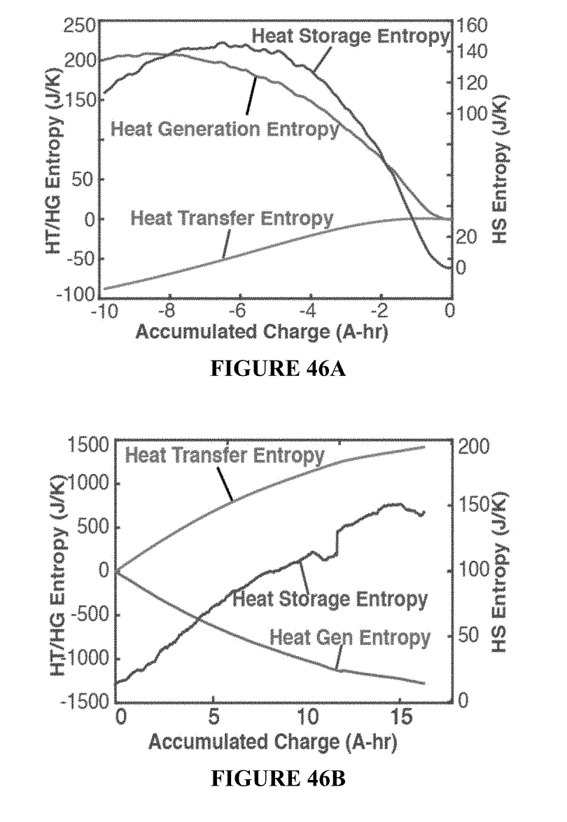

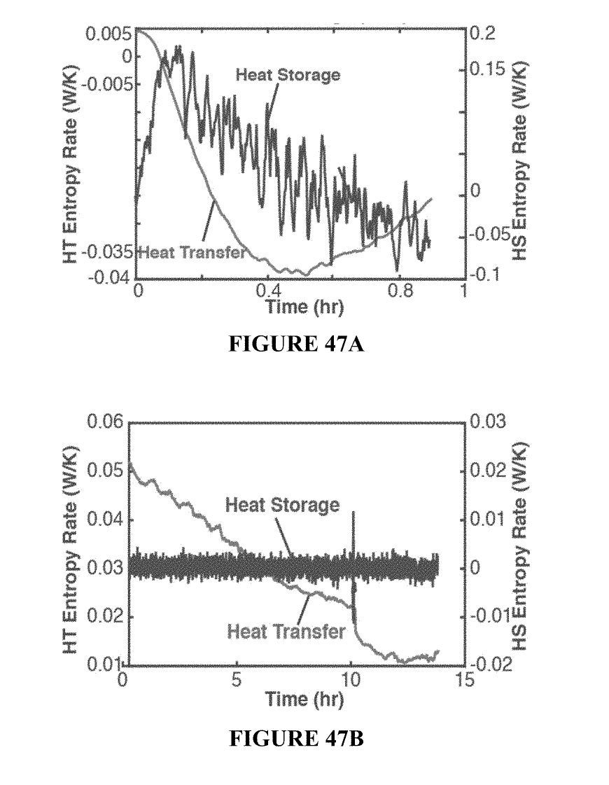

The heat transfer entropy during discharge was mostly out of the battery (negative in FIG. 46A and Table 4.4). For charge shown in FIG. 46B, the initial negative temperature differential is not fully recovered so heat transfer entropy is into the battery throughout the process. Accumulation is observed to reduce with increased discharge rate. The heat storage entropy was smaller, similar in behavior to the Gibbs analysis, but contributed significantly more to the battery degradation measure (than li-ion batteries) since magnitudes were comparable to the concurrent heat transfer entropy. Heat generation entropy proceeded in direction opposite to heat transfer, as prescribed by equation (Equation 1.41). The lack of linearity between charge/discharge accumulation and only one component's entropy contribution was more evident for lead-acid batteries than lithium-ion. FIGS. 47A-47B plot the heat entropy rates.

DEG Coefficients and the Degradation Surface

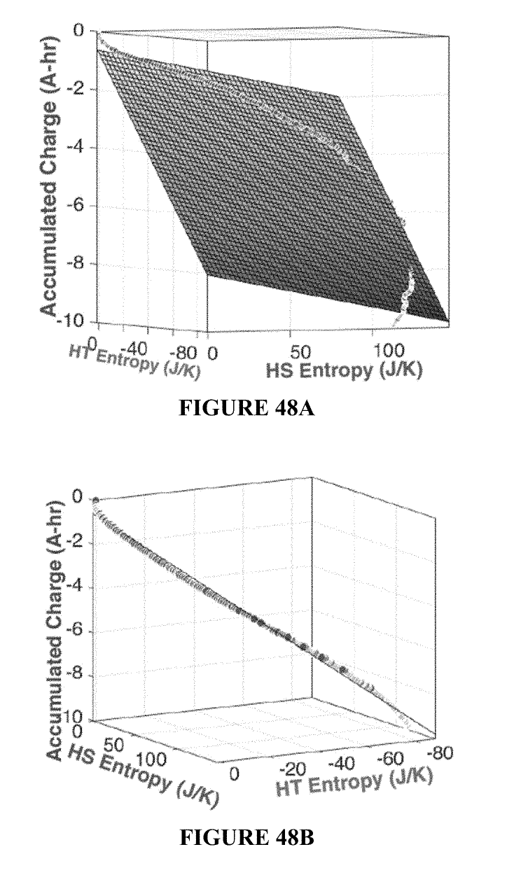

The DEG surface, FIGS. 48A-48B, from cycle 2 data had R²>0.96 with coefficient predictions at 95% confidence interval.

Similar to lithium-ion batteries, B_{Q} is sensitive to ambient conditions (during cycling) and to process rates. Irregular changes in heat transfer rates due to uncontrolled ambient conditions make trends in the data less apparent. B_{T} values in Table 4.4 show consistent order, with variations in magnitude from cycle to cycle consistently less than B_{Q} for all cycles at the initial discharge current of 11 A. With current tripled, B_{T} increased by 1 to 2 orders of magnitude, a behavior similar to that of Gibbs B_{T}. While subject to temperature measurement errors, B_{T} proceeds faster than the spontaneous free convection heat transfer, is more impervious to ambient conditions (a dependency on heat capacity implies high material-dependence). Hence it is impacted directly by the increase in internal heat generation (ohmic heating) from an increase in discharge rate, as seen in Table 4.4.

Overdischarging

Batteries were overdischarged to accelerate degradation. Minimal floating charge applied after the battery reached full charge rendered effects of overcharging negligible.

Lead-Acid Battery

With lead-acid batteries a precipitous “cliff” drop in voltage occurs after sufficient discharge. Output current also drops, the battery stabilizes at this new rate, and continues to discharge until another cliff drop in voltage (about 1V for this battery type).

TABLE 4.5
Parameters for Deka lead-acid starter battery for normal charge and over discharge. Bold line denotes
the start of the 35 A discharge rate. Cycles above the bold line were discharged at 11 A.

| | Normal Discharge | | | | | | | | | | Overdischarge |
|---|---|---|---|---|---|---|---|---|---|---|---|
| | [symbol] | G_{N}\|W | ΔG_{N}\|T | ΔG_{N} | S′_{N}\|W | ΔS′_{N}\|T | S′_{N} | B_{W} | B_{T} | [symbol] ᵣₑₛ | [symbol] |
| N | A-hr | kJ | kJ | kJ | J/K | J/K | J/K | AhrK/J | AhrK/J | A-hr | A-hr |
| 1 | −54.56 | −961.1 | 2.3 | −958.9 | 3096.9 | −7.5 | 3089.4 | −0.018 | 0.204 | 0.005 | −61.35 |
| 2 | −9.87 | −176.1 | −34.8 | −210.9 | 569.2 | 112.9 | 682.1 | −0.018 | 0.019 | 0.001 | −20.46 |
| 3 | −10.22 | −176.6 | −11.9 | −188.5 | 576.0 | 38.8 | 614.8 | −0.018 | 0.014 | 0.001 | −12.40 |
| 4 | −12.43 | −224.3 | −40.2 | −264.5 | 711.9 | 129.9 | 841.8 | −0.017 | 0.045 | 0.000 | −23.26 |
| 5 | −8.31 | −145.0 | −43.7 | −188.6 | 465.7 | 140.9 | 606.6 | −0.018 | 0.052 | 0.001 | −15.53 |
| 6 | −14.75 | −266.7 | −9.6 | −276.3 | 853.1 | 31.0 | 884.1 | −0.017 | 0.026 | 0.000 | −23.48 |
| 7 | −12.80 | −230.9 | −30.9 | −261.9 | 730.7 | 99.7 | 830.5 | −0.018 | 0.028 | 0.000 | −19.47 |
| 8 | −11.16 | −189.8 | 3.7 | −186.1 | 615.0 | −12.0 | 603.0 | −0.018 | 0.006 | 0.031 | −45.49 |
| 9 | −9.00 | −175.1 | 13.9 | −161.2 | 571.5 | −45.2 | 526.3 | −0.017 | 0.018 | −0.019 | −15.65 |
| 10 | −13.10 | −258.7 | 13.1 | −245.6 | 844.3 | −42.7 | 801.7 | −0.016 | 0.025 | −0.010 | −24.17 |
| 11 | −10.21 | −197.1 | 15.0 | −182.1 | 644.4 | −48.9 | 595.5 | −0.015 | 0.048 | 0.004 | −29.41 |
| 12 | −11.16 | −218.2 | 16.6 | −201.7 | 711.6 | −54.0 | 657.6 | −0.015 | 0.059 | 0.008 | −21.64 |
| 13 | −9.93 | −190.2 | 17.7 | −172.5 | 619.7 | −57.6 | 562.1 | −0.015 | −0.010 | 0.015 | −29.13 |
| 14 | −7.68 | −145.4 | 14.8 | −130.6 | 476.8 | −48.7 | 428.1 | −0.015 | −0.005 | 0.012 | −21.64 |
| 15 | −4.86 | −90.3 | 16.1 | −74.2 | 297.3 | −53.0 | 244.3 | −0.015 | −0.008 | 0.007 | −10.80 |
| 16 | −3.95 | −72.4 | 14.1 | −58.3 | 237.4 | −46.4 | 191.0 | −0.016 | −0.001 | 0.005 | −9.55 |
| 17 | −2.51 | −45.6 | 8.8 | −36.7 | 149.4 | −29.0 | 120.4 | −0.016 | −0.003 | 0.006 | −5.86 |

| | Overdischarge | | | | | | | | |
|---|---|---|---|---|---|---|---|---|---|
| | G_{N}\|W | ΔG_{N}\|T | ΔG_{N} | S′_{N}\|W | ΔS′_{N}\|T | S′_{N} | B_{W} | B_{T} | [symbol] ᵣₑₛ |
| N | kJ | kJ | kJ | J/K | J/K | J/K | AhrK/J | AhrK/J | A-hr |
| 1 | −1004.8 | 21.0 | −983.8 | 3239.5 | −68.7 | 3170.8 | −0.018 | −0.503 | 0.013 |
| 2 | −234.8 | 32.1 | −202.8 | 761.1 | −105.4 | 655.7 | −0.020 | −1.435 | 0.027 |
| 3 | −189.1 | −2.0 | −191.2 | 616.9 | 6.6 | 623.5 | −0.019 | −0.881 | 0.028 |
| 4 | −281.0 | 26.4 | −254.6 | 896.5 | −86.4 | 810.1 | −0.020 | −1.746 | 0.013 |
| 5 | −176.6 | 40.8 | −135.8 | 568.4 | −133.8 | 434.7 | −0.021 | −0.825 | 0.017 |
| 6 | −313.5 | 34.9 | −278.6 | 1005.3 | −113.5 | 891.7 | −0.017 | −2.005 | 0.020 |
| 7 | −266.1 | 27.1 | −239.0 | 844.9 | −88.4 | 756.5 | −0.019 | −0.888 | 0.007 |
| 8 | −212.7 | 27.3 | −185.3 | 689.3 | −88.9 | 600.3 | −0.018 | 0.111 | 0.045 |
| 9 | −242.2 | 142.2 | −100.0 | 793.3 | −475.1 | 318.2 | −0.021 | 1.990 | 0.025 |
| 10 | −351.3 | 99.8 | −251.5 | 1150.0 | −330.5 | 819.5 | −0.019 | 2.212 | 0.030 |
| 11 | −246.5 | 73.7 | −172.8 | 807.3 | −243.4 | 563.9 | −0.017 | 1.035 | 0.036 |
| 12 | −293.6 | 124.5 | −169.1 | 960.9 | −414.1 | 546.8 | −0.022 | 2.903 | 0.022 |
| 13 | −220.2 | 87.2 | −133.1 | 719.3 | −287.9 | 431.5 | −0.010 | −0.260 | 0.044 |
| 14 | −169.2 | 86.4 | −82.8 | 555.2 | −287.0 | 268.2 | −0.021 | 1.674 | 0.020 |
| 15 | −109.6 | 54.1 | −55.6 | 361.3 | −179.3 | 181.9 | −0.017 | 0.588 | 0.024 |
| 16 | −88.3 | 52.4 | −35.9 | 289.9 | −173.2 | 116.6 | −0.016 | 0.423 | 0.026 |
| 17 | −57.2 | 19.4 | −37.8 | 187.8 | −63.7 | 124.0 | −0.021 | 0.951 | 0.037 |

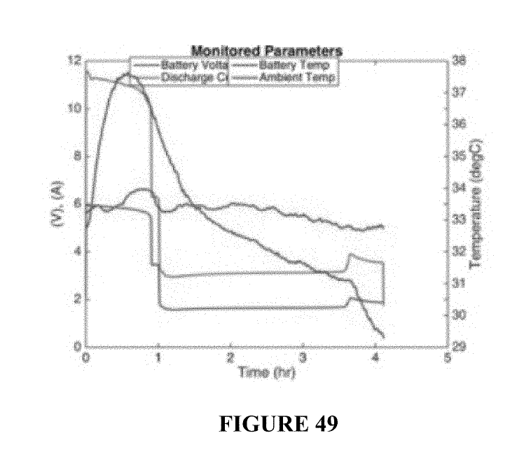

FIG. 49 continues the discharge from normal discharge (time 1 hr) into deep overdischarge (1 hour≤time≤4 hours), wherein significant irreversibilities accelerated degradation for multiple cycles on starter batteries (not designed for deep discharge). Temperatures initially rose due to ohmic-dominated heating; thereafter the battery recovered thermally (after 0.7 hr) and temperatures declined, even in the intermediate constant-current (3 A) region, until the external load was removed. This endothermic process explains the initially sub-ambient temperatures in the charge plots, and underscores need to consider thermal effects.

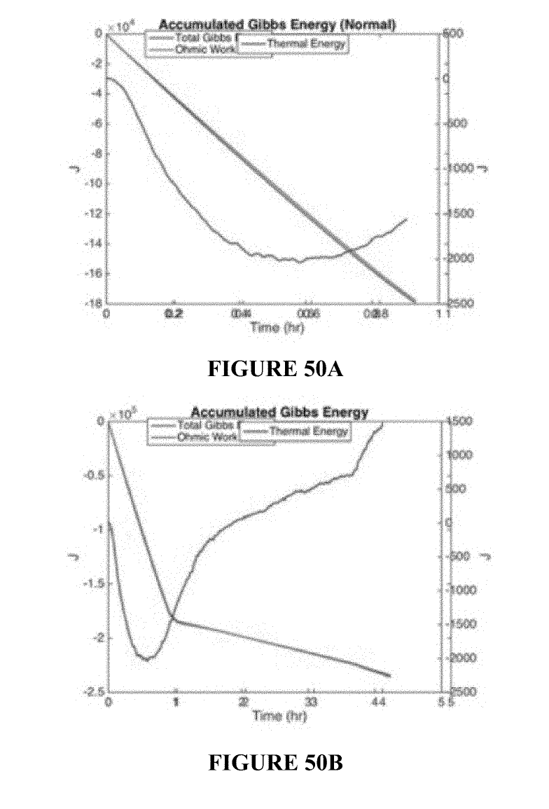

The observed cliff drop in voltage and current (FIG. 49) is evident in the abrupt slope reduction of ohmic work accumulation in FIG. 50B. The large drop in current from 11 A to 3 A makes the slope reduction more conspicuous (curve elbow at 1-hr). The battery's thermal energy followed temperature signal trends. The initial exothermic interval was overcompensated by the ensuing endothermic process, dropping battery temperature below initial value. The rate of change of thermal energy appears influenced by the rate of change of ohmic work via ohmic heating.

Table 4.5 and FIGS. 50A-50B show ohmic work, the most significant active process during the lead-acid battery cycling, determined the trend in total Gibbs energy change.

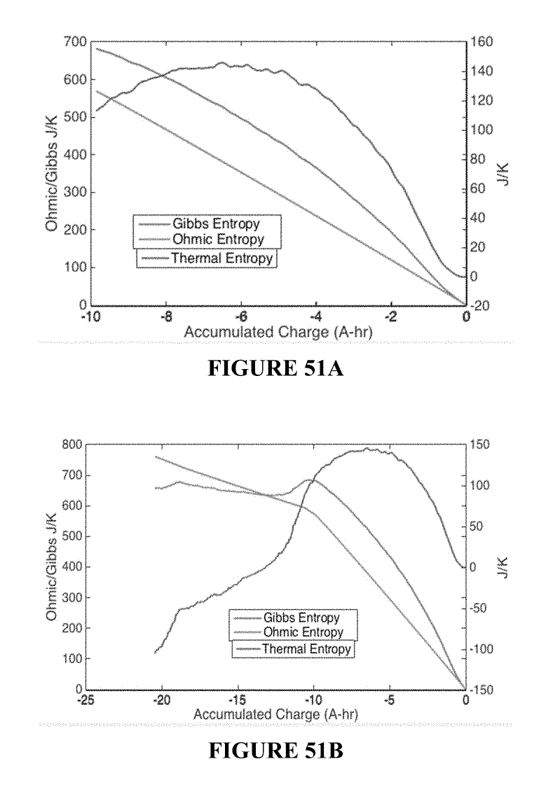

The linear relationship between ohmic entropy and accumulated charge/discharge (FIGS. 51A-51B) showed a reduction in slope (ohmic work dominated the thermal process, hence linearity). The over-discharge of 3 hours added about 200 J/K to the ohmic entropy (which was already 570 J/K in less than an hour, due to faster initial rates). The extra accumulated discharge (11 A-hr) was of same magnitude as previously accumulated (10 A-hr). This suggests that the entropy production varied significantly with discharge rate. Thermal entropy produced during the entire discharge behaved similar to the thermal energy. Unlike normal discharge, overdischarging caused overall entropy from heat storage to be negative. Total Gibbs entropy mimicked the dominant ohmic entropy, with thermal entropy about 25% less. Thermal entropy in the full discharge interval reduced the total entropy (albeit insignificantly in this case). The severe non-linearity of the overdischarge regime compared to the normal discharge suggests that the Bᵢ from a deeply overdischarged cycle should differ from that estimated from the normal cycle.

Degradation Coefficients

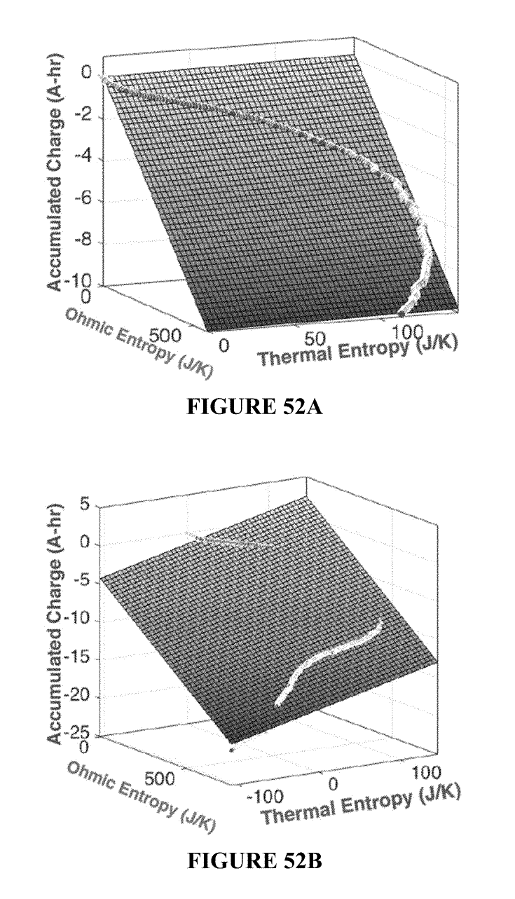

FIGS. 52A-52B, wherein the DEG trajectory reverses direction in the DEG plane, shows significant nonlinearity on the goodness of fit for overdischarging (FIG. 52B), as anticipated.

Table 4.5 shows variation in overdischarge B_{W} values. A B_{W} evaluated as an average over a DEG trajectory with normal and overdischarge regions, as in FIGS. 52A-52B, is inaccurate in the normal region, since discharge rates change during overdischarge. Values of overdischarge B_{T} in Table 4.5 show significant cyclic variations compared to normal B_{T}. Consistent initial ohmic heating-dominated temperature rise was observed from cycle to cycle during normal discharge. With the same discharge rate once activated and with long hours of overdischarge, negative entropies were recorded from the endothermic process. Higher discharge rate after cycle 9 can be seen to induce even more thermal recovery. The heavy-duty starter battery was designed for 625 CCA (Cold Cranking Amps). Recovery allows the battery to discharge quickly at high current, followed by a quick recharge, while maintaining health. While normal B_{T} transitioned to negative values with discharge rate for cycles 10-18, no obvious response is observed in the overdischarge coefficients due to the cyclic averaged values (Table 4.5).

Lithium-Ion Battery

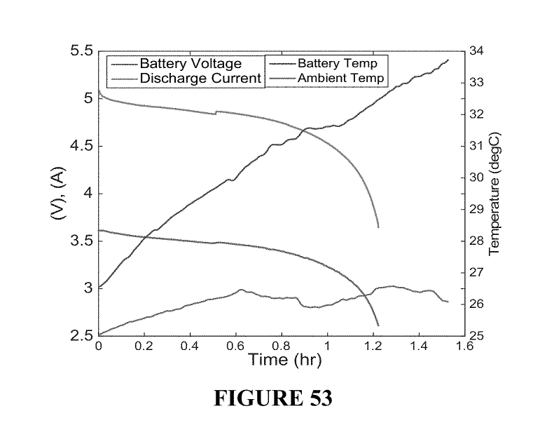

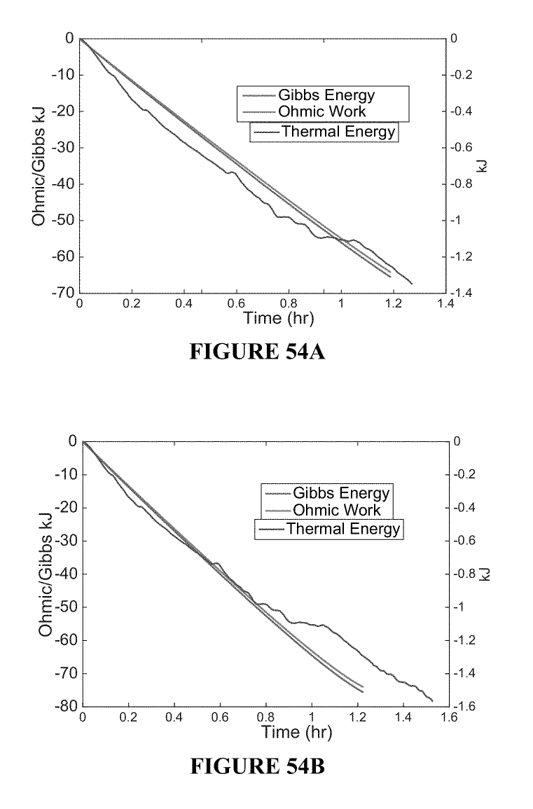

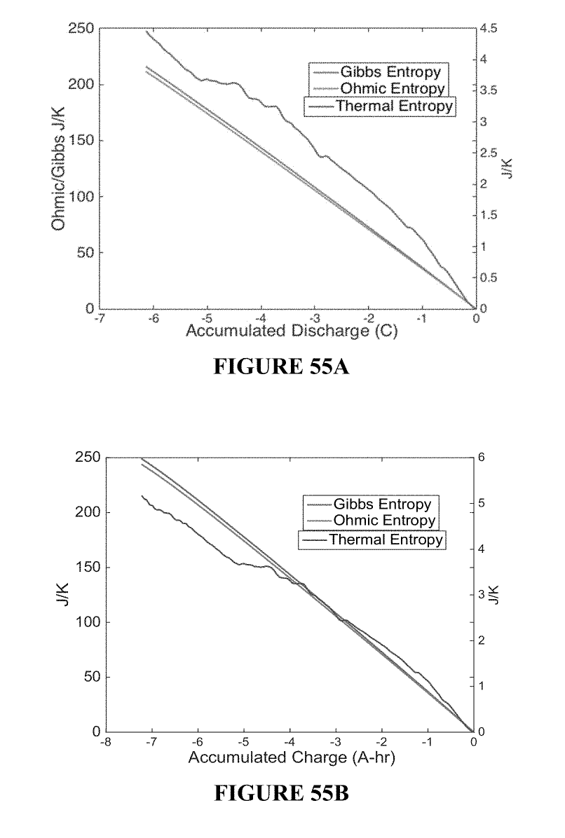

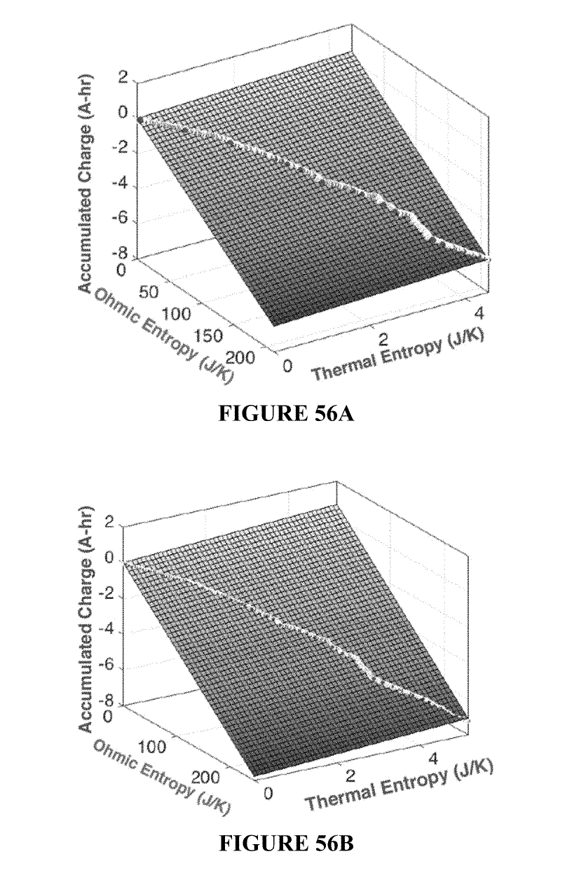

Due to the limited overdischarge capability, severe deviation from linearity was not experienced with the lithium ion batteries, see Table 4.6. As seen in FIG. 53, minimal depth of overdischarge only induced extra curvature in the DEG trajectory (FIGS. 56A-56B); the overdischarge DEG surface has a slightly different orientation for those cycles in which the predicted coefficients are different. The coefficients from the overdischarge data are not significantly different from those in the normal analysis, with the difference depending directly on the overdischarge duration for that cycle. FIGS. 54A-54B, 55A-55B, and 56A-56B show trends from over-discharging for cycle 4.

TABLE 4.6
Parameters for lithium-ion battery showing effects of over discharge.

| | Normal Discharge | | | | | | | | | | Overdischarge |
|---|---|---|---|---|---|---|---|---|---|---|---|
| | [symbol] | G_{N}\|W | ΔG_{N}\|T | ΔG_{N} | S′_{N}\|W | ΔS′_{N}\|T | S′_{N} | B_{W} | B_{T} | [symbol] ᵣₑₛ | [symbol] |
| N | A-hr | kJ | kJ | kJ | J/K | J/K | J/K | AhrK/J | AhrK/J | A-hr | A-hr |
| 1 | −4.84 | −51.8 | −1.2 | −53.0 | 170.2 | 4.0 | 174.1 | −0.030 | −0.018 | 0.073 | −6.09 |
| 2 | −6.70 | −70.1 | −1.9 | −72.0 | 232.1 | 6.2 | 238.4 | −0.031 | −0.012 | 0.107 | −8.36 |
| 3 | −8.26 | −86.8 | −1.1 | −87.9 | 281.8 | 3.7 | 285.5 | −0.030 | 0.019 | 0.049 | −9.52 |
| 4 | −6.15 | −64.2 | −1.4 | −65.5 | 211.6 | 4.5 | 216.1 | −0.031 | 0.006 | 0.102 | −7.22 |
| 5 | −5.83 | −61.6 | −1.3 | −62.9 | 203.9 | 4.2 | 208.1 | −0.030 | −0.003 | 0.092 | −6.71 |
| 6 | −8.52 | −90.3 | −1.7 | −92.0 | 299.1 | 5.6 | 304.7 | −0.030 | −0.024 | 0.112 | −9.74 |
| 7 | −5.31 | −55.6 | −1.2 | −56.8 | 184.3 | 4.1 | 188.4 | −0.030 | 0.041 | 0.038 | −6.39 |
| 8 | −8.58 | −91.5 | −1.6 | −93.1 | 300.4 | 5.1 | 305.5 | −0.029 | 0.068 | −0.006 | −8.70 |
| 9 | −4.31 | −45.7 | −1.5 | −47.2 | 152.5 | 5.2 | 157.7 | −0.030 | 0.027 | 0.045 | −5.18 |
| 10 | −7.04 | −74.5 | −1.1 | −75.7 | 245.3 | 3.7 | 249.1 | −0.030 | 0.035 | 0.091 | −8.51 |
| 11 | −9.59 | −104.9 | −1.2 | −106.1 | 340.9 | 3.9 | 344.8 | −0.028 | 0.070 | 0.038 | −10.54 |
| 12 | −5.09 | −53.4 | −1.6 | −55.0 | 177.7 | 5.2 | 182.9 | −0.031 | 0.041 | 0.070 | −6.24 |
| 13 | −7.05 | −75.3 | −1.4 | −76.7 | 249.4 | 4.8 | 254.2 | −0.031 | −0.003 | 0.145 | −8.26 |
| 14 | −6.81 | −72.5 | −1.6 | −74.1 | 239.9 | 5.3 | 245.2 | −0.030 | 0.007 | 0.078 | −7.97 |
| 15 | −8.46 | −91.8 | −1.2 | −92.9 | 302.0 | 3.8 | 305.9 | −0.029 | 0.028 | 0.102 | −10.01 |
| 16 | −5.36 | −56.9 | −0.9 | −57.9 | 188.0 | 3.1 | 191.1 | −0.030 | 0.012 | 0.111 | −6.41 |
| 17 | −7.11 | −76.5 | −1.2 | −77.7 | 252.3 | 4.0 | 256.3 | −0.029 | 0.033 | 0.078 | −8.94 |
| 18 | −7.37 | −79.7 | −1.4 | −81.0 | 262.1 | 4.5 | 266.6 | −0.030 | −0.013 | 0.155 | −9.44 |
| 19 | −5.21 | −55.0 | −1.2 | −56.2 | 181.5 | 3.8 | 185.3 | −0.029 | 0.033 | 0.034 | −6.77 |
| 20 | −6.08 | −62.4 | −0.9 | −63.3 | 205.8 | 3.0 | 208.8 | −0.030 | 0.004 | 0.041 | −7.84 |
| 21 | −6.36 | −66.4 | −1.8 | −68.2 | 220.9 | 6.1 | 227.0 | −0.030 | 0.030 | 0.064 | −8.18 |
| 22 | −3.31 | −35.2 | −0.7 | −35.9 | 116.3 | 2.4 | 118.7 | −0.029 | 0.010 | 0.034 | −3.35 |
| 23 | −9.96 | −105.0 | −1.1 | −106.1 | 342.9 | 3.7 | 346.6 | −0.031 | 0.004 | 0.164 | −12.36 |
| 24 | −5.36 | −54.8 | −1.0 | −55.9 | 181.1 | 3.4 | 184.5 | −0.031 | 0.008 | 0.087 | −8.82 |
| 25 | −3.57 | −36.1 | −1.0 | −37.1 | 118.8 | 3.2 | 122.0 | −0.032 | 0.003 | 0.062 | −6.40 |
| 26 | −2.56 | −26.9 | −1.7 | −28.6 | 89.0 | 5.6 | 94.6 | −0.032 | −0.005 | 0.049 | −3.74 |
| 27 | −3.92 | −40.1 | −1.2 | −41.3 | 132.3 | 3.9 | 136.2 | −0.033 | −0.001 | 0.117 | −5.75 |
| 28 | −2.14 | −22.3 | −1.7 | −24.0 | 73.9 | 5.6 | 79.5 | −0.033 | −0.002 | 0.049 | −3.18 |
| 29 | −2.06 | −21.4 | −1.7 | −23.1 | 70.9 | 5.6 | 76.6 | −0.033 | −0.001 | 0.045 | −3.02 |

| | Overdischarge | | | | | | | | |
|---|---|---|---|---|---|---|---|---|---|
| | G_{N}\|W | ΔG_{N}\|T | ΔG_{N} | S′_{N}\|W | ΔS′_{N}\|T | S′_{N} | B_{W} | B_{T} | [symbol] ᵣₑₛ |
| N | kJ | kJ | kJ | J/K | J/K | J/K | AhrK/J | AhrK/J | A-hr |
| 1 | −60.2 | −1.8 | −62.0 | 197.5 | 6.0 | 203.6 | −0.025 | 0.299 | −0.233 |
| 2 | −85.1 | −2.2 | −87.3 | 281.2 | 7.3 | 288.6 | −0.034 | −0.030 | 0.190 |
| 3 | −98.3 | −1.3 | −99.6 | 318.9 | 4.4 | 323.3 | −0.030 | 0.074 | 0.028 |
| 4 | −74.0 | −1.6 | −75.6 | 243.7 | 5.2 | 248.9 | −0.032 | 0.020 | 0.137 |
| 5 | −69.7 | −1.5 | −71.2 | 230.4 | 4.9 | 235.3 | −0.030 | 0.036 | 0.071 |
| 6 | −101.4 | −1.9 | −103.3 | 335.5 | 6.3 | 341.8 | −0.031 | −0.036 | 0.157 |
| 7 | −65.4 | −1.4 | −66.8 | 216.4 | 4.6 | 221.0 | −0.032 | 0.062 | 0.121 |
| 8 | −92.8 | −1.6 | −94.3 | 304.5 | 5.1 | 309.6 | −0.029 | 0.069 | −0.005 |
| 9 | −53.5 | −2.0 | −55.5 | 178.5 | 6.7 | 185.1 | −0.030 | 0.061 | 0.041 |
| 10 | −87.0 | −1.5 | −88.5 | 286.0 | 4.9 | 290.9 | −0.027 | 0.151 | −0.151 |
| 11 | −113.4 | −1.4 | −114.8 | 368.2 | 4.5 | 372.7 | −0.028 | 0.174 | −0.086 |
| 12 | −63.7 | −1.9 | −65.6 | 211.4 | 6.4 | 217.8 | −0.033 | 0.065 | 0.114 |
| 13 | −86.0 | −1.7 | −87.7 | 284.4 | 5.7 | 290.1 | −0.033 | −0.013 | 0.240 |
| 14 | −82.8 | −1.9 | −84.8 | 273.6 | 6.4 | 280.0 | −0.031 | 0.043 | 0.088 |
| 15 | −104.0 | −1.6 | −105.6 | 342.0 | 5.2 | 347.3 | −0.025 | 0.243 | −0.262 |
| 16 | −66.1 | −1.2 | −67.3 | 218.1 | 4.1 | 222.1 | −0.026 | 0.119 | −0.189 |
| 17 | −90.2 | −1.8 | −91.9 | 297.0 | 5.8 | 302.8 | −0.023 | 0.245 | −0.390 |
| 18 | −94.6 | −2.0 | −96.5 | 310.5 | 6.4 | 316.9 | −0.022 | 0.339 | −0.430 |
| 19 | −67.6 | −1.6 | −69.2 | 222.5 | 5.3 | 227.9 | −0.029 | 0.134 | −0.060 |
| 20 | −77.4 | −1.3 | −78.7 | 254.8 | 4.3 | 259.1 | −0.028 | 0.151 | −0.153 |
| 21 | −81.6 | −2.4 | −84.0 | 270.8 | 8.0 | 278.9 | −0.031 | 0.105 | 0.066 |
| 22 | −35.6 | −0.7 | −36.3 | 117.7 | 2.4 | 120.1 | −0.029 | 0.010 | 0.036 |
| 23 | −124.9 | −1.7 | −126.6 | 407.3 | 5.7 | 412.9 | −0.027 | 0.343 | −0.246 |
| 24 | −79.9 | −1.5 | −81.4 | 263.1 | 4.9 | 268.0 | −0.024 | 0.413 | −0.454 |
| 25 | −55.5 | −1.8 | −57.3 | 181.8 | 6.0 | 187.8 | −0.021 | 0.267 | −0.422 |
| 26 | −35.6 | −2.8 | −38.4 | 117.3 | 9.3 | 126.7 | −0.022 | 0.147 | −0.143 |
| 27 | −55.6 | −1.8 | −57.4 | 182.7 | 6.0 | 188.6 | −0.025 | 0.159 | −0.193 |
| 28 | −30.3 | −2.7 | −33.0 | 100.1 | 8.8 | 108.9 | −0.023 | 0.138 | −0.113 |
| 29 | −29.7 | −2.3 | −32.0 | 98.0 | 7.7 | 105.7 | −0.035 | 0.014 | 0.068 |

Discussion

DEG Trajectories, Surfaces, Domains and Changing Process Rates

Cycles 10-18 of the lead-acid battery data had different coefficient values after the discharge rate tripled. DEG coefficients, defined by equation (Equation 4.257)

$${B}_{i} = \frac{\partial C}{\partial {S}_{i}^{′}}$$

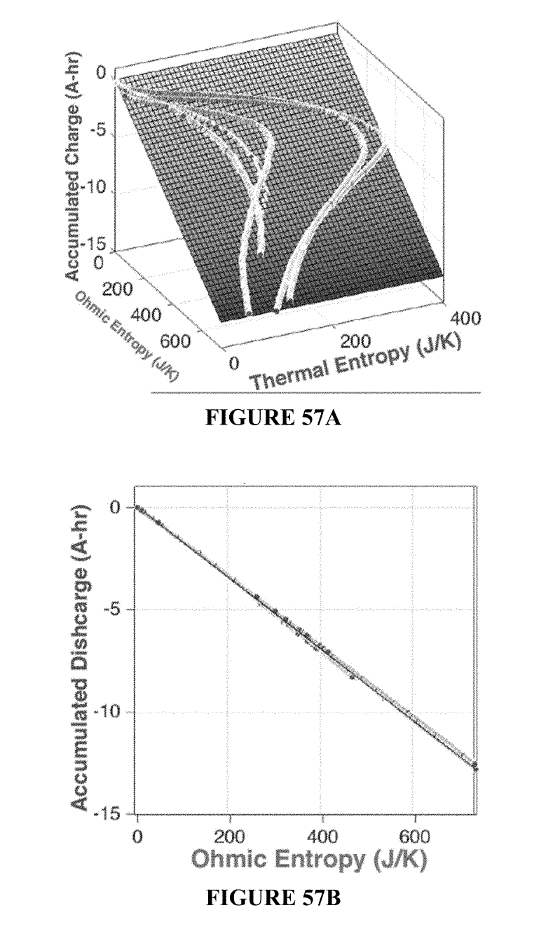

were sensitive to changing process rates. When a discharge (∂ [symbol] ) is unmatched by an entropy production (∂Sᵢ′), the DEG coefficient changes, which suggests a new orientation for the DEG surface. For a range of discharge rates, a set of DEG surfaces exist which define all possible DEG trajectories during operation. FIGS. 57A-57B, which plot DEG lines from cycles 1-9 of the lead-acid battery (same discharge rate) supports a characteristic DEG surface containing all DEG lines the battery can “draw” at a given charge/discharge rate. For cycles 10-18 at higher rate, a new surface developed with orientation defined by reduced B_{W} and increased B_{T}.

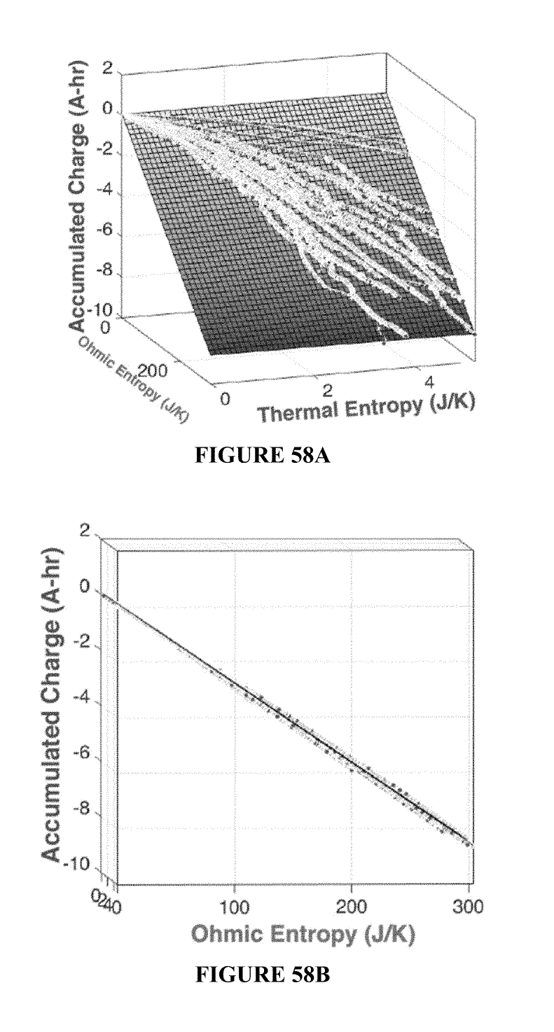

FIGS. 58A-58B plot DEG trajectories for Li-ion batteries cycles 1-29.

FIGS. 57A-57B and 58A-58B show the different thermal evolution of each cycle giving them their different characteristic and separating the trajectories. This is especially true for the lead-acid batteries as both thermal and ohmic entropies are similar in scale. In the case of Li-ion batteries, the thermal entropy scale, a hundredth of ohmic entropy scale, suggests these trajectories are almost the same and would overlap under controlled conditions. The Li-ion battery underwent primarily natural ohmic heating, while the lead-acid starter battery design has the thermal recovery feature discussed previously.

FIGS. 57A-57B and 58A-58B indicate that the Li-ion battery delivers more ohmic work with less accompanying thermal work, another explanation for the higher specific energy in Li-ion batteries than lead-acid batteries and the reason the thermal recovery feature is needed for lead-acid batteries. Electrolyte mass (3 kg for lead-acid and 0.23 kg for Li-ion battery) impacts heat capacity, thus thermal entropy. Specific heat capacity, a material property, additionally corroborates the significance of battery materials research.

The dependence of the DEG coefficients on process rate is further observed in the similar magnitudes of both discharge and charge ohmic coefficients B_{W} (=−0.03 AhrK/J and 0.02 AhrK/J respectively) for the li-ion battery, with similar discharge and charge currents I (=5 A and 4 A respectively), see Table 4.1. For the lead-acid battery, Table 4.2 shows that for discharge and charge currents of 11 A and 1.2 A respectively, B_{W}=−0.018 AhrK/J and 0.17 AhrK/J.

Important Features of DEG Coefficients
A pair of DEG coefficients determined from any cycle can predict accumulated charge/discharge in subsequent cycles. This suggests that consistent degradation coefficients can be determined at any point in a battery's life using simple measurements, without knowledge of history or capacity information from the manufacturer/supplier.
DEG trajectories appear to be characteristic of cycle conditions, DEG surfaces appear to be characteristic of a battery's discharge rates (all cycles at that rate) and the DEG domain seems to characterize the battery (all cycles and all rates). A battery having a domain with large capacity dimension and small thermal and ohmic entropy dimensions delivers power more efficiently.
For charging, DEG coefficients have opposite signs to their discharging counterparts to predict reverse-degradation (or positive transformation).

### Summary and Conclusion

Thermodynamic breakdown of the active processes in batteries during cycling were presented, including Gibbs-based and heat-based energy and entropy formulations during cycling. To these formulations was applied the DEG theorem to analyze battery degradation. Experimental results were applied to the DEG model.

A combination of thermodynamic analysis and the DEG theorem can be used by manufacturers to directly compare technologies, designs and materials used in battery manufacture. Also, without any prior information from the manufacturer about the battery, measurements and appropriate data analyses through the DEG theorem give a user an effective and consistent tool to compare various batteries to determine which is indeed most suitable for the intended application.

When conducted under controlled environments, data from a sample of same-model batteries can be used by manufacturers to minimize errors and defects similar to six sigma approach, or used in conjunction with the latter.

The nature of the coefficients obtained from the charging process may also provide insight into the use of the DEG theorem for transformation/healing processes.

### Example 5. General Fatigue and the DEG Theorem

All non-fluid matter yields or fails under continuous loading, static or dynamic. In solids, this failure is typically accelerated when subjected to dynamic loading. For static loading, static equilibrium conditions enable easy evaluation of required component strength for intended application. However, in dynamic loading conditions, accurate determination of degradation, eventually leading to fatigue failure can often be difficult. Various forms of dynamic loading are experienced in practice and component response varies based on a number of factors including material composition and loading conditions. With the use of metals in heavy-duty structural loading applications, a sudden failure can be catastrophic. Hence, of particular importance is cyclic loading of metallic components, attributed to about 90% of all metallic failures. Thermal cycling, as observed in electronic components, is also a significant area of fatigue analysis.

Existing approaches, most of which are empirical, sometimes give inconsistent results and failure measures are usually system or process-specific, hence not universally applicable. In this example, currently used approaches are reviewed and the DEG theorem applied to general fatigue analysis.

Existing Thermodynamic Models

Recent thermodynamic-based formulations to estimate damage in mechanical components and correlate entropy to a damage parameter are reviewed. Entropy has been related to fatigue via extensive experimental data. Naderi and Khonsari for low-cycle fatigue (LCF) assumed negligible heat dissipation during loading and formulated entropy generation from Morrow's cyclic plastic energy dissipation equation

$${W}_{p} = 2 {σ}_{f}^{′} {ɛ}_{f}^{′} ( \frac{1 - {n}^{′}}{1 + {n}^{′}} ) {( 2 {N}_{f} )}^{1 + b + c} \qquad ( Equation 5.277 )$$

giving entropy generation as

$${s}_{g} = \frac{{W}_{p}}{T} \qquad ( Equation$$

Using experimental torsional and bending fatigue data, they showed a linear relationship between normalized entropy generation and normalized number of cycles, as done for wear,

$$\frac{{s}_{i}}{{s}_{g}} \approx \frac{N}{{N}_{f}} \qquad ( Equation$$

Through equation (Equation 5.279), damage accumulation parameter D, based on continuum damage mechanics (CDM), was also related to entropy generation. Entropy generation from plastic energy dissipation can be replaced with entropy transfer out of the loaded sample via heat. With an energy balance, similar to the heat energy equation in Example 1 (equation (Equation 1.40)), heat transfer out of the sample into the surroundings was evaluated from measurements of sample temperature during loading.

$$( ∮ {σ}_{ij} d {ɛ}_{ij} ) \cdot f = ( {H}_{c d} + {H}_{cv} + {H}_{ic} ) + ( ρ {c}_{p} \frac{\partial T}{\partial t} + {\dot{E}}_{p} ) \qquad ( Equation 5.280 )$$

This approach has been applied to variable loading, from which a universally consistent damage accumulation model was proposed.

These works directly linked entropy generation with fatigue. Here total accumulated strain energy

Wᵢ=Wₚ+W_{∞}=AN_{f}^{α}+BN_{f}^{β} (Equation 5.281)

applicable to both low- and high-cycle fatigue, led to failure, where A, α, B and β are obtained from test data. In terms of material properties and measurable parameters,

$${W}_{t} = \frac{2 ( 1 + v ) {σ}_{f}^{′2} {N}^{2 b}}{3 E} + \frac{4 {ɛ}_{f}^{′} \frac{( 1 - {n}^{′} )}{( 1 + {n}^{′} )} {σ}_{a}^{( 1 + {n}^{′} ) / n}}{{σ}_{f}^{′1 / {n}^{′}}} \qquad ( Equation 5.282 )$$

They proposed the existence of a constant material property, the fracture fatigue entropy FFE, independent of cycle frequency, amplitude or sample size. Using thermodynamic formulations, they presented entropy generation rate

$$\dot{γ} = σ : \frac{{\dot{ɛ}}_{p}}{T} - \frac{{A}_{k} {\dot{V}}_{k}}{T} - {J}_{q} \cdot \frac{grad T}{{T}^{2}} \geq 0. \qquad ( Equation 5.283 )$$

Assuming negligible non-recoverable energy, the second term on the RHS was set to zero. To obtain heat generation they introduced heat capacity for reversible entropy content, yielding, for low cycle fatigue (LCF),

$$\begin{matrix}ρ C \dot{T} - k {∇}^{2} T = {W}_{p} \\ and & ( Equation 5.284 ) \\ \dot{γ} = \frac{{W}_{p}}{T} - \frac{{J}_{q} \cdot grad T}{{T}^{2}} & ( Equation 5.285 )\end{matrix}$$

where the first RHS term in equation (Equation 5.285) is the plastic strain entropy obtained from Wₚ, plastic strain energy; the second term is the heat conduction entropy. FFE is obtained by integrating equation (Equation 5.285) up to time of failure. For LCF, they neglected heat conduction within the sample (second term on LHS of equation (Equation 5.284) and second term on RHS of equation (Equation 5.285)) to give a lumped capacity model,

$${γ}_{f} = {\int }_{0}^{{t}_{f}} ( \frac{{W}_{p}}{T} ) dt \qquad ( Equation 5.286 )$$

Using experimental data and Finite Element Analysis, they validated their theory of the existence of a constant process-independent, material-dependent FFE, and showed a linear dependence between normalized entropy generation and normalized number of cycles (equation (Equation 5.279)).

Later, a real-time fatigue monitoring system was developed. With FFE (γ_{f}) as failure parameter and failure criterion, γ≤0.9γ_{f}, they consistently predicted failure with about 10% error, attributed to the difference between where on sample temperature was obtained and where actual failure occurred. Naderi and Khonsari demonstrated superiority, in terms of consistency under varying load conditions, of entropy-based fatigue analysis method over stress- and hysteresis energy-based models. Naderi and Khonsari applied their fatigue failure formulations to composite laminate. They indicated stored energy significant in composite laminate, comparable to dissipated heat, leading to the inclusion in total entropy generation, of heat storage entropy and a crack-initiating damage entropy, the latter being negligible in metals. Using hysteresis energy balance, entropy accumulation was

$${S}^{′} = {\int }_{0}^{tf} \frac{{E}_{th}}{T} + {\int }_{0}^{tf} \frac{{E}_{diss}}{T} + {\int }_{0}^{tf} \frac{{E}_{d}}{T} \qquad ( Equation$$

where Eₜₕ is heat stored, E_{diss} is heat dissipated, and Ed is damage energy. Combining the first two terms of equation (Equation 5.287) as mechanical entropy, experimental results compared each entropy component to the total entropy. Plots of mechanical entropy and damage entropy versus number of cycles are non-linear (more obvious in the cyclic entropy plots).

Others defined a complex damage of tribo-fatigue systems based on simultaneously occurring degradation mechanisms, e.g. sliding friction, fretting, impact, corrosion, heating, etc., that make using any one damage formulation inadequate. Using a cumulative general damage term ω′ (0<ω′<1) which includes mechanical, thermal and electrochemical energy changes, a tribo-fatigue entropy was proposed

$${S}_{TF}^{′} = {ω}^{′} \frac{{dW}_{D}}{T} \qquad ( Equation$$

where W_{D} is the absorbed damage energy at the failure section. Total entropy in the system is a sum of thermodynamic entropy (from combined first and second laws) and tribo-fatigue entropy (equation (Equation 5.288)).

$${dS}_{T} = dS + {dS}_{TF} = \frac{dU}{T} + \frac{δ W}{T} - \frac{μ {dN}^{′}}{T} + {ω}^{′} \frac{{dW}_{D}}{T} \qquad ( Equation$$

The damage parameter was related to normalized time and predicted human death by stress/damage accumulation from birth, depicting an exponential relationship. A human life version of the logarithmic S-N curve was also developed with similar profile as the S-N curves of metals. Equations (Equation 5.287) and (Equation 5.289) are equivalent formulations of entropy. Direct comparison shows damage energy

dE_{D}=ω′dW_{D} (Equation

which measures crack initiation and propagation, leading to eventual failure.

The above formulations were expanded and combined with continuum damage mechanics to form a basis for their proposed mechanothermodynamics (MTD) principles. Data for the isothermal fatigue of steel indicated an error of +/−15%.

The Problem

Extensive data shows the consistency of entropy measurements in estimating damage and failure in cyclically loaded members. With exception of the CDM damage parameter of Khonsar, most studies introduced a new damage parameter to link fatigue to entropy works. The following analysis uses the DEG theorem to relate existing damage accumulation measures to the individual active process entropies. Data will be used to compare the DEG approach to the existing approaches. Also, data from the lengthwise loading of composite laminate will be used to demonstrate the linearity between number of cycles and appropriate combination of entropy components. Currently, most fatigue-entropy formulations apply to metal fatigue under mechanical loading. The formulations that follow apply to all forms of cyclic loading.

Analysis

A component undergoing concurrent cyclic work interactions will be analyzed. As for grease and batteries, formulations for entropy generation combined with the DEG theorem will render fatigue failure criteria.

Thermodynamic Analysis

The current investigation establishes all three modes of interaction—mechanical, thermal and chemical.

Infinitesimal Model—Maximum Work Model

Helmholtz Analysis

Assumptions:
1. The system is the sample only.
2. System is closed.
3. Heat transfers with surroundings.

The infinitesimal loss of Helmholtz free energy in a component in loaded operation is given by equation (Equation 1.30)

dA=−SdT−Xdζ+μdN′ (Equation

where thermal energy change SdT=CdT, see equation (Equation 1.43). The thermodynamic work

Xdζ=δW_{F} (Equation

for small-deformation stress σ—strain ε loading is δW_{F}=σ:ε. Term μdN′ defines energy loss due to corrosion, for corrosion-enhanced fatigue, where

$${dN}^{′} = \frac{d m}{{M}_{m}} \qquad ( Equation$$

and Mₘ is the specimen molecular mass. Combining gives the maximum Helmholtz free energy loss in a solid component undergoing dynamic loading

$$dA = - CdT - δ {W}_{F} + \frac{μ}{{M}_{m}} d m \qquad ( Equation$$

To satisfy the required dA≤0 as the sample energy decreases, dT≥0, δW_{F}≥0 and dm≤0, and equation (Equation 5.294) follows the IUPAC sign convention.

Entropy generation from equation (Equation 1.34) is

$$δ {S}^{′} = \frac{SdT}{T} + \frac{X d ζ}{T} - \frac{μ {dN}^{′}}{T} \qquad ( Equation$$

Substituting heating, cyclic loading and chemical degradation terms,

$$δ {S}^{′} = \frac{CdT}{T} + \frac{δ {W}_{F}}{T} - \frac{μ d m}{{M}_{m} T} \qquad ( Equation$$

Equation (Equation 5.296) accumulates entropy generation of three simultaneous independent processes. For the more common mechanical and thermal loading-dominated fatigue cases,

$$δ {S}^{′} = \frac{CdT}{T} + \frac{δ {W}_{F}}{T} \qquad ( Equation$$

Equations (Equation 5.296) and (Equation 5.297) suggest that during loading, the terms on the RHS increase entropy production as dT≥0 and dm≤0.

Infinitesimal Model—Heat-Only Model

Assumptions:
1. The system is the sample only.
2. System is closed.
3. Heat transfers between sample and immediate surroundings via free convection.

From equation (Equation 1.40), energy dissipation via heat is the heat generation

δE′=CdT−δQ (Equation

From equation (Equation 1.41), entropy generation from heat

$$δ {S}^{′} = \frac{CdT}{T} - \frac{δ Q}{T} \qquad ( Equation$$

where the RHS terms are sample thermal energy storage and heat transfer entropies respectively. The heat storage term is equivalent to the thermal energy term in the Helmholtz formulation in equation (Equation 5.294). Heat transfer out of the component is negative, according to IUPAC convention. Rate of heat transfer out of the component

\dot{Q}=ΔT/Rₜ (Equation

is the ratio of the difference between component and ambient temperatures to the thermal resistance in between. The heat formulation (equation (Equation 1.41)) applies to all processes and loading conditions.

Experimental Model—Work and Heat

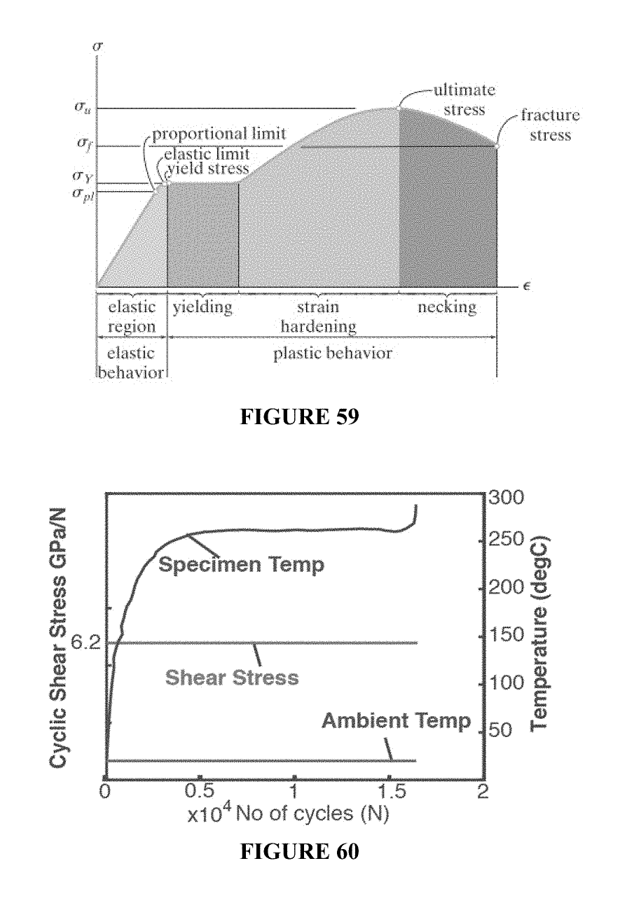

Here rate forms of the above models are presented. Parameters can be directly measured to determine energy changes and entropy production. FIG. 59 shows non-linearity in loaded steel behavior before failure, hence limiting steady state approaches.

Control Parameters:
1. The sample is a closed system.
2. Heat transfers with the surroundings via natural convection.
   Rewriting equations (Equation 5.294) and (Equation 5.296) in rate form,

$$\begin{matrix}\dot{A} = - C \dot{T} - {\dot{W}}_{F} - \frac{μ}{{M}_{m}} \dot{m} & ( Equation \\ {\dot{S}}^{′} = \frac{C \dot{T}}{T} + \frac{{\dot{W}}_{F}}{T} + \frac{μ \dot{m}}{{M}_{m} T} & ( Equation\end{matrix}$$

The rate of irreversible entropy production in the sample undergoing cyclic mechanical, thermal and chemical interactions is the sum of the individual rates of work inputs and process energies divided by the temperature at the heat exchange boundary. In the absence of chemical interaction, the chemical term drops out to give the rate form of (Equation 5.297)

$${\dot{S}}^{′} = \frac{C \dot{T}}{T} + \frac{{\dot{W}}_{F}}{T} \qquad ( Equation$$

If thermal equilibrium is reached after a short period, as shown later for high cycle fatigue (HCF), the first RHS term eventually vanishes to give the steady state entropy generation

$${\dot{S}}_{HCF}^{′} = \frac{C \dot{T}}{T} - \frac{\dot{Q}}{T} \qquad ( Equation$$

Using heat generation entropy from equation (Equation 5.299),

$$\dot{{S}^{′}} = \frac{C \dot{T}}{T} - \frac{\dot{Q}}{T} \qquad ( Equation$$

Cycle Analysis

Accumulated entropy production after N cycles from equation (Equation 5.302),

$${S}_{N}^{′} = {\int }_{0}^{N} \frac{C \dot{T}}{T} dN + {\int }_{0}^{N} \frac{{\dot{W}}_{F}}{T} dN + {\int }_{0}^{N} \frac{μ \dot{m}}{{M}_{m} T} dN \qquad ( Equation$$

From equation (Equation 5.305), accumulated entropy generation from heat generation,

$${S}_{N}^{′} = {\int }_{0}^{N} \frac{C \dot{T}}{T} dN - {\int }_{0}^{N} \frac{\dot{Q}}{T} dN \qquad ( Equation$$

Degradation Entropy Generation (DEG) Analysis

DEG formulations and approach are applied to fatigue. Chemical degradation-related fatigue has been neglected for simplicity. Identifying entropy production for active processes via equation (Equation 5.302), and applying this to the DEG theorem gives

$$\frac{dw}{dt} = {B}_{T} \frac{C \dot{T}}{T} + {B}_{W} \frac{{\dot{W}}_{F}}{T} \qquad ( Equation$$

In terms of entropy generation from heat analysis, equation (Equation 5.305) and the DEG theorem gives

$$\frac{dw}{dt} = {B}_{T} \frac{C \dot{T}}{T} - {B}_{Q} [ \frac{( T - {T}_{∞} )}{RT} ] \qquad ( Equation$$

B can be evaluated using appropriate measurements of parameters from

$${B}_{i} = \frac{\partial w}{\partial {S}_{i}^{′}} \qquad ( Equation$$

the ratio of the slope of the rate of w to the specific process entropy production rate.

Cyclic Analysis

For dynamic loading conditions, duration in time is given by

$$dt = \frac{dN}{f} \qquad ( Equation$$

where f is cycle frequency. In fatigue, cyclic loads are defined per cycle, hence dt is replaced by dN in accumulation integrals. Entropy accumulates with cyclic loads, hence degradation over N cycles relates to entropy production through an integral

$${w}_{N} = {B}_{T} {\int }_{0}^{N} \frac{C \dot{T}}{T} dN + {B}_{w} {\int }_{0}^{N} \frac{{\dot{W}}_{F}}{T} dN \qquad ( Equation$$

In heat generation terms from equation (Equation 5.309),

$${w}_{N} = {B}_{T} {\int }_{0}^{N} \frac{C \dot{T}}{T} dN - {B}_{Q} {\int }_{0}^{N} [ \frac{( T - {T}_{∞} )}{RT} ] dN \qquad ( Equation$$

DEG Coefficients from Existing Models

Normal Bending Stress

Under isothermal conditions, instantaneous normal bending stress a in a component is given by the flexure formula

$$σ = - \frac{My}{I} and {σ}_{max} = \frac{Mc}{I} \qquad ( Equation 5.314 )$$

where M is the resultant internal moment from applied bending, y is any location on a locus perpendicular to the neutral axis and I is cross-sectional area moment of inertia. Maximum stress σₘₐₓ is obtained at c, the location on the perpendicular locus farthest from the neutral axis. For cylinders, c is the outer radius r. Comparing σₘₐₓ to equation (Equation 5.318) and dropping the first term on the RHS due to isothermal condition (dT=0) give the Helmholtz-normal stress coefficient

$${B}_{{W}_{σ}} = \frac{McT}{I {\dot{W}}_{F}} \qquad ( Equation$$

Torsional Shear Stress

Under isothermal conditions, instantaneous torsional shear stress τ in a component is given by the torsion formula

$$τ = \frac{{M}_{t} y}{J} and {τ}_{max} = \frac{{M}_{t} c}{J} \qquad ( Equation 5.316 )$$

where Mₜ is the resultant internal torque from applied torsion, y is any location on a locus perpendicular to the neutral axis and J is the cross-sectional area polar moment of inertia. Maximum shear stress τₘₐₓ is obtained at c, the location on the perpendicular locus farthest from the neutral axis. For cylinders, c is the outer radius r. Comparing τₘₐₓ to equation (Equation 5.320) and dropping the first term on the RHS due to isothermal condition (dT=0) give the Helmholtz-shear stress coefficient

$${B}_{{W}_{τ}} = \frac{{M}_{t} cT}{J {\dot{W}}_{F}} \qquad ( Equation$$

Equations (Equation 5.315) and (Equation 5.317) imply that measurements of stress and entropy before onset of failure give the DEG coefficient. For degradation and entropy generation purposes, it is noted that equations (Equation 5.315) and (Equation 5.317) include only the plastic component of the applied load. Formulations for this component such as given in equation (Equation 5.282) are readily available. As shown later, isothermal conditions prevail during high-cycle fatigue, hence equations (Equation 5.315) and (Equation 5.317) are valid in the HCF region for normal bending and torsional loads. This coefficient determined for a sample component can be used to estimate onset of failure for subsequent samples of the same material under fatigue loading.

Fatigue Analysis Using Common Fatigue Measures

Commonly used fatigue parameters are combined with entropy generation to unify current practices with the DEG approach.

Stress as Degradation Measure

In material science, stress is one of the most widely used parameters in component health analysis. Accumulated stress as degradation parameter, equation (Equation 5.312) becomes, for normal bending stress,

$${σ}_{N} = {\int }_{0}^{N} σ dN = {B}_{T} {\int }_{0}^{N} \frac{C \dot{T}}{T} dN + {B}_{W} {\int }_{0}^{N} \frac{{\dot{W}}_{F}}{T} dN \qquad ( Equation$$

where σ is the instantaneous normal bending stress and the Helmholtz-normal stress coefficients

$${B}_{T} = \frac{\partial {σ}_{N}}{\partial {S}_{T}^{′}} ; {B}_{W} = \frac{\partial {σ}_{N}}{\partial {S}_{W}^{′}} \qquad ( Equation$$

pertain to thermal entropy

$${S}_{T}^{′} \int \frac{C \dot{T}}{T} dN$$

and plastic strain entropy

$${S}_{W}^{′} = \int \frac{{\dot{W}}_{F}}{T} dN$$

respectively. For torsional shear stress as another degradation parameter,

$${τ}_{N} = {\int }_{0}^{N} τ dN = {B}_{T} {\int }_{0}^{N} \frac{C \dot{T}}{T} dN + {B}_{W} {\int }_{0}^{N} \frac{{\dot{W}}_{F}}{T} dN ( Equation$$

where τ is the instantaneous torsional shear stress, and Helmholtz-shear stress coefficients

$${B}_{T} = \frac{\partial {τ}_{N}}{\partial {S}_{T}^{′}} ; {B}_{W} = \frac{\partial {τ}_{N}}{\partial {S}_{W}^{′}} ( Equation$$

For simultaneously occurring loads such as combined bending and torsion, equations (Equation 5.318) and (Equation 5.320) can be combined using the von Mises criterion

σ′=(σ²+3τ²)^{1/2}(Equation

where σ′ is the combined stress.

Similarly, via equation (Equation 5.313),

$${σ}_{N} = {\int }_{0}^{N} σ dN = {B}_{T} {\int }_{0}^{N} \frac{C \dot{T}}{T} dN - {B}_{Q} {\int }_{0}^{N} [ \frac{( T - {T}_{∞} )}{RT} ] dN ( Equation$$

with heat generation-normal stress coefficients

$${B}_{T} = \frac{\partial {σ}_{N}}{\partial {S}_{T}^{′}} ; {B}_{Q} = \frac{\partial {σ}_{N}}{\partial {S}_{Q}^{′}} ( Equation$$

### Also

$${τ}_{N} = {\int }_{0}^{N} τ dN = {B}_{T} {\int }_{0}^{N} \frac{C \dot{T}}{T} dN - {B}_{Q} {\int }_{0}^{N} [ \frac{( T - {T}_{∞} )}{RT} ] dN ( Equation$$

with heat generation-shear stress coefficients

$${B}_{T} = \frac{\partial {τ}_{N}}{\partial {S}_{T}^{′}} ; {B}_{Q} = \frac{\partial {τ}_{N}}{\partial {S}_{Q}^{′}} ( Equation$$

that pertain to entropies from thermal energy change and heat transfer respectively.

Normalized Number of Cycles N/N_{f}

Equation (Equation 5.279) by normalizing entropy and number of cycles indicates

N=f(S) (Equation

If the failure point N_{f} is known for the component, normalized number of load cycles as degradation parameter

$$\frac{N}{{N}_{f}} = {\int }_{0}^{N} \frac{dN}{{N}_{f}} = {B}_{{T}_{N}} {\int }_{0}^{N} \frac{C \dot{T}}{T} dN + {B}_{{W}_{N}} {\int }_{0}^{N} \frac{{\dot{W}}_{F}}{T} dN ( Equation$$

where N is the number of cycles from start of loading and the Helmholtz-normal stress coefficients

$${B}_{{T}_{N}} = \frac{\partial ( N / {N}_{f} )}{\partial {S}_{T}^{′}} ; {B}_{{W}_{N}} = \frac{\partial ( N / {N}_{f} )}{\partial {S}_{W}^{′}} ( Equation$$

pertain to thermal entropy

$${S}_{T}^{′} = \int \frac{C \dot{T}}{T} dN$$

and plastic strain entropy

$${S}_{W}^{′} = \int \frac{{\dot{W}}_{F}}{T} dN$$

respectively.

Via equation (Equation 5.313),

$$\frac{N}{{N}_{f}} = {\int }_{0}^{N} \frac{dN}{{N}_{f}} = {B}_{{T}_{N}} {\int }_{0}^{N} \frac{C \dot{T}}{T} dN - {B}_{{Q}_{N}} {\int }_{0}^{N} [ \frac{( T - {T}_{∞} )}{RT} ] dN ( Equation$$

with heat generation-normal stress coefficients

$${B}_{{T}_{N}} = \frac{\partial ( N / {N}_{f} )}{\partial {S}_{T}^{′}} ; {B}_{{Q}_{N}} = \frac{\partial ( N / {N}_{f} )}{\partial {S}_{Q}^{′}} ( Equation$$

that pertain to entropies from thermal energy change and heat transfer respectively.

CDM Damage Parameter D

A damage parameter was proposed based on depletion of static toughness energy of a loaded component which was showed via experimental data to consistently represent fatigue damage evolution. Other researchers have shown more data consistent with the damage parameter. In terms of number of cycles,

$$D = - \frac{{D}_{{N}_{f} - 1}}{ln {N}_{f}} ln [ 1 - \frac{N}{{N}_{f}} ] ( Equation$$

where D_{N}_{_{f}}₋₁ is the fatigue-related critical damage, just before failure, N_{f} is the number of cycles to failure. D has been estimated as a function of entropy generation. Via equation (Equation 5.279),

$$D = - \frac{{D}_{{N}_{f} - 1}}{ln {S}_{f}^{′}} ln [ 1 - \frac{{S}^{′}}{{S}_{f}^{′}} ] \qquad ( Equation$$

where S′ is total irreversible entropy accumulated and S′_{f} is the total irreversible entropy accumulated at failure.

Equations (Equation 5.332) and (Equation 5.333) show that D has a logarithmic relationship with time N and entropy accumulation S′. The fundamental laws of thermodynamics define changes in a system with time. Hence a direct linearity does not exist between D and the components of entropy as seen with other measures. Rewriting equation (Equation 5.333),

D=B_{D}S′D (Equation

where logarithmic DEG-D coefficient

$${B}_{D} = - \frac{{D}_{{N}_{f} - 1}}{ln {S}_{f}^{′}} \qquad ( Equation$$

and logarithmic rest of life entropy accumulation

$${S}_{D}^{′} = ln [ 1 - \frac{{S}^{′}}{{S}_{f}^{′}} ] \qquad ( Equation$$

Using the DEG approach, equation (Equation 5.308) gives a breakdown into component terms

D=B_{D}_{_{T}}S′_{D}_{_{T}}+B_{D}_{_{W}}S′_{D}_{_{W}} (Equation

where from equation (Equation 5.336),

$$\begin{matrix}{S}_{{D}_{T}}^{′} = ln [ 1 - \frac{{S}_{T}^{′}}{{S}_{{T}_{f}}^{′}} ] \\ and & ( Equation \\ {S}_{{D}_{W}}^{′} = ln [ 1 - \frac{{S}_{W}^{′}}{{S}_{{W}_{f}}^{′}} ] & ( Equation\end{matrix}$$

where

$${S}_{T}^{′} = \int \frac{C \dot{T}}{T} dN$$

is the thermal entropy and

$${S}_{W}^{′} = \int \frac{{\dot{W}}_{F}}{T} dN$$

is plastic work entropy. In isothermal loading, B_{D}_{_{W}}=B_{D}.

Using heat-only terms,

$$\begin{matrix}D = {B}_{{D}_{T}} {S}_{{D}_{T}}^{′} - {B}_{{D}_{Q}} {S}_{{D}_{Q}}^{′} \\ where & ( Equation \\ {S}_{{D}_{Q}}^{′} = ln [ 1 - \frac{{S}_{Q}^{′}}{{S}_{{Q}_{f}}^{′}} ] & ( Equation\end{matrix}$$

The above formulations are verified experimentally below.

Fatigue Strength

Another commonly used measure is the fatigue strength S_{Nf}, defined as the peak stress before failure. It has been showed, via number of load cycles N, that

S_{Nf}=S_{Nf}(S′) (Equation

a function of accumulated irreversible entropy. The fatigue strength relates to the number of cycles via the S-N curve and from equation (Equation 5.328), a relationship to entropy can also be anticipated.

For low-cycle fatigue, N≤10³, Shigley obtains from the S-N curve

S_{Nf}|LCF=σ′_{f}(2N)^{b} (Equation

where σ′_{f} is the combined stress at fracture including all loading modes,

$$b = - \frac{log ( {σ}_{f}^{′} / {S}_{e} )}{log ( 2 {N}_{e} )} \qquad ( Equation$$

For high-cycle fatigue (second segment in Error! Reference source not found.), 10³<N<10⁶, Shigley gives

$${S}_{Nf} \vert HCF = \frac{{{σ}_{f}^{′2} ( 4 * {10}^{6} )}^{b} {N}^{b}}{{S}_{e}} \qquad ( Equation$$

At failure,

$${\int }_{0}^{N} {σ}^{′} dN = {S}_{Nf} \qquad ( Equation$$

Using instantaneous entropy formulations, equations (Equation 5.318) and (Equation 5.320) apply to all fatigue cycles. For LCF, combining equations (Equation 5.318) and (Equation 5.343),

$${{σ}_{f}^{′} ( 2 N )}^{b} = {B}_{T} {\int }_{0}^{{N}_{lcf}} \frac{C \dot{T}}{T} dN + {B}_{W} {\int }_{0}^{{N}_{lcf}} \frac{{\dot{W}}_{F}}{T} dN \qquad ( Equation$$

which requires knowledge of the temperature rise during loading, characteristic of low-cycle fatigue as shown by Khonsari et al. The upper limit N_{lcf} counts the cycles to failure.

For high cycle fatigue (HCF), equating (Equation 5.318) to (Equation 5.345) yields

$$\frac{{σ}_{f}^{′2} {4}^{b} {N}^{3 b}}{{S}_{e}} = {B}_{T} {\int }_{{N}_{lcf}}^{{N}_{hcf}} \frac{C \dot{T}}{T} dN + {B}_{W} {\int }_{{N}_{lcf}}^{{N}_{hcf}} \frac{{\dot{W}}_{F}}{T} dN \qquad ( Equation$$

where N_{lcf} counts cycles to failure. If dT=0 in the HCF region, equation (Equation 5.348) gives

$${B}_{W} = [ \frac{{{σ}_{f}^{′2} ( 4 * {10}^{6} )}^{b} {N}^{b}}{{S}_{e}} ] / [ {\int }_{{N}_{lcf}}^{{N}_{hcf}} \frac{{\dot{W}}_{F}}{T} dN ] \qquad ( Equation$$

a more general form of equations (Equation 5.315) and (Equation 5.317), applicable to all simultaneously occurring mechanical loading conditions. Equation (Equation 5.349) implies that with a known endurance limit Se, measurements of stress, and accumulated entropy before onset of failure give the Helmholtz-fatigue strength coefficient.

For HCF,

$${S}_{Nf} = {{S}_{0} ( \frac{1 + N / {N}_{e}}{N} )}^{α} \qquad ( Equation$$

where S₀ is initial strength, Nₑ is endurance limit cycle number, α is the slope of the logarithmic S-N curve before infinite life point 10⁶ cycles.

As in equation (Equation 5.349), combining with equation (Equation 5.318) gives

$${B}_{W} = [ {{S}_{0} ( \frac{1 + N / {N}_{e}}{N} )}^{α} ] / [ {\int }_{{N}_{lcf}}^{{N}_{hcf}} \frac{{\dot{W}}_{F}}{T} dN ] \qquad ( Equation$$

Factor of Safety for Variable Loading

Oftentimes, components are subjected to varying irregular loads. The above formulations apply to all dynamic loads including fully reversed, combined modes and variable loads. To account for variable loading in real-life applications, Shigley gives a Soderberg criterion for factor of safety n

$$\frac{1}{n} = \frac{{σ}_{a}}{{S}_{e}} + \frac{{σ}_{m}}{{S}_{y}} \\ \text{?} \text{indicates text missing or illegible when filed} \qquad ( Equation \text{?}$$

and a modified Goodman factor of safety

$$\frac{1}{n} = \frac{{σ}_{a}}{{S}_{e}} + \frac{{σ}_{m}}{{S}_{ut}} \\ \text{?} \text{indicates text missing or illegible when filed} \qquad ( Equation \text{?}$$

Here, a similar criterion is proposed using accumulated stress and fatigue stress as

$$\frac{1}{n} = \frac{{σ}_{N}^{′}}{{S}_{Nf}} \\ \text{?} \text{indicates text missing or illegible when filed} \qquad ( Equation \text{?}$$

which, from equation (Equation 3.144) and Shigley's equation (Equation 5.345), gives

$$\frac{1}{n} = [ {\int }_{{N}_{lcf}}^{{N}_{hcf}} \frac{{\dot{W}}_{F}}{T} dN ] / [ \frac{{{σ}_{f}^{′2} ( 4 * {10}^{6} )}^{b} {N}^{b}}{{S}_{e}} ] . \qquad ( Equation 5.355 )$$

Comparing equation (5.64) to equation (Equation 5.349) gives n=B_{W}, showing that Fatigue DEG coefficients appear as factors of safety in designing components for cyclic loading. The DEG coefficients, arising from the second law of thermodynamics, should be universally applicable to all forms of interactions, including combined modes of irregular and transient loading conditions.

Comparison to Existing Energy Models

Energy-based models are compared to the prior analysis and results. While the heat approach appears similar to the heat-only approach (section 5.2.1.2) described herein, it makes a fundamental assumption that has since been carried over since the DEG theorem was first verified experimentally. By assuming ΔS=0, entropy generation by heat equates to entropy transfer by heat. However, to simplify entropy generation evaluation, a steady state entropy assumption, justified by a stationary state of the object, was used. In thermodynamic and heat analysis, this implies isothermal conditions. As discussed above, the DEG implies more than one concurrent process for the predicted linearity. In Examples 3 and 4, it was shown that high-rate boundary work produces entropy two or more orders of magnitude higher than the resulting thermal entropy change, hence the isothermal assumption works in many practical thermodynamic formulations. In real applications and to understand the effect of a potential increase in thermal entropy, this is not applicable. Significant temperature changes in low-cycle fatigue-tested metals. Also, Amiri et al related the initial temperature rise to cycle life and reviewed other studies correlating changes in sample temperature to fatigue. Hence a complete formulation of heat entropy generation from both components (heat transfer and thermal storage) is expected to give more accurate results.

A close look at equations (Equation 5.284) and (Equation 5.285) shows that combining both equations gives the thermal entropy balance in equation (Equation 1.41) neglecting heat conduction within sample. As shown in heat generation analysis and discussed above, both the storage and the heat transfer terms are of the same order and significant in instantaneous evaluation of energy dissipation via heat. If strain energy accumulation is determined from the work interaction, as intended by the authors and given in equation (Equation 5.282), evaluating entropy generation using equation (Equation 5.286) does not include the thermal entropy introduced by Helmholtz free energy. As noted in earlier examples, while this component of total entropy is often negligible based on relative order of magnitude, it is significant in DEG analysis as well as processes with significant temperature variation, like thermal cycling. Including it gives a more consistent and universal formulation.

Equation (Equation 5.287) is equivalent to the entropy balance in equation (Equation 1.41), where entropy change within the sample

$$Δ S = {\int }_{0}^{tf} \frac{{E}_{th}}{T} + {\int }_{0}^{tf} \frac{{E}_{d}}{T} \\ \text{?} \text{indicates text missing or illegible when filed} \qquad ( Equation \text{?}$$

While the stored entropy might be insignificant compared to entropy generated by the boundary work interaction, its contribution to actual degradation as determined by DEG coefficients could be quite significant. Hence inclusion at all times is useful to adequately capture the separate effect of heating on failure, fatigue or otherwise. With the DEG theorem, a consistent thermodynamic approach is used for all components and all materials under all loading conditions (including thermal and chemical cycling).

Fatigue Experiments

Procedure for fatigue experiments can be found in ASTM standards. Manufacturers routinely modify these processes according to specific component design requirements.

Data for steel and aluminum fatigue experiments (bending and torsion) were obtained from Khonsari et al. Using high-resolution infra-red thermography, temperature evolution of the loaded sample was monitored. Plastic cyclic load W_{F} was determined using equation (Equation 5.282). For the steel torsion, sample dimensions are given in mm.

Results and Data Analysis

Data processing and analysis will be presented. Using equations for energy loss in sample and entropy production via work and thermal energy changes, the columns in Tables 5.2 and 5.3 were evaluated. Where available, already evaluated entropy components were used directly, e.g. plastic strain entropy. Unavailable components were evaluated from measured parameters, e.g. thermal entropy calculated from temperature measurements. Observed trends will be discussed and compared to previous work.

Constants Used

Appropriate constants required in the above formulations include:

TABLE 5.1
Material properties used in calculating plastic strain energy from torsional loading

| | |
|---|---|
| Material: Stainless Steel SS 304 | 0.2% offset yield strength Sy = 325 MPa |
| Torsional modulus G: 82.8 GPa | Endurance limit Se = 128 MPa |
| Fatigue strength coefficient σ′_{f} = 709 MPa | Fatigue ductility coefficient ε′_{f} = 0.171 |
| Fatigue strength exponent b = −0.121 | Fatigue ductility exponent c = −0.353 |
| Cyclic strain hardening exponent n' = 0.296 | Heat transfer coefficient of air, hₐᵢᵣ = 25 W/m²K |
| $Specific heat capacity {Cp}_{ss} = 500 \frac{J}{kg} K$ | |

For a 1-dimensional lumped-capacity heat transfer model, thermal resistance including via free convection with the surroundings is given by

$${R}_{t} = ( \frac{1}{{h}_{air} {A}_{s}} ) \\ \text{?} \text{indicates text missing or illegible when filed} \qquad ( Equation \text{?}$$

Helmholtz Thermodynamic Analysis (Maximum Work)

Using equations (Equation 5.306) and (Equation 5.307), processed parameters are presented in Tables 5.2 and 5.3. Parameters with labels on the right side of the legend are plotted on the right axis, and vice versa.

### Torsional Fatigue Testing of Steel SS304

TABLE 5.2
Helmholtz-based experimental analysis results. In Bᵢ units, w =
degradation measure. Damage D-based entropies are logarithmic.

| Degradation | Value at | ΔA_{N}\|W | ΔA_{N}\|T | ΔA_{N} | S′_{N}\|W | S′_{N}\|T | S′_{N} | B_{W} | B_{T} | |
|---|---|---|---|---|---|---|---|---|---|---|
| Measure w | failure | MJ | MJ | MJ | MJ/K | MJ/K | MJ/K | w/J/K | w/J/K | wᵣₑₛ |
| Shear Stress τ (GPa) | 1.02E+05 | −514.8 | −1062 | −1576 | 0.996 | 2.532 | 3.528 | 0.106 | −0.00171 | 4451 |
| Damage D | 1 | | | | −6.17 | −3.44 | −3.74 | −0.1635 | 0.00173 | −0.00105 |
| N/N_{f} | 1 | −514.8 | −1062 | −1576 | 0.996 | 2.532 | 3.528 | 1.04E−06 | −1.67E−08 | 0.00437 |

From plastic work W_{F} induced by cyclic loads, plastic torque

$${M}_{F} = \frac{{\dot{W}}_{F}}{2 π}$$

and plastic (residual) shear stress amplitude

$${τ}_{F} = \frac{{M}_{F} r}{J}$$

were obtained, where

$$J = \frac{π {r}^{4}}{16}$$

is the polar moment of inertia. FIG. 60 shows monitored temperature during torsional loading of steel at a constant shear stress amplitude of 62 MPa and constant frequency of 10 Hz. An initial steep rise in temperature during the first 5000 cycles, via an initially large dissipation from hysteresis, eventually steadies with heat transfer to the surroundings. Ambient temperature was assigned as Tₐ=T_{(N=0)}=20 degC.

Degradation measures evaluated from the extracted data are in column 1. Damage parameter D was evaluated from equation (Equation 5.332) with D_{N}_{_{f}}₋₁=1. Trapezoidal quadratures estimated integrals of cyclic shear stress, plastic strain energy and plastic strain entropy. For a process occurring over cycles 0 to N_{f}, accumulated shear stress

$${τ}_{N} = {\int }_{0}^{{N}_{f}} {τ}_{F} dN = {\sum }_{1}^{{N}_{f}} [ {τ}_{N} + {τ}_{N - 1} ] \\ \text{?} \text{indicates text missing or illegible when filed} \qquad ( Equation \text{?}$$

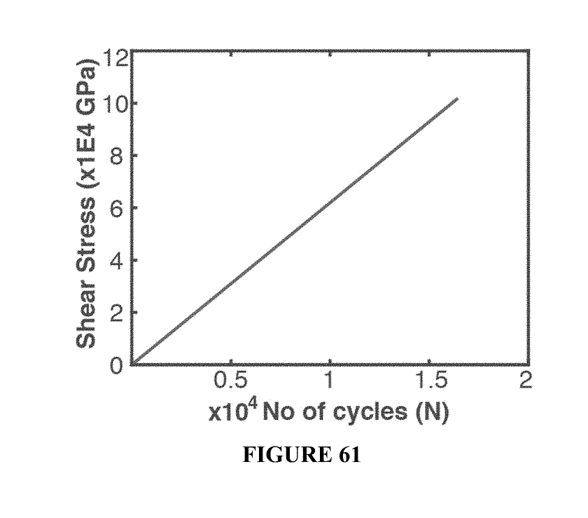

The estimated cycles to failure number from extracted data N_{f}=16444. Accumulated shear stress varies linearly with N (FIG. 61).

Accumulated loss from plastic strain energy, column 3,

$$Δ {A}_{N} \vert W = {\int }_{0}^{{N}_{f}} {\dot{W}}_{F} dN = {\sum }_{1}^{{N}_{f}} [ {W}_{N} + {W}_{N - 1} ] \\ \text{?} \text{indicates text missing or illegible when filed} \qquad ( Equation \text{?}$$

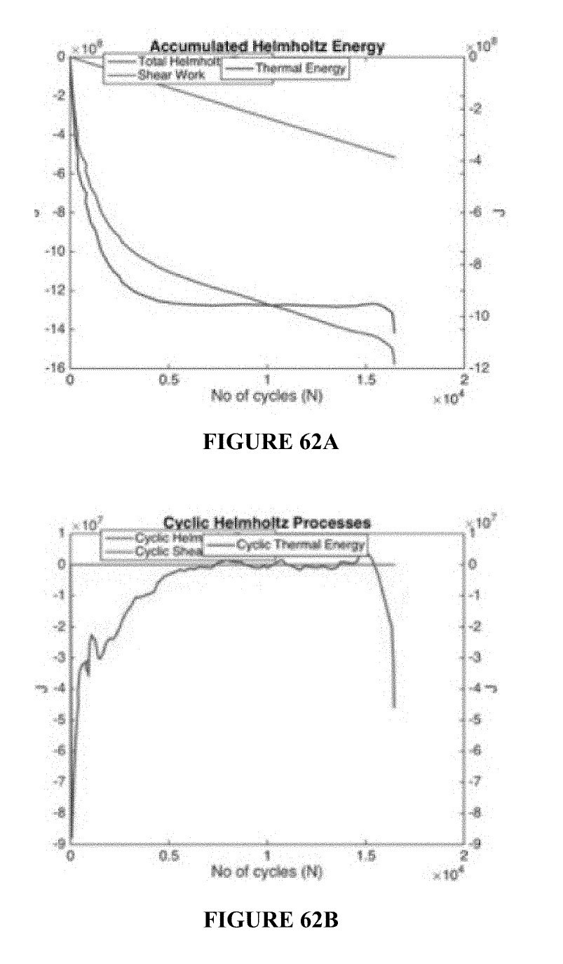

which represents the useful work (for mechanical applications), linearly decreases during loading (FIGS. 62A-62B). With constant load frequency and amplitude, the cyclic work amplitude is constant throughout, hence linear decrease in available plastic strain energy with cycle. Plastic strain energy loss before fatigue depends on sample material (Table 5.1) and load amplitude.

Thermal energy loss, column 4,

ΔA_{N}|T=∫₀^{N}^{_{f}}C\dot{T}dt=C(T_{f}−T₀) (Equation

is the change in the sample's available Helmholtz energy due to thermal energy changes during loading. With energy dissipation via heat dominating other thermal processes, including free convection to the environment (especially at high work rates), the thermal energy increases in magnitude, and thus has a negative effect (decreasing energy indicated by Helmholtz fundamental relation is depicted by plots on negative axis) on available total energy. Thermal energy changes depend directly on sample material (vis-à-vis the heat capacity) and the overall change in sample temperature during loading. A relationship between the gradient of the initial temperature rise and fatigue life has been previously demonstrated by Amiri and Khonsari.

Accumulated Helmholtz energy loss during operation, column 5:

ΔA_{N}=−ΔA_{N}|W−ΔA_{N}|T (Equation

A Taylor series with a forward difference to approximate the time derivative estimated thermal energy changes, where the first term

ΔA₁|T=∫₀^{N}^{₁}C\dot{T}dN=C(T₁−T₀) (Equation

and the nth term

ΔAₙ|T=∫ₜ_{₀}^{t}^{ₙ}C\dot{T}dt=ΔA₁|T+ . . . +ΔAₙ₋₁|T+C(Tₙ−Tₙ₋₁) (Equation

Total Helmholtz energy decreases during loading. Thermal energy loss is more significant than accumulated plastic energy loss during sample loading. The contribution from both plastic and thermal components are comparable.

Accumulated entropy production from plastic strain energy, column 6:

$${S}_{N}^{′} \vert W = {\int }_{0}^{{N}_{f}} \frac{{\dot{W}}_{F}}{T} dN = {\sum }_{1}^{{N}_{f}} \frac{[ {W}_{N} + {W}_{N - 1} ]}{{T}_{ave}} \\ where \\ {T}_{ave} = \frac{{T}_{N} + {T}_{N - 1}}{2} \\ \text{?} \text{indicates text missing or illegible when filed} \qquad ( Equation \text{?}$$

Accurate determination of thermal entropy should include effects of instantaneous change of temperature, when not isothermal. Many entropy formulations inappropriately assume an average constant temperature only observed in high cycle fatigue. At every instant, shear stress entropy and an accompanying thermal entropy are generated, both at the instantaneous temperature.

| Degradation | Value at | ΔA_{N}\|W | ΔA_{N}\|T | ΔA_{N} | S′_{N}\|W | S′_{N}\|T | S′_{N} | BW | BT | |
|---|---|---|---|---|---|---|---|---|---|---|
| Measure w | failure | MJ | MJ | MJ | MJ/K | MJ/K | MJ/K | w/J/K | w/J/K | wᵣₑₛ |
| Shear Stress τ (GPa) | 1.02E+05 | −514.8 | −1062 | −1576 | 0.996 | 2.532 | 3.528 | 0.106 | −0.00171 | 4451 |
| Damage D | 1 | | | | −6.17 | −3.44 | −3.74 | −0.1635 | 0.00173 | −0.00105 |
| N/Nf | 1 | −514.8 | −1062 | −1576 | 0.996 | 2.532 | 3.528 | 1.04E−06 | −1.67E−08 | 0.00437 |

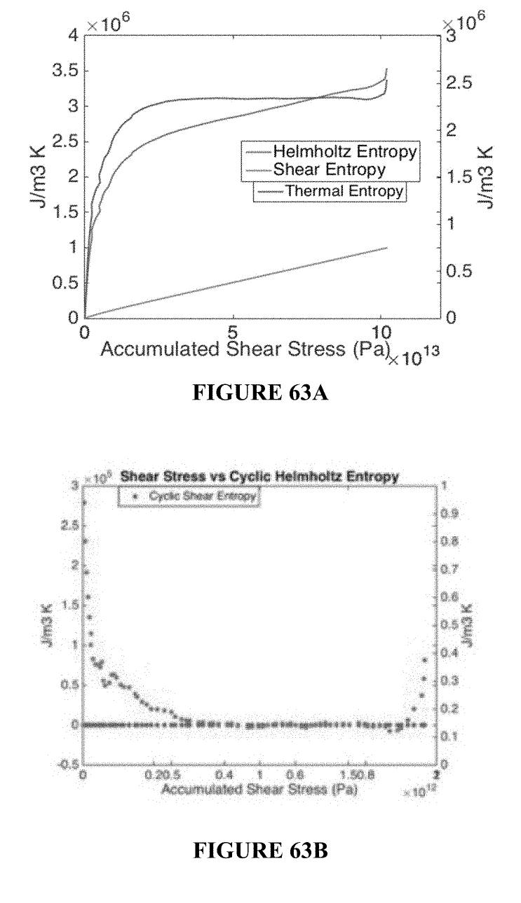

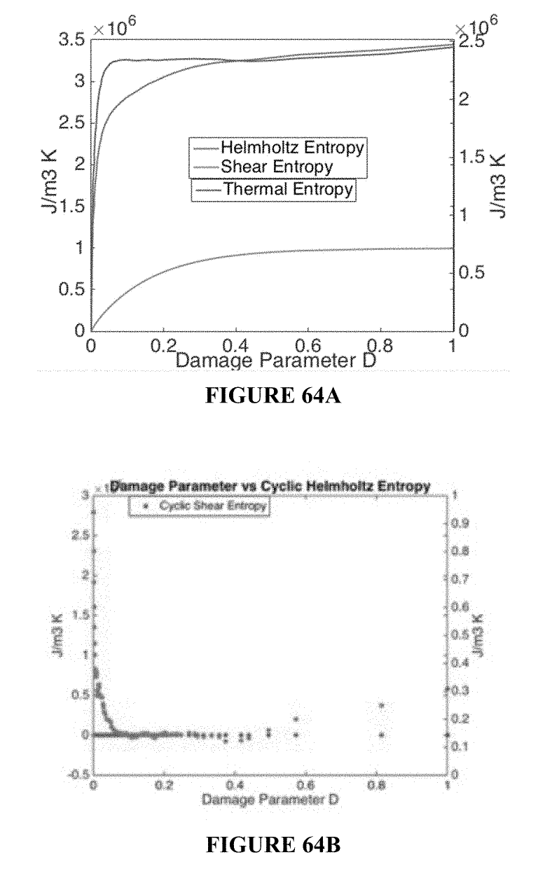

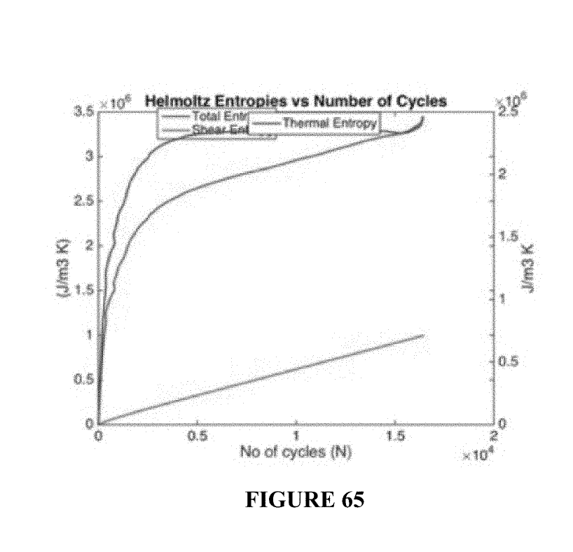

Table 5. and FIGS. 63A-63B, FIGS. 64A-64B, and FIG. 65 show the irreversible plastic strain entropy. A linear relationship is observed between the plastic strain entropy and accumulated stress/loading. As expected for constant loading, constant cyclic plastic strain entropy generation is observed in the sample loading.

Thermal entropy, column 7:

$${S}_{N}^{′} \vert T = {\int }_{0}^{{t}_{f}} \frac{C \dot{T}}{T} dt = {\sum }_{1}^{n} [ \frac{C ( {T}_{n} - {T}_{n - 1} )}{{T}_{ave}} ] \\ \text{?} \text{indicates text missing or illegible when filed} \qquad ( Equation \text{?}$$

where integrals were treated similar to equations (Equation 5.360) and (Equation 5.362). Thermal entropy change progresses similar to temperature change, see FIGS. 63A-63B, FIGS. 64A-64B, and FIG. 65. Thermal entropy progresses at the same rate as sample temperature change rate. With the initially high temperature change rate, the thermal entropy change rate is high and eventually stabilizes (FIGS. 63A-63B, FIGS. 64A-64B, and FIG. 65).

Total Helmholtz entropy generation from equation (Equation 5.306) (no compositional change), column 8,

S′_{N}=S′_{N}|W+S′_{N}|T

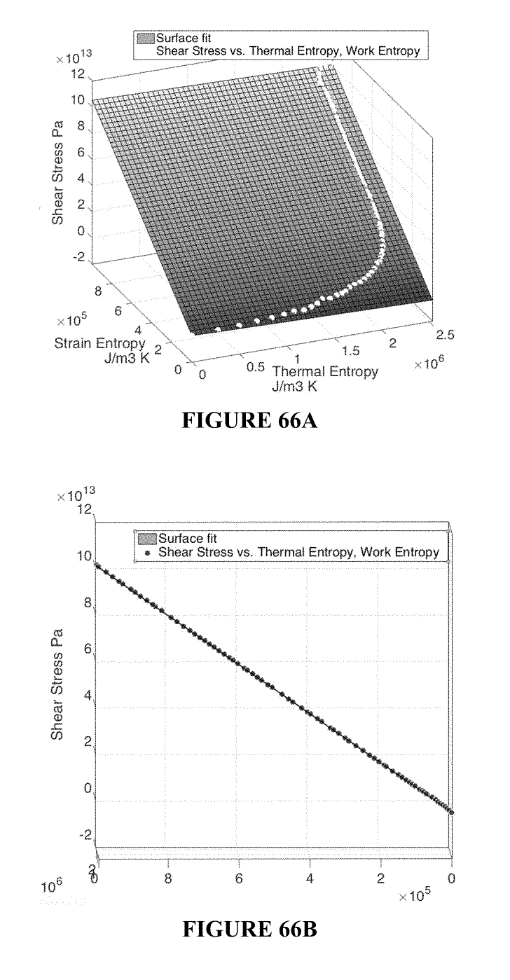

shown, with components, in FIGS. 63A-63B, FIGS. 64A-64B, and FIG. 65. As with energy, the thermal contribution, as a result of the initially transient dissipation, is higher than the plastic strain contribution and determines the profile of the total entropy. The partial contributions better visualize in the 3D surface plot in FIGS. 66A-66B.

Degradation Coefficients Bᵢ

Plastic strain energy degradation coefficients from equation (Equation 5.321), (Equation 5.329) and (Equation 5.337) respectively, column 9:

$${B}_{{W}_{τ}} = \frac{\partial τ}{\partial {S}_{W}^{′}} ; {B}_{{W}_{N}} = \frac{\partial N}{\partial {S}_{W}^{′}} ; {B}_{{D}_{W}} = \frac{\partial D}{\partial {S}_{W}^{′}}$$

The DEG theorem suggests a constant B_{W} during loading. A low B_{W} implies low impact of plastic strain entropy on degradation accumulation. From Table 5.2, shear stress coefficient B_{W}_{_{τ}}=0.106, B_{D}_{_{W}}=−0.164 and B_{W}_{_{N}}=1.04 E-6. For positive entropy components positive values of B indicate a degradation measure and a negative value indicates a transformation measure, a reverse verification of the second law. Hence according to entropy values in Table 5.2, B_{W}_{_{τ}} predicts degradation as plastic stress and entropy accumulate in the component, B_{D}_{_{W}} predicts degradation as damage increases with the logarithm of the remaining life and B_{W}_{_{N}} predicts degradation as cycle number increases with entropy accumulation.

Thermal degradation coefficients from equation (Equation 5.321), (Equation 5.329) and (Equation 5.337) respectively, column 10:

$${B}_{{T}_{τ}} = \frac{\partial τ}{\partial {S}_{T}^{′}} ; {B}_{{T}_{N}} = \frac{\partial N / {N}_{f}}{\partial {S}_{T}^{′}} ; {B}_{{D}_{T}} = \frac{\partial D}{\partial {S}_{T}^{′}}$$

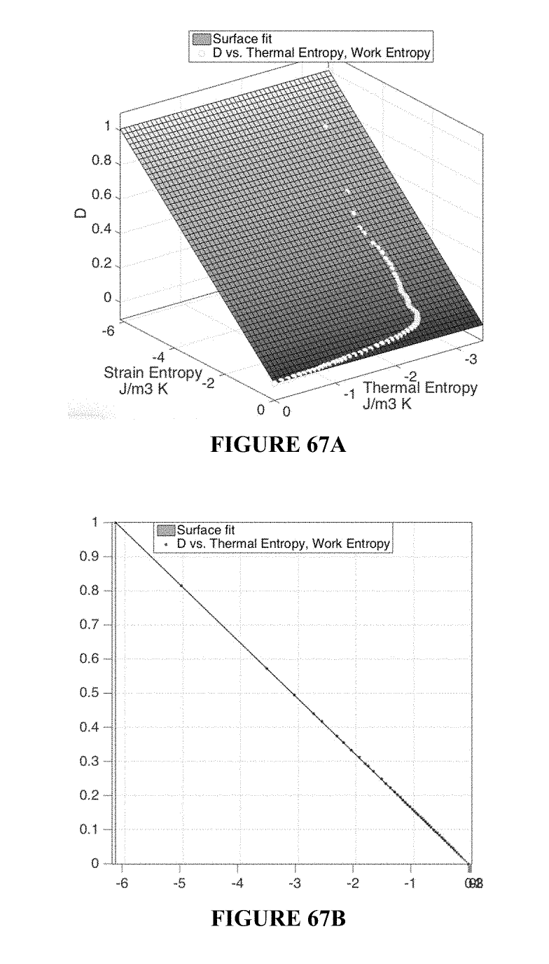

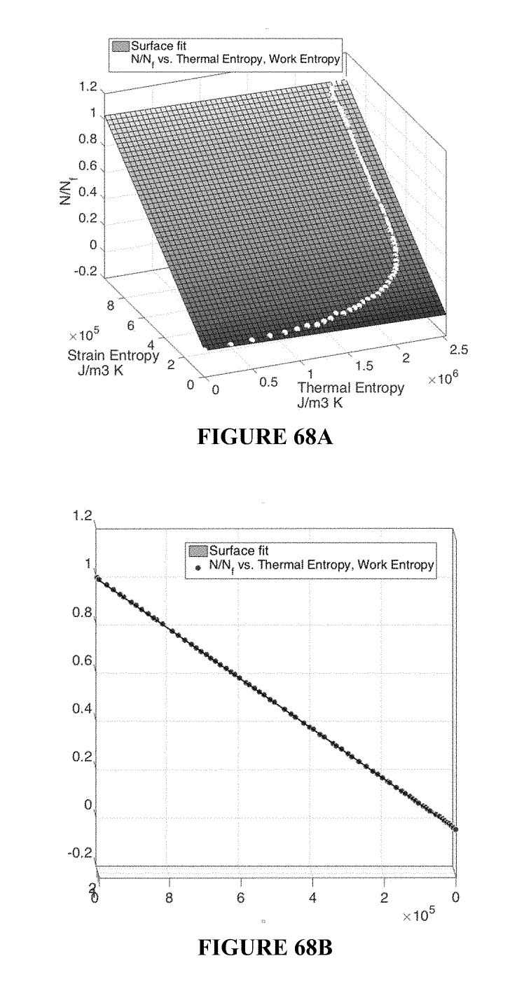

Table 5.2 shows B_{T} consistently 2 orders of magnitude less than B_{W} for all the fatigue measures considered. This implies the thermal entropy contribution was relatively insignificant and verifies the high accuracy recorded in metal fatigue experiments that neglected thermal entropy. Using a curve fitting tool, accumulation vectors (a series of sum of adjacent values) obtained from equations (Equation 5.364) and (Equation 5.365) were simultaneously fitted to accumulated shear stress from equation (Equation 5.358), damage parameter D from equation (Equation 5.332) and normalized number of cycles N/N_{f} to obtain the DEG relations formulated in equations (Equation 5.320), (Equation 5.328) and (Equation 5.337). A combined linear dependence of the degradation measures on both entropy components is observed. Shear stress t (FIGS. 66A-66B), damage parameter D (FIGS. 67A-67B), and N/N_{f} (FIGS. 68A-68B) plot the measure versus entropy data in three-dimensional spaces, to separate out individual entropies. These show an almost perfect coincidence of all plastic strain entropy data points with a linear 2D surface fit (goodness of fit R²=1). Shear stress and normalized cycles hence show similar trajectory, representative of their similar time-based accumulation. Residual stress from each fit, column 11, is the difference between the measured fatigue parameter w and that computed via the DEG theorem,

$${w}_{res} = w - {\sum }_{i} {B}_{i} {S}_{i} \\ \text{?} \text{indicates text missing or illegible when filed} \qquad ( Equation \text{?}$$

Degradation coefficients B_{W} and B_{T}, partial derivatives of the degradation measures to plastic strain and thermal entropies respectively via the DEG theorem (see equations (5.88) and (5.89)), were estimated as coefficients from the surface fit. FIGS. 66A-66B, 67A-67B, and 68A-68B show the loaded sample draws a path—its Degradation Entropy Generation DEG trajectory—marked by the measured points characteristically coincident with a linear plane—its DEG surface. The DEG surface suggests a linear dependence of degradation accumulation on both plastic strain and thermal entropies. The 3-D space, the sample's DEG domain enclosing the DEG surface characterizes the complete regime in which the sample can be loaded. The DEG domain, spanned by a degradation measure, plastic strain entropy and thermal entropy can define consistent parameters for identifying desired characteristics of sample. These plots are direct visual verifications of the DEG theorem. The views show all measured points on the surface, hence R²=1 goodness of fit, rare for most experiments under uncontrolled conditions, especially dissipation measurements.

Fatigue Analysis of Experimental Data Using DEG

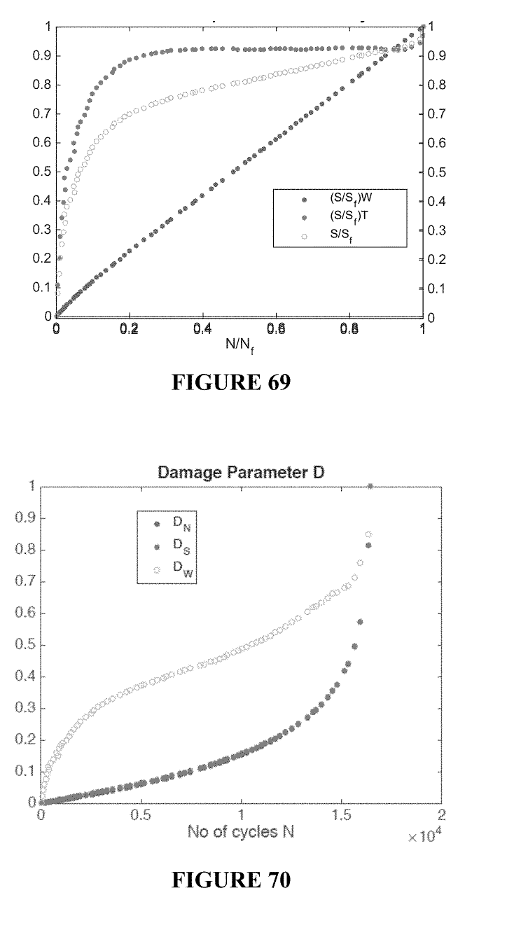

FIG. 69 shows normalized Helmholtz (total) entropy and components vs normalized number of cycles. A linear relationship exists between normalized plastic strain entropy and number of cycles, verifying equation (Equation 5.279). Actual total entropy as prescribed by the Helmholtz formulation equation (Equation 5.303) when not isothermal includes a significant thermal component.

In FIG. 70, D varies logarithmically with N. FIG. 70 shows D calculated from plastic strain energy S_{W} (D_{S}), total entropy S (D_{W}) and N (D_{N}), further verifying the above linearity between normalized plastic strain entropy S′ and N′ but not with total entropy.

Heat-Only Analysis—Mechanical Loading

Thermal analysis-based degradation coefficients will be evaluated using data from the torsional fatigue experiment described above. Heat transfer is free convection spontaneously driven by the difference between sample and ambient temperatures. As before, the trapezoidal rule estimated integrals of accumulated heat transfer and heat transfer entropy. Tables and figures follow the same convention as the Helmholtz analysis. Signs indicate direction of the energy or entropy process. Plots show actual process directions.

TABLE 5.3
Heat-only based experimental analysis results, w = degradation measure. Damage D-based entropies are logarithmic.

| Degradation | Value at | Q_{N} | ΔE_{N} | E′_{N} | S′_{N}\|Q | ΔS′_{N}\|T | S′_{N} | B_{Q} | B_{T} | |
|---|---|---|---|---|---|---|---|---|---|---|
| Measure w | failure | MJ | MJ | MJ | MJ/K | MJ/K | MJ/K | w/J/K | w/J/K | wᵣₑₛ |
| Shear Stress τ (GPa) | 1.02E+05 | −26460 | 1062 | 27522 | −50.41 | 2.532 | 52.94 | −0.00194 | 0.00207 | −543 |
| Damage D | 1 | | | | −6.01 | −3.44 | −5.55 | −0.1646 | −0.0022 | 0.00139 |
| N/N_{f} | 1 | −26460 | 1062 | 27522 | −50.41 | 2.532 | 52.94 | −1.9E−08 | 2.03E−08 | −0.00533 |

In this analysis, temperature is the only changing parameter.

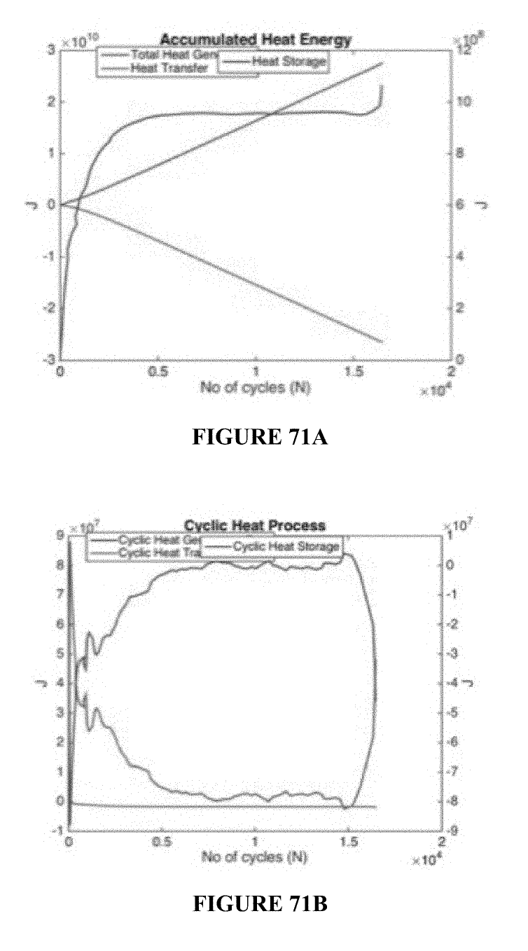

Heat transfer was predominantly out of the sample. Initially high rates eventually stabilize to a steady rate as sample achieved thermal equilibrium after 5000 cycles. The heat storage component is the same as the thermal component in the Helmholtz formulations. FIGS. 71A-71B show that most of the heat generated is transferred out of the sample. This implies that the measurement sensitivities discussed earlier, which transfer to the heat generation values, are more significant in this approach. Note that heat transfer into the surroundings and heat generation within the sample proceed in opposite directions.

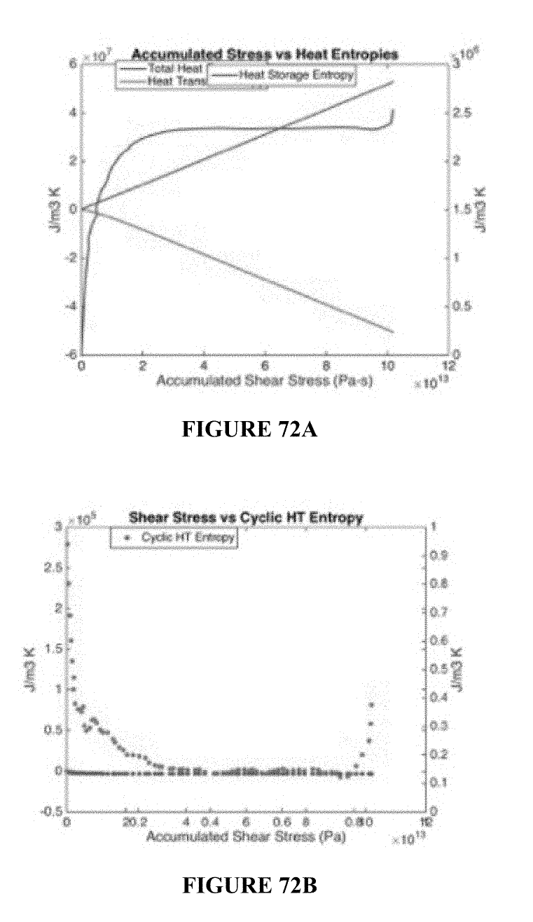

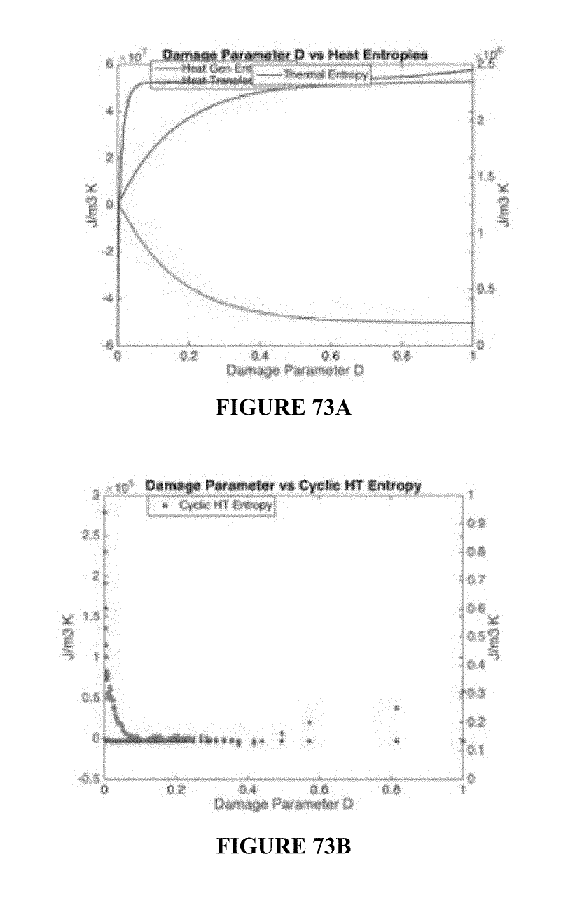

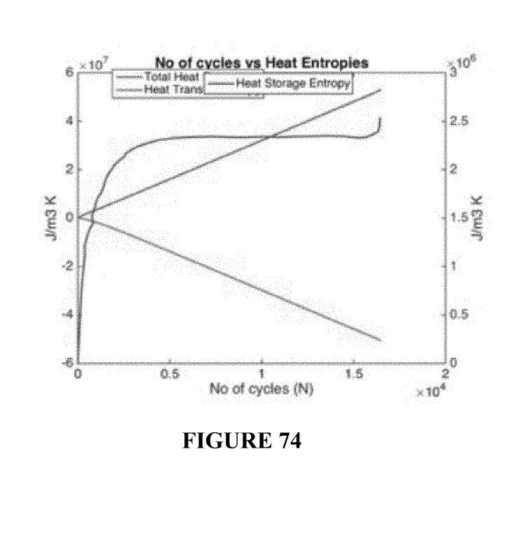

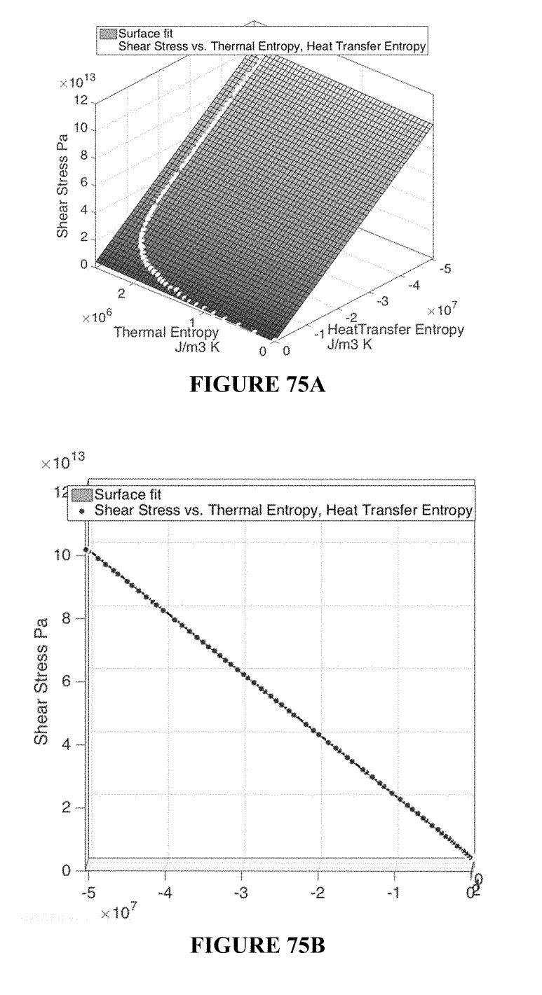

FIGS. 72A-72B show significance of entropy transfer by heat. A slightly linear relationship with accumulated stress is observed. With D, a logarithmic relationship is observed with the heat transfer entropy as was the case for plastic strain entropy, indicating the appearance of a relationship between the heat transfer process and the plastic strain process. Heat entropies vary with N (FIG. 74) similar to shear stress in FIGS. 72A-72B. The heat storage contribution to total entropy generation during loading is same as the Helmholtz analysis. However, its partial contribution to the overall degradation measure is more significant in the heat-only analysis, as indicated by coefficient magnitudes (FIG. 74, right axis)). Heat generation entropy proceeds opposite heat transfer entropy, as prescribed by equation (Equation 5.307). With both active heat processes significant, the linear partial variation of degradation measures is shown in FIGS. 75A-75B.

Degradation Coefficients and the Degradation Surface

The surface models each had about R²=1 with coefficient predictions at 95% confidence interval. Similar fit as in the Helmholtz DEG elements for the three degradation measurements is anticipated here given the same dataset was used for all three. Hence for brevity, only the shear stress plots are shown in FIGS. 75A-75B.

From Table 5.3, the shear stress coefficients B_{Q}_{_{τ}}=−0.00194, B_{D}_{_{Q}}=−0.164 and B_{Q}_{_{N}}=−1.9 E-08. Again, for positive entropy components positive values of B indicate a degradation measure and a negative value indicates a transformation measure. According to the entropy balance, heat transfer out of the sample reduces its total entropy change, hence is a transformation (positive enhancement) measure as indicated by B_{Q}_{_{τ}}, B_{Q}_{_{N}} and B_{D}_{_{Q}} in Table 5.3.

Table 5.3 shows B_{T} is the same order of magnitude as B_{W} for the fatigue measures considered except damage parameter D. According to the DEG theorem, this implies that during active mechanical loading of the sample by torsion, both heat storage and heat transfer entropy entropies contribute similarly to degradation. This indicates likelihood of error in heat-only analysis of fatigue experiments that have neglected the thermal entropy.

Discussion

Thermal Entropy

The effect of the initial temperature rise is observed in the thermal entropy dimension of the DEG domain. If steady state was not reached, this component would have been the dominant mechanism as indicated by the significantly higher initial increase rate. In bending fatigue, temperature rise is less.

True linearity between degradation measures (including normalized number of cycles) and normalized entropy generation is observed through entropy generation components. The observed linearity between plastic strain entropy and number of cycles is explained by B_{T}˜0.01 B_{W}; an error of +/−0.01 was also reported previously. The limited impact of the thermal entropy in the mechanical loading of steel can be verified using the Coffin-Manson relationship modified for thermal cycling. Steel has a melting point over 1500 degC and is widely used in applications with continuous operating temperature ˜1000 degC, so a temperature rise of 250 degrees as in the torsional fatigue results analyzed above, from experience, will not significantly impact its microstructure.

Damage Parameter D and the DEG Coefficients

Tables 5.2 and 5.3 show that for D, B_{D}_{_{Q}}≈B_{D}_{_{W}}. The similarity is also observed in the heat transfer entropy and plastic strain entropy vs D profiles. This verifies the above lack of dependence of the torsional fatigue of SS 304 as measured by the damage parameter D on thermal entropy. Comparing equations (Equation 5.337) and (Equation 5.340) shows

B_{D}_{_{W}}S′_{D}_{_{W}}=B_{D}_{_{Q}}S′_{D}_{_{Q}} (Equation

and hence

S′_{D}_{_{W}}≈S′_{D}_{_{Q}} (Equation

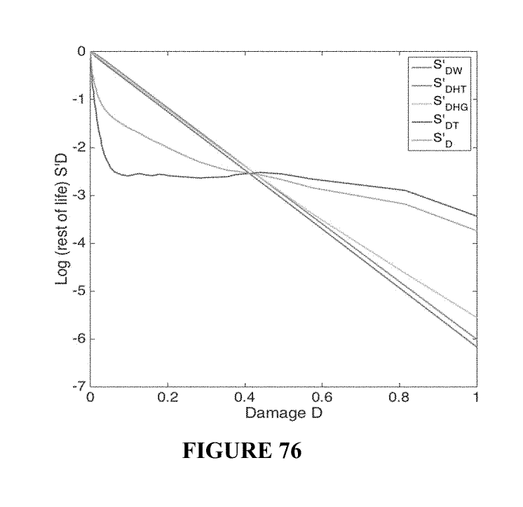

This is shown in FIG. 76. It is observed that thermal and total entropies do not correlate linearly with D. Logarithmic rest of life S′_{D}_{ᵢ} from normalized entropy generation terms is given in equations (Equation 5.334)-(Equation 5.341).

FIG. 76 also shows that the logarithmic heat generation (viscous dissipation) entropy S′_{D}_{_{HG}} correlates with D. By definition, this can be explained as the viscous dissipation is a component of the plastic work. Hence the apparent correlation between B_{D}_{_{Q}} and B_{D}_{_{W}} appears to be emanate from the expected correlation between B_{D}_{_{HG}} and B_{D}_{_{W}}.

It is noted that by normalizing entropy generation components, the directionality of entropy transfer by heat and entropy change by thermal energy change is lost in the analysis. Hence, D only measures plastic strain entropy effect on the component and its heat-only coefficients B_{D}_{_{Q}} and B_{D}_{_{T}} might not adequately analyze the effects of the surroundings in addition to insensitivity to system temperature change. This deficiency is inherited by other normalized entropy measures.

The difference in the observed behavior of D and other degradation parameters gives an insight into proper interpretation of the DEG coefficients. Coefficients derived from logarithmic degradation measures differ in meaning from those derived from linear degradation accumulation measures. This is anticipated with the time base of entropy accumulation.

### Summary and Conclusion

In this Example, the Degradation-Entropy Generation Theorem was applied to fatigue analysis. The results show a direct agreement between existing fatigue parameters and the predicted linearity by the DEG theorem. The importance of degradation parameter used in analysis was also demonstrated.

### Example 6. Further Discussion

Having applied the DEG approach to three non-linear systems with significant differences in composition and boundary interaction types (grease, battery and fatigue), this Example reviews findings from the analytical and experimental results.

Instantaneous energy changes in real systems involve contributions from thermal energy changes CdT, boundary work interactions δW and compositional changes ΣμdN occurring at different rates. The DEG theorem successfully constructed failure models in Examples 3-5 of the general form

$$\begin{matrix} Maximum Work \text{:} \\ w = {B}_{T} {\int }_{{t}_{0}}^{{t}_{f}} \frac{C \dot{T}}{T} dt + {B}_{W} {\int }_{{t}_{0}}^{{t}_{f}} \frac{\dot{W}}{T} dt + {B}_{\dot{N}} {\int }_{{t}_{0}}^{{t}_{f}} \frac{μ \dot{N}}{T} dt & ( Equation 6.369 ) \\ Heat \text{:} w = {B}_{T} \int \frac{C \dot{T}}{T} dt - {B}_{HT} \int [ \frac{( T - {T}_{∞} )}{RT} ] dt & ( Equation 6.370 )\end{matrix}$$

where w is generalized degradation, defined by performance/failure parameters for
Grease shearing: thermal, mechanical, chemical
Battery cycling: thermal, electrochemical (coupled electrical and chemical)
General fatigue: thermal, mechanical, chemical

Highlights

Consistent results from all three studies verify the following generalized characteristics.
DEG theorem provides structured approach to degradation modeling.
   Discover underlying dissipative processes pᵢ, entropy generations Sᵢ′, degradation measure w, and apply DEG to get degradation coefficients Bᵢ
      from direct measurements;
      from prior models, as given in grease degradation coefficients table (Table 3.2) and the various fatigue measure coefficients in Example 5,
   instead of heuristic empirical methods of measure everything, plot everything versus everything, find correlations, then do numerous curve fits.
DEG theorem supports disorganization implied by entropy and second law, becoming a reverse confirmation of second law.
Entropy generation components serve as basis functions for a multi-dimensional degradation function space of w versus Sᵢ′, as implied by DEG.
   Trajectory in DEG space is almost always on planar DEG surface.
   Degradation coefficients Bᵢ act as basis vectors in space and always lie in DEG plane.
   Degradation coefficients Bᵢ are functions of generalized variable ζᵢ rates for a conjugate pair representation Xᵢdζᵢ/dt of the dissipative process (for constant Xᵢ), e.g. shear stress \dot{γ} in a constant-shear rate process (Example 3), discharge rate I in constant-voltage V battery cycling (Example 4); stress rate (amplitude/cycle) in fatigue loading (Example 5), e.g. plastic strain energy in variable stress rate loading.
   Plane surface inclination Bᵢ (direction) depends on rates of entropy production (dSᵢ/dt). Net heat transfer out of the system in heat-only analyses gives a negative Bᵢ.
   The DEG theorem requires a linear degradation accumulation degradation measure for predicted linearity of entropy generation components.
   Degradation coefficients Bᵢ are sensitive to data used in obtaining them.
   Degradation coefficients Bᵢ obtained from an existing degradation parameter give an indication of the component of actual degradation measured by that parameter.
The DEG theorem converts degradation failure design into a multi-dimensional geometry problem. The volume spanned by normal trajectories define normal operating region and normal ageing region.

A few of the above features are explored below.

Degradation, a Dimensional Geometry Problem

In this work, by using the maximum work formulations and the heat energy balance, the linearity predicted by the DEG theorem was observed. This linearity was neither observed with respect to any one entropy production component nor with a sum of both components as evident in the heat-only plots for grease. However, in the DEG space linearity was observed. The entropy generation components define the dimensions of the base plane and the projected normal height is dimensioned by the degradation accumulation vector.

With one dominant process, as seen in many applications, an apparent linearity with the dominant process is seen. However, as shown above, actual contributions to damage will not be known if only one process is used. As indicated in the lead-acid battery discussion, this is more crucial in certain processes than others.

The Thermodynamic Simple System Vs the Single-Variable System

To understand two or more process interactions by the DEG theorem, recall The Thermodynamic State Postulate: the state of a simple system is completely specified by r+1 independent, intensive properties where r is the number of significant work interactions. Hence no real system fully defined by one work interaction exists in nature. This corollary of the second law is evident in the DEG formulations. A single-variable system will not represent all instances of the process, especially for naturally occurring processes such as entropy generation which measures irreversibility. In previous applications of the DEG theorem, representation of systems undergoing one dominant process as a single-variable system may have arisen from the use of internal energy, discussed in Example 2. This formulation is common in mechanics with systems dominated by one work interaction, hence system degradation—e.g. fatigue strength for dynamically loaded systems, capacity for energy storage systems, etc. The local equilibrium assumption by Prigogine which gave the equality version of the entropy balance (equation (Equation 1.17)) also prescribes the above.

As with other features of entropy generation inherited by the DEG theorem, the State Postulate imposes a condition on the use of the DEG theorem: the entropy generation of a simple system is completely specified by r+1 independent, intensive properties where r is the number of significant work interactions. This would also be called the Entropy Generation Postulate.

Hence, the DEG domain is an artefact of the Entropy Generation Postulate.

DEG Coefficients and Maxwell Coefficients

In Thermodynamics, partial fraction geometry, as with the DEG theorem is applied to the natural variables of the fundamental relations of thermodynamic energies. From the Helmholtz equation,

dA=−SdT−Xdζ+μdN′ (Equation

can be rewritten as

$$dA = - {( \frac{\partial A}{\partial T} )}_{V , {N}^{′}} dT - {( \frac{\partial A}{\partial V} )}_{T , {N}^{′}} dV + {( \frac{\partial A}{\partial {N}^{′}} )}_{T , V} {dN}^{′} \\ \text{?} \text{indicates text missing or illegible when filed} \qquad ( Equation \text{?}$$

Using second derivative symmetry, Maxwell's relations give

$${( \frac{\partial S}{\partial V} )}_{T , {N}^{′}} = {( \frac{\partial P}{\partial T} )}_{V , {N}^{′}} ; {( \frac{\partial μ}{\partial T} )}_{V , {N}^{′}} = - {( \frac{\partial S}{\partial {N}^{′}} )}_{T , V} ; \\ {( \frac{\partial μ}{\partial V} )}_{T , {N}^{′}} = - {( \frac{\partial P}{\partial {N}^{′}} )}_{T , V} \\ \text{?} \text{indicates text missing or illegible when filed} \qquad ( Equation \text{?}$$

In this study, the degradation theorem was expressed using thermal, work and chemical terms and their respective degradation coefficients as

dw=B_{T}dS′_{T}+B_{W}S′_{W}+B_{N}S′_{N} (Equation

With the above entropy generation postulate, the DEG relations

$${( \frac{\partial {B}_{T}}{\partial {S}_{W}^{′}} )}_{{S}_{T}^{′} , {S}_{N}^{′}} = {( \frac{\partial {B}_{W}}{\partial {S}_{T}^{′}} )}_{{S}_{W}^{′} , {S}_{N}^{′}} ; \\ {( \frac{\partial {B}_{T}}{\partial {S}_{N}^{′}} )}_{{S}_{T}^{′} , {S}_{W}^{′}} = {( \frac{\partial {B}_{N}}{\partial {S}_{T}^{′}} )}_{{S}_{N}^{′} , {S}_{W}^{′}} ; \\ {( \frac{\partial {B}_{W}}{\partial {S}_{N}^{′}} )}_{{S}_{T}^{′} , {S}_{W}^{′}} = {( \frac{\partial {B}_{N}}{\partial {S}_{W}^{′}} )}_{{S}_{T}^{′} , {S}_{N}^{′}} \\ \text{?} \text{indicates text missing or illegible when filed} \qquad ( Equation \text{?}$$

were obtained.

DEG Line=f(Thermal Entropy Line, Work Entropy Line)

Another feature arising from the imposed r+1 entropy generation constraint is the DEG trajectory. In Thermodynamics a “heat” line in addition to the ideal “work” line led to the origin of the State Postulate. While the original formulation of the thermodynamic first law was for heat engines (energy transfers by work and heat, hence an internal energy formulation), it can be applied to other forms via the thermodynamic potentials. In the entropy plane (the 2D horizontal plane), a coordinate is defined by the “work entropy” and “thermal entropy” lines.

Ageing Tests

With the Helmholtz potential as the maximum work obtainable from a system or process, if the boundary work is the required work interaction, equation (Equation 6.371) becomes

dAₘₐₓ=Xdζ (Equation

The irreversibilities associated with equation (Equation 6.376) are

$$δ {S}^{′} = \frac{Xd ζ}{T} \\ \text{?} \text{indicates text missing or illegible when filed} \qquad ( Equation \text{?}$$

It has been established that the above equation does not fully define a real process, but represents the reversibility limit imposed by the second law, as maximum work is not achievable in reality. Comparing to equation (Equation 1.34) indicates the significance of the thermal and/or compositional entropy terms in real systems. For homogenous systems, reversible change in thermal entropy

$${dS}_{T} = \frac{SdT}{T} \\ \text{?} \text{indicates text missing or illegible when filed} \qquad ( Equation \text{?}$$

which can be significantly minimized to lower total entropy accumulation, or as seen with the starter lead-acid battery thermal recovery phenomenon discussed in chapter 4, reversed via an endothermic process; note that the second law requires total entropy (system+surroundings)≥0 so equation (Equation 6.378) can be negative. This can be employed in ageing tests.

With the above consistent characteristic of the DEG elements, the DEG theorem prescribes a dimensional solution to degradation analysis. Optimum operating points can be determined in a DEG space by adjusting appropriate dimensions of the entropy generation plane.

Heat-Only Analysis Versus Maximum Work Analysis

In Example 1, formulations for heat-only analysis were presented in addition to the thermodynamic maximum work formulations. The heat-only formulations also showed conformity with the dimensionality of the DEG theorem. As stated earlier, it is important to note that if the dominant boundary work process is well defined, as is the case with most engineering systems, the maximum work approach will give the more accurate representation of the system. In addition to measuring the useful work out of the system, the primary aim of most degradation analysis, the maximum work components typically have higher orders of magnitude than the heat components (e.g. natural convection), hence less prone to measurement error.

The benefits of the heat-only analysis in determining entropy generation components with only temperature measurements, including thermally dominated processes, have been discussed in earlier chapters. The DEG heat transfer coefficients B_{Q} sensitivity to heat transfer entropy rate also indicate its utility in determining the effect of the surroundings on the system's degradation. This would imply that when available, a full system-process analysis would involve:
Maximum work analysis to obtain the actual degradation from the boundary work using
   Helmholtz Potential A
   Gibbs Potential G
   Enthalpy H
Heat-Only analysis to obtain the system's interaction with the surroundings.

Both approaches, while not required at the same time for many systems, take advantage of the natural interactions in system-process-surroundings relationships. Information from these analyses can be used in material selection and process optimization, in addition to system design.

Degradation Measures

Results from Examples 3, 4 and 5 show that DEG coefficients can relate actual degradation from entropy accumulation to existing degradation measures. However, it was also shown that by this relationship, the values of the coefficients are subject to the same shortcomings as the degradation measure used. For example, degradation parameter formulated with isothermal assumption, is likely to show minimal contribution from thermal entropy, unlike degradation determined from actual degradation measurements that include all failure mechanisms.

Also, as shown by damage parameter D, a logarithmic degradation measure does not have a direct linear relationship with entropy generation components, imposing the requirement of a time-based degradation measure, as anticipated by the mathematical basis of the DEG theorem (also by the time basis of entropy generation accumulation as prescribed by the second law).

Hence in addition to proper formulation of active processes taking place in evaluating entropy generation components, an understanding of the degradation measure used is necessary for parameter selection as well as interpretation of results.

Residuals

Each application of the DEG theorem in Examples 3-5 showed the existence of a residual term that appears more significant in the heat analysis approach. This residual term is understood to be a term that transfers the system onto an irreversible path. Indeed, the residual, as described in relation to Example 7 improves significantly the battery formulations as described in Example 4 and demonstrated the universality of the theorem when applied to real systems.

### Concluding Remarks

Notwithstanding the mathematical and dissipative mechanics-based definitions of the parameters in the DEG theorem, validity of the theorem is inherent in the combined statements of the first and second laws of Thermodynamics. As shown above, the DEG theorem confirms and verifies long established Thermodynamic principles. By inheriting features of its deductive formulations, the DEG theorem is instantaneously valid and hence, the B coefficients, its intensive variables, are instantaneous. The DEG theorem gives a linear path between irreversibilities accumulated and the resulting damage in systems using dimensional entropy generation components. This has been successfully applied to vastly different and severely non-linear systems, with similar results.

### Example 7. A Thermodynamic Model for Lead-Acid Battery Degradation—Application of the DEG Theorem

Lead-acid battery issues include low specific energy, self-discharge and ageing. Models to predict performance over time have limitations. The battery industry lacks a consistent and effective approach to monitor and predict performance and ageing across all battery types and configurations. This Example further develops a new universally consistent approach for characterizing lead-acid batteries of all configurations. An instantaneous model for analyzing battery degradation based on irreversible thermodynamics and the Degradation-Entropy Generation theorem is formulated and experimentally verified using commonly measured lead-acid battery operational parameters.

Background

Capacity [symbol] (in coulombs or ampere hours) is the amount of charge a battery can hold. Charging and discharging lead acid batteries involve chemical reactions. At the negative electrode

PbO₂+3H⁺+HSO₄⁻+2e⁻⇄PbSO₄+2H₂O (Equation

with a potential of +1.69V. At the positive electrode

Pb+HSO₄⁻⇄PbSO₄+H⁺+2e⁻ (Equation 7.380)

with a potential of −0.358V. This gives an overall reversible reaction

PbO₂+Pb+2H₂SO_{4⇄}2PbSO₄+2H₂O (Equation

with an overall cell voltage of +2.048V.

Both chemical and electrical models of the battery can be coupled via the Gibbs relation

$$- \frac{dG}{dt} = \sum {μ}_{i} {\dot{N}}_{i} = A \dot{ξ} = VI \\ \text{?} \text{indicates text missing or illegible when filed} \qquad ( Equation \text{?}$$

where A is de Donder's reaction affinity and is reaction extent. The entropy from power or energy dissipated by Ohmic or chemical reaction work was represented as

$${\dot{S}}^{′} = \frac{A \dot{ξ}}{T} = \frac{VI}{T} . \qquad ( Equation 7.383 )$$

This approximate model describes an isothermal operation.

Feinberg with an extended system boundary that included battery and sources summed entropy change over an entire discharge/charge cycle to get

$${dS}_{tot} = ( \frac{dU}{T} - \frac{Vdq}{T} ) + ( \frac{{dU}_{en}}{{T}_{en}} - \frac{{Edq}_{en}}{{T}_{en}} ) . \qquad ( Equation 7.384 )$$

Equation (Equation 4.189), which considers total entropy change of the extended system using internal energy change, is inconvenient for battery only analysis.

Esperilla et al's bond graph models of lead-acid battery dynamics during cycling include primary and secondary electrochemical reactions at both electrodes, and thermal energy dissipation. The CIEMAT model for lead-acid batteries gives voltage as a function of capacity, state of charge (SOC) and temperature, and has been verified experimentally with reasonable accuracy. Others modeled the lead-acid battery considering charge conservation and transport, using the effective diffusion coefficient and the Butler-Volmer equation for charge transfer.

The afore-mentioned models often fail under unsteady operation, over-discharging and other nonlinear system interactions; often cannot accurately predict useful life; cannot adequately account for battery ageing and/or parasitic losses; and cannot be easily adapted to other battery types without significant corrections.

In line with Rayleigh's dissipation function of mechanics, Onsager's classical dissipative thermodynamics and Prigogine's extensive work in non-equilibrium thermodynamics, the DEG theorem established a direct relationship between degradation rate of elements or systems and rates of entropy generation. Here, the loss of lead acid battery capacity is related to the irreversible entropy produced during charge and discharge cycles, by chemical, electrical and thermal dissipative processes that occur in the battery. The first and second laws will be combined with Gibbs potential to formulate the entropy productions. The DEG theorem will then relate the permanent and transient loss of battery capacity to these entropies produced. The model shows excellent agreement between theory and measurements.

Degradation-Entropy Generation Theorem

The degradation entropy generation theorem relates material/system degradation w to the irreversible entropy S′ᵢ produced by the underlying dissipative physical processes pᵢ that drive the degradation.

Statement:

Given an irreversible material transformation caused by i=1, 2, . . . , n underlying dissipative processes and characterized by an energy, work, or heat pᵢ. Assume effects of the mechanism can be described by a parameter or state variable that measures the effects of the degradation transformation, i.e.

w=w(pᵢ)=w(p₁,p₂, . . . , pₙ), i=1,2, . . . , n (Equation

and is monotonic in each pᵢ. Then the rate of degradation

$$\dot{w} = {\sum }_{i} {B}_{i} {\dot{S}}_{i}^{′} \\ \text{?} \text{indicates text missing or illegible when filed} \qquad ( Equation \text{?}$$

is a linear combination of the rates of irreversible entropies \dot{S}′ᵢ generated by the dissipative processes pᵢ, where the degradation transformation process coefficients

$${ {B}_{i} = \frac{\partial w}{\partial {S}_{i}^{′}} \vert}_{{p}_{i}} \\ \text{?} \text{indicates text missing or illegible when filed} \qquad ( Equation \text{?}$$

are slopes of degradation w with respect to irreversible entropy generation S′ᵢ; the |ₚᵢ notation refers to the process pᵢ being active.

Integrating equation (Equation 2.53) over time, composed of cycles wherein Bᵢ is constant, yields the total accumulated degradation

Δw=ΣᵢBᵢS′ᵢ, (Equation 7.388)

which is also a linear combination of the entropy accumulation components, S′ᵢ generated by the dissipative processes pᵢ.

Generalized Degradation Analysis Procedure

The structured approach to degradation analysis using the DEG theorem embeds the physics of the dissipative processes into the energies pᵢ=pᵢ(ζᵢⱼ), j=1, 2, . . . , m; derives entropy generation \dot{S}ᵢ′=dS′ᵢ/dt as a function of pᵢ and expresses the rate of degradation \dot{w}, as a linear combination of all entropy generation terms, see equation (Equation 2.53). pᵢ can be energy dissipated, work lost, heat transferred, change in thermodynamic energy (internal energy, enthalpy, Helmholtz or Gibbs free energy), or some other functional form of energy. ζᵢⱼ are time-dependent phenomenological variables associated with the dissipative processes pᵢ. The degradation coefficients Bᵢ must be measured using equation (Equation 2.54). The approach
1. identifies the degradation measure w, dissipative process energies pᵢ and phenomenological variables ζᵢⱼ,
2. finds entropy generation S′ caused by the pᵢ,
3. evaluates coefficients Bᵢ by measuring increments/accumulation or rates of degradation versus increments/accumulation or rates of entropy generation, with process pᵢ active.

This approach can solve problems consisting of one or many variegated dissipative processes. The DEG theorem has analyzed friction and wear and metal fatigue degradation.

Thermodynamic Formulations—Closed System

This section reviews the first and second laws of thermodynamics for application to degradation of batteries.

First Law—Energy Conservation

The first law

dU=δQ−δW+ΣμₖdNₖ (Equation

for a closed stationary thermodynamic system, neglecting gravity, balances dU the change in internal energy, δQ the heat exchange across the system boundary, δW the energy transfer across the system boundary by work, and ΣμₖdNₖ the internal energy changes due to chemical reactions and diffusion. For chemical reactions governed by a stoichiometric equation such as equations (Equation 4.176), (Equation 7.380) and (Equation 4.178),

ΣμₖdNₖ=Adξ (Equation

where A is reaction affinity and dξ is reaction extent.

Second Law and Entropy Balance

Irreversible Entropy Generation

Irreversible entropy S′ generated by dissipative processes measures the permanent changes in a system when the process constraint is removed or reversed. For a closed system, the second law of thermodynamics can be stated as

$$dS = \frac{δ Q}{T} + δ {S}^{′} , \qquad ( Equation 7.391 )$$

where the first term on the right, the entropy transfer by heat δQ, may be positive or negative. Here dS is the entropy change in the system and T is the temperature of the boundary where the energy/entropy transfer takes place. The second law asserts that entropy generated δS′≥0.

In some embodiments, temperature sensors is placed to determine the instantaneous boundary temperature (in some embodiments, as the only temperature sensor), which can be determined as the hottest external spot that can be identified. In some embodiments, this spot is closest to the heat generation points to which the temperature can be placed. For example, for the Pb-acid battery, the instantaneous boundary temperature is acquired at the electrolyte through a hole in the cap (which facilitate direct measurement of temperature of the electrolyte exclude the plastic housing). Without access to the electrolyte, as in the case of the Li-ion battery, the thermocouple can be placed on the battery housing (external).

Combining First and Second Laws with Gibbs Potential

For a system undergoing quasi-static heat transfer and compression work, equation (Equation 1.2) is restated as

dU=TdS−PdV+ΣμₖdNₖ (Equation

often referred to as the TdS equation. Here P is pressure, V volume, T temperature and S entropy. Electrochemical energy storage devices are conveniently characterized using the Gibbs free energy, an alternate form of the first law derived from Legendre Transforms,

G=U+PV−TS, (Equation 7.393)

which can measure process-initiating energy changes in a thermodynamic system. Differentiating equation (Equation 7.393) and substituting equation (Equation 7.392) for dU into the result gives the Gibbs fundamental relation

dG=−SdT+VdP+ΣμₖdNₖ (Equation

the change in Gibbs energy of the system, according to the first law.

For reactions such as phase transitions and chemical formation/decomposition of substances, changes in Gibbs energy along reversible and irreversible paths between states can determine entropy changes in the system. Eliminating δQ from equation (Equation 1.2) with equation (Equation 7.391) gives, for compression work PdV,

dU=TdS−TδS′−PdV+ΣμₖdNₖ (Equation

the irreversible combined form of the first and second laws, or the TδS′ equation. Differentiating equation (Equation 7.393) and substituting equation (Equation 7.395) for dU into the resulting equation gives the irreversible form of the Gibbs fundamental relation

dG=dGᵣₑᵥ=−SdT+VdP+ΣμₖdNₖ−TδS′≤0 (Equation

where dGᵣₑᵥ is the reversible (or ideal) electrochemical energy change in the system (maximum for energy transfer out of the system and minimum for energy transfer into the system), obtained by adding energy lost due to entropy production TδS′ to the actual Gibbs energy extracted, e.g, from a battery. Rearranging gives the fundamental Gibbs-based entropy production relation

$$δ {S}^{′} = - \frac{SdT}{T} + \frac{V dP}{T} + \frac{Σ {μ}_{k} {dN}_{k}}{T} - \frac{{dG}_{rev}}{T} \geq 0 \\ \text{?} \text{indicates text missing or illegible when filed} \qquad ( Equation \text{?}$$

which satisfies the second law. For an energy-extraction process such as discharging a battery, dT≥0, dP≤0, dNₖ≤0 and dGᵣₑᵥ≤0, rendering δS′≥0. For an energy-adding process such as battery charging, dT≤0, dP≥0, dNₖ≥0 and dGᵣₑᵥ≥0, reversing the signs of the middle terms in equation (Equation 7.397) to preserve accordance with the second law δS′≥0.

Equation (Equation 7.397) defines entropy production as the difference between irreversible

$$δ {S}_{irr} = \frac{{dG}_{irr}}{T}$$

and reversible

$${dS}_{rev} = \frac{{dG}_{rev}}{T}$$

Gibbs entropies

δS′=δSᵢᵣᵣ−dSᵣₑᵥ≥0 (Equation 7.398)

where for energy extraction, δSᵢᵣᵣ<0, dSᵣₑᵥ<0 and |δSᵢᵣᵣ|≤|dSᵣₑᵥ|; and for energy addition, δSᵢᵣᵣ>0, dSᵣₑᵥ>0 and δSᵢᵣᵣ≥dSᵣₑᵥ. Here irreversible (or actual) Gibbs energy change dGᵢᵣᵣ is obtained from equation (Equation 7.394), with its terms as the numerators of the first three terms after the equality sign in equation (Equation 7.397). Equation (Equation 7.398) indicates that a portion of the system's energy is always unavailable for external work and a portion of the energy added from an external source is always unavailable to the system. Equation (Equation 7.397) and its abbreviated form in equation (Equation 7.398), give the entropy generated by the system's internal irreversibilities alone and is in accordance with experience, similar to the Gouy-Stodola theorem obtained from availability analysis. The foregoing equations are in accordance with the IUPAC convention of positive energy into a system. Inexact differential δ indicates path-dependent variables.

Battery Analysis

Batteries degrade chemically through electrode corrosion and evolution of gases; electrically as observed through capacity fade; and thermally via hot environments and joule heating, which often accelerate electrochemical degradation.

Thermodynamic Analysis—Gibbs Energy and Entropy

Assumptions:
1. The system boundary encloses the battery only.
2. System is closed (battery mass stays in the battery).
3. Heat transfers between battery and surroundings.
4. The system is at equilibrium before and after discharging/charging.

The change in the Gibbs energy, equation (Equation 7.394), occurs at constant pressure

dG=−SdT+ΣμₖdNₖ (Equation

where

ΣμₖdNₖ=ΣμᵣdNᵣ+Σ(μ_{high}−μ_{low})dN_{D} (Equation

accounts for chemical reaction r and diffusion D. μ_{high} and μ_{low} are chemical potentials for diffusion in the high and low potential regions respectively, and dN_{D} is the amount of active species transported between both regions. For convenience, the chemical reaction and diffusion energy changes in the battery are replaced by the directly coupled electrical boundary work given by the Ohmic process Vdq,

ΣμₖdNₖ=Vdq (Equation

where V is the battery terminal voltage and dq=kit is the charge transferred. For the discharge/charge process, equation (Equation 4.200) becomes

dG=−SdT+Vdq (Equation

where dT≥0, dq≤0 for discharge and dT≤0, dq≥0 for charge. Equation (Equation 4.203) gives the quasi-static change in Gibbs potential, during discharge and charge respectively. From equation (Equation 7.397) with pressure constant, entropy generation during discharge/charge is

$$δ {S}^{′} = - \frac{SdT}{T} + \frac{Vdq}{T} - \frac{{dG}_{rev}}{T} \qquad ( Equation$$

Equation (Equation 7.403) suggests S′=(T, q, G). Here dGᵣₑᵥ can be evaluated via

dGᵣₑᵥ=V_{OC}dqᵣₑᵥ=V_{OC}n′FdNᵣₑᵥ (Equation

where V_{OC} is the battery's open-circuit voltage (or standard potential), dGᵣₑᵥ is reversible charge transfer, n′ is number of species, e.g. electrons involved in charge transfer (2 for lead-acid batteries), F=96,485 C/mol is Faraday's constant and dNᵣₑᵥ is reversible mole transfer.

Relaxation/Settling

During active discharging/charging, any heat generated or internal dissipation and not instantaneously transferred out builds up. Upon load removal, the battery settles and that heat transfers out as the battery's Gibbs energy and entropy proceed to a new equilibrium state. During settling, the cell voltage relaxes and the battery transfers entropy to the atmosphere spontaneously. To apply equation (Equation 7.397), the internal diffusion process energy (second right hand side RHS term in equation (Equation 7.400)) replaces the combined reaction and diffusion term ΣμₖdNₖ for settling (having no external charge transfer and thus negligible active chemical reaction effects). Entropy production during settling becomes

$$δ {S}_{r}^{′} = - \frac{SdT}{T} + \frac{( {μ}_{high} - {μ}_{low} ) {dN}_{D}}{T} - \frac{{dG}_{rev}}{T} ( relaxation ) \qquad ( Equation 7.405 )$$

Here −SdT, positive for decreasing temperature dT≤0, represents both voltage and thermal relaxation, and (μ_{high}−μ_{low})dN_{D} represents diffusion during settling, all of which proceed spontaneously and significantly slower than the active Ohmic processes.

With the voltage relaxation component proceeding in opposite directions for discharge and charge, hence subtracting out during a balanced discharge-charge cycle, entropy production during settling proceeds at the same rate as spontaneous cooling and diffusion of the charge species, which, for entropy analysis involving active Ohmic interactions, is negligible. Relaxation equilibrium is approached asymptotically, taking several hours to days, so that the entropy produced by discharging and charging far exceeds that of relaxation, i.e.,

δS′>>δS′ᵣ. (Equation 7.406)

Also, some batteries in operation continue to supply power while charging, removing the settling step from the cycling schedule and hence, analysis.

Rate and Cyclic Analysis

Using rate forms of equations (Equation 4.203) and (Equation 7.403), Gibbs energy change and entropy production in the battery during discharge/charge is given by

$$\begin{matrix}\dot{G} = - S \dot{T} + VI & ( Equation \\ {\dot{S}}^{′} = - \frac{S \dot{T}}{T} + \frac{VI}{T} - \frac{{\dot{G}}_{rev}}{T} . & ( Equation 7.408 )\end{matrix}$$

Via equations (Equation 4.203) and (Equation 7.403), total change in Gibbs energy and entropy generation during cycling, from time t₀ to t, with Ġᵣₑᵥ=V_{OC}Iᵣₑᵥ substituted via equation (Equation 7.404), is

$$\begin{matrix}Δ G = - {\int }_{{t}_{0}}^{t} S \dot{T} dt + {\int }_{{t}_{0}}^{t} VIdt & ( Equation \\ {S}^{′} = - {\int }_{{t}_{0}}^{t} \frac{S \dot{T}}{T} dt + {\int }_{{t}_{0}}^{t} \frac{VI}{T} dt - {\int }_{{t}_{0}}^{t} \frac{{V}_{OC} {I}_{rev}}{T} dt . & ( Equation 7.410 )\end{matrix}$$

For a complete cycle, considering the effect on signs of the direction of current I and temperature change rate \dot{T} during discharge and charge mentioned previously,

$${S}_{cycle}^{′} = - {\int }_{{t}_{c}}^{{t}_{d}} \frac{S \dot{T}}{T} dt - {\int }_{{t}_{c}}^{{t}_{d}} \frac{VI}{T} dt + {\int }_{{t}_{c}}^{{t}_{d}} \frac{{V}_{0} {I}_{rev}}{T} dt + {\int }_{{t}_{d}}^{{t}_{c}} \frac{S \dot{T}}{T} dt + {\int }_{{t}_{d}}^{{t}_{c}} \frac{VI}{T} dt - {\int }_{{t}_{d}}^{{t}_{c}} \frac{{V}_{0} {I}_{rev}}{T} dt \qquad ( Equation$$

where t_{c} and t_{d} are the end times of the charge and discharge steps respectively. Integral limits t_{c} to t_{d} pertain to discharging whereas integral limits t_{d} to t_{c} pertain to charging. In active cycling analysis, settling is negligible as discussed previously.

Entropy Content S and Internal Energy Dissipation −SdT

The Gibbs equation (Equation 7.394) introduced −SdT, the energy dissipated and accumulated internally, i.e. not instantaneously transferred out during the work interaction, which includes primarily the effects of Ohmic/reaction heat generation and in some cases, contribution from an external heat source. The temperature change dT is driven by the entropy content S of the system. Resolving system entropy S into its component terms indicates that S=S(μ,T), a function of chemical potential μ and temperature T, for a chemically reactive system. Invoking the famous Gibbs-Duhem equation

SdT−VdP+ΣNₖdμₖ=0 (Equation

where ΣNₖdμₖ=Ndμ for a system comprised of one active species, at constant pressure gives

−SdT=Ndμ, (Equation 7.413)

which relates reactive system transformation to temperature change. The molar Gibbs energy at constant temperature and pressure gives the chemical potential of the system. From equation (Equation 7.394),

$$μ = {( \frac{\partial G}{\partial N} )}_{T , P} . \qquad ( Equation 7.414 )$$

Substituting equation (Equation 7.413) in rate form into equations (Equation 4.240) and (Equation 4.241),

$$\begin{matrix}Δ G = {\int }_{{t}_{0}}^{t} N \dot{μ} dt + {\int }_{{t}_{0}}^{t} VIdt & ( Equation \\ {S}^{′} = {\int }_{{t}_{0}}^{t} \frac{N \dot{μ}}{T} dt + {\int }_{{t}_{0}}^{t} \frac{VI}{T} dt - {\int }_{{t}_{0}}^{t} \frac{{V}_{0} {I}_{rev}}{T} dt . & ( Equation 7.416 )\end{matrix}$$

From Faraday's first law,

$$N = \frac{{n}^{′} F}{} . \qquad ( Equation 7.417 )$$

Here [symbol] is the instantaneous charge in the battery at time t given as

[symbol]

(t)= [symbol] ₀+ [symbol] ₜ(Δt)≥0 (Equation

where [symbol] ₀ is the battery's initial charge content at t=t₀, and

[symbol]

ₜ(Δt)=∫ₜ_{₀}^{t}I(t)dt (Equation

is the total charge transferred/accumulated at time t. Equations (Equation 7.418) and (Equation 7.419) imply that during discharge, I<0 and [symbol] ₜ<0 giving [symbol] ₀= [symbol] ₘₐₓ while during charge, I>0, [symbol] ₜ>0 implying [symbol] ₀= [symbol] ₘᵢₙ. Differentiating equation (Equation 7.417) and combining with equation (Equation 7.401) (where both q and [symbol] represent charge content) for a single species gives

μ=n′FV, (Equation 7.420)

relating the chemical potential to the battery's voltage. Using the electrochemical affinity A via equation (Equation 7.390), Kondepudi and Prigogine obtained an alternate form of equation (Equation 7.420). Substituting equation (Equation 7.417) for N and equation (Equation 7.420) for μ in equation (Equation 7.413) gives, in rate form,

−S\dot{T}=N\dot{μ}= [symbol] \dot{V}. (Equation 7.421)

Substituting equation (Equation 7.421) into equations (Equation 4.240) and (Equation 4.241),

$$\begin{matrix}Δ G = {\int }_{{t}_{0}}^{t} \dot{V} dt + {\int }_{{t}_{0}}^{t} VIdt & ( Equation \\ {S}^{′} = {\int }_{{t}_{0}}^{t} \frac{ \dot{V}}{T} dt + {\int }_{{t}_{0}}^{t} \frac{VI}{T} dt - {\int }_{{t}_{0}}^{t} \frac{{V}_{0} {I}_{rev}}{T} dt & ( Equation\end{matrix}$$

Equation (Equation 7.423) gives entropy production as a function of instantaneous voltage V, current I and temperature T. For multi-cells battery packs, if temperature variation across all the cells is insignificant during normal operation, as is likely the case for balanced loading of same-type cells used in a battery pack, then it is likely unnecessary to measure temperature of each individual cell. Indeed, a few points (only one point in some instance can be sufficient). In some embodiments, temperatures sensors can be placed at corners and central locations of the pack.

Within the multi-cell battery packs, measurements of each cell's voltage and current would be beneficial to the estimation of capacity fade, particularly in the determination of electrochemicothermal ECT term, which may be sensitive to each cell's response and will detect instabilities in any of the cells (e.g., even with just one boundary temperature).

The Gibbs-Duhem formulation at constant pressure, expressed in rate form in equation (Equation 7.421), shows that battery voltage and temperature are not independent: a rise in temperature results in a drop in voltage and a drop in voltage as charge transfers out of the battery causes a rise in its temperature—typically and easily experienced during active discharge. This is predicted by equations (Equation 7.394) and (Equation 7.396) in which the first term—SdT reduces the Gibbs energy for positive S and dT.

With a dependency on voltage change, charge content and temperature, the internal accumulation of energy dissipation −SdT term is more appropriately named electrochemicothermal (ECT) energy change, which for a reactive system depends on heat capacity, chemical potential, number of moles and changes in temperature of the reactive components. In some embodiments, wherein the ECT entropy is evaluated as a charge content multiplied by voltage change, divided by temperature.

\dot{V} and I are negative during discharge and positive during charge, establishing similar directional signs for the Gibbs energy change and entropy generation components. The Ohmic and reversible Gibbs entropies account for the boundary work and overall minimum possible losses respectively, while the ECT entropy accounts for the loss due to rise in internal entropy content and temperature that occurs as a result of drop in battery potential and charge content.

During relaxation/settling, entropy generation, equation (Equation 7.423), is obtained by applying the Gibbs-Duhem formulation at constant P (equation (Equation 7.413)) to equation (Equation 7.405). Without active charge transfer during settling, equation (Equation 7.423) indicates that ECT entropy, first right hand side (RHS) term, representing entropy accumulation from both voltage and thermal relaxation via equation (Equation 7.421), is the most significant component of entropy generation. Similar to relaxation/settling, equation (Equation 7.423) can also be used to evaluate entropy generation during battery storage including the effects of self-discharge, with the Ohmic term representing spontaneous charge leakage.

Degradation-Entropy Generation (DEG) Analysis

Using a procedure similar to that outlined above, this section applies the above Gibbs-based formulations to the DEG theorem as follows:
1 Let available battery capacity/charge content [symbol] be a degradation transformation measure and capacity fade (lost discharge/charge capacity) Δ [symbol] be the observed/measured degradation. The DEG equation (Equation 7.388) with Δ [symbol] replacing Δw becomes

Δ [symbol] C=ΣᵢBᵢS′ᵢ. (Equation 7.424)
2 Entropy generation, via equation (Equation 7.423), is

S′=S′{V(t),I(t),T(t)} (Equation 7.425)

a function of voltage, current and temperature evolution in which the difference between reversible and irreversible Maximum work entropies is the entropy generated in the system is shown as:

S′=∫ₜ_{₀}^{t}\dot{S}ᵢᵣᵣₑᵥdt−∫ₜ_{₀}^{t}\dot{S}ᵣₑᵥdt (Equation 7.47A)
3 Equations (Equation 7.424) and (Equation 7.425) suggest [symbol] = [symbol] {V(t), I(t), T(t)}. Substituting the individual entropy generation terms of equation (Equation 7.423) into equation (Equation 7.424) gives

$$Δ = {B}_{VT} {\int }_{{t}_{0}}^{t} \frac{ \dot{V}}{T} dt + {B}_{Ω} {\int }_{{t}_{0}}^{t} \frac{VI}{T} dt - {B}_{G} {\int }_{{t}_{0}}^{t} \frac{{V}_{0} {I}_{rev}}{T} dt , \qquad ( Equation 7.426 )$$

which can be rewritten as

Δ [symbol] =B_{VT}S′_{VT}+B_{Ω}S′_{Ω}−B_{G}S′ᵣₑᵥ (Equation

where B_{VT}, B_{Ω} and B_{G} are coefficients pertaining to respective entropy generation terms in equation (Equation 7.423). Equation (Equation 7.426), as well as its abbreviated form in equation (Equation 7.427), is the fundamental capacity fade-entropy generation relation. Via equation (Equation 2.54) with C replacing w, DEG coefficients

$${B}_{VT} = \frac{\partial }{\partial {S}_{VT}^{′}} ; {B}_{Ω} = \frac{\partial }{\partial {S}_{Ω}^{′}} ; {B}_{G} = \frac{\partial }{\partial {S}_{rev}} \qquad ( Equation$$

pertain to ECT entropy

$${S}_{VT}^{′} = \int \frac{ \dot{V}}{T} dt ,$$

Ohmic entropy

$${S}_{Ω}^{′} = \int \frac{VI}{T} dt$$

and reversible Gibbs entropy

$${S}_{rev}^{′} = \int \frac{{V}_{OC} {I}_{rev}}{T} dt$$

respectively, and can be evaluated from measurements as slopes of charge [symbol] versus irreversible entropy production components S′ᵢ for process pᵢ as shown in subsequent sections.

Capacity Fade and Entropy Generation in Rechargeable Batteries

In primary cells, degradation in the form of capacity/charge loss is simply the difference between initial charge content at time t₀ and charge content at later time t as the battery discharges irreversibly. In secondary cells, the charge step reverses this ‘loss’, making the prior definition unsuitable for describing irreversible loss of capacity. Over time, secondary cells lose their ability to hold charge, resulting in capacity fade. This capacity fade is defined and estimated as

Δ [symbol] = [symbol] ₁(Δt)− [symbol] _{N}(Δt), (Equation 7.429)

the difference between the first cycle's available capacity or charge [symbol] ₁(Δt) and the Nth cycle's capacity [symbol] _{N}(Δt) (Capacity [symbol] _{N}(Δt) can be replaced by the accumulated discharge measured after a discharge step). Using Coulomb/charge counting, equation (Equation 7.429) requires a consistent cycling schedule and constant discharge rate for all cycles between 1 and N. With equation (Equation 7.429), a derivation and breakdown of equation (Equation 7.426) is presented for practical application to rechargeable batteries.

In line with a corollary constraint of the second law, the Carnot limitation, which governs the availability of a system's energy for work, expressed for a heat source as

Energy added=Available energy+Unavailable energy, (Equation 7.430)

equations (Equation 7.426) describes Δ [symbol] , which can be decomposed into an irreversible component [symbol] ᵢᵣᵣ that establishes the battery's actual path, and a reversible component [symbol] ᵣₑᵥ that establishes its ideal path. The irreversible charge transfer [symbol] ᵢᵣᵣ can be correlated directly with irreversible entropy terms (first two RHS terms) in equation (Equation 7.423) as

$${irr}_{} = + Δ {irr}_{} = {B}_{VT} {\int }_{{t}_{0}}^{t} \frac{ \dot{V}}{T} dt + {B}_{Ω} {\int }_{{t}_{0}}^{t} \frac{VI}{T} dt \qquad ( Equation$$

where the irreversible capacity fade Δ [symbol] ᵢᵣᵣ, a portion of [symbol] ᵢᵣᵣ not available for external/boundary work during cycling due to instantaneous dissipation from battery heating and loss of charge and potential, is the difference between irreversible charge transfer [symbol] ᵢᵣᵣ and measured charge transfer [symbol] ₜ=∫ₜ_{₀}^{t}I(t)dt. Similarly, relating [symbol] ᵣₑᵥ to the reversible component of equation (Equation 7.423),

$${rev}_{} = {t}_{} + Δ {rev}_{} = {B}_{G} {\int }_{{t}_{0}}^{t} \frac{{V}_{OC} {I}_{rev}}{T} dt \qquad ( Equation$$

where the reversible capacity fade Δ [symbol] ᵣₑᵥ is the difference between reversible charge transfer

[symbol]

ᵣₑᵥ=∫ₜ_{₀}^{t}Iᵣₑᵥdt (Equation

and measured charge transfer [symbol] ₜ. Here [symbol] ᵣₑᵥ is maximum during discharge (maximum discharge capacity or charge transfer) and minimum during charge (minimum charge capacity or charge transfer); Δ [symbol] ᵣₑᵥ is the portion of [symbol] ᵣₑᵥ unavailable due to previous permanent degradation and instantaneous dissipation; and Iᵣₑᵥ is the constant reversible current, maximum during discharge and minimum during charge. Hence [symbol] ᵣₑᵥ=IᵣₑᵥΔt.

Following equation (Equation 7.398), the difference between equations (Equation 7.431) and (Equation 7.432) derives equation (Equation 7.426) which defines actual capacity fade from degradation

$$Δ ( Δ t ) = {irr}_{} - {rev}_{} = {B}_{VT} {\int }_{{t}_{0}}^{t} \frac{ \dot{V}}{T} dt + {B}_{Ω} {\int }_{{t}_{0}}^{t} \frac{VI}{T} dt - {B}_{G} {\int }_{{t}_{0}}^{t} \frac{{V}_{OC} {I}_{rev}}{T} dt \qquad ( Equation 7.434 )$$

as the difference between irreversible and reversible charge transfer components. With a known Iᵣₑᵥ, the last RHS term in equation (Equation 7.434) can be replaced with equation (Equation 7.433) to give

$$Δ ( Δ t ) = {B}_{VT} {\int }_{{t}_{0}}^{t} \frac{ \dot{V}}{T} dt + {B}_{Ω} {\int }_{{t}_{0}}^{t} \frac{VI}{T} dt - {I}_{rev} Δ t . \qquad ( Equation 7.435 )$$

In equation (7.57), the first term corresponds to B_{VT}S′_{VT} as an internal measure of battery's ability to deliver its charge content; the second term corresponds to B_{Ω}S′_{Ω} as a boundary measure of the effects of the battery's work interaction with external load; and the third term corresponds to BᵣₑᵥS′ᵣₑᵥ as an ideal/total measure of the limit (minimum/maximum) of battery's input/output energy. The first and second terms can be designated as irreversible charge, and the third term as a reversible charge.

Equation (Equation 7.435) gives the DEG model for instantaneous evaluation of operational capacity fade irrespective of discharge rate or depth of discharge. While [symbol] ₜ and [symbol] ᵣₑᵥ are determined from currents I and Iᵣₑᵥ, [symbol] ᵢᵣᵣ is not directly measurable, making equation (Equation 7.435), which requires only measurements of V, I and T, convenient for practical applications.

During discharge, [symbol] ᵣₑᵥ in equation (Equation 7.432) represents the overall maximum charge available in the battery, only obtained from new batteries (at t=t₁, [symbol] ₜ= [symbol] ᵣₑᵥ= [symbol] ᵢᵣᵣ) or if the battery operates as a perfect energy source or sink (wherein no output or input power converts to heat or degrades the battery). The measured discharge [symbol] ₜ is the instantaneous (local) minimum from the battery during cycling (i.e. at t>t₁, [symbol] ₜ< [symbol] ᵢᵣᵣ< [symbol] ᵣₑᵥ). The nonlinear effects of temperature and voltage changes are not readily observed in measured operational capacity [symbol] ₜ. As the battery degrades, the amount of energy required to restore its original charged state continues to increase. Hence during charge, [symbol] ᵣₑᵥ is the overall minimum charge required to restore the battery to its initial state, realizable in new batteries (i.e. at t=t₁, [symbol] ₜ= [symbol] ᵣₑᵥ= [symbol] ᵢᵣᵣ) or a thermodynamically ideal battery, while [symbol] ₜ is the instantaneous maximum charge received from the battery charger (i.e. at t>t₁, [symbol] ₜ> [symbol] ᵢᵣᵣ> [symbol] ᵣₑᵥ). This implies Δ [symbol] >0 during discharge and charge represents capacity fade, in accordance with equation (Equation 7.398). The next section will experimentally verify the prior analyses and formulations, and will present a detailed procedure for evaluating capacity fade Δ [symbol] in lead-acid batteries from equation (Equation 7.435).

In linear and consistent cycling in which [symbol] ᵣₑᵥ is defined by the first cycle, Coulombic capacity fade in subsequent cycles, equation (Equation 7.429), is equal to DEG's capacity fade, equation (Equation 7.435).

Experimental Data Analysis and Discussion

Lead-acid battery cycling data measured is used herein. Monitored parameters changed with time at unsteady rates. In tables will be data for discharge on the left side of a table, and charge on the right side of a table. Signs indicate a decrease or increase in a parameter during a process or the direction of the process; for example, ECT energy/entropy, charge transfer and Ohmic work/entropy are negative for discharge, and positive for charge. Path-dependent integrals used the trapezoidal rule. Data was sampled at 0.1 Hz (Δt=10 s). Plots pertaining to discharge are the “a” part of the figure on the left, and plots pertaining to charge are the “b” part of the figure on the right.

Using equations for estimating charge, Gibbs energy and entropy, data from lead-acid battery cycling experiments are presented in Tables 7.1 and 7.2 for the Deka 6 V starter battery #1. Table 7.1 presents the energy-entropy data and Table 7.2 presents the DEG data. In these tables, the column 1 variable N_{c} numbers the discharge-charge cycles. Other column variables in Table 7.1 are:
Columns 2, 8, 14: Measured charge transfer, equation (Equation 7.419), [symbol] ₜ(Δt)=∫ₜ_{₀}^{t}I(t)dt
Columns 3, 9, 15: Ohmic work from equation (Equation 7.422), G_{Ω}=∫ₜ_{₀}^{t} VI dt.
Columns 4, 10, 16: ECT energy from equation (Equation 7.422), G_{VT}=∫ₜ_{₀}^{t} [symbol] V dt.
Columns 5, 11, 17: From equation (Equation 7.423), entropy contribution from Ohmic work

$${S}_{Ω}^{′} = {\int }_{{t}_{0}}^{t} \frac{VI}{T} dt .$$

Columns 6, 12, 18: ECT entropy

$${S}_{VT}^{′} = {\int }_{{t}_{0}}^{t} \frac{ \dot{V}}{T} dt ,$$

from equation (Equation 7.423).
Columns 7, 13, 19: Reversible Gibbs entropy from equation (Equation 7.423),

$${S}_{rev}^{′} = {\int }_{{t}_{0}}^{t} \frac{{V}_{OC} {I}_{rev}}{T} dt .$$

Other Table 2 column variables are:

From equation (Equation 4.257),
Columns 2, 8, 10, 16: Charge-Ohmic entropy coefficient

$${B}_{Ω} = \frac{\partial }{\partial {S}_{Ω}^{′}}$$

and
Columns 3, 9, 11, 17: Charge-ECT entropy coefficient

$${B}_{VT} = \frac{\partial }{\partial {S}_{VT}^{′}} .$$

Columns 4, 12, 18: Measured charge transfer [symbol] ₜ.
Columns 5 13, 19: Irreversible charge, equation (Equation 7.431), [symbol] ᵢᵣᵣ.
Columns 6, 14, 20: Reversible charge, equation (Equation 7.433), [symbol] ᵣₑᵥ=IᵣₑᵥΔt.
Columns 7, 15, 21: Capacity fade, equation (Equation 7.435), Δ [symbol] .

In Table 7.1, the relatively linear portion of the discharge data (values when battery terminal voltage drops to 5 V during discharge, considered 100% discharge) termed “Normal Discharge” is followed by the “Total Discharge” data (values at the end of total discharge, including over-discharge). In Table 7.2, the discharge data is split into normal discharge, transition and over-discharge datasets. Only coefficients are presented for the transition region; details in subsequent sections. Vertical line separates discharge and charge datasets in both tables.

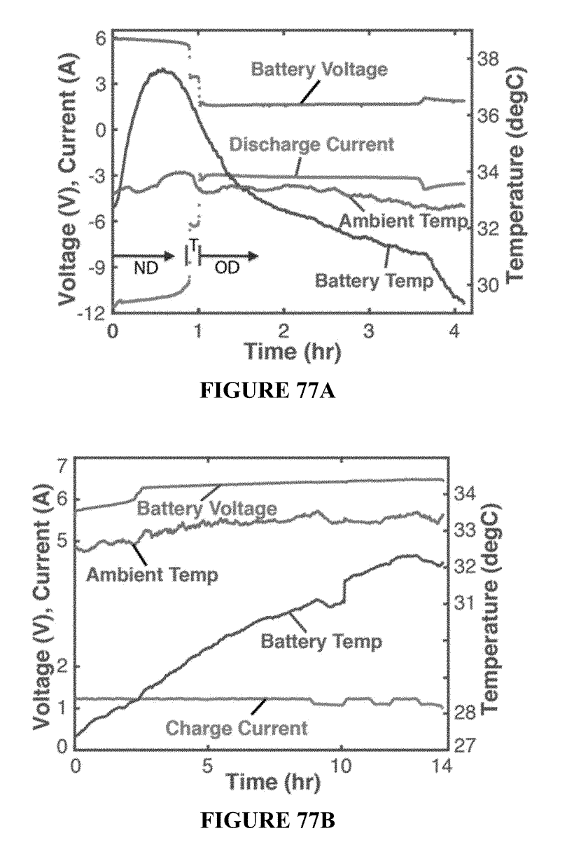

Voltage, current and temperatures versus time during cycling are shown in FIGS. 77A-77B. Trends from a randomly selected sample dataset, cycle 2, highlighted in Tables 7.1 and 7.2 in bold font, are shown in plots and discussed. Data and plots for discharge are to the left, and data and plots for charge to the right. Similar trends were observed for all four lead-acid batteries tested.

Note that irregular, inconsistent and abusive—severe over-discharge followed by insufficient recharge—cycling schedule was used to show robustness of the model. In accordance with battery industry, Ah, Wh and Wh/K are used for charge, energy and entropy respectively (1 Ah=3600 As=3600 C and 1 Wh=3600 Ws=3600 J and 1 Wh/K=3600 J/K), giving B coefficients units of Ah K/Wh (1 Ah K/Wh=1 K/V. DEG's B coefficients are given in Ah K/W to differentiate them from the reciprocal of the voltage-temperature coefficient which has units of V/K).

TABLE 7.1
Processed Gibbs energy and entropy parameters for lead-acid starter battery (Initial discharge rate: ~11 A, line marks
the start of ~35 A discharge rate. Charge rate: 1.2 A). Cycle 2 (in bold) is used in the detailed breakdown in this section.

| | Normal Discharge | | | | | | Total Discharge | | | | | | Charge | | | | | |
|---|---|---|---|---|---|---|---|---|---|---|---|---|---|---|---|---|---|---|
| | | | | S′_{Ω} | S′_{VT} | S′ᵣₑᵥ | | | | S′_{Ω} | S′_{VT} | S′ᵣₑᵥ | | | | S′_{Ω} | S′_{VT} | S′ᵣₑᵥ |
| | [symbol] | G_{Ω} | G_{VT} | Wh/ | Wh/ | Wh/ | [symbol] | G_{Ω} | G_{VT} | Wh/ | Wh/ | Wh/ | [symbol] | G_{Ω} | G_{VT} | Wh/ | Wh/ | Wh/ |
| N_{c} | Ah | Wh | Wh | K | K | K | Ah | Wh | Wh | K | K | K | Ah | Wh | Wh | K | K | K |
| 1 | −54.6 | −315.1 | −12.9 | −1.02 | −0.041 | −1.35 | −61.3 | −329.1 | −33.4 | −1.06 | −0.11 | −1.85 | 6.1 | 38.5 | 0.5 | 0.13 | 0.002 | 0.11 |
| 2 | −9.9 | −57.5 | −7.7 | −0.19 | −0.025 | −0.22 | −20.4 | −76.6 | −44.8 | −0.25 | −0.14 | −1.03 | 16.5 | 104.1 | 2.9 | 0.34 | 0.010 | 0.29 |
| 3 | −10.3 | −58.4 | −4.2 | −0.19 | −0.014 | −0.24 | −12.3 | −62.2 | −11.3 | −0.20 | −0.04 | −0.40 | 15.2 | 97.0 | 1.8 | 0.32 | 0.006 | 0.28 |
| 4 | −12.4 | −72.6 | −9.7 | −0.23 | −0.031 | −0.28 | −23.2 | −90.9 | −49.2 | −0.29 | −0.16 | −1.16 | 15.3 | 95.6 | 3.0 | 0.32 | 0.010 | 0.27 |
| 5 | −8.3 | −47.5 | −2.3 | −0.15 | −0.007 | −0.19 | −15.5 | −57.7 | −29.4 | −0.19 | −0.09 | −0.91 | 25.3 | 161.6 | 6.1 | 0.53 | 0.020 | 0.28 |
| 6 | −14.8 | −85.8 | −6.4 | −0.27 | −0.021 | −0.32 | −23.5 | −100.9 | −36.2 | −0.32 | −0.12 | −1.01 | 19.1 | 121.2 | 3.6 | 0.40 | 0.012 | 0.34 |
| 7 | −12.8 | −74.2 | −4.6 | −0.23 | −0.014 | −0.28 | −19.5 | −85.6 | −27.9 | −0.27 | −0.09 | −0.83 | 16.9 | 106.7 | 3.1 | 0.35 | 0.010 | 0.29 |
| 8 | −11.5 | −65.4 | −5.9 | −0.21 | −0.019 | −0.26 | −16.4 | −74.1 | −23.0 | −0.24 | −0.07 | −0.68 | 16.0 | 101.2 | 2.0 | 0.33 | 0.007 | 0.28 |
| 9 | −11.2 | −63.6 | −5.8 | −0.21 | −0.019 | −0.26 | −15.6 | −71.2 | −21.1 | −0.23 | −0.07 | −0.66 | 21.3 | 132.2 | 4.9 | 0.43 | 0.016 | 0.23 |
| 10 | −8.9 | −50.9 | −10.1 | −0.17 | −0.033 | −0.21 | −24.0 | −70.9 | −68.9 | −0.23 | −0.23 | −1.98 | 34.8 | 223.5 | 5.2 | 0.73 | 0.017 | 0.59 |
| 11 | −13.0 | −75.0 | −12.0 | −0.24 | 0.039 | −0.28 | −29.2 | −102.1 | −69.9 | −0.33 | −0.23 | −1.67 | 19.5 | 122.8 | 4.4 | 0.41 | 0.015 | 0.32 |
| 12 | −10.1 | −57.7 | −8.8 | −0.19 | −0.029 | −0.23 | −21.5 | −72.7 | −53.0 | −0.24 | −0.17 | −1.64 | 26.7 | 170.3 | 7.8 | 0.56 | 0.025 | 0.48 |
| 13 | −11.1 | −63.5 | −17.0 | −0.21 | −0.056 | −0.26 | −29.1 | −85.9 | −86.4 | −0.28 | −0.28 | −3.41 | 27.1 | 172.3 | 2.7 | 0.56 | 0.009 | 0.43 |
| 14 | −9.5 | −53.7 | −9.4 | −0.17 | −0.031 | −0.22 | −22.9 | −64.5 | −66.5 | −0.21 | −0.22 | −4.59 | 19.6 | 123.3 | 3.0 | 0.40 | 0.010 | 0.29 |
| 15 | −7.6 | −42.8 | −8.3 | −0.14 | −0.027 | −0.18 | −16.8 | −50.3 | −47.4 | −0.17 | −0.16 | −3.26 | 20.7 | 131.8 | 8.5 | 0.44 | 0.028 | 0.39 |
| 16 | −10.5 | −59.4 | −8.5 | −0.20 | −0.028 | −0.23 | −21.6 | −70.3 | −53.4 | −0.23 | −0.18 | −2.92 | 10.9 | 67.7 | 1.8 | 0.23 | 0.006 | 0.19 |
| 17 | −4.4 | −23.8 | −3.1 | −0.08 | −0.010 | −0.10 | −10.8 | −31.3 | −28.1 | −0.10 | −0.09 | −2.33 | 10.6 | 66.4 | 1.5 | 0.22 | 0.005 | 0.20 |
| 18 | −3.5 | −19.0 | −3.1 | −0.06 | −0.010 | −0.08 | −9.5 | −26.1 | −26.6 | −0.09 | −0.09 | −2.46 | 7.9 | 49.6 | 1.0 | 0.16 | 0.003 | 0.14 |
| 19 | −2.1 | −11.5 | −2.2 | −0.04 | −0.007 | −0.05 | −5.8 | −17.0 | −15.8 | −0.06 | −0.05 | −1.19 | 11.2 | 71.1 | 1.4 | 0.23 | 0.005 | 0.20 |

SUMMARY

| | | | | | | | | |
|---|---|---|---|---|---|---|---|---|
| −4.20 | −0.383 | −5.24 | −4.99 | −2.59 | −33.98 | 7.10 | 0.216 | 5.60 |

TABLE 7.2
Processed DEG parameters for lead-acid starter battery (Initial discharge
rate: ~11 A, line marks the start of ~35 A discharge rate. Charge rate:
1.2 A). Cycle 2 (in bold) is used in the detail breakdown in this section.

| | Discharge | | | | | | | | | |
|---|---|---|---|---|---|---|---|---|---|---|
| | | | | | | | | | Over- | |
| | Normal Discharge | | | | | | Transition | | Discharge | |
| N_{c} | B_{Ω} | B_{VT} | [symbol] | [symbol] ᵢᵣᵣ | [symbol] ᵣₑᵥ | Δ [symbol] | B_{Ω} | B_{VT} | B_{Ω} | B_{VT} |
| N_{c} | KV⁻¹ | KV⁻¹ | Ah | Ah | Ah | Ah | KV⁻¹ | KV⁻¹ | KV⁻¹ | KV⁻¹ |
| 1 | 54.3 | −7.9 | −54.6 | −54.8 | −64.7 | 9.9 | 90.4 | 1.5 | 191.3 | 0.4 |
| 2 | 51.9 | 13.8 | −9.9 | −10.0 | −11.0 | 1.0 | 89.4 | 0.0 | 184.3 | 32.6 |
| 3 | 54.0 | −2.1 | −10.3 | −10.3 | −11.5 | 1.2 | 95.1 | 0.2 | 214.1 | −20.6 |
| 4 | 52.9 | 12.5 | −12.4 | −12.6 | −13.8 | 1.3 | 91.4 | 0.0 | 190.1 | −3.7 |
| 5 | 53.5 | 12.3 | −8.3 | −8.3 | −9.5 | 1.1 | 94.9 | −0.2 | 225.0 | 7.6 |
| 6 | 53.2 | 9.2 | −14.8 | −14.8 | −16.0 | 1.2 | 90.1 | 0.1 | 192.2 | −3.3 |
| 7 | 54.2 | 10.1 | −12.8 | −12.9 | −14.0 | 1.1 | 91.0 | 0.0 | 195.1 | 0.7 |
| 8 | 53.1 | 9.0 | −11.5 | −11.5 | −12.8 | 1.3 | 90.8 | −0.1 | 198.5 | 9.0 |
| 9 | 53.1 | 14.2 | −11.2 | −11.2 | −12.7 | 1.5 | 93.1 | 0.1 | 204.3 | 41.4 |

1ST HALF SUMMARY

| | | | | | | | | | | |
|---|---|---|---|---|---|---|---|---|---|---|
| | | | | | | 19.6 | | | | |
| 10 | 52.4 | 6.4 | −8.9 | −8.9 | −9.9 | 1.0 | 98.4 | −0.4 | 249.3 | −3.1 |
| 11 | 51.9 | 8.8 | −13.0 | −13.0 | −13.9 | 0.9 | 93.7 | −0.2 | 198.0 | 14.0 |
| 12 | 52.2 | 10.1 | −10.1 | −10.1 | −11.3 | 1.1 | 96.5 | −0.2 | 282.8 | −9.9 |
| 13 | 52.2 | 6.4 | −11.1 | −11.2 | −12.4 | 1.3 | 94.3 | 0.1 | 275.0 | 21.3 |
| 14 | 52.5 | 9.1 | −9.5 | −9.5 | −10.8 | 1.3 | 152.5 | 2.0 | 637.2 | 52.3 |
| 15 | 52.2 | 11.7 | −7.6 | −7.6 | −8.6 | 1.0 | 96.6 | 5.4 | 655.1 | 392.1 |
| 16 | 51.9 | 11.6 | −10.5 | −10.6 | −11.5 | 0.9 | 97.8 | 4.2 | 621.4 | 33.0 |
| 17 | 54.6 | 4.6 | −4.4 | −4.4 | −5.1 | 0.7 | 157.3 | −3.3 | 1229.5 | −203.8 |
| 18 | 54.1 | 6.4 | −3.5 | −3.5 | −4.0 | 0.5 | 148.9 | 0.4 | 1896.6 | −639.8 |
| 19 | 53.9 | 8.9 | −2.1 | −2.1 | −2.4 | 0.3 | 72.1 | 4.7 | 1526.4 | −699.8 |

2ND HALF SUMMARY

9.0

| | Discharge | | | | | | | | | |
|---|---|---|---|---|---|---|---|---|---|---|
| | Total Discharge | | | | Charge | | | | | |
| N_{c} | [symbol] | [symbol] ᵢᵣᵣ | [symbol] ᵣₑᵥ | Δ [symbol] | B_{Ω} | B_{VT} | [symbol] | [symbol] ᵢᵣᵣ | [symbol] ᵣₑᵥ | Δ [symbol] |
| N_{c} | Ah | Ah | Ah | Ah | KV⁻¹ | KV⁻¹ | Ah | Ah | Ah | Ah |
| 1 | −61.3 | −203.0 | −243.7 | 40.6 | 47.6 | 39.6 | 6.1 | 6.1 | 5.3 | 0.8 |
| 2 | −20.4 | −50.5 | −78.2 | 27.8 | 46.7 | 41.7 | 16.5 | 16.4 | 14.0 | 2.4 |
| 3 | −12.3 | −42.7 | −56.0 | 13.3 | 48.2 | −40.0 | 15.2 | 15.1 | 13.4 | 1.8 |
| 4 | −23.2 | −54.5 | −97.3 | 42.8 | 46.8 | 43.0 | 15.3 | 15.2 | 13.1 | 2.0 |
| 5 | −15.5 | −42.5 | −72.1 | 29.6 | 47.8 | −6.3 | 25.3 | 25.2 | 22.0 | 3.2 |
| 6 | −23.5 | −61.8 | −95.9 | 34.1 | 48.0 | −9.5 | 19.1 | 19.0 | 16.2 | 2.8 |
| 7 | −19.5 | −53.1 | −80.6 | 27.5 | 48.2 | −14.4 | 16.9 | 16.7 | 13.9 | 2.8 |
| 8 | −16.4 | −48.6 | −69.8 | 21.2 | 48.7 | −29.1 | 16.0 | 16.0 | 13.5 | 2.4 |
| 9 | −15.6 | −50.0 | −67.8 | 17.9 | 47.8 | −0.4 | 21.3 | 20.9 | 17.4 | 3.6 |
| 1ST HALF SUMMARY | | | | | | | | | | |
| | | | | 254.8 | | | | | | 21.8 |
| 10 | −24.0 | −57.2 | −117.1 | 59.9 | 46.5 | 16.4 | 34.8 | 34.4 | 27.6 | 6.8 |
| 11 | −29.2 | −69.4 | −128.1 | 58.7 | 47.5 | 4.5 | 19.5 | 19.4 | 15.2 | 4.2 |
| 12 | −21.5 | −65.6 | −115.7 | 50.1 | 48.6 | −26.5 | 26.7 | 26.5 | 22.7 | 3.9 |
| 13 | −29.1 | −83.3 | −197.1 | 113.8 | 47.7 | −1.6 | 27.1 | 26.9 | 20.5 | 6.4 |
| 14 | −22.9 | −145.7 | −338.4 | 192.7 | 48.0 | 0.4 | 19.6 | 19.4 | 13.8 | 5.7 |
| 15 | −16.8 | −169.3 | −251.6 | 82.4 | 48.1 | −16.7 | 20.7 | 20.5 | 18.2 | 2.3 |
| 16 | −21.6 | −150.0 | −286.0 | 136.1 | 46.7 | 36.1 | 10.9 | 10.8 | 9.4 | 1.4 |
| 17 | −10.8 | −107.9 | −138.3 | 30.4 | 47.0 | 42.4 | 10.6 | 10.5 | 9.4 | 1.1 |
| 18 | −9.5 | −106.2 | −137.7 | 31.6 | 47.3 | 47.3 | 7.9 | 7.9 | 7.0 | 0.9 |
| 19 | −5.8 | −48.7 | −74.8 | 26.2 | 48.7 | −47.0 | 11.2 | 11.2 | 9.5 | 1.7 |

2ND HALF SUMMARY

| | |
|---|---|
| 781.9 | 34.4 |

FIGS. 77A-77B plot battery voltage V, discharge/charge current I and temperature T versus time as the battery discharged (FIG. 77A) and charged (FIG. 77B) during cycle 2 (randomly selected for instantaneous breakdown of the discharge and charge steps). With a constant resistive load R_{load}, V and I trend similarly during discharge. The sudden drops in voltage and current mark the 100% (full) discharge point and the transition (T) from normal discharge (ND) to over-discharge (OD) in FIG. 77A. Current negative during discharge indicates charge outflow, and positive during charge indicates charge inflow.

In some embodiments, the temperature is determined via temperature sensors placed to determine the instantaneous boundary temperature (in some embodiments, as the only temperature sensor), which can be determined as the hottest external spot that can be identified. In some embodiments, this spot is closest to the heat generation points to which the temperature can be placed. For example, for the Pb-acid battery, the instantaneous boundary temperature is acquired at the electrolyte through a hole in the cap (which facilitate direct measurement of temperature of the electrolyte exclude the plastic housing). Without access to the electrolyte, as in the case of the Li-ion battery, the thermocouple can be placed on the battery housing (external).

In FIG. 77A, battery temperature initially rises fast, starts to drop while still at the same discharge rate, then continues to drop in the OD region until falling below initial temperature. This thermal optimization feature appears to be used by the battery manufacturer to prevent thermal instability and minimize degradation of battery components, as starter batteries are used to output very high current over a short duration. During charge (FIG. 77B), the battery temperature rises only by a few degrees over a 14-hour period, particularly due to the slow charge rate. Measured charge transfer, columns 2, 8, 14 of Table 7.1 and columns 4, 12, 18 of Table 7.2,

$${t}_{} ( Δ t ) = {\int }_{{t}_{0}}^{t} I ( t ) dt \approx {\sum }_{1}^{n} [ {I}_{n} + {I}_{n - 1} ] \frac{Δ t}{2} \qquad ( Equation$$

where 1, 2, 3, . . . , n is a vector index corresponding to times t₁, t₂, t₃, . . . , tₙ and Δt=tₙ−tₙ₋₁.

Gibbs Energy and Entropy

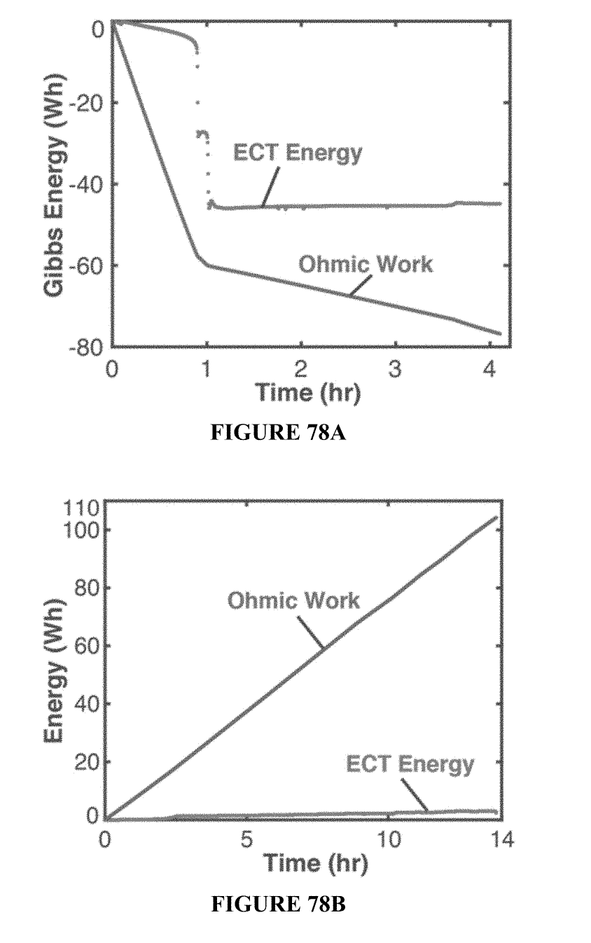

FIGS. 78A and 78B plot Gibbs energy components during discharge (FIG. 78A) and charge (FIG. 78B), with Ohmic work, columns 3, 9 and 15 of Table 7.1,

$${G}_{Ω} = {\int }_{{t}_{0}}^{t} IVdt \approx {\sum }_{1}^{n} [ {I}_{n} {V}_{n} + {I}_{n - 1} {V}_{n - 1} ] \frac{Δ t}{2} , \qquad ( Equation 7.437 )$$

ECT energy, columns 4, 10 and 16,

$${G}_{VT} = {\int }_{{t}_{0}}^{t} \dot{V} dt \approx {\sum }_{1}^{n} [ ( {n}_{} {\dot{V}}_{n} + {n - 1}_{} {\dot{V}}_{n - 1} ) ] \frac{Δ t}{2} . \qquad ( Equation 7.438 )$$

Ohmic work G_{Ω} linearly decreases available Gibbs energy during discharge with a change in slope at the transition to over-discharge, and linearly increases during charge. With Ohmic heating dominating heat removal mechanisms, and voltage drop during discharge, electrochemicothermal (ECT) energy G_{VT} in the battery decreases during discharge, contributing to the overall loss of available Gibbs energy. During charge, ECT energy increases slightly, an order of magnitude less than Ohmic work, the latter dominating the total Gibbs energy change. For both discharge and charge, ECT energy correlates directly with the battery's voltage and available charge content.

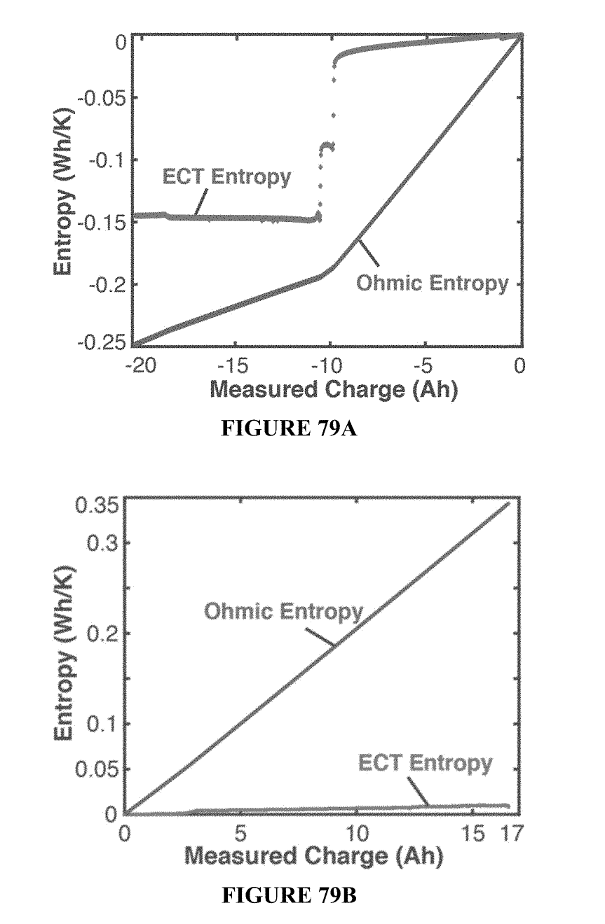

FIGS. 79A and 79B plot Ohmic S′_{Ω} (columns 5, 11 and 17), ECT S′_{T} (columns 6, 12 and 18) and reversible Gibbs S′ᵣₑᵥ (columns 7, 13 and 19) entropies

$$\begin{matrix}{S}_{Ω}^{′} = {\int }_{{t}_{0}}^{t} \frac{IV}{T} dt \approx {\sum }_{1}^{n} [ \frac{{I}_{n} {V}_{n} + {I}_{n - 1} {V}_{n - 1}}{{T}_{n , ave}} ] \frac{Δ t}{2} & ( Equation \\ {S}_{VT}^{′} = {\int }_{{t}_{0}}^{t} \frac{ \dot{V}}{T} dt \approx {\sum }_{1}^{n} [ \frac{( {n}_{} {\dot{V}}_{n} + {n - 1}_{} {\dot{V}}_{n - 1} )}{{T}_{n , ave}} ] \frac{Δ t}{2} & ( Equation \\ {S}_{rev}^{′} = {\int }_{{t}_{0}}^{t} \frac{{V}_{OC} {I}_{rev}}{T} dt \approx {\sum }_{1}^{n} [ \frac{{V}_{OC} {I}_{rev}}{{T}_{n , ave}} ] \frac{Δ t}{2} & ( Equation\end{matrix}$$

for a process from t₀ to t versus measured charge. Equations (Equation 7.439), (Equation 7.440) and (Equation 7.441) are evaluated at the instantaneous boundary temperature, estimated via an average

$${T}_{ave} = \frac{{T}_{Batt} + {T}_{air}}{2} .$$

Equation (Equation 7.441) implies instantaneous reversibility. In FIGS. 79A and 79B, Ohmic entropy, similar to Ohmic work, appears linear against measured charge, with a change in slope in the transition region for discharge. Both Ohmic work G_{Ω} and Ohmic entropy S′_{Ω} are more significant in the ND region than the OD region; cycle 2 (Table 7.1) shows that 75% of total G_{Ω} and S′_{Ω} accumulated is in the normal discharge (ND) region (95% in cycle 1) even though over-discharge (OD) was thrice as long as ND in duration. ECT entropy, similar to ECT energy, trends with voltage. Unlike their Ohmic counterparts, both ECT energy drop G_{T} and entropy accumulated S′_{VT} are minimal in the ND region but significant in the OD region. In cycle 2 (Table 7.1), only 17% of G_{T} and S′_{T} lie in the ND region. With Ohmic work as the useful work out of the battery, this confirms the ND region to be more favorable for efficient operation; with over-discharge well known to be adverse to battery health, this also confirms above analysis regarding the contribution of S′_{VT} to process irreversibility and hence degradation. Note that while in FIGS. 78A-78B, horizontal coordinate is time, the horizontal coordinate in FIGS. 79A-79B is measured charge.

Total S′_{Ω} during discharge is slightly less than during charge, whereas total S′_{VT} during discharge is significantly higher than during charge, primarily due to over-discharging and secondarily voltage relaxation after discharge, i.e., when the external load is disconnected from the battery, the battery's potential V immediately starts to rise, indicating voltage elasticity and causing the voltage change during active charge to be less than during discharge. This contributes to the higher Coulombic efficiency of the charge step and indicates the charge process to be more favorable to the battery than the discharge process. This also indicates that the charge step is more reversible than the discharge step and hence generates less entropy—a reversible (ideal) step is one that proceeds at a constant current and no voltage drop. With each charge step proceeding at the low steady rate of 1.2 A, the charge entropy generation is determined primarily by the duration of charge.

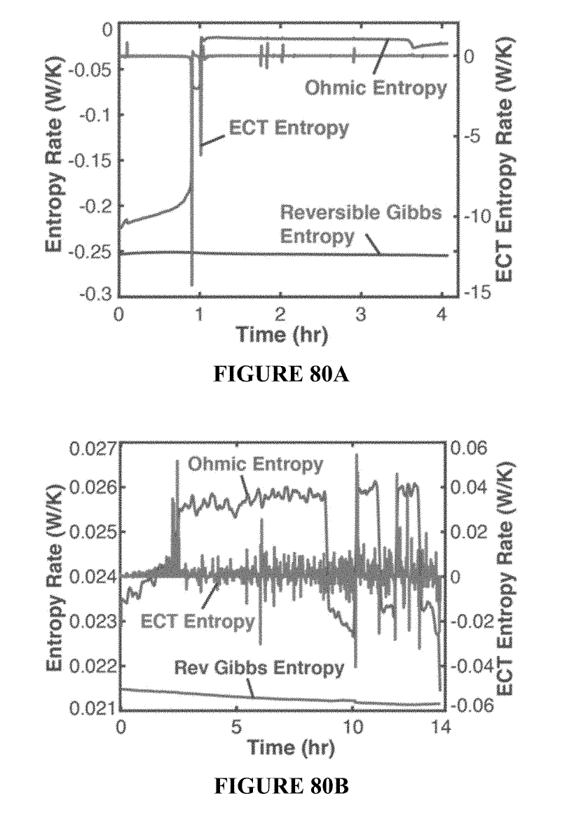

Ohmic entropy rate (FIGS. 80A-80B, left vertical axis) trends with voltage and current, starting relatively high during discharge (FIG. 80A) at 0.23 W/K with a significant drop in the T region to about 0.02 W/K in OD. ECT entropy generation rate (FIGS. 80A-80B, right axis label), correlating with voltage change rate, shows transients coinciding with sudden drops in voltage, starting at 0.02 W/K in the normal region and dropping to 0.003 W/K in the OD region. During charge (FIG. 80B), both Ohmic and ECT entropy rates are relatively slow—0.025 W/K and 0.0005 W/K respectively—with fluctuations, artefacts of the charge circuitry and battery response. Reversible Gibbs entropy rate (left vertical axis), showing the effects of instantaneous boundary temperature, is 0.253 W/K during discharge and 0.021 W/K during charge.

DEG Analysis

Capacity Versus Entropy-Degradation Coefficients

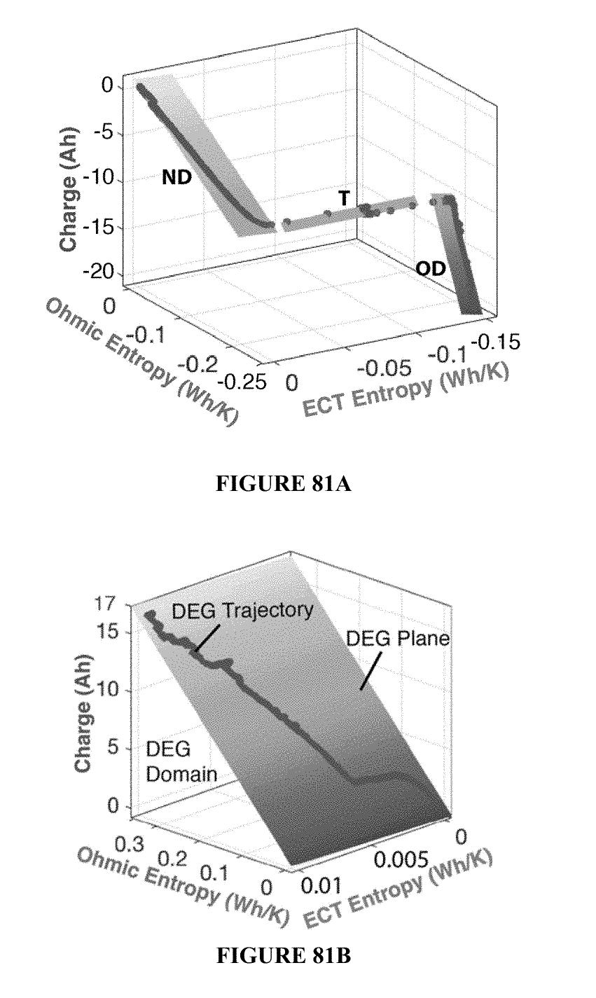

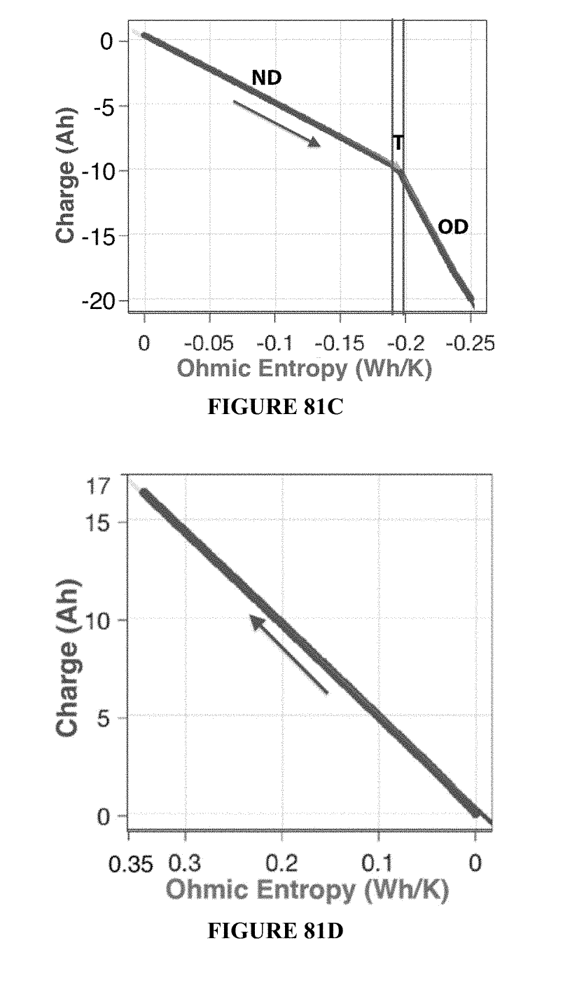

By associating data from various time instants, measured charge from equation (Equation 7.436) was plotted versus accumulated entropies S′_{Ω} and S′_{VT} in FIGS. 81A-81D for discharge (FIG. 81A) and charge (FIG. 81B). In these 3D plots, the [symbol] (or [symbol] ₜ) versus S′_{Ω} and S′_{VT} trajectories lie in planes whose orientations define the degradation coefficients B_{Ω} and B_{VT} of equation (Equation 7.426). FIGS. 81C-81D show coincidence of all the data points with planar 2D surfaces. A goodness of fit R²=1 with the 2D surfaces in these 3D plots for all cycles studied suggests the linear dependence of charge/capacity on both Ohmic and ECT entropies at every instant of the discharge/charge process, consistent with equation (Equation 7.431). The battery's Degradation-Entropy Generation (DEG) trajectory during discharge/charge lies solely on (coincides with) planar surface(s)—DEG plane(s)/surface(s). The 3D space of the DEG surface(s), the battery's DEG domain (here Charge versus Ohmic and ECT Entropies) seem(s) to characterize the allowable regime in which the battery can operate and can define consistent parameters for identifying desired characteristics of batteries of all configurations. In FIGS. 81C and 81D, end views of the discharge and charge DEG domains in FIGS. 81A and 81B respectively, suggest a perfect coincidence of the DEG trajectories with the DEG planes.

Degradation coefficients B_{Ω} and B_{VT}, partial derivatives of charge with respect to Ohmic and ECT entropies respectively, equation (Equation 4.257), were estimated as the partial slopes of the surfaces in FIGS. 81A-81D. The rate dependency of the coefficients necessitates a split of the entire discharge trajectory into 3 separate regions to accurately represent the cause/effect of the sudden drops in voltage:
normal discharge ND region, from start of discharge to 5V;
transition T region, from 5V to overdischarge voltage (the latter varying from cycle to cycle);
over-discharge OD region, from end of transition to end of over-discharge.

Hence three DEG planes characterize the entire discharge process, FIGS. 81A and 81C, one for each region, while the entire charge step is fully defined on one DEG plane (FIG. 81B, 81D). Vertical lines in 81C demarcate the ND, T and OD regions. Presented in Table 7.22, columns 2, 3, 8, 9, 10, 11 (discharge) and 16, 17 (charge) are values of coefficients obtained from entropy data in Table 7.1.

With the abusive and irregular discharge-charge cycling schedule (to accelerate and test battery degradation), Table 7.2 shows Ohmic degradation coefficient B_{Ω} is relatively consistent in the ND region for all cycles (53±1.5 Ah K/Wh), becomes larger in the T and largest in the OD regions with significant cycle-to-cycle variation in the latter. For charge, B_{Ω} is nearly constant over all cycles (48±1 Ah K/Wh). B_{Ω}>0 for both discharge and charge. B_{VT} shows significant cycle to cycle variations for both discharge and charge—predominantly positive for both discharge/charge steps with a few cycles reversing signs—indicating that the ECT characteristic of the battery is easily altered with every cycle, especially under severe irregular cycling as in these experiments. The T region has the lowest B_{VT} values, as predicted by equation (Equation 4.257): the sudden increase in entropy generation during transition is accompanied with minimal charge transfer. With the exception of a few over-discharge steps especially in the last few cycles ((15, 17-19), highest B_{VT} is observed during charge, also from equation (Equation 4.257): minimal ECT entropy accumulation with high (long duration) charge transfer. This is another confirmation that the charge step is more reversible than the discharge step. It is noted that commercial battery chargers often include temperature compensation during charging to further improve the reversibility/Coulombic efficiency of the charge process.

Capacity (Charge) Fade Δ [symbol] and Components

Capacity fade Δ [symbol] (columns 7, 15 and 21 in Table 7.2) defined in equation (Equation 7.435) as the difference between reversible and irreversible charge was obtained as follows:
Using B_{Ω} and B_{T} (Table 7.22's columns 2, 3, 10, 11, 16, 17) from the DEG domain, obtain the step's irreversible charge [symbol] ᵢᵣᵣ (columns 5, 13 and 19) from equation (Equation 7.431).

For cycle 2's normal discharge, row 2 of Table 7.2, B_{Ω}=51.9 Ah K/Wh, B_{VT}=13.8 Ah K/Wh, S′_{Ω}=−0.19 Wh/K, S′_{VT}=−0.03 Wh/K, giving irreversible charge, equation, [symbol] ᵢᵣᵣ=−10.0 Ah. (Equation 7.431)
Obtain the cycle's reversible charge [symbol] ᵣₑᵥ (columns 6, 14 and 20) from equation (Equation 7.433), with Iᵣₑᵥ as the reversible current. For the discharge step, Iᵣₑᵥ is estimated using the battery's measured open-circuit voltage V_{OC} before start of discharge and the resistance across the battery's terminals immediately after start of discharge (start of the ND region, where t=t₁) R_{B}=V_{B}/I where I is the externally measured discharge current. Similarly for the charge step, Iᵣₑᵥ=V_{OC}/R_{B} is estimated using R_{B} obtained at the end of charge (lowest current during the charge step is typically at the end of the saturation charge phase).

For cycle 2's discharge, Iᵣₑᵥ=−11.9 A, hence in the ND region, [symbol] ᵣₑᵥ=Iᵣₑᵥ*Δt₂=11.0 Ah (where cycle 2's ND duration Δt₂=0.924 hr [28]). Measured charge [symbol] ₜ=−9.9 Ah, row 2 of Table 7.2.
Evaluate capacity fade Δ [symbol] from the first equality in equation (Equation 7.434), [symbol] ᵢᵣᵣ− [symbol] ᵣₑᵥ.

For cycle 2's ND, Δ [symbol] =−10.0−−11.0=1.0 Ah. OD, Δ [symbol] =27.8 Ah and for charge, Δ [symbol] =2.4 Ah.

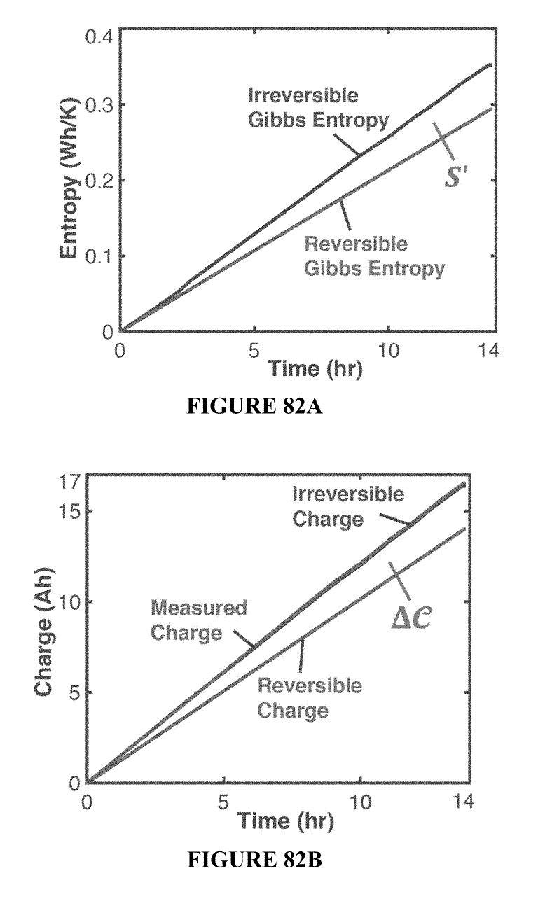

Table 7.2 shows capacity fade in the battery during discharge and charge for all 19 cycles measured. To visualize and directly compare the instantaneous in-operation trends in entropy and capacity during the charge step, FIGS. 82A-82B plot the components of entropy generation (FIG. 82A)—irreversible Gibbs entropy S′ᵢᵣᵣ and reversible Gibbs entropy S′ᵣₑᵥ—and capacity fade (FIG. 82B)—measured charge [symbol] ₜ, irreversible charge [symbol] ᵢᵣᵣ, and reversible charge [symbol] ᵣₑᵥ—over time during the charge step of cycle 2. The regions between reversible and irreversible components are the entropy generation S′ (FIG. 82A) and capacity fade Δ [symbol] (FIG. 82B). It is observed that, as anticipated in the theoretical formulations discussed above, during charge, S′ᵢᵣᵣ>S′ᵣₑᵥ and [symbol] ₜ> [symbol] ᵢᵣᵣ> [symbol] ᵣₑᵥ. The charge step had minimal capacity fade with practically linear components as anticipated by entropy analyses in previous sections, again verifying the relative reversibility of this step as it proceeds at a low quasi-steady current of 1.2 A (low entropy generation rate). Also, with reduced irreversibilities (low ECT entropy), [symbol] ᵢᵣᵣ≈ [symbol] ₜ during charge (FIG. 82B and Table 7.2's columns 18 and 19). A breakdown of the discharge step follows in the next subsection.

Entropy Generation and Capacity Fade Breakdown-Discharge Regions

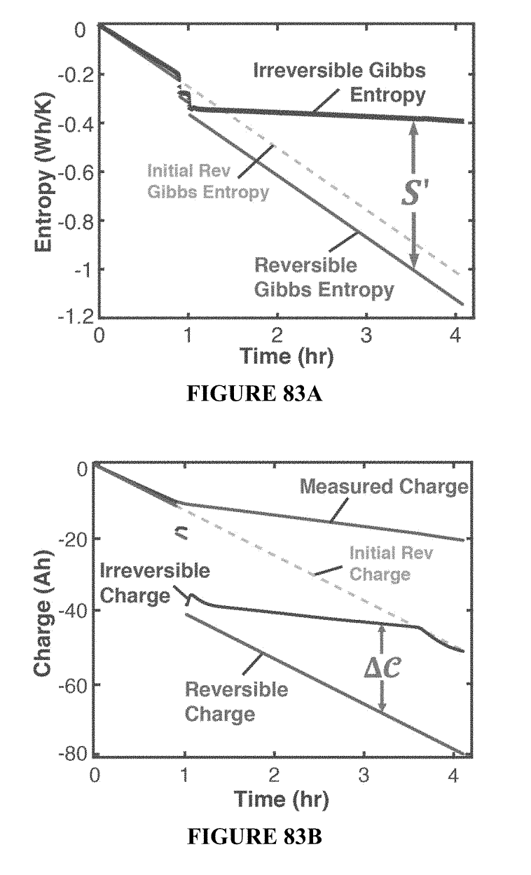

The severe nonlinearities during discharge are observed in the correspondingly nonlinear entropy generation accumulation. FIGS. 83A-83B, 84A-84B, 85A-85B, and 86A-86B plot the components of entropy generation—irreversible Gibbs entropy S′ᵢᵣᵣ and reversible Gibbs entropy S′ᵣₑᵥ along with the latter's initial trend—and (FIGS. 83B, 84B, 85B, and 86B) capacity fade—measured charge [symbol] ₜ, irreversible charge [symbol] ᵢᵣᵣ, and reversible charge [symbol] ᵣₑᵥ along with the latter's initial trend—over time during cycle 2's discharge step. The regions between reversible and irreversible components are the entropy generation S′ and capacity fade Δ [symbol] . As in the case of the charge step in FIGS. 82A-82B, observed trends during discharge S′ᵢᵣᵣ<S′ᵣₑᵥ and [symbol] ₜ< [symbol] ᵢᵣᵣ< [symbol] ᵣₑᵥ are in agreement with prior formulations and analyses. Sudden increase corresponding to the transition from normal to over-discharge is observed in all the plots except measured charge [symbol] ₜ, making the latter unreliable for nonlinear degradation or state of health analysis. (Note that signs are for direction of process not magnitude).

Normal Discharge

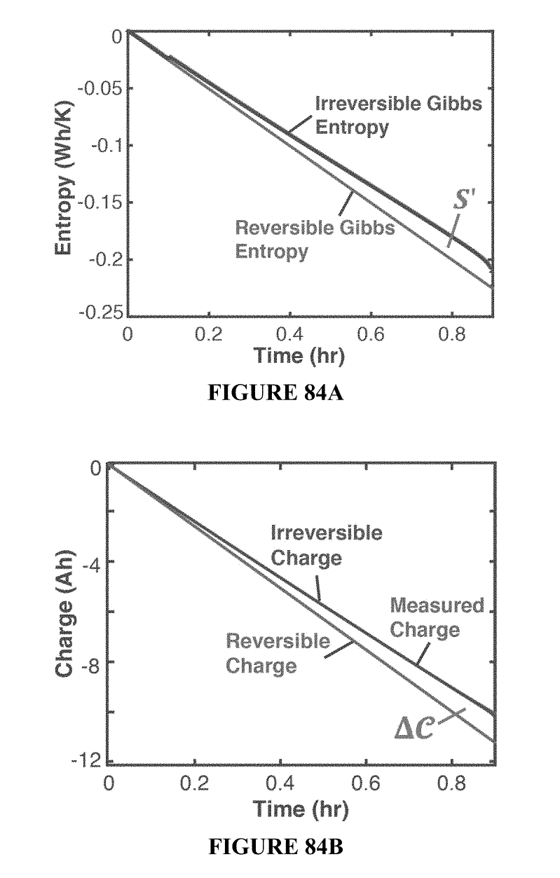

In the normal discharge ND region, with relatively slow changes in voltage and current, FIG. 84A shows that S′ᵢᵣᵣ and S′ᵣₑᵥ are relatively close and hence entropy generation is relatively low. Table 7.2 (columns 4, 5 and 6) and FIG. 84B also show that all forms of charge— [symbol] ₜ, [symbol] ᵢᵣᵣ, and [symbol] ᵣₑᵥ—are relatively close in magnitude and hence capacity fade is low. As in the charge step, reduced irreversibilities (low ECT entropy) give [symbol] ᵢᵣᵣ≈ [symbol] ₜ in the ND region (FIG. 84B and Table 7.2's columns 4 and 5), verifying that Coulombic capacity fade, equation (Equation 7.429), is indeed the same as DEG's capacity fade, equation (Equation 7.435), applied to consistent linear cycling, as earlier anticipated by a comparison of both equations in which [symbol] ᵣₑᵥ is defined by the first cycle and subsequently held constant. The observed difference in reversible and irreversible components in FIGS. 84A-84B is primarily due to the voltage and current drop from battery's initial state (the point of [symbol] ᵣₑᵥ establishment defined by V_{OC}, Iᵣₑᵥ) when the external load is connected to the battery; actual loss during normal discharge is minimal and the battery recovers more easily and lasts longer when used in this region only, as required by most applications. An ideal constant-voltage, constant-current source generates no entropy and is fully reversible.

Transition

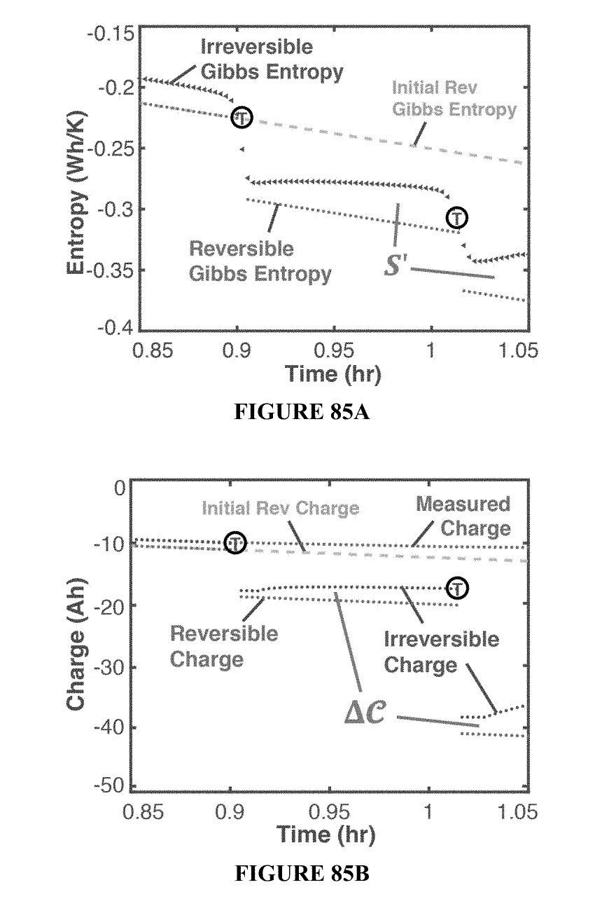

In the transition T region, the abrupt increase in ECT entropy due to the sudden voltage drop is subsequently observed in entropy generation components. For the starter batteries tested, a 2-step transition was observed. The first transition drop in voltage to 3.5 V (and corresponding drop in current) was followed by an attempt by the battery to stabilize. Unable to do so, another abrupt drop in battery voltage to ˜1.7 V occurs and the battery stabilizes at this voltage for the over-discharge duration. In FIGS. 85A-85B, this is shown to cause a 2-step sudden increase in magnitude of S′ᵢᵣᵣ corresponding to both transition steps. B coefficients obtained from the transition T DEG surface are used in conjunction with irreversible entropy components in this region (equation (Equation 7.431)) to obtain irreversible charge [symbol] ᵢᵣᵣ (FIG. 85B) as demonstrated above. As mentioned earlier, measured charge [symbol] ₜ does not indicate this sudden transition; the change of slope observed in [symbol] ₜ is the same as if the external load was reduced to lower the discharge rate with no drop in battery voltage, hence the mere change in slope of measured charge does not adequately represent the actual and more significant change that occurred in the battery in the transition region. Via the DEG theorem, the severe nonlinearities in irreversible Gibbs entropy S′ᵢᵣᵣ are transferred to irreversible charge [symbol] ᵢᵣᵣ, the latter fully characterizing the battery at every instant and under all conditions.

After the transition S′ᵢᵣᵣ and [symbol] ᵢᵣᵣ have been evaluated, the observed shift is applied to reversible terms S′ᵣₑᵥ (FIG. 85A) and [symbol] ᵣₑᵥ (FIG. 85B) to adjust them to the battery's new state, while maintaining the same slopes as the initial reversible terms, an instantaneous reference to the initial state.

Over-Discharge

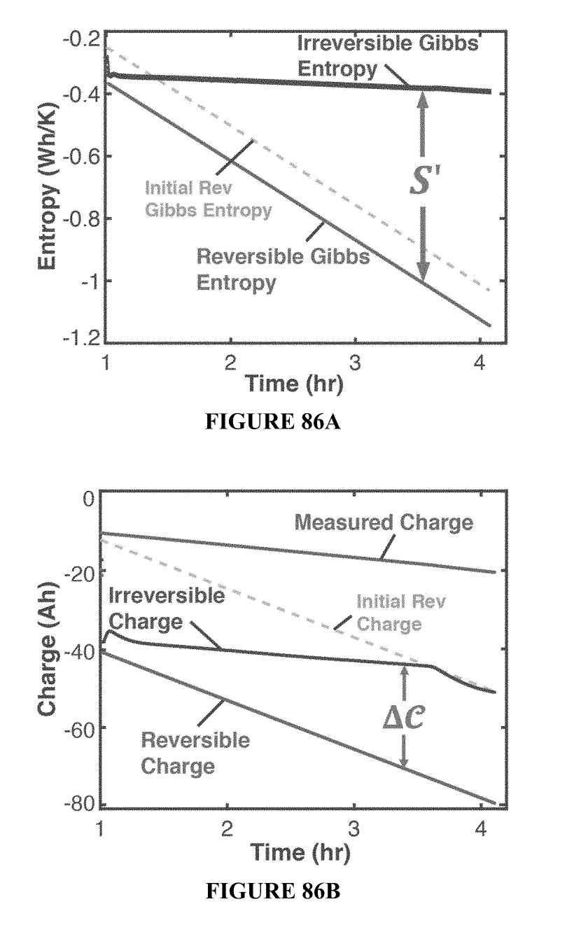

At the end of the transition region, the battery stabilizes to the new voltage and current and continues to over-discharge for a long duration (3 hours in cycle 2). S′ᵢᵣᵣ and [symbol] ᵢᵣᵣ continue to accumulate at a slower rate than the normal region while S′ᵣₑᵥ and [symbol] ᵣₑᵥ proceed at the same initial rate (FIGS. 86A-86B). This significantly increases entropy generation S′ and capacity fade Δ [symbol] in the OD region; also in accordance with experience as the battery is severely stressed here. Measured charge [symbol] ₜ proceeds linearly at the reduced discharge rate or current I(t) (slope of [symbol] ₜ(Δt)) with no indication of the significant drop in voltage.

The battery's discharge breakdown is analogous to an athlete who starts to sprint with a 10-kg bag strapped to their back. The athlete sprints (normal discharge at high discharge current) until they can no longer sustain their initial speed due to exhaustion (loss of potential or transition to lower potential) and rather than stop to re-energize (recharge step), continues the race at a jog (over-discharge at lower discharge current) carrying the same load (still connected to the same initial external resistive load). Note that for most operations, the battery has failed at the end of normal (full) discharge, i.e. the battery is no longer capable of powering the device at the required voltage and the sudden power loss from the drop in voltage and current could be harmful to electrical/electronic components. Also, in addition to degrading the battery faster, over-discharging the battery is not safe—catastrophic thermal events could occur as the battery gets progressively unstable.

Cycling Summary

Tables 7.1 and 7.2 show that steps with high electrochemicothermal (ECT) entropy (relative to Ohmic entropy) have high capacity fade (discharge steps 10-16). Cycle 1's discharge step has a significantly longer normal discharge ND region than subsequent cycles, hence highest discharge capacity fade in this region, see Table 7.2's column 7. Overall, lowest total discharge fades are observed in the first half of the cycling experiments (cycles 1-9) while highest fades are observed in the second half after discharge rate was tripled (cycles 10-19). With each charge step proceeding at the relatively low current, the charge entropy generation is determined primarily by the duration of charge, hence charge steps with more accumulated charge tend to have higher accumulated entropy generation and hence, charge capacity fade, evident in the first half (cycles 2-9) of the cycling experiments. Note that cycle 1's charge step is significantly shorter in duration than other charge steps to accelerate degradation—a discharge (including over-discharge) of 61.3 Ah was followed by a recharge of 6.1 Ah in cycle 1.

SUMMARY rows of Tables 7.1 and 7.2 show the overall changes in the battery throughout cycling. In Table 7.1, the ratio of total Ohmic entropy S′_{Ω} to total ECT entropy S′_{VT} for all the discharge steps throughout cycling is 1.9:1 (9.1:1 for the ND region only) while all the charge steps collectively accumulated S′_{Ω}:S′_{VT} at a much more thermodynamically reversible ratio of about 33:1. The SUMMARY row of Table 7.1 also shows that total discharge S′_{VT} is 15 times charge S′_{VT} (viz higher entropy generation rate from discharge rate >>charge rate and severe over-discharging) while total S′_{Ω} is higher for charge than discharge (viz significantly longer and more efficient charge step). Total reversible entropy S′ᵣₑᵥ is slightly higher than irreversible entropy in the ND region and much more in the OD region during discharge, but slightly less during charge in accordance with above discussions.

Due to the severely inconsistent cycling in the experiments, Coulomb-counted capacity fade (equation (Equation 7.429)) is not applicable to the measured data and hence was not evaluated for comparison to DEG capacity fade. In Table 7.2, DEG's ND/OD discharge capacity fades of 19.7 Ah/254.7 Ah (first half) and 8.9 Ah/781.9 Ah (second half) suggest that most of the battery's loss of capacity (92%—first half and 98%—second half) occurred in the over-discharge region. Hence the entire step is required for accurate capacity fade estimate.

Discussion

In Prigogine's study of time-dependent entropy generation and nonlinearities, Prigogine introduced a universally non-positive (for macroscopic systems undergoing spontaneous processes) and interaction-specific “local potential” analogous to the ECT energy introduced in this study, which he obtained from a decomposition of his irreversible entropy generation formulation—the product of thermodynamic force and flow. Building on Prigogine's successful extension of hitherto reversible thermodynamic formulations to irreversible and non-equilibrium processes and states, this study, using an entirely different approach, derived and experimentally verified, in above sections, universally consistent system-based time-dependent entropy generation. It was shown that
entropy generation is the difference between irreversible S′ᵢᵣᵣ and reversible S′ᵣₑᵥ Gibbs entropies at every instant;
entropy generation is always non-negative in accordance with the second law whereas both its components S′ᵢᵣᵣ and S′ᵣₑᵥ are directional, positive during charge and negative during discharge. This implies |S′ᵢᵣᵣ|≥|S′ᵣₑᵥ| during charge and |S′ᵢᵣᵣ|≤|S′ᵣₑᵥ| during discharge in accordance with experience and established thermodynamic laws. (Modulus sign emphasizes magnitude only).

Features of the DEG Theorem and B Coefficients
The DEG theorem provides a structured approach to battery degradation modeling, instead of heuristic empirical methods of measure everything, plot everything versus everything, find correlations, then do numerous curve fits.
The methods of the DEG theorem can accurately describe the battery's charge levels within a discharge-charge cycle versus entropy components, and the capacity fade over multiple discharge-charge cycles, since the dissipative entropy generating processes underlie discharge-charge cycles.
DEG coefficients relate accumulated entropy generation to operational capacity fade in a rechargeable battery at any point in the battery's life using simple non-destructive and non-intrusive measurements, without prior history or capacity information from the manufacturer/supplier. These coefficients show the battery's true response to prevalent processes and conditions by quantifying the processes' individual contributions to the battery's degradation.
Boundary interaction DEG coefficients such as Ohmic coefficient B_{Ω} are always positive by definition, equation (Equation 4.257)—irreversible entropy components and charge transfers are negative during discharge and positive during charge—indicating positive contribution towards a transformation vector. ECT coefficient B_{VT} has varying sign characteristic.
DEG theorem supports disorganization implied by entropy and the second law of thermodynamics, becoming a reverse confirmation of the second law.
The DEG theorem converts degradation failure design into a multi-dimensional geometry problem. The volume spanned by entropy trajectories defines the operating and ageing region. Irreversible entropy components serve as basis functions for the multi-dimensional irreversible transformation function space of [symbol] versus S′ᵢ (where i=number of active processes), as implied by DEG.

A combination of equation (Equation 7.398) and the DEG theorem fully defines the irreversible and reversible transformation paths for all batteries.

DEG Trajectories, Surfaces and Domains

Thermodynamics authors have used multi-dimensional orthogonal space to describe thermodynamic states of reversible processes—Callen's thermodynamic configuration space, Messerle's energy surface, and Burghardt's equilibrium surface. This study introduces the DEG domain, a multi-dimensional space that characterizes a system's irreversible (actual and possible) and reversible (ideal or at the limit of possibility) transformation paths. Proper formulation of the governing entropies of the active processes is required to accurately determine their contributions to overall accumulation and degradation during each half of a discharge-charge cycle.

DEG trajectories appear to be characteristic of cycle conditions, DEG surfaces appear to be characteristic of a battery's discharge/charge rates and the DEG domain seems to characterize the battery for all cycles and all rates. A battery having a domain with large accumulated charge dimension and small ECT entropy dimension (relative to Ohmic entropy dimension) delivers power more efficiently. Of the three surfaces for the discharge step, the ND surface has the longest Ohmic entropy dimension and a short ECT dimension, while the transition T (failure) region has the longest ECT dimension and shortest Ohmic entropy dimension.

For a range of discharge rates, a set of DEG surfaces exists which defines all possible DEG trajectories during operation. This is observed, where the ND-OD transition from 11 A (6 V) to 3 A (1.7 V) discharge rate caused a rotation in the DEG trajectory, with the T and OD portions of the trajectory laying on other DEG planes with orientations different from the ND DEG surface. Plots of the DEG trajectories for all the discharge steps in Tables 7.1 and 7.2 (cycles 1-19) and a DEG surface from cycle 1's discharge step, support a characteristic DEG surface defining the operational path and about which are all the DEG lines the battery can “draw” at a given discharge/charge rate.

Degradation Measure-Operational Capacity (Charge Transfer)

The DEG theorem is flexible for choice of degradation/transformation measure. Here charge transfer, also a direct measure of instantaneous charge levels in the battery relative to a constant initial state [symbol] ₀, was used to quantify the effects of voltage, current and temperature changes in the battery. Evaluated via Coulomb/current counting, measured charge transfer [symbol] ₜ(Δt)=∫ₜ_{₀}^{t}I(t) dt, often termed capacity, has always been considered inadequate for estimating all the actual changes taking place in a battery, but continues in industry and consumer goods for simplicity in rating a battery's utility (how much charge can the battery deliver at a specific voltage or voltage range). To align with this simplicity of utility, researchers and engineers use Coulomb-counted capacity fade, equation (Equation 7.429), to estimate battery degradation, with specific constraints: consistent cyclic depth of discharge DoD, constant cycling rate/current I (preferably low to minimize temperature change), linear cycling only (no over-charge or over-discharge), and only computes at end states (after entire discharge or charge step). Via the DEG coefficients, irreversible entropy is transformed onto the charge axis resulting in the irreversible charge transfer [symbol] ᵢᵣᵣ, without any of the afore-listed limitations of measured charge transfer [symbol] ₜ. Any degradation/transformation measure with a time basis can be used in conjunction with the DEG theorem to determine instantaneous nonlinear transformations in any system.

ECT Coefficient B_{VT}

ECT coefficient B_{VT} can be positive or negative, as observed in experimental data, Table 7.2. To understand B_{VT} sign changes, recall equation (Equation 7.431) rewritten as

[symbol]

ᵢᵣᵣ=B_{VT}S′_{VT}+B_{Ω}S′_{Ω} (Equation

Rearranging,

$${B}_{VT} = \frac{1}{{S}_{VT}^{′}} ( {irr}_{} - {B}_{Ω} {S}_{Ω}^{′} ) \qquad ( Equation$$

In equation (Equation 7.443), both irreversible charge [symbol] ᵢᵣᵣ and Ohmic charge B_{Ω}S′_{Ω} proceed in the same direction—negative during discharge and positive during charge. The expression in the bracket is the ECT charge, which changes signs depending on conditions in the battery (positive when | [symbol] ᵢᵣᵣ|<|B_{Ω}S′_{Ω}| during discharge, and vice versa) as anticipated by the fluctuations in ECT entropy generation rate and the initial temperature rise followed by endothermic cooling that took place during the discharge steps for the starter batteries tested.

It is noted that studies have shown the dependency of the battery's open-circuit voltage V_{OC} on temperature T via an entropic voltage-temperature coefficient (dV_{OC}/dT) measured at equilibrium points (during relaxation, before and after an active Ohmic step). These studies use path-independent reversible entropy change formulations, giving experimentally verified linear relationship between voltage and temperature. However, ECT coefficient B_{VT}=∂ [symbol] /∂S′_{VT}, obtained from path-dependent charge and ECT entropy accumulation, shows a nonlinear instantaneous battery voltage response at all times and under all conditions.

Critical Failure Entropy S′_{CF}

The DEG theorem also establishes that if a critical value of degradation measure exists, at which failure occurs, there must also exist critical values of accumulated irreversible entropies, and the relationship between them has also been hypothesized in an independent study by Sosnovskiy and Sherbakov. Naderi and Khonsari using exhaustive experimental data, showed the existence of a material-dependent fatigue fracture entropy FFE.

Above, the abrupt drops in voltage at the transition from ND to OD are shown to coincide with abrupt increases in irreversible Gibbs entropy S′ᵢᵣᵣ. With reversible Gibbs entropy S′^{rev}, proceeding steadily, S′ᵢᵣᵣ suddenly exceeds S′ᵣₑᵥ, making entropy generation S′, equation (Equation 7.423), negative at the transition points. Recall previous discussion of how the end of ND region is considered full discharge and hence transition points considered failure points. As mentioned earlier, the dashed lines plot the discharge step's original reversible path before the first transition. In FIG. 85A, the irreversible path is seen to intersect this original reversible path and hence a negative entropy generation is observed momentarily. The second law of thermodynamics prohibits negative entropy generation for a real process to continuously occur, verifying that the point of negative entropy generation coincides with the battery's critical failure—sudden loss of ability/potential (voltage) to supply power to the external load. This point of entropy generation transition from positive to negative is the Gibbs-based DEG's Critical Failure Entropy. In this example, the point in time at which the battery's voltage is just above 5 V consistently marks the start of rapid decline in voltage during each cycle's discharge step and hence the cyclic S′_{CF} point. A monitoring mechanism with a >5 V criterion will consistently prevent critical failure (transition to over-discharge), explaining why battery management/optimizing systems rely on voltage level as the over-charge and over-discharge limiter.

The Thermal Entropy Component in the Maximum Work Formulations.

In the equation for Gibbs energy changes in a battery, dG=−SdT+Vdq, the entropy content S is typically known to depend on thermal and chemical changes in a system. In a non-reactive system, S varies as the heat capacity C of the system, so that the SdT term approximates thermal changes only. As used herein, thermal energy/entropy may be discussed in relation to a thermal approach. However, it is noted, via the Gibbs Duhem formulation, that the internal dissipation term can be posed in every system—whether reactive or non-reactive. For example, for a reactive system such as batteries, the ECT term can be specified. As such, methodology and formulation, as discussed herein (e.g, in Example 7) can be applied to grease analysis and fatigue analysis, particularly, in which the ST (structuro-thermal) term is used to account for microstructure and thermal changes.

### SUMMARY AND CONCLUSION

In this study, irreversible thermodynamics and the Degradation-Entropy Generation theorem were applied to lead-acid battery degradation, particularly to the evaluation of capacity fade. Thermodynamic breakdown of the active processes in batteries during cycling was presented, using Gibbs energy-based formulations. Via anticipatory Maximum work-based entropy generation evaluation, DEG's battery capacity fade model was formulated and experimentally verified with consistent results. Severely nonlinear experimental data were processed and analyzed using the DEG's capacity fade model with consistent results. All four batteries tested, including those in the Appendix, showed similar trends. The lead-acid batteries showed only slight degradation from the charge steps but significant degradation from the abusive discharge steps.

A thermodynamic potential—Gibbs free energy—replaced steady state assumptions in previous DEG applications, and employed the instantaneous applicability of the first and second laws of thermodynamics. These form the deductive apparati upon which the validity of the DEG theorem has been proven and experimentally demonstrated in this study, reverse-verifying the second law of thermodynamics. The usual problem emanating from the often nonlinear nature of battery cycling, which renders state of health SoH and capacity fade estimation via Coulomb counting inappropriate for estimating cycle to cycle changes, was solved using instantaneous irreversible thermodynamic formulations. The significance of ECT entropy in establishing the battery's true irreversible path, hence entropy generation, was shown and is underscored by the need to avoid over-discharging and keep batteries cool during operation, for better and longer performance.

The methodology can directly compare technologies, designs and materials used in lithium-ion battery manufacture. Without any prior information from the manufacturer, measurements and appropriate data analyses via the DEG theorem can determine the most suitable battery for an application. The DEG theorem relates accumulated irreversibilities to the resulting damage in systems using dimensional entropy generation components. This study successfully applied DEG to very nonlinear lead-acid battery cycling using non-intrusive measurements of only temperature and the primary interaction's conjugate parameters—in this case, voltage and current—with consistent results. Without being material- or system-dependent, the DEG theorem is material- and system-specific making it easily and consistently adaptable to all systems undergoing real processes.

Further, in addition to design analysis and optimization, the methodology can be directly employed for non-intrusive and non-destructive real-time monitoring of battery's State of Health (SoH). Further, in addition to design analysis and optimization, the methodology can be directly employed for non-intrusive and non-destructive real-time monitoring of grease and its State of Health (SoH). Further, in addition to design analysis and optimization, the methodology can be directly employed for non-intrusive and non-destructive real-time monitoring of structure (with respect to mechanical life of a structure) and its State of Health (SoH).

The systems, and methods of the appended claims are not limited in scope by the specific systems, and methods described herein, which are intended as illustrations of a few aspects of the claims. Any systems, and methods that are functionally equivalent are intended to fall within the scope of the claims. Various modifications of the systems, and methods in addition to those shown and described herein are intended to fall within the scope of the appended claims. Further, while only certain representative systems and method steps disclosed herein are specifically described, other combinations of the systems, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

As used herein, “computer” may include a plurality of computers. The computers may include one or more hardware components such as, for example, a processor, a random access memory (RAM) module, a read-only memory (ROM) module, a storage, a database, one or more input/output (I/O) devices, and an interface. Alternatively and/or additionally, computer may include one or more software components such as, for example, a computer-readable medium including computer executable instructions for performing a method associated with the exemplary embodiments. It is contemplated that one or more of the hardware components listed above may be implemented using software. For example, storage may include a software partition associated with one or more other hardware components. It is understood that the components listed above are exemplary only and not intended to be limiting.

Processor may include one or more processors, each configured to execute instructions and process data to perform one or more functions associated with a computer for indexing images. Processor may be communicatively coupled to RAM, ROM, storage, database, I/O devices, and interface. Processor may be configured to execute sequences of computer program instructions to perform various processes. The computer program instructions may be loaded into RAM for execution by processor.

RAM and ROM may each include one or more devices for storing information associated with operation of processor. For example, ROM may include a memory device configured to access and store information associated with the computer including information for identifying, initializing, and monitoring the operation of one or more components and subsystems. RAM may include a memory device for storing data associated with one or more operations of processor. For example, ROM may load instructions into RAM for execution by processor.

Storage may include any type of mass storage device, including network-based storage, configured to store information that processor may need to perform processes consistent with the disclosed embodiments. For example, storage may include one or more magnetic and/or optical disk devices, such as hard drives, CD-ROMs, DVD-ROMs, or any other type of mass media device.

Database may include one or more software and/or hardware components that cooperate to store, organize, sort, filter, and/or arrange data used by the computer and/or processor. For example, database may store the source CAD model and parameters to generate the three-dimensional meta-structure models therefrom. It is contemplated that database may store additional and/or different information than that listed above.

I/O devices may include one or more components configured to communicate information with a user associated with computer. For example, I/O devices may include a console with an integrated keyboard and mouse to allow a user to maintain a database of images, update associations, and access digital content. I/O devices may also include a display including a graphical user interface (GUI) for outputting information on a monitor. I/O devices may also include peripheral devices such as, for example, a printer for printing information associated with controller, a user-accessible disk drive (e.g., a USB port, a floppy, CD-ROM, or DVD-ROM drive, etc.) to allow a user to input data stored on a portable media device, a microphone, a speaker system, or any other suitable type of interface device.

Interface may include one or more components configured to transmit and receive data via a communication network, such as the Internet, a local area network, a workstation peer-to-peer network, a direct link network, a wireless network, or any other suitable communication platform. For example, interface may include one or more modulators, demodulators, multiplexers, demultiplexers, network communication devices, wireless devices, antennas, modems, and any other type of device configured to enable data communication via a communication network.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect.

While the methods and systems have been described in connection with certain embodiments and specific examples, it is not intended that the scope be limited to the particular embodiments set forth, as the embodiments herein are intended in all respects to be illustrative rather than restrictive.

Unless defined otherwise, all technical and scientific terms used herein have the same meanings as commonly understood by one of skill in the art to which the disclosed invention belongs. Publications cited herein and the materials for which they are cited are specifically incorporated by reference.

What is claimed is:

1. A method to estimate entropy in a dissipative process of a system, wherein the estimation is used to measure degradation and/or expected failure of a system, the method comprising:
obtaining, by a processor, in-situ control data set or experimental data set associated with a dissipative or thermal process of a system, wherein the control or experimental data set is acquired to assess a degradation measure and to assess an entropy production for the dissipative process;
determining, by the processor, one or more degradation coefficients from the control or experimental data, wherein each of the one or more degradation coefficients is determined as a rate of change of one or more assessed degradation measure parameters with respect to one or more assessed entropy production parameters for the dissipative or thermal process, wherein the rate of change is determined as a slope of a first coordinate axis associated with the one or more assessed degradation measure parameters and of one or more second coordinate axes each associated with an assessed entropy production parameter associated with the dissipative or thermal process; and
determining, by the processor, one or more parameters associated with a measure of degradation and/or expected failure of the system, wherein the determination of the one or more parameters is based on an assessed estimated entropy parameter associated with estimated entropy produced by the dissipative process by linearly combining each of the one or more determined degradation coefficients with at least one corresponding assessed accumulated irreversible entropy parameter, and
wherein the one or more parameters associated with the degradation and/or expected failure, or value(s) associated therewith, of the system is used in an evaluation of the system for use in engineering application or in the control, optimization, or maintenance of said system in said engineering application.

2. The method of claim 1, wherein the one or more assessed degradation measure parameters associated with the first coordinate axis and the one or more assessed entropy production parameters associated with the one or more second coordinate axes, collectively, correspond to a multi-dimensional surface, and wherein the slope assessed on said multi-dimensional surface corresponds to a degradation entropy generation (DEG) trajectory.

3. The method of claim 1, further comprising:
collecting, in a control loop of the system, the in-situ the control data associated with the dissipative process.

4. The method of claim 1, further comprising:
performing the experiment to collect experimental data for estimation of entropies in the dissipative process of the system.

5. The method of claim 1, wherein the dissipative process is selected from the group consisting of battery degradation, grease degradation, and structural degradation due to fatigue.

6. The method of claim 1, wherein the dissipative process is selected from the group consisting of degradation associated with friction, degradation associated with turbulence, degradation associated with spontaneous chemical reaction, degradation associated with inelastic deformation, degradation associated with fretting, degradation associated with free expansion of gas or liquid, degradation associated with flow of electric current through a resistance, and degradation associated with hysteresis, and wherein the estimation is used to measure degradation and/or expected failure of a system.

7. The method of claim 1, wherein the dissipative process is associated with battery degradation,
wherein the obtained in-situ control data set or experimental data set is used to determine, by the processor, a first set of degradation coefficients based on linear dependence of capacity accumulation on irreversible entropies;
wherein the measure of degradation and/or expected failure of the system derived based on the first degradation coefficients set used to assess battery cycle life or remaining battery cycle life.

8. The method of claim 1, the obtained in-situ control data set or experimental data set is associated with active thermal process of the system with respect to battery degradation, the method comprising:
determining, by the processor, a second degradation set of coefficients based on linear dependence of capacity accumulation on thermal entropies;
wherein the measure of degradation and/or expected failure of the system derived based on the second degradation coefficients set used to assess battery cycle life or remaining battery cycle life.

9. The method of claim 1, wherein the obtained in-situ control data set or experimental data set is associated with active dissipative or thermal process(es) of the system with respect to battery degradation, the method comprising
determining, by the processor, a first set of degradation coefficients based on linear dependence of capacity accumulation on irreversible entropies; and
determining, by the processor, a second degradation set of coefficients based on linear dependence of capacity accumulation on thermal entropies;
wherein the measure of degradation and/or expected failure of the system derived based on the first and second degradation coefficients sets are used to assess battery cycle life or remaining battery cycle life.

10. The method of claim 7, wherein the system comprises a lead-acid battery or a lithium-ion battery.

11. The method of claim 1, wherein the dissipative process is associated with grease degradation,
wherein the obtained in-situ control data set or experimental data set is used to determine, by the processor, a first set of degradation coefficients based on linear dependence between assessed shear stress and irreversible entropies;
wherein the measure of degradation and/or expected failure of the system derived based on the first degradation coefficients set is used to assess grease life or remaining grease life.

12. The method of claim 1, wherein the obtained in-situ control data set or experimental data set is associated with an active thermal process of the system with respect to grease degradation, the method comprising:
determining, by the processor, a second set of degradation coefficients based on linear dependence of shear stress on thermal entropies;
wherein the measure of degradation and/or expected failure of the system derived based on the second degradation coefficients set is used to assess grease life or remaining grease life.

13. The method of claim 1, wherein the obtained in-situ control data set or experimental data set is associated with active dissipative or thermal process(es) of the system with respect to grease degradation, the method comprising
determining, by the processor, a first set of degradation coefficients based on linear dependence between assessed shear stress and irreversible entropies;
determining, by the processor, a second set of degradation coefficients based on linear dependence of shear stress on thermal entropies;
wherein the measure of degradation and/or expected failure of the system derived based on the first and second degradation coefficients sets are used to assess grease life or remaining grease life.

14. The method of claim 1, wherein the obtained in-situ control data set or experimental data set is associated with the active dissipative process(es) of the system with respect to structural degradation due to fatigue, the method comprising:
determining, by the processor, a first set of degradation coefficients based on linear dependence between assessed mechanical stress and irreversible entropies;
wherein the measure of degradation and/or expected failure of the system derived based on the first degradation coefficients set is used to assess mechanical life or remaining mechanical life of a structure.

15. The method of claim 1, wherein the obtained in-situ control data set or experimental data set is associated with the active dissipative process(es) of the system with respect to structural degradation due to fatigue, the method comprising:
determining, by the processor, a second set of degradation coefficients (e.g., BW_{D} and BT_{D}) based on linear dependence between assessed CDM damage and irreversible entropies;
wherein the measure of degradation and/or expected failure of the system derived based on the second degradation coefficients set is used to assess mechanical life or remaining mechanical life of a structure.

16. The method of claim 1, wherein the obtained in-situ control data set or experimental data set is associated with the active dissipative process(es) of the system with respect to structural degradation due to fatigue, the method comprising:
determining, by the processor, a third set of degradation coefficients based on linear dependence between assessed normalized cycles (N/N_{f}) and irreversible entropies;
wherein the measure of degradation and/or expected failure of the system derived based on the third degradation coefficients set is used to assess mechanical life or remaining mechanical life of a structure.

17. The method of claim 1, wherein the obtained in-situ control data set or experimental data set is associated with an active thermal process of the system with respect to structural degradation due to fatigue, the method comprising:
determining, by the processor, a fourth set of degradation coefficients based on linear dependence between assessed stress and thermal entropies;
wherein the measure of degradation and/or expected failure of the system derived based on the fourth degradation coefficients set is used to assess mechanical life or remaining mechanical life of a structure.

18. The method of claim 1, wherein the obtained in-situ control data set or experimental data set is associated with active dissipative or thermal process(es) of the system with respect to structural degradation due to fatigue, the method comprising:
determining, by the processor, a first set of degradation coefficients based on linear dependence between assessed mechanical stress and irreversible entropies;
determining, by the processor, a second set of degradation coefficients based on linear dependence between i) assessed CDM damage and irreversible entropies;
determining, by the processor, a third set of degradation coefficients based on linear dependence between assessed normalized cycles (N/N_{f}) and irreversible entropies;
determining, by the processor, a fourth set of degradation coefficients based on linear dependence between assessed stress and thermal entropies;
wherein the measure of degradation and/or expected failure of the system derived based on the first, second, third, and fourth degradation coefficients sets are used to assess mechanical life or remaining mechanical life of a structure.

19. The method of claim 1, wherein the obtained in-situ control data set or experimental data set is associated with active dissipative or thermal process(es) of the system with respect to structural degradation due an assessed fatigue measure, and
wherein the assessed fatigue measure is selected from the group consisting of: mechanical stress (e.g. normal or torsional), thermal stress, normalized number of cycles (N/N_{f}), Continuum Damage Mechanics-based damage parameter (D), and chemical degradation.

20. The method of claim 1, wherein the estimation of entropy includes an estimation of entropy production/generation.

21. The method of claim 20 further comprising:
determining, by the processor, one or more irreversible entropy parameters for the dissipative process by combining an assessed active boundary work parameter associated with active boundary work with an internal dissipation parameter associated with internal dissipation of the system, wherein the internal dissipation parameter is estimated as a change in a potential of the system; and
determining, by the processor, one or more reversible entropy parameters for the dissipative process based on assessed standard/ideal values of intensive and extensive phenomenological conjugate variables that define the dissipative process and an instantaneous boundary temperature associated with the active boundary work parameter,
wherein the one or more reversible entropy parameters and the one or more irreversible entropy parameters are used to determine an entropy production parameter directly related to degradation and/or expected failure of the system.

22. The method of claim 21 further comprising:
determining, by the processor, the entropy production parameter, wherein the entropy production parameter is determined as a difference between the one or more reversible entropy parameters and the one or more irreversible entropy parameters.

23. The method of claim 21 further comprising;
determining, by the processor, a critical failure entropy parameter associated with a critical failure entropy, wherein the critical failure entropy parameter, or a value associated therewith, is used to detect instability in the system.

24. The method of claim 23, wherein the critical failure entropy parameter is estimated as a value of the irreversible entropy parameter when the entropy production parameter transitions abruptly

25. The method of claim 21 further comprising:
determining, by the processor, a parameter associated with a measure of the system ideal state, wherein the determination is based on the estimated one or more reversible entropy parameters by linearly combining a determined reversible degradation coefficient with an assessed accumulated reversible entropy parameter, or values associated therewith, wherein the ideal state is used as an instantaneous reference in a real-time monitoring system and/or an evaluation of the system for use in engineering application and/or in the control, or optimization, or maintenance of said system in said engineering application.

26. The method of claim 20, wherein the dissipative process is associated with battery degradation,
wherein the obtained in-situ control data set or obtained experimental data set is used to determine a first set of degradation coefficients based on linear dependence of i) capacity on ii) ohmic entropy and on electro-chemico-thermal (ECT) entropy, respectively;
wherein an assessed battery ideal/reversible state is determined by i) measured open-circuit voltage values measured from the system and ii) estimated reversible current values determined as initial current values measured from the system having been adjusted by the measured open-circuit voltage values.

27. The method of claim 26, wherein the measure of degradation and/or expected failure of the system derived based on the first set of degradation coefficients is used to assess battery cycle life or remaining battery cycle life.

28. The method of claim 27, wherein the dissipative process is associated with rechargeable battery degradation, the method further comprises:
determining, by the processor, a parameter associated with a measure of degradation and/or expected failure of the system based on a difference between an estimated degraded state and the assessed battery ideal state,
wherein determination is used to assess battery cycle life or remaining battery cycle life.

29. A system comprising:
a processor; and
a memory having instructions stored thereon, wherein execution of the instructions by the processor, cause the processor to:
obtain in-situ control data set or experimental data set associated with a dissipative or thermal process of a system, wherein the control or experimental data set is acquired to assess a degradation measure and to assess an entropy production for the dissipative process;
determine one or more degradation coefficients from the control or experimental data, wherein each of the one or more degradation coefficients is determined as a rate of change of one or more assessed degradation measure parameters with respect to one or more assessed entropy production parameters for the dissipative or thermal process, wherein the rate of change is determined as a slope of a first coordinate axis associated with the one or more assessed degradation measure parameters and of one or more second coordinate axes each associated with an assessed entropy production parameter associated with the dissipative or thermal process; and
determine one or more parameters associated with a measure of degradation and/or expected failure of the system, wherein the determination of the one or more parameters is based on an assessed estimated entropy parameter associated with estimated entropy produced by the dissipative process by linearly combining each of the one or more determined degradation coefficients with at least one corresponding assessed accumulated irreversible entropy parameter, and wherein the one or more parameters associated with the degradation and/or expected failure, or value(s) associated therewith, of the system is used in an evaluation of the system for use in engineering application or in the control, optimization, or maintenance of said system in said engineering application.

30. A non-transitory computer readable medium having instructions stored thereon, wherein execution of the instructions by a processor, cause the processor to:
obtain in-situ control data set or experimental data set associated with a dissipative or thermal process of a system, wherein the control or experimental data set is acquired to assess a degradation measure and to assess an entropy production for the dissipative process;
determine one or more degradation coefficients from the control or experimental data, wherein each of the one or more degradation coefficients is determined as a rate of change of one or more assessed degradation measure parameters with respect to one or more assessed entropy production parameters for the dissipative or thermal process, wherein the rate of change is determined as a slope of a first coordinate axis associated with the one or more assessed degradation measure parameters and of one or more second coordinate axes each associated with an assessed entropy production parameter associated with the dissipative or thermal process; and
determine one or more parameters associated with a measure of degradation and/or expected failure of the system, wherein the determination of the one or more parameters is based on an assessed estimated entropy parameter associated with estimated entropy produced by the dissipative process by linearly combining each of the one or more determined degradation coefficients with at least one corresponding assessed accumulated irreversible entropy parameter, and wherein the one or more parameters associated with the degradation and/or expected failure, or value(s) associated therewith, of the system is used in an evaluation of the system for use in engineering application or in the control, optimization, or maintenance of said system in said engineering application.
